(19) United States
(12) Patent Application Publication
(10) Pub. No.: US 2003/0228597 A1
(43) Pub. Date: Dec. 11, 2003

(54) Identification of genetic targets for modulation by oligonucleotides and generation of oligonucleotides for gene modulation

(76) Inventors: Lex M. Cowsert, Pittsburgh, PA (US); Brenda F. Baker, Carlsbad, CA (US); John McNeil, La Jolla, CA (US); Susan M. Freier, San Diego, CA (US); Henri M. Sasmor, Oceanside, CA (US); Douglas G. Brooks, Carlsbad, CA (US); Cara Ohashi, San Francisco, CA (US); Jacqueline R. Wyatt, Encinitas, CA (US); Alexander H. Borchers, Encinitas, CA (US); Timothy A. Vickers, Oceanside, CA (US)

Correspondence Address: Daniel D. Biesterveld, WOODCOCK WASHBURN LLP, 46th Floor, One Liberty Place, Philadelphia, PA, 19103, US

(21) Appl. No.: 10/388,263

(22) Filed: Mar. 12, 2003

Related U.S. Application Data
(63) Continuation-in-part of application No. 09/295,463, filed on Apr. 13, 1999.
Application No. 09/295,463 is a continuation-in-part of application No. 09/067,638, filed on Apr. 28, 1998.
(60) Provisional application No. 60/081,483, filed on Apr. 13, 1998.

(51) Int. Cl. C12Q 1/68

(52) U.S. Cl. 435/6

(57) ABSTRACT

Iterative, preferably computer based iterative processes for generating synthetic compounds capable of modulation of target expression are provided. During iterations of the processes, a target nucleic acid sequence is provided or selected, and a library of candidate nucleobase sequences is generated in silico according to defined criteria. A “virtual” oligonucleotide chemistry is chosen and a library of virtual oligonucleotide compounds having the selected nucleobase sequences is generated. These virtual compounds are reviewed and compounds predicted to have particular properties are selected. The selected compounds are robotically synthesized and are preferably robotically assayed for a desired physical, chemical or biological activity. Compounds exhibiting the ability to modulate target expression are identified as target modulators. Target modulators thus generated are used in assays of parameters indicative of biological processes to effect gene function analysis and in assays of parameters indicative of diseases or disorders to effect target valid

### CROSS REFERENCE TO RELATED APPLICATIONS

The present application is a continuation-in-part of U.S. Ser. No. 09/295,463 filed Apr. 13, 1999, which is a continuation-in-part of U.S. Ser. No. 09/067,638 filed Apr. 28, 1998, which claims priority to provisional application Ser. No. 60/081,483 filed Apr. 13, 1998, each of which is incorporated herein by reference in its entirety.

### FIELD OF THE INVENTION

The present invention relates generally to the generation and identification of synthetic compounds having defined physical, chemical or bioactive properties. More particularly, the present invention relates to the automated generation of oligonucleotide compounds targeted to a given nucleic acid sequence via computer-based, iterative robotic synthesis of synthetic oligonucleotide compounds and robotic or robot-assisted analysis of the activities of such compounds. Information gathered from assays of such compounds is used to identify nucleic acid sequences that are tractable to a variety of nucleotide sequence-based technologies, for example, gene function analysis and target validation.

### BACKGROUND OF THE INVENTION

1. Oligonucleotide Technology

Synthetic oligonucleotides of complementarity to targets are known to hybridize with particular, target nucleic acids in a sequence-specific manner. In one example, compounds complementary to the “sense” strand of nucleic acids that encode polypeptides, are referred to as “antisense oligonucleotides.” A subset of such compounds may be capable of modulating the expression of a target nucleic acid; such synthetic compounds are described herein as “active oligonucleotide compounds.”

Oligonucleotide compounds are commonly used in vitro as research reagents and diagnostic aids, and in vivo as therapeutic and bioactive agents. Oligonucleotide compounds can exert their effect by a variety of means. One such means takes advantage of an endogenous nuclease, such as RNase H in eukaryotes or RNase P in prokaryotes, to degrade the DNA/RNA hybrid formed between the oligonucleotide sequence and mRNA (Chiang et al., J. Biol. Chem., 1991, 266, 18162; Forster et al., Science, 1990, 249, 783). Another means involves covalently linking of a synthetic moiety having nuclease activity to an oligonucleotide having an antisense sequence. This does not rely upon recruitment of an endogenous nuclease to modulate target activity. Synthetic moieties having nuclease activity include, but are not limited to, enzymatic RNAs, lanthanide ion complexes, and other reactive species. (Haseloff et al., Nature, 1988, 334, 585; Baker et al., J. Am. Chem. Soc., 1997, 119, 8749).

Despite the advances made in utilizing antisense technology to date, it is still common to identify target sequences amenable to antisense technologies through an empirical approach (Szoka, Nature Biotechnology, 1997, 15, 509). Accordingly, the need exists for systems and methods for efficiently and effectively identifying target nucleotide sequences that are suitable for antisense modulation. The present disclosure answers this need by providing systems and methods for automatically identifying such sequences via in silico, robotic or other automated means.

2. Identification of Active Oligonucleotide Compounds

Traditionally, new chemical entities with useful properties are generated by (1) identifying a chemical compound (called a “lead compound”) with some desirable property or activity, (2) creating variants of the lead compound, and (3) evaluating the property and activity of such variant compounds. The process has been called “SAR,” i.e., structure activity relationship. Although “SAR” and its handmaiden, rational drug design, has been utilized with some degree of success, there are a number of limitations to these approaches to lead compound generation, particularly as it pertains to the discovery of bioactive oligonucleotide compounds. In attempting to use SAR with oligonucleotides, it has been recognized that RNA structure can inhibit duplex formation with antisense compounds, so much so that “moving” the target nucleotide sequence even a few bases can drastically decrease the activity of such compounds (Lima et al., Biochemistry, 1992, 31, 12055).

Heretofore, the preferred method of searching for lead antisense compounds has been the manual synthesis and analysis of such compounds. Consequently, a fundamental limitation of the conventional approach is its dependence upon the availability, number and cost of antisense compounds produced by manual, or at best semi-automated, means. Moreover, the assaying of such compounds has traditionally been performed by tedious manual techniques. Thus, the traditional approach to generating active antisense compounds is limited by the relatively high cost and long time required to synthesize and screen a relatively small number of candidate antisense compounds.

Accordingly, the need exists for systems and methods for efficiently and effectively generating new active antisense and other oligonucleotide compounds targeted to specific nucleic acid sequences. The present disclosure answers this need by providing systems and methods for automatically generating and screening active antisense compounds via robotic and other automated means.

3. Gene Function Analysis

Efforts such as the Human Genome Project are making an enormous amount of nucleotide sequence information available in a variety of forms, e.g., genomic sequences, cDNAs, expressed sequence tags (ESTs) and the like. This explosion of information has led one commentator to state that “genome scientists are producing more genes than they can put a function to” (Kahn, Science, 1995, 270, 369). Although some approaches to this problem have been suggested, no solution has yet emerged. For example, methods of looking at gene expression in different disease states or stages of development only provide, at best, an association between a gene and a disease or stage of development (Nowak, Science, 1995, 270, 368). Another approach, looking at the proteins encoded by genes, is developing but “this approach is more complex and big obstacles remain” (Kahn, Science, 1995, 270, 369). Furthermore, neither of these approaches allows one to directly utilize nucleotide sequence information to perform gene function analysis.

In contrast, antisense technology does allow for the direct utilization of nucleotide sequence information for gene function analysis. Once a target nucleic acid sequence has been selected, antisense sequences hybridizable to the sequence can be generated using techniques known in the art. Typically, a large number of candidate antisense oligonucleotides (ASOs) are synthesized having sequences that are more-or-less randomly spaced across the length of the target nucleic acid sequence (e.g., a “gene walk”) and their ability to modulate the expression of the target nucleic acid is assayed. Cells or animals can then be treated with one or more active antisense oligonucleotides, and the resulting effects determined in order to determine the function(s) of the target gene. Although the practicality and value of this empirical approach to determining gene function has been acknowledged in the art, it has also been stated that this approach “is beyond the means of most laboratories and is not feasible when a new gene sequence is identified, but whose function and therapeutic potential are unknown” (Szoka, Nature Biotechnology, 1997, 15, 509).

Accordingly, the need exists for systems and methods for efficiently and effectively determining the function of a gene that is uncharacterized except that its nucleotide sequence, or a portion thereof, is known. The present disclosure answers this need by providing systems and methods for automatically generating active antisense compounds to a target nucleotide sequence via robotic means. Such active antisense compounds are contacted with cells, cell-free extracts, tissues or animals capable of expressing the gene of interest and subsequent biochemical or biological parameters are measured. The results are compared to those obtained from a control cell culture, cell-free extract, tissue or animal which has not been contacted with an active antisense compound in order to determine the function of the gene of interest.

4. Target Validation

Determining the nucleotide sequence of a gene is no longer an end unto itself; rather, it is “merely a means to an end. The critical next step is to validate the gene and its [gene] product as a potential drug target” (Glasser, Genetic Engineering News, 1997, 17, 1). This process, i.e., confirming that modulation of a gene that is suspected of being involved in a disease or disorder actually results in an effect that is consistent with a causal relationship between the gene and the disease or disorder, is known as target validation.

Efforts such as the Human Genome Project are yielding a vast number of complete or partial nucleotide sequences, many of which might correspond to or encode targets useful for new drug discovery efforts. The challenge represented by this plethora of information is how to use such nucleotide sequences to identify and rank valid targets for drug discovery. Antisense technology provides one means by which this might be accomplished; however, the many manual, labor-intensive and costly steps involved in traditional methods of developing active antisense compounds has limited their use in target validation (Szoka, Nature Biotechnology, 1997, 15, 509). Nevertheless, the great target specificity that is characteristic of antisense compounds makes them ideal choices for target validation, especially when the functional roles of proteins that are highly related are being investigated (Albert et al., Trends in Pharm. Sci., 1994, 15, 250).

Accordingly, the need exists for systems and methods for developing compounds efficiently and effectively that modulate a gene, wherein such compounds can be directly developed from nucleotide sequence information. Such compounds are needed to confirm that modulation of a gene that is thought to be involved in a disease or disorder will in fact cause an in vitro or in vivo effect indicative of the origin, development, spread or growth of the disease or disorder.

The present disclosure answers this need by providing systems and methods for automatically generating active oligonucleotide and other compounds, especially antisense compounds, to a target nucleotide sequence via robotic or other automated means. Such active compounds are contacted with a cell culture, cell-free extract, tissue or animal capable of expressing the gene of interest, and subsequent biochemical or biological parameters indicative of the potential gene product function are measured. These results are compared to those obtained with a control cell system, cell-free extract, tissue or animal which has not been contacted with an active antisense compound in order to determine whether or not modulation of the gene of interest affects a specific cellular function. The resulting active antisense compounds may be used as positive controls when other, non antisense-based agents directed to the same target nucleic acid, or to its gene product, are screened.

It should be noted that embodiments of the invention drawn to gene function analysis and target validation have parameters that are shared with other embodiments of the invention, but also have unique parameters. For example, antisense drug discovery naturally requires that the toxicity of the antisense compounds be manageable, whereas, for gene function analysis or target validation, overt toxicity resulting from the antisense compounds is acceptable unless it interferes with the assay being used to evaluate the effects of treatment with such compounds.

U.S. Pat. No. 5,563,036 to Peterson et al. describes systems and methods of screening for compounds that inhibit the binding of a transcription factor to a nucleic acid. In a preferred embodiment, an assay portion of the process is stated to be performed by a computer controlled robot.

U.S. Pat. No. 5,708,158 to Hoey describes systems and methods for identifying pharmacological agents stated to be useful for diagnosing or treating a disease associated with a gene the expression of which is modulated by a human nuclear factor of activated T cells. The methods are stated to be particularly suited to high-thoughput screening wherein one or more steps of the process are performed by a computer controlled robot.

U.S. Pat. Nos. 5,693,463 and 5,716,780 to Edwards et al. describe systems and methods for identifying non-oligonucleotide molecules that specifically bind to a DNA molecule based on their ability to compete with a DNA-binding protein that recognizes the DNA molecule.

U.S. Pat. Nos. 5,463,564 and 5,684,711 to Agrafiotis et al. describe computer based iterative processes for generating chemical entities with defined physical, chemical and/or bioactive properties.

5. Compounds of the Invention

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid.

One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

The first evidence that dsRNA could lead to gene silencing in animals came in 1995 from work in the nematode, Caenorhabditis elegans (Guo and Kempheus, Cell, 1995, 81, 611-620). Montgomery et al. have shown that the primary interference effects of dsRNA are posttranscriptional (Montgomery et al., Proc. Natl. Acad. Sci. USA, 1998, 95, 15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels (Fire et al., Nature, 1998, 391, 806-811). Recently, it has been shown that it is, in fact, the single-stranded RNA oligomers of antisense polarity of the dsRNAs which are the potent inducers of RNAi (Tijsterman et al., Science, 2002, 295, 694-697).

In the context of this invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of this invention, the present invention comprehends other families of compounds as well, including but not limited to oligonucleotide analogs and mimetics such as those described herein.

The compounds in accordance with this invention preferably comprise from about 8 to about 80 nucleobases (i.e. from about 8 to about 80 linked nucleosides). One of ordinary skill in the art will appreciate that the invention embodies compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In one preferred embodiment, the compounds of the invention are 12 to 50 nucleobases in length. One having ordinary skill in the art will appreciate that this embodies compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In another preferred embodiment, the compounds of the invention are 15 to 30 nucleobases in length. One having ordinary skill in the art will appreciate that this embodies compounds of 15, 16, 17, 18, 19, 20, 21, 22, 23, 24, 25, 26, 27, 28, 29, or 30 nucleobases in length.

In another preferred embodiment, the compounds of the invention are 20 to 25 nucleobases in length. One having ordinary skill in the art will appreciate that this embodies compounds of 20, 21, 22, 23, 24 or 25 nucleobases in length.

Particularly preferred compounds are oligonucleotides from about 12 to about 50 nucleobases, even more preferably those comprising from about 15 to about 30 nucleobases.

Antisense compounds 8-80 nucleobases in length comprising a stretch of at least eight (8) consecutive nucleobases selected from within the illustrative antisense compounds are considered to be suitable antisense compounds as well.

Exemplary preferred antisense compounds include oligonucleotide sequences that comprise at least the 8 consecutive nucleobases from the 5′-terminus of one of the illustrative preferred antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Similarly preferred antisense compounds are represented by oligonucleotide sequences that comprise at least the 8 consecutive nucleobases from the 3′-terminus of one of the illustrative preferred antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about 8 to about 80 nucleobases). One having skill in the art armed with the preferred antisense compounds illustrated herein will be able, without undue experimentation, to identify further preferred antisense compounds.

### SUMMARY OF THE INVENTION

The present invention is directed to methods of effecting gene function analysis and target validation by generating in silico a library of nucleobase sequences targeted to the gene and robotically assaying a plurality of synthetic compounds having at least some of the nucleobase sequences to identify target modulators. These modulators are then assayed for effects on biological function to effect gene function analysis and for effects on diseases or disorders to effect target validation.

Further features and advantages of the present invention, as well as the structure and operation of various embodiments of the present invention, are described in detail below with reference to the accompanying drawings. In the drawings, like reference numbers indicate identical or functionally similar elements.

### BRIEF DESCRIPTION OF THE DRAWINGS

The present invention will be described with reference to the accompanying drawings, wherein:

FIGS. 1 and 2 are a flow diagram of one method according to the present invention depicting the overall flow of data and materials among various elements of the invention.

FIG. 3 is a flow diagram depicting the flow of data and materials among elements of step 200 of FIG. 1.

FIGS. 4 and 5 are a flow diagram depicting the flow of data and materials among elements of step 300 of FIG. 1.

FIG. 6 is a flow diagram depicting the flow of data and materials among elements of step 306 of FIG. 4.

FIG. 7 is another flow diagram depicting the flow of data and materials among elements of step 306 of FIG. 4.

FIG. 8 is a another flow diagram depicting the flow of data and materials among elements of step 306 of FIG. 4.

FIG. 9 is a flow diagram depicting the flow of data and materials among elements of step 350 of FIG. 5.

FIGS. 10 and 11 are flow diagrams depicting a logical analysis of data and materials among elements of step 400 of FIG. 1.

FIG. 12 is a flow diagram depicting the flow of data and materials among the elements of step 400 of FIG. 1.

FIGS. 13 and 14 are flow diagrams depicting the flow of data and materials among elements of step 500 of FIG. 1.

FIG. 15 is a flow diagram depicting the flow of data and materials among elements of step 600 of FIG. 1.

FIG. 16 is a flow diagram depicting the flow of data and materials among elements of step 700 of FIG. 1.

FIG. 17 is a flow diagram depicting the flow of data and materials among the elements of step 1100 of FIG. 2.

FIG. 18 is a block diagram showing the interconnecting of certain devices utilized in conjunction with a preferred method of the invention;

FIG. 19 is a flow diagram showing a representation of data storage in a relational database utilized in conjunction with one method of the invention;

FIG. 20 is a flow diagram depicting the flow of data and materials in effecting a preferred embodiment of the invention as set forth in Example 16;

FIG. 21 is a flow diagram depicting the flow of data and materials in effecting a preferred embodiment of the invention as set forth in Example 17;

FIG. 22 is a flow diagram depicting the flow of data and materials in effecting a preferred embodiment of the invention as set forth in Example 2;

FIG. 23 is a pictorial elevation view of a preferred apparatus used to robotically synthesize oligonucleotides; and

FIG. 24 is a pictorial plan view of an apparatus used to robotically synthesize oligonucleotides.

### DETAILED DESCRIPTION OF THE INVENTION

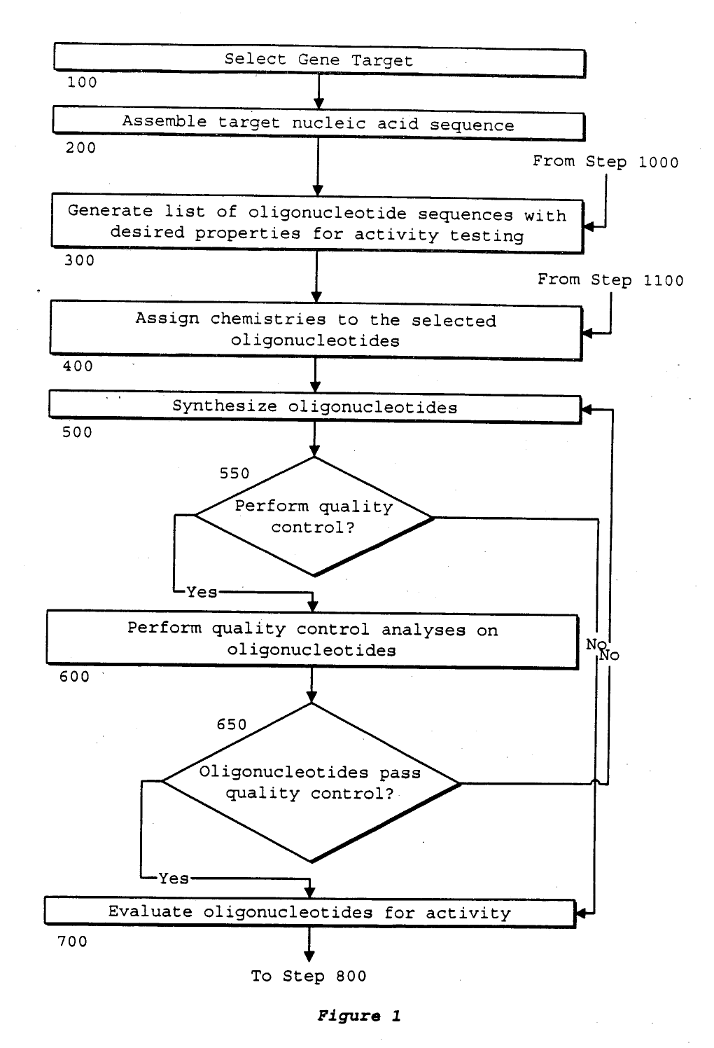

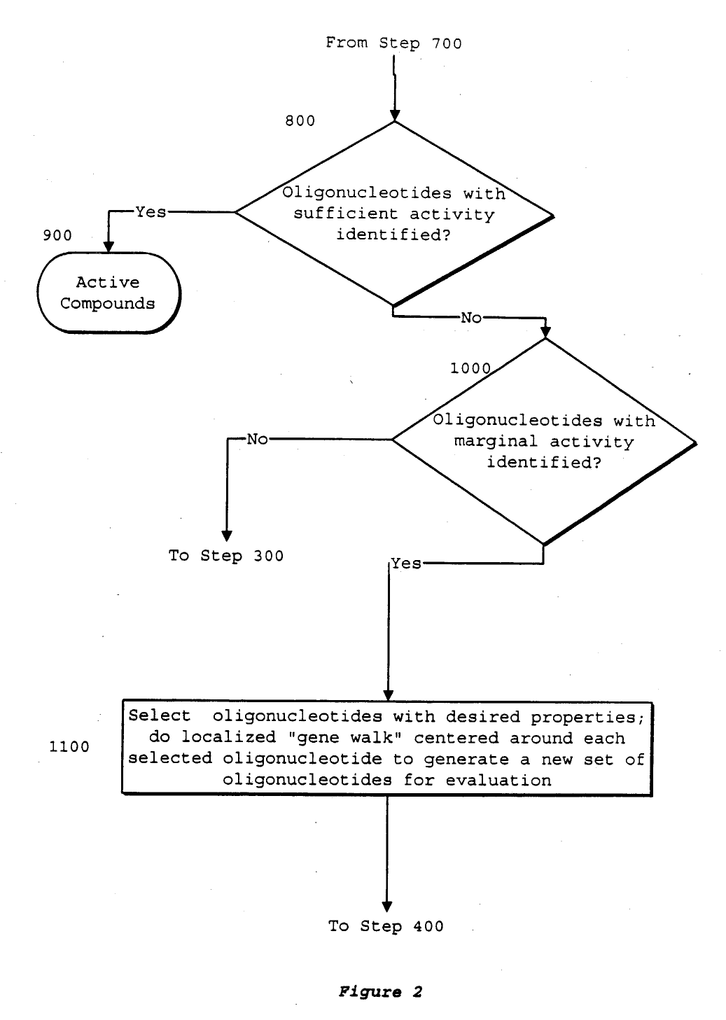

Certain preferred methods of this invention are now described with reference to the flow diagram of FIGS. 1 and 2.

1. Target Nucleic Acid Selection.

The target selection process, process step 100, provides a target nucleotide sequence that is used to help guide subsequent steps of the process. It is generally desired to modulate the expression of the target nucleic acid for any of a variety of purposes, such as, e.g., drug discovery, target validation and/or gene function analysis.

One of the primary objectives of the target selection process, step 100, is to identify molecular targets that represent significant therapeutic opportunities, to provide new and efficacious means of drug discovery and to determine the function of genes that are uncharacterized except for nucleotide sequence. To meet these objectives, genes are classified based upon specific sets of selection criteria.

One such set of selection criteria concerns the quantity and quality of target nucleotide sequence. There must be sufficient target nucleic acid sequence information available for oligonucleotide design. Moreover, such information must be of sufficient quality to give rise to an acceptable level of confidence in the data to perform the methods described herein. Thus, the data must not contain too many missing or incorrect base entries. In the case of a target sequence that encodes a polypeptide, such errors can often be detected by virtually translating all three reading frames of the sense strand of the target sequence and confirming the presence of a continuous polypeptide sequence having predictable attributes, e.g., encoding a polypeptide of known size, or encoding a polypeptide that is about the same length as a homologous protein. In any event, only a very high frequency of sequence errors will frustrate the methods of the invention; most oligonucleotides to the target sequence will avoid-such errors unless such errors occur frequently throughout the entire target sequence.

Another preferred criterion is that appropriate culturable cell lines or other source of reproducible genetic expression should be available. Such cell lines express, or can be induced to express, the gene comprising the target nucleic acid sequence. The oligonucleotide compounds generated by the process of the invention are assayed using such cell lines and, if such assaying is performed robotically, the cell line is preferably tractable to robotic manipulation such as by growth in 96 well plates. Those skilled in the art will recognize that if an appropriate cell line does not exist, it will nevertheless be possible to construct an appropriate cell line. For example, a cell line can be transfected with an expression vector comprising the target gene in order to generate an appropriate cell line for assay purposes.

For gene function analysis, it is possible to operate upon a genetic system having a lack of information regarding, or incomplete characterization of, the biological function(s) of the target nucleic acid or its gene product(s). This is a powerful agent of the invention. A target nucleic acid for gene function analysis might be absolutely uncharacterized, or might be thought to have a function based on minimal data or homology to another gene. By application of the process of the invention to such a target, active compounds that modulate the expression of the gene can be developed and applied to cells. The resulting cellular, biochemical or molecular biological responses are observed, and this information is used by those skilled in the art to elucidate the function of the target gene.

For target validation and drug discovery, another selection criterion is disease association. Candidate target genes are placed into one of several broad categories of known or deduced disease association. Level 1 Targets are target nucleic acids for which there is a strong correlation with disease. This correlation can come from multiple scientific disciplines including, but not limited to, epidemiology, wherein frequencies of gene abnormalities are associated with disease incidence; molecular biology, wherein gene expression and function are associated with cellular events correlated with a disease; and biochemistry, wherein the in vitro activities of a gene product are associated with disease parameters. Because there is a strong therapeutic rationale for focusing on Level 1 Targets, these targets are most preferred for drug discovery and/or target validation.

Level 2 Targets are nucleic acid targets for which the combined epidemiological, molecular biological, and/or biochemical correlation with disease is not so clear as for Level 1. Level 3 Targets are targets for which there is little or no data to directly link the target with a disease process, but there is indirect evidence for such a link, i.e., homology with a Level 1 or Level 2 target nucleic acid sequence or with the gene product thereof. In order not to prejudice the target selection process, and to ensure that the maximum number of nucleic acids actually involved in the causation, potentiation, aggravation, spread, continuance or after-effects of disease states are investigated, it is preferred to examine a balanced mix of Level 1, 2 and 3 target nucleic acids.

In order to carry out drug discovery, experimental systems and reagents shall be available in order for one to evaluate the therapeutic potential of active compounds generated by the process of the invention. Such systems may be operable in vitro (e.g., in vitro models of cell:cell association) or in vivo (e.g., animal models of disease states). It is also desirable, but not obligatory, to have available animal model systems which can be used to evaluate drug pharmacology.

Candidate targets nucleic acids can also classified by biological processes. For example, programmed cell death (“apoptosis”) has recently emerged as an important biological process that is perturbed in a wide variety of diseases. Accordingly, nucleic acids that encode factors that play a role in the apoptotic process are identified as candidate targets. Similarly, potential target nucleic acids can be classified as being involved in inflammation, autoimmune disorders, cancer, or other pathological or dysfunctional processes.

Moreover, genes can often be grouped into families based on sequence homology and biological function. Individual family members can act redundantly, or can provide specificity through diversity of interactions with downstream effectors, or through expression being restricted to specific cell types. When one member of a gene family is associated with a disease process then the rationale for targeting other members of the same family is reasonably strong. Therefore, members of such gene families are preferred target nucleic acids to which the methods and systems of the invention may be applied. Indeed, the potent specificity of antisense compounds for different gene family members makes the invention particularly suited for such targets (Albert et al., Trends Pharm. Sci., 1994, 15, 250). Those skilled in the art will recognize that a partial or complete nucleotide sequence of such family members can be obtained using the polymerase chain reaction (PCR) and “universal” primers, i.e., primers designed to be common to all members of a given gene family.

PCR products generated from universal primers can be cloned and sequenced or directly sequenced using techniques known in the art. Thus, although nucleotide sequences from cloned DNAs, or from complementary DNAs (cDNAs) derived from mRNAs, may be used in the process of the invention, there is no requirement that the target nucleotide sequence be isolated from a cloned nucleic acid. Any nucleotide sequence, no matter how determined, of any nucleic acid, isolated or prepared in any fashion, may be used as a target nucleic acid in the process of the invention.

Furthermore, although polypeptide-encoding nucleic acids provide the target nucleotide sequences in one embodiment of the invention, other nucleic acids may be targeted as well. Thus, for example, the nucleotide sequences of structural or enzymatic RNAs may be utilized for drug discovery and/or target validation when such RNAs are associated with a disease state, or for gene function analysis when their biological role is not known.

2. Assembly of Target Nucleotide Sequence.

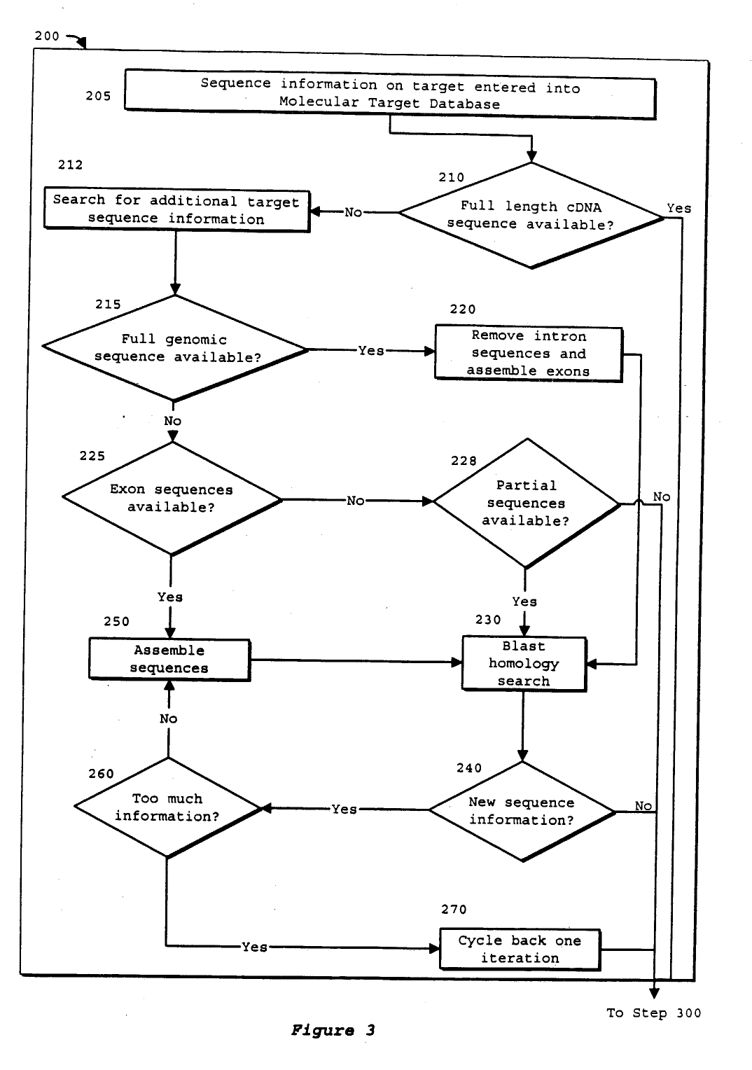

FIG. 3 is a block diagram detailing the steps of the target nucleotide sequence assembly process, process step 200 in acccordance with one embodiment of the invention. The oligonucleotide design process, process step 300, is facilitated by the availability of accurate target sequence information. Because of limitations of automated genome sequencing technology, gene sequences are often accumulated in fragments. Further, because individual genes are often being sequenced by independent laboratories using different sequencing strategies, sequence information corresponding to different fragments is often deposited in different databases. The target nucleic acid assembly process take advantage of computerized homology search algorithms and sequence fragment assembly algorithms to search available databases for related sequence information and incorporate available sequence information into the best possible representation of the target nucleic acid molecule, for example a RNA transcript. This representation is then used to design oligonucleotides, process step 300, which can be tested for biological activity in process step 700.

In the case of genes directing the synthesis of multiple transcripts, i.e. by alternative splicing, each distinct transcript is a unique target nucleic acid for purposes of step 300. In one embodiment of the invention, if active compounds specific for a given transcript isoform are desired, the target nucleotide sequence is limited to those sequences that are unique to that transcript isoform. In another embodiment of the invention, if it is desired to modulate two or more transcript isoforms in concert, the target nucleotide sequence is limited to sequences that are shared between the two or more transcripts.

In the case of a polypeptide-encoding nucleic acid, it is generally preferred that full-length cDNA be used in the oligonucleotide design process step 300 (with full-length cDNA being defined as reading from the 5′ cap to the poly A tail). Although full-length cDNA is preferred, it is possible to design oligonucleotides using partial sequence information. Therefore it is not necessary for the assembly process to generate a complete cDNA sequence. Further in some cases it may be desirable to design oligonucleotides targeting introns. In this case the process can be used to identify individual introns at process step 220.

The process can be initiated by entering initial sequence information on a selected molecular target at process step 205. In the case of a polypeptide-encoding nucleic acid, the full-length cDNA sequence is generally preferred for use in oligonucleotide design strategies at process step 300. The first step is to determine if the initial sequence information represents the full-length cDNA, decision step 210. In the case where the full-length cDNA sequence is available the process advances directly to the oligonucleotide design step 300. When the full-length cDNA sequence is not available, databases are searched at process step 212 for additional sequence information.

The algorithm preferably used in process steps 212 and 230 is BLAST (Altschul, et al., J. Mol. Biol., 1990, 215, 403), or “Gapped BLAST” (Altschul et al., Nucl. Acids Res., 1997, 25, 3389). These are database search tools based on sequence homology used to identify related sequences in a sequence database. The BLAST search parameters are set to only identify closely related sequences. Some preferred databases searched by BLAST are a combination of public domain and proprietary databases. The databases, their contents, and sources are listed in Table 1.

TABLE 1
Database Sources of Target Sequences

| Database | Contents | Source |
|---|---|---|
| NR | All non-redundant | National Center for |
| | GenBank, EMBL, DDBJ | Biotechnology Information at the |
| | and PDB sequences | National Institutes of Health |
| Month | All new or revised | National Center for |
| | GenBank, EMBL, DDBJ | Biotechnology Information at the |
| | and PDB sequences | National Institutes of Health |
| | released in the | |
| | last 30 days | |
| Dbest | Non-redundant | National Center for |
| | database of GenBank, | Biotechnology Information at the |
| | EMBL, DDBJ and | National Institutes of Health |
| | EST divisions | |
| Dbsts | Non-redundant | National Center for |
| | database of GenBank, | Biotechnology Information at the |
| | EMBL, DDBJ and | National Institutes of Health |
| | STS divisions | |
| Htgs | High throughput | National Center for |
| | genomic sequences | Biotechnology Information at the |
| | | National Institutes of Health |

When genomic sequence information is available at decision step 215, introns are removed and exons are assembled into continuous sequence representing the cDNA sequence in process step 220. Exon assembly occurs using the Phragment Assembly Program “Phrap” (Copyright University of Washington Genome Center, Seattle, Wash.). The Phrap algorithm analyzes sets of overlapping sequences and assembles them into one continuous sequence referred to as a “contig.” The resulting contig is preferably used to search databases for additional sequence information at process step 230. When genomic information is not available, the results of process step 212 are analyzed for individual exons at decision step 225. Exons are frequently recorded individually in databases. If multiple complete exons are identified, they are prferably assembled into a contig using Phrap at process step 250. If multiple complete exons are not identified at decision step 225, then sequences can be analyzed for partial sequence information in decision step 228. ESTs identified in the database dbEST are examples of such partial sequence information. If additional partial information is not found, then the process is advanced to process step 230 at decision step 228. If partial sequence information is found in process 212 then that information is advanced to process step 230 via decision step 228.

Process step 230, decision step 240, decision step 260 and process step 250 define a loop designed to extend iteratively the amount of sequence information available for targeting. At the end of each iteration of this loop, the results are analyzed in decision steps 240 and 260. If no new information is found then the process advances at decision step 240 to process step 300. If there is an unexpectedly large amount of sequence information identified, suggesting that the process moved outside the boundary of the gene into repetitive genomic sequence, then the process is preferably cycled back one iteration and that sequence is advanced at decision step 240 to process step 300. If a small amount of new sequence information is identified, then the loop is iterated such as by taking the 100 most 5-prime (5′) and 100 most 3-prime (3′) bases and interating them through the BLAST homology search at process step 230. New sequence information is added to the existing contig at process step 250.

3. In Silico Generation of a Set of Nucleobase Sequences and Virtual Oligonucleotides.

For the following steps 300 and 400, they may be performed in the order described below, i.e., step 300 before step 400, or, in an alternative embodiment of the invention, step 400 before step 300. In this alternate embodiment, each oligonucleotide chemistry is first assigned to each oligonucleotide sequence. Then, each combination of oligonucleotide chemistry and sequence is evaluated according to the parameters of step 300. This embodiment has the desirable feature of taking into account the effect of alternative oligonucleotide chemistries on such parameters. For example, substitution of 5-methyl cytosine (5MeC or m5c) for cytosine in an antisense compound may enhance the stability of a duplex formed between that compound and its target nucleic acid. Other oligonucleotide chemistries that enhance oligonucleotide:[target nucleic acid] duplexes are known in the art (see for example, Freier et al., Nucleic Acids Research, 1997, 25, 4429). As will be appreciated by those skilled in the art, different oligonucleotide chemistries may be preferred for different target nucleic acids. That is, the optimal oligonucleotide chemistry for binding to a target DNA might be suboptimal for binding to a target RNA having the same nucleotide sequence.

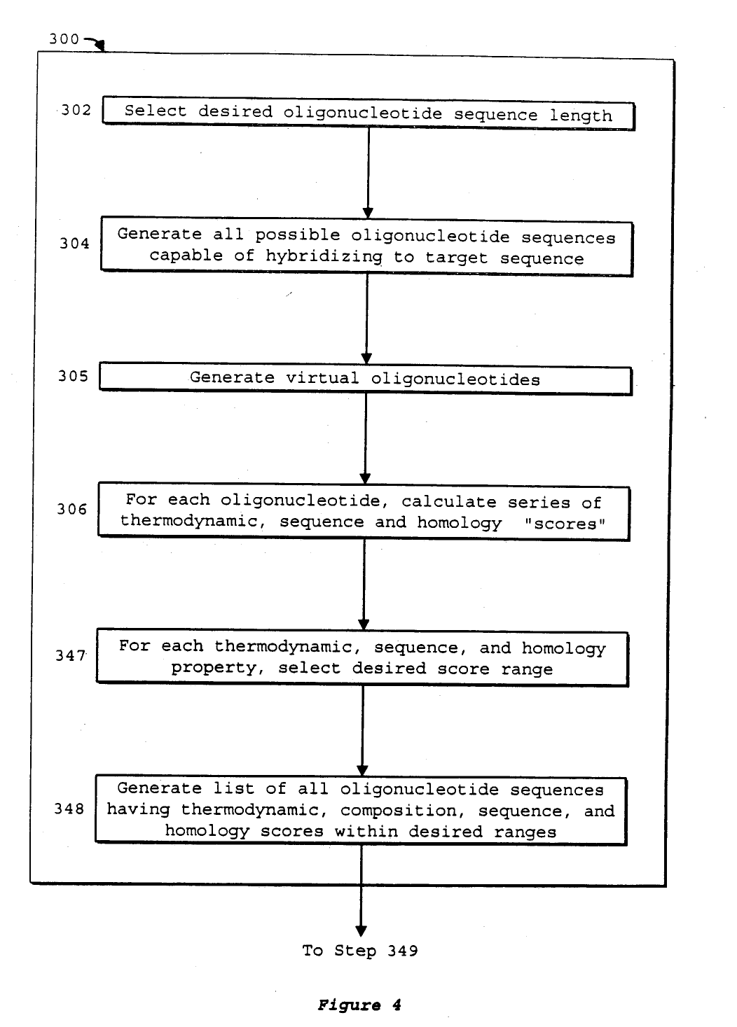

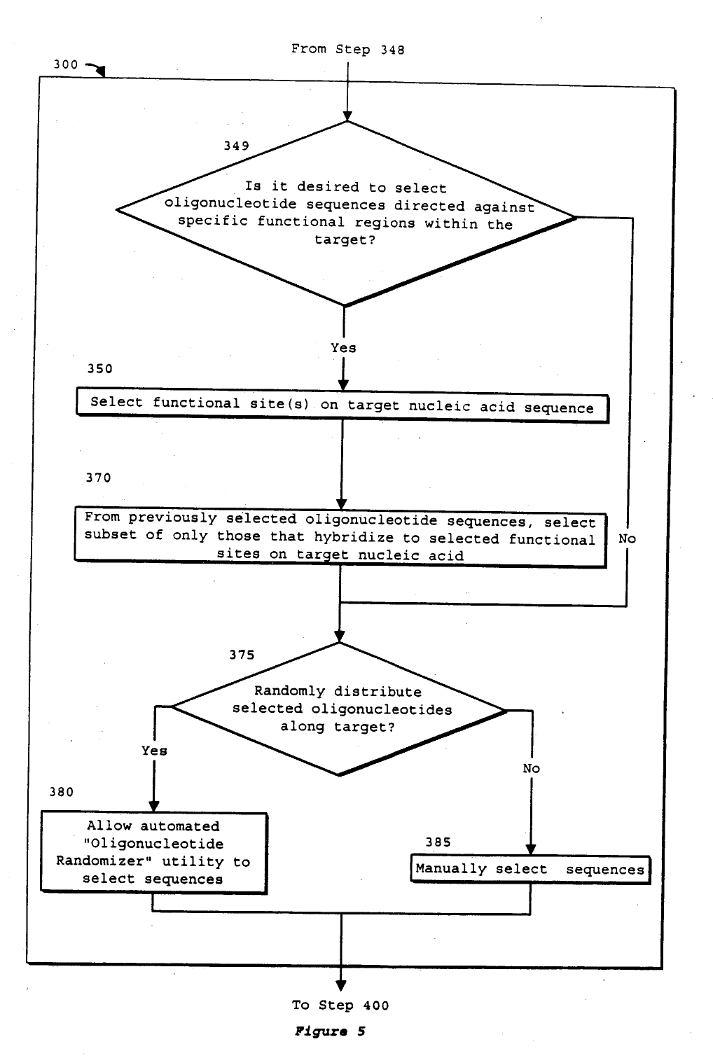

In effecting the process of the invention in the order step 300 before step 400 as seen in FIG. 1, from a target nucleic acid sequence assembled at step 200, a list of oligonucleotide sequences is generated as represented in the flowchart shown in FIGS. 4 and 5. In step 302, the desired oligonucleotide length is chosen. In a preferred embodiment, oligonucleotide length is between from about 8 to about 30, more preferably from about 12 to about 25, nucleotides. In step 304, all possible oligonucleotide sequences of the desired length capable of hybridizing to the target sequence obtained in step 200 are generated. In this step, a series of oligonucleotide sequences are generated, simply by determining the most 5′ oligonucleotide possible and “walking” the target sequence in increments of one base until the 3′ most oligonucleotide possible is reached.

In step 305, a virtual oligonucleotide chemistry is applied to the nucleobase sequences of step 304 in order to yield a set of virtual oligonucleotides that can be evaluated in silico. Default virtual oligonucleotide chemistries include those that are well-characterized in terms of their physical and chemical properties, e.g., 2′-deoxyribonucleic acid having naturally occurring bases (A, T, C and G), unmodified sugar residues and a phosphodiester backbone.

4. In Silico Evaluation of Thermodynamic Properties of Virtual Oligonucleotides.

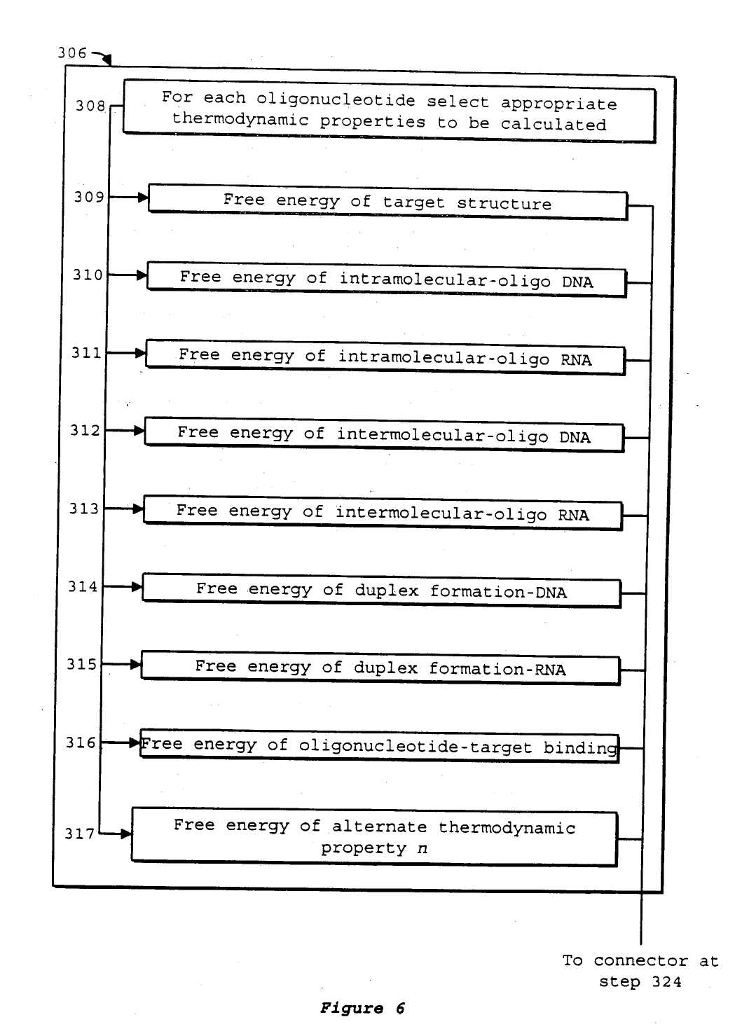

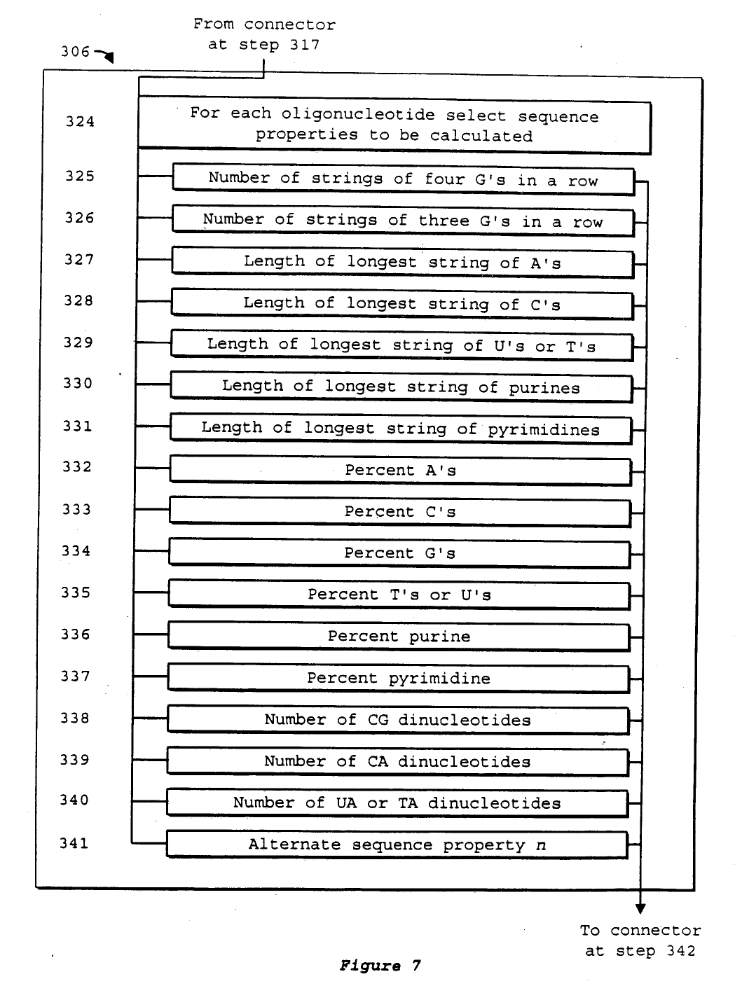

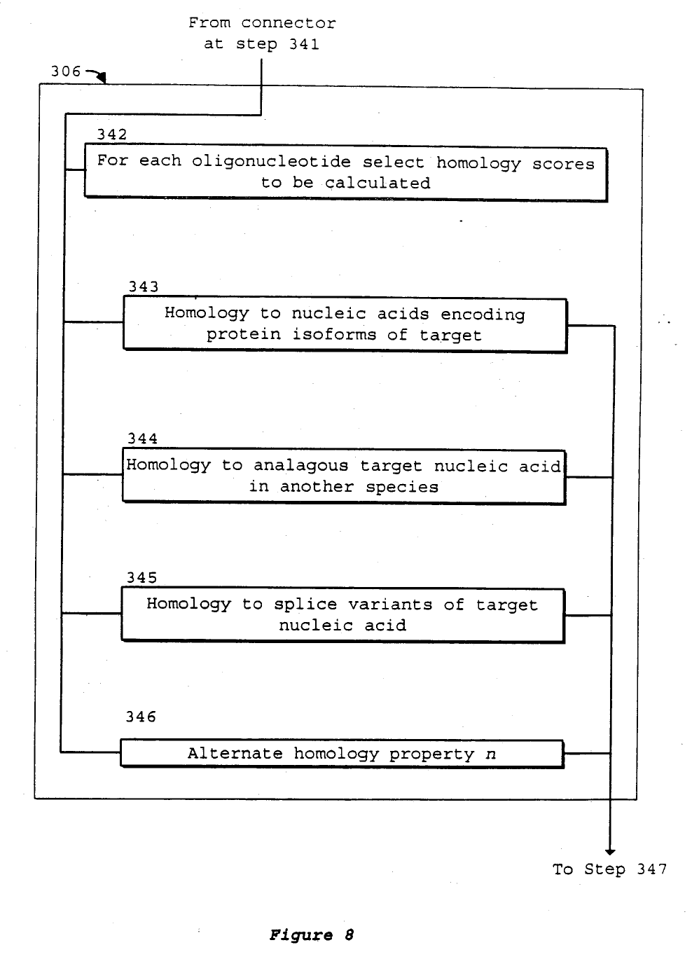

In step 306, a series of thermodynamic, sequence, and homology scores are preferably calculated for each virtual oligonucleotide obtained from step 305. Thermodynamic properties are calculated as represented in FIG. 6. In step 308, the desired thermodynamic properties are selected. As many or as few as desired can be selected; optionally, none will be selected. The desired properties will typically include step 309, calculation of the free energy of the target structure. If the oligonucleotide is a DNA molecule, then steps 310, 312, and 314 are performed. If the oligonucleotide is an RNA molecule, then steps 311, 313 and 315 are performed. In both cases, these steps correspond to calculation of the free energy of intramolecular oligonucleotide interactions, intermolecular interactions and duplex formation. In addition, a free energy of oligonucleotide-target binding is preferably calculated at step 316.

Other thermodynamic and kinetic properties may be calculated for oligonucleotides as represented at step 317. Such other thermodynamic and kinetic properties may include melting temperatures, association rates, dissociation rates, or any other physical property that may be predictive of oligonucleotide activity.

The free energy of the target structure is defined as the free energy needed to disrupt any secondary structure in the target binding site of the targeted nucleic acid. This region includes any intra-target nucleotide base pairs that need to be disrupted in order for an oligonucleotide to bind to its complementary sequence. The effect of this localized disruption of secondary structure is to provide accessibility by the oligonucleotide. Such structures will include double helices, terminal unpaired and mismatched nucleotides, loops, including hairpin loops, bulge loops, internal loops and multibranch loops (Serra et al., Methods in Enzymology, 1995, 259, 242).

The intermolecular free energies refer to inherent energy due to the most stable structure formed by two oligonucleotides; such structures include dimer formation. Intermolecular free energies should also be taken into account when, for example, two or more oligonucleotides, of different sequence are to be administered to the same cell in an assay.

The intramolecular free energies refer to the energy needed to disrupt the most stable secondary structure within a single oligonucleotide. Such structures include, for example, hairpin loops, bulges and internal loops. The degree of intramolecular base pairing is indicative of the energy needed to disrupt such base pairing.

The free energy of duplex formation is the free energy of denatured oligonucleotide binding to its denatured target sequence. The oligonucleotide-target binding is the total binding involved, and includes the energies involved in opening up intra- and inter-molecular oligonucleotide structures, opening up target structure, and duplex formation.

The most stable RNA structure is predicted based on nearest neighbor analysis (Xia, T., et al., Biochemistry, 1998, 37, 14719-14735; Serra et al., Methods in Enzymology, 1995, 259, 242). This analysis is based on the assumption that stability of a given base pair is determined by the adjacent base pairs. For each possible nearest neighbor combination, thermodynamic properties have been determined and are provided. For double helical regions, two additional factors need to be considered, an entropy change required to initiate a helix and a entropy change associated with self-complementary strands only. Thus, the free energy of a duplex can be calculated using the equation: where:

ΔG is the free energy of duplex formation,

ΔH is the enthalpy change for each nearest neighbor,

ΔS is the entropy change for each nearest neighbor, and T is temperature.

The ΔH and ΔS for each possible nearest neighbor combination have been experimentally determined. These letter values are often available in published tables. For terminal unpaired and mismatched nucleotides, enthalpy and entropy measurements for each possible nucleotide combination are also available in published tables. Such results are added directly to values determined for duplex formation. For loops, while the available data is not as complete or accurate as for base pairing, one known model determines the free energy of loop formation as the sum of free energy based on loop size, the closing base pair, the interactions between the first mismatch of the loop with the closing base pair, and additional factors including being closed by AU or UA or a first mismatch of GA or UU. Such equations may also be used for oligoribonucleotide-target RNA interactions.

The stability of DNA duplexes is used in the case of intra- or intermolecular oligodeoxyribonucleotide interactions. DNA duplex stability is calculated using similar equations as RNA stability, except experimentally determined values differ between nearest neighbors in DNA and RNA and helix initiation tends to be more favorable in DNA than in RNA (SantaLucia et al., Biochemistry, 1996, 35, 3555).

Additional thermodynamic parameters are used in the case of RNA/DNA hybrid duplexes. This would be the case for an RNA target and oligodeoxynucleotide. Such parameters were determined by Sugimoto et al. (Biochemistry, 1995, 34, 11211). In addition to values for nearest neighbors, differences were seen for values for enthalpy of helix initiation.

5. In Silico Evaluation of Target Accessibility

Target accessibility is believed to be an important consideration in selecting oligonucleotides. Such a target site will possess minimal secondary structure and thus, will require minimal energy to disrupt such structure. In addition, secondary structure in oligonucleotides, whether inter- or intra-molecular, is undesirable due to the energy required to disrupt such structures. Oligonucleotide-target binding is dependent on both these factors. It is desirable to minimize the contributions of secondary structure based on these factors. The other contribution to oligonucleotide-target binding is binding affinity. Favorable binding affinities based on tighter base pairing at the target site is desirable.

Following the calculation of thermodynamic properties ending at step 317, the desired sequence properties to be scored are selected at step 324. As many or as few as desired can be selected; optionally, none will be selected. These properties include the number of strings of four guanosine residues in a row at step 325 or three guanosine in a row at step 326, the length of the longest string of adenosines at step 327, cytidines at step 328 or uridines or thymidines at step 329, the length of the longest string of purines at step 330 or pyrimidine at step 331, the percent composition of adenosine at step 332, cytidine at step 333, guanosine at step 334 or uridines or thymidines at step 335, the percent composition of purines at step 336 or pyrimidines at step 337, the number of CG dinucleotide repeats at step 338, CA dinucleotide repeats at step 339 or UA or TA dinucleotide repeats at step 340. In addition, other sequence properties may be used as found to be relevant and predictive of antisense efficacy, as represented at step 341.

These sequence properties may be important in predicting oligonucleotide activity, or lack thereof. For example, U.S. Pat. No. 5,523,389 discloses oligonucleotides containing stretches of three or four guanosine residues in a row. Oligonucleotides having such sequences may act in a sequence-independent manner. For an antisense approach, such a mechanism is not usually desired. In addition, high numbers of dinucleotide repeats may be indicative of low complexity regions which may be present in large numbers of unrelated genes. Unequal base composition, for example, 90% adenosine, can also give non-specific effects. From a practical standpoint, it may be desirable to remove oligonucleotides that possess long stretches of other nucleotides due to synthesis considerations. Other sequences properties, either listed above or later found to be of predictive value may be used to select oligonucleotide sequences.

Following step 341, the homology scores to be calculated are selected in step 342. Homology to nucleic acids encoding protein isoforms of the target, as represented at step 343, may be desired. For example, oligonucleotides specific for an isoform of protein kinase C can be selected. Also, oligonucleotides can be selected to target multiple isoforms of such genes. Homology to analogous target sequences, as represented at step 344, may also be desired. For example, an oligonucleotide can be selected to a region common to both humans and mice to facilitate testing of the oligonucleotide in both species. Homology to splice variants of the target nucleic acid, as represented at step 345, may be desired. In addition, it may be desirable to determine homology to other sequence variants as necessary, as represented in step 346.

Following step 346, from which scores were obtained in each selected parameter, a desired range is selected to select the most promising oligonucleotides, as represented at step 347. Typically, only several parameters will be used to select oligonucleotide sequences. As structure prediction improves, additional parameters may be used. Once the desired score ranges are chosen, a list of all oligonucleotides having parameters falling within those ranges will be generated, as represented at step 348.

6. Targeting Oligonucleotides to Functional Regions of a Nucleic Acid.

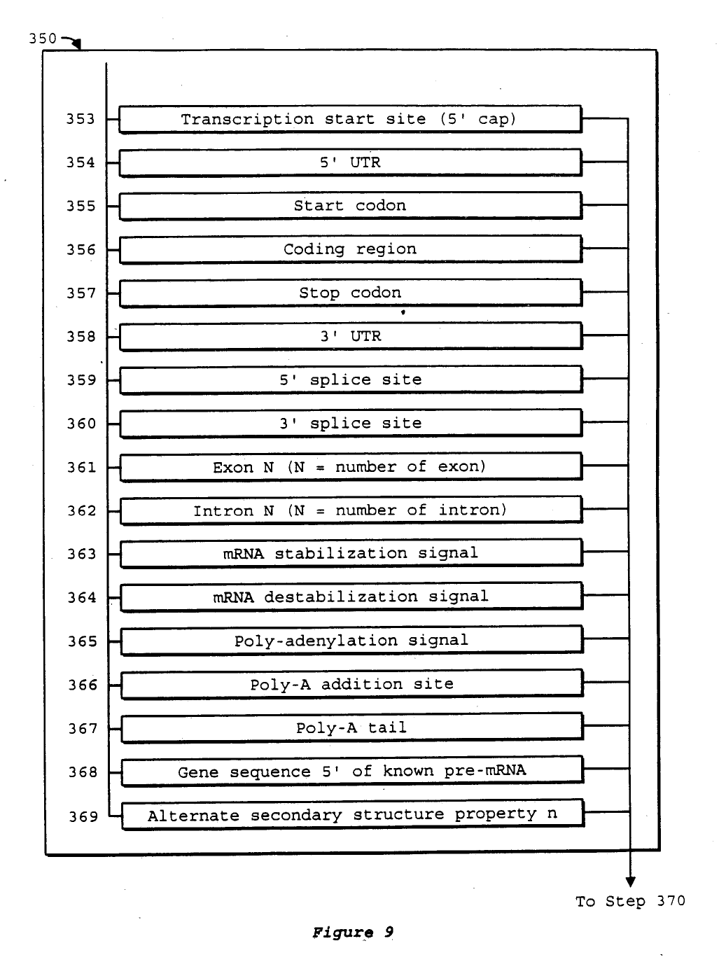

It may be desirable to target oligonucleotide sequences to specific functional regions of the target nucleic acid. A decision is made whether to target such regions, as represented in decision step 349. If it is desired to target functional regions then process step 350 occurs as seen in greater detail in FIG. 9. If it is not desired then the process proceeds to step 375.

In step 350, as seen in FIG. 9, the desired functional regions are selected. Such regions include the transcription start site or 5′ cap at step 353, the 5′ untranslated region at step 354, the start codon at step 355, the coding region at step 356, the stop codon at step 357, the 3′ untranslated region at step 358, 5′ splice sites at step 359 or 3′ splice sites at step 360, specific exons at step 361 or specific introns at step 362, mRNA stabilization signal at step 363, mRNA destabilization signal at step 364, poly-adenylation signal at step 365, poly-A addition site at step 366, poly-A tail at step 367, or the gene sequence 5′ of known pre-mRNA at step 368. In addition, additional functional sites may be selected, as represented at step 369.

Many functional regions are important to the proper processing of the gene and are attractive targets for antisense approaches. For example, the AUG start codon is commonly targeted because it is necessary to initiate translation. In addition, splice sites are thought to be attractive targets because these regions are important for processing of the mRNA. Other known sites may be more accessible because of interactions with protein factors or other regulatory molecules.

After the desired functional regions are selected and determined, then a subset of all previously selected oligonucleotides are selected based on hybridization to only those desired functional regions, as represented by step 370.

7. Uniform Distribution of Oligonucleotides.

Whether or not targeting functional sites is desired, a large number of oligonucleotide sequences may result from the process thus far. In order to reduce the number of oligonucleotide sequences to a manageable number, a decision is made whether to uniformly distribute selected oligonucleotides along the target, as represented in step 375. A uniform distribution of oligonucleotide sequences will aim to provide complete coverage throughout the complete target nucleic acid or the selected functional regions. A computer-based program is used to automate the distribution of sequences, as represented in step 380. Such a program factors in parameters such as length of the target nucleic acid, total number of oligonucleotide sequences desired, oligonucleotide sequences per unit length, number of oligonucleotide sequences per functional region. Manual selection of oligonucleotide sequences is also provided for by step 385. In some cases, it may be desirable to manually select oligonucleotide sequences. For example, it may be useful to determine the effect of small base shifts on activity. Once the desired number of oligonucleotide sequences is obtained either from step 380 or step 385, then these oligonucleotide sequences are passed onto step 400 of the process, where oligonucleotide chemistries are assigned.

8. Assignment of Actual Oligonucleotide Chemistry.

Once a set of select nucleobase sequences has been generated according to the preceding process and decision steps, actual oligonucleotide chemistry is assigned to the sequences. An “actual oligonucleotide chemistry” or simply “chemistry” is a chemical motif that is common to a particular set of robotically synthesized oligonucleotide compounds. Preferred chemistries include, but are not limited to, oligonucleotides in which every linkage is a phosphorothioate linkage, and chimeric oligonucleotides in which a defined number of 5′ and/or 3′ terminal residues have a 2′-methoxyethoxy modification.

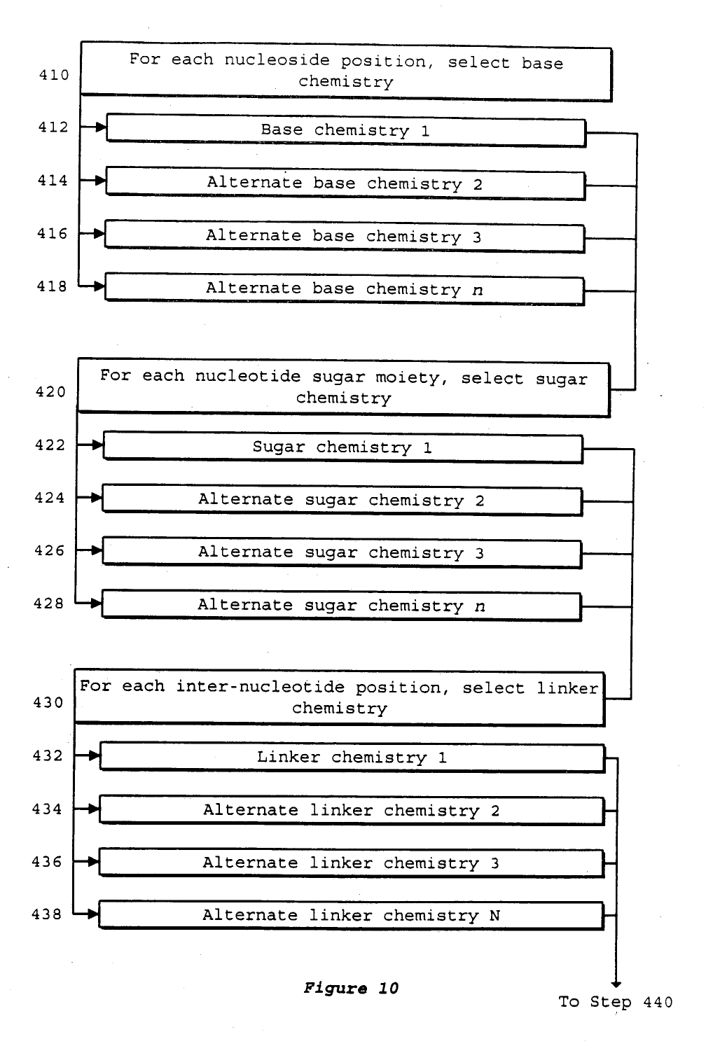

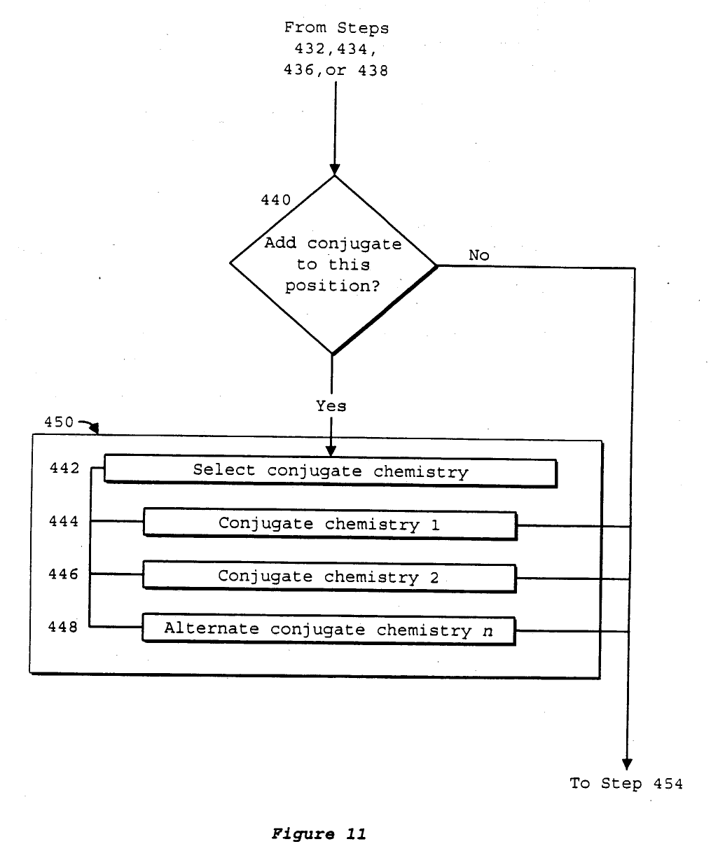

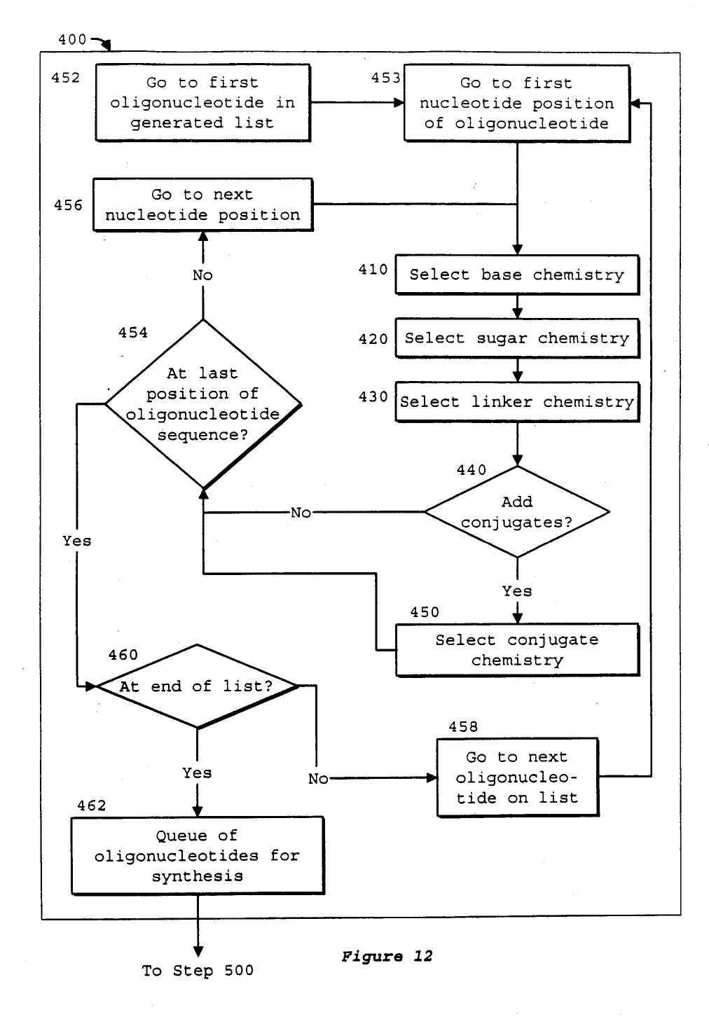

Chemistries can be assigned to the nucleobase sequences during general procedure step 400 (FIG. 1). The logical basis for chemistry assignment is illustrated in FIGS. 10 and 11 and an iterative routine for stepping through an oligonucleotide nucleoside by nucleoside is illustrated in FIG. 12. Chemistry assignment can be effected by assignment directly into a word processing program, via an interactive word processing program or via automated programs and devices. In each of these instances, the output file is selected to be in a format that can serve as an input file to automated synthesis devices.

9. Oligonucleotide Compounds.

In the context of this invention, in reference to oligonucleotides, the term “oligonucleotide” is used to refer to an oligomer or polymer of ribonucleic acid (RNA) or deoxyribonucleic acid (DNA) or mimetics thereof. Thus this term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms, i.e., phosphodiester linked A, C, G, T and U nucleosides, because of desirable properties such as, for example, enhanced cellular uptake, enhanced affinity for nucleic acid target and increased stability in the presence of nucleases.

The oligonucleotide compounds in accordance with this invention can be of various lengths depending on various parameters, including but not limited to those discussed above in reference to the selection criteria of general procedure 300. For use as antisense oligonucleotides compounds of the invention preferably are from about 8 to about 30 nucleobases in length (i.e. from about 8 to about 30 linked nucleosides). Particularly preferred are antisense oligonucleotides comprising from about 12 to about 25 nucleobases. A discussion of antisense oligonucleotides and some desirable modifications can be found in De Mesmaeker et al., Acc. Chem. Res., 1995, 28, 366. Other lengths of oligonucleotides might be selected for non-antisense targeting strategies, for instance using the oligonucleotides as ribozymes. Such ribozymes normally require oligonucleotides of longer length as is known in the art.

A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a normal (where normal is defined as being found in RNA and DNA) pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred oligonucleotides useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

10. Selection of Oligonucleotide Chemistries.

In a general logic sheme as illustrated in FIGS. 10 and 11, for each nucleoside position, the user or automated device is interrogated first for a base assignment, followed by a sugar assignment, a linker assignment and finally a conjugate assignment. Thus for each nucleoside, at process step 410 a base is selected. In selecting the base, base chemistry 1 can be selected at process step 412 or one or more alternative bases are selected at process steps 414, 416 and 418. After base selection is effected, the sugar portion of the nucleoside is selected. Thus for each nucleoside, at process step 420 a sugar is selected that together with the select base will complete the nucleoside. In selecting the sugar, sugar chemistry 1 can be selected at process 422 or one or more alternative sugars are selected at process steps 424, 426 and 428. For each two adjacent nucleoside units, at process step 430, the internucleoside linker is selected. The linker chemistry for the internucleoside linker can be linker chemistry 1 selected at process step 432 or one or more alternative internucleoside linker chemistries are selected at process steps 434, 436 and 438.

In addition to the base, sugar and internucleoside linkage, at each nucleoside position, one or more conjugate groups can be attached to the oligonucleotide via attachment to the nucleoside or attachment to the internucleoside linkage. The addition of a conjugate group is integrated at process step 440 and the assignment of the conjugate group is effected at process step 450.

For illustrative purposes in FIGS. 10 and 11, for each of the bases, the sugars, the internucleoside linkers, or the conjugates, chemistries 1 though n are illustrated. As described in this specification, it is understood that the number of alternate chemistries between chemistry 1 and alternative chemistry n, for each of the bases, the sugars, the internucleoside linkages and the conjugates, is variable and includes, but is not limited to, each of the specific alternative bases, sugar, internucleoside linkers and conjugates identified in this specification as well as equivalents known in the art.

Utilizing the logic as described in conjunction with FIGS. 10 and 11, chemistry is assigned, as is shown in FIG. 12, to the list of oligonucleotides from general procedure 300. In assigning chemistries to the oligonucleotides in this list, a pointer can be set at process step 452 to the first oligonucleotide in the list and at step 453 to the first nucleotide of that first oligonucleotide. The base chemistry is selected at step 410, as described above, the sugar chemistry is selected at step 420, also as described above, followed by selection of the internucleoside linkage at step 430, also as described above. At decision 440, the process branches depending on whether a conjugate will be added at the current nucleotide position. If a conjugate is desired, the conjugate is selected at step 450, also as described above.

Whether or not a conjugate was added at decision step 440, an inquiry is made at decision step 454. This inquiry asks if the pointer resides at the last nucleotide in the current oligonucleotide. If the result at decision step 454 is “No,” the pointer is moved to the next nucleotide in the current oligonucleotide and the loop including steps 410, 420, 430, 440 and 454 is repeated. This loop is reiterated until the result at decision step 454 is “Yes.”

When the result at decision step 454 is “Yes,” a query is made at decision step 460 concerning the location of the pointer in the list of oligonucleotides. If the pointer is not at the last oligonucleotide of the list, the “No” path of the decision step 460 is followed and the pointer is moved to the first nucleotide of the next oligonucleotide in the list at process step 458. With the pointer set to the next oligonucleotide in the list, the loop that starts at process steps 453 is reiterated. When the result at decision step 460 is “Yes,” chemistry has been assigned to all of the nucleotides in the list of oligonucleotides.

11. Description of Oligonucleotide Chemistries.

As is illustrated in FIG. 10, for each nucleoside of an oligonucleotide, chemistry selection includes selection of the base forming the nucleoside from a large palette of different base units available. These may be “modified” or “natural” bases (also reference herein as nucleobases) including the natural purine bases adenine (A) and guanine (G), and the natural pyrimidine bases thymine (T), cytosine (C) and uracil (U). They further can include modified nucleobases including other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo uracils and cytosines particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred for selection as the base. These are particularly useful when combined with a 2′-O-methoxyethyl sugar modifications, described below.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, each of which is incorporated herein by reference in its entirety. Reference is also made to allowed U.S. patent application Ser. No. 08/762,488, filed on Dec. 10, 1996, commonly owned with the present application and which is incorporated herein by reference in its entirety.

In selecting the base for any particular nucleoside of an oligonucleotide, consideration is first given to the need of a base for a particular specificity for hybridization to an opposing strand of a particular target. Thus if an “A” base is required, adenine might be selected however other alternative bases that can effect hybridization in a manner mimicking an “A” base such as 2-aminoadenine might be selected should other consideration, e.g., stronger hybridization (relative to hybridization achieved with adenine), be desired.

As is illustrated in FIG. 10, for each nucleoside of an oligonucleotide, chemistry selection includes selection of the sugar forming the nucleoside from a large palette of different sugar or sugar surrogate units available. These may be modified sugar groups, for instance sugars containing one or more substituent groups. Preferred substituent groups comprise the following at the 2′ position: OH; F; O-, S-, or N-alkyl; O-, S-, or N-alkenyl; or O, S- or N-alkynyl; wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred substituent groups comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl), 2′-O-methoxyethyl, or 2′-MOE) (Martin et al., Helv. Chim. Acta, 1995, 78, 486) i.e., an alkoxyalkoxy group. A further preferred modification includes 2′-dimethylamino oxyethoxy, i.e., a O(CH₂)₂ON(CH₃)₂ group, also known as 2′-DMAOE, as described in co-owned U.S. patent application Ser. No. 09/016,520, filed on Jan. 30, 1998, which is incorporated herein by reference in its entirety.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the sugar group, particularly the 3′ position of the sugar on the 3′ terminal nucleotide or in 2′-5′ linked oligonucleotides and the 5′ position of 5′ terminal nucleotide. The nucleosides of the oligonucleotides may also have sugar mimetics such as cyclobutyl moieties in place of the pentofuranosyl sugar. Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053 5,639,873; 5,646,265; 5,658,873; 5,670,633; and 5,700,920, certain of which are commonly owned with the present application, each of which is incorporated herein by reference in its entirety, together with allowed U.S. patent application Ser. No. 08/468,037, filed on Jun. 5, 1995, which is commonly owned with the present application and which is incorporated herein by reference in its entirety.

As is illustrated in FIG. 10, for each adjacent pair of nucleosides of an oligonucleotide, chemistry selection includes selection of the internucleoside linkage. These internucleoside linkages are also referred to as linkers, backbones or oligonucleotide backbones. For forming these nucleoside linkages, a palette of different internucleoside linkages or backbones is available. These include modified oligonucleotide backbones, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalklyphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,625,050; and 5,697,248, certain of which are commonly owned with this application, each of which is incorporated herein by reference in its entirety.

Preferred internucleoside linkages for oligonucleotides that do not include a phosphorus atom therein, i.e., for oligonucleosides, have backbones that are formed by short chain alkyl or cycloalkyl intersugar linkages, mixed heteroatom and alkyl or cycloalkyl intersugar linkages, or one or more short chain heteroatomic or heterocyclic intersugar linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, certain of which are commonly owned with this application, each of which is incorporated herein by reference in its entirety.

In other preferred oligonucleotides, i.e., oligonucleotide mimetics, both the sugar and the intersugar linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-phosphate backbone of an oligonucleotide is replaced with an amide-containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is incorporated herein by reference in its entirety. Further teaching of PNA compounds can be found in Nielsen et al., Science, 1991, 254, 1497.

For the internucleoside linkages, the most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— (wherein the native phosphodiester backbone is represented as —O—P—O—CH₂—) of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

In attaching a conjugate group to one or more nucleosides or internucleoside linkages of an oligonucleotide, various properties of the oligonucleotide are modified. Thus modification of the oligonucleotides of the invention to chemically link one or more moieties or conjugates to the oligonucleotide are intended to enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553), cholic acid (Manoharan et al., Bioorg. Med. Chem. Let., 1994, 4, 1053), a thioether, e.g., hexyl-S-tritylthiol (Manoharan et al., Ann. N.Y Acad. Sci., 1992, 660,306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the present application, and each of which is herein incorporated by reference in its entirety.

12. Chimeric Compounds.

It is not necessary for all positions in a given compound to be uniformly modified. In fact, more than one of the aforementioned modifications may be incorporated in a single compound or even at a single nucleoside within an oligonucleotide. The present invention also includes compounds which are chimeric compounds. “Chimeric” compounds or “chimeras,” in the context of this invention, are compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids.

By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures representing the union of two or more oligonucleotides, modified oligonucleotides, oligonucleosides and/or oligonucleotide mimetics as described above. Such compounds have also been referred to in the art as “hybrids” or “gapmers”. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the present application and each of which is incorporated herein by reference in its entirety, together with commonly owned and allowed U.S. patent application Ser. No. 08/465,880, filed on Jun. 6, 1995, which is incorporated herein by reference in its entirety.

13. Description of Automated Oligonucleotide Synthesis.

In the next step of the overall process (illustrated in FIGS. 1 and 2), oligonucleotides are synthesized on an automated synthesizer. Although many devices may be employed, the synthesizer is preferably a variation of the synthesizer described in U.S. Pat. Nos. 5,472,672 and 5,529,756, each of which is incorporated herein by reference in its entirety. The synthesizer described in those patents is modified to include movement in along the Y axis in addition to movement along the X axis. As so modified, a 96-well array of compounds can be synthesized by the synthesizer. The synthesizer further includes temperature control and the ability to maintain an inert atmosphere during all phases of synthesis. The reagent array delivery format employs orthogonal X-axis motion of a matrix of reaction vessels and Y-axis motion of an array of reagents. Each reagent has its own dedicated plumbing system to eliminate the possibility of cross-contamination of reagents and line flushing and/or pipette washing. This in combined with a high delivery speed obtained with a reagent mapping system allows for the extremely rapid delivery of reagents. This further allows long and complex reaction sequences to be performed in an efficient and facile manner.

The software that operates the synthesizer allows the straightforward programming of the parallel synthesis of a large number of compounds. The software utilizes a general synthetic procedure in the form of a command (.cmd) file, which calls upon certain reagents to be added to certain wells via lookup in a sequence (.seq) file. The bottle position, flow rate, and concentration of each reagent is stored in a lookup table (.tab) file. Thus, once any synthetic method has been outlined, a plate of compounds is made by permutating a set of reagents, and writing the resulting output to a text file. The text file is input directly into the synthesizer and used for the synthesis of the plate of compounds. The synthesizer is interfaced with a relational database allowing data output related to the synthesized compounds to be registered in a highly efficient manner.

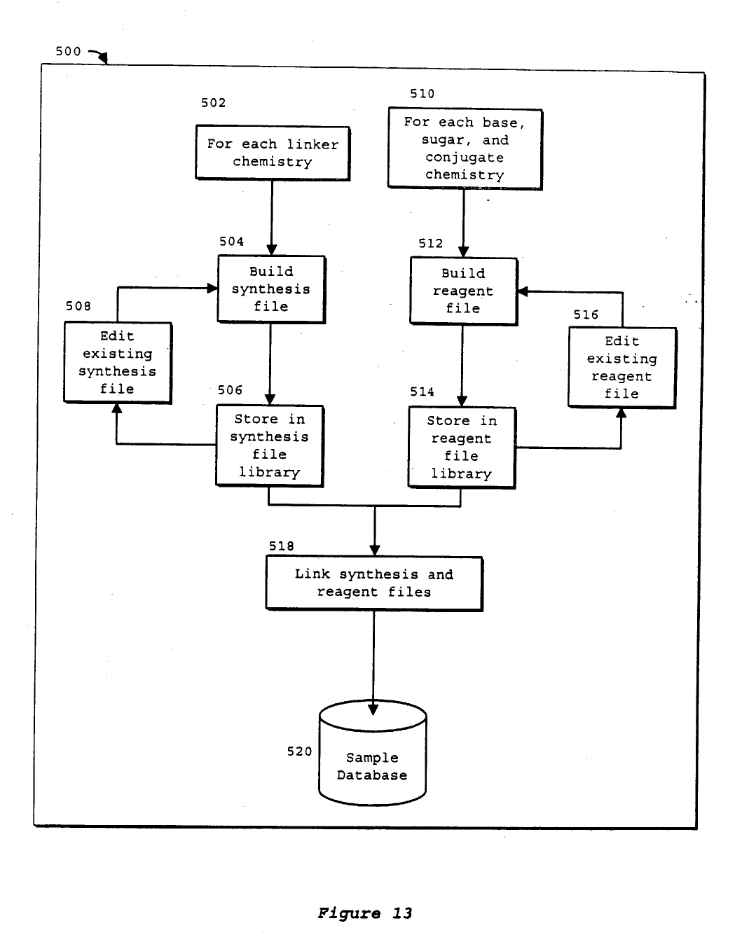

Building of the .seq, .cmd and .tab files is illustrated in FIG. 13. Thus as a part of the general oligonucleotide synthesis procedure 500, for each linker chemistry at process step 502, a synthesis file, i.e., a .cmd file, is built at process step 504. This file can be built fresh to reflect a completely new set of machine commands reflecting a set of chemical synthesis steps or it can modify an existing file stored at process step 504 by editing that stored file in process step 508. The .cmd files are built using a word processor and a command set of instructions as outlined below.

It will be appreciated that the preparation of control software and data files is within the routine skill of persons skilled in annotated nucleotide synthesis. The same will depend upon the hardware employed, the chemistries adopted and the design paradigm selected by the operator.

In a like manner to the building the .cmd files, .tab files are built to reflect the necessary reagents used in the automatic synthesizer for the particular chemistries that have been selected for the linkages, bases, sugars and conjugate chemistries. Thus for each of a set of these chemistries at process step 510, a .tab file is built at process step 512 and stored at process step 514. As with the .cmd files, an existing .tab file can be edited at process step 516.

Both the .cmd files and the .tab files are linked together at process step 518 and stored for later retrieval in an appropriate sample database 520. Linking can be as simple as using like file names to associate a .cmd file to its appropriate .tab file, e.g., synthesis_{—}1.cmd is linked to synthesis_{—}1.tab by use of the same preamble in their names.

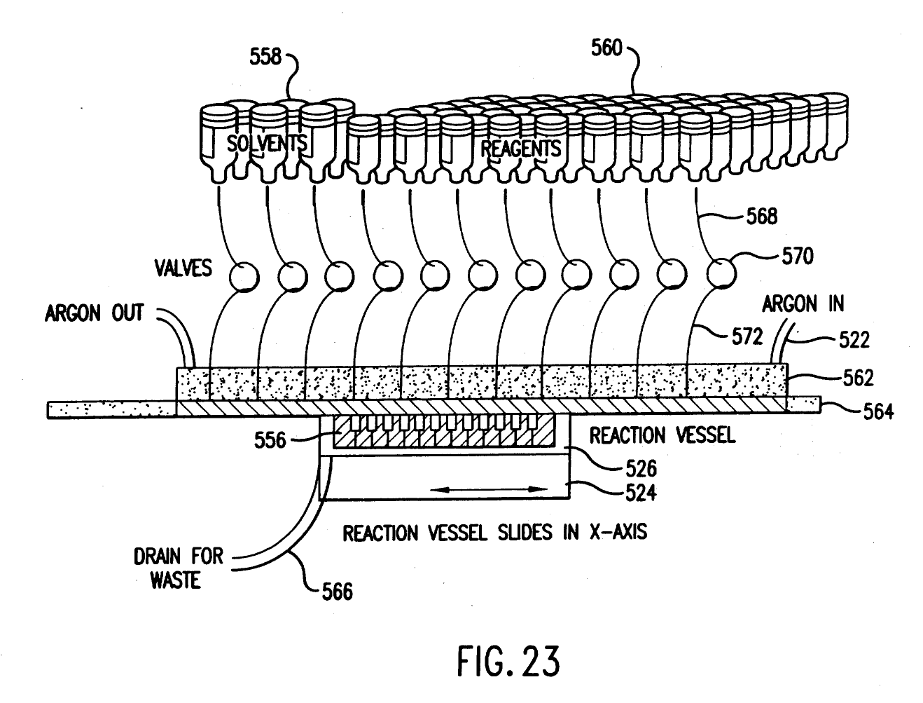

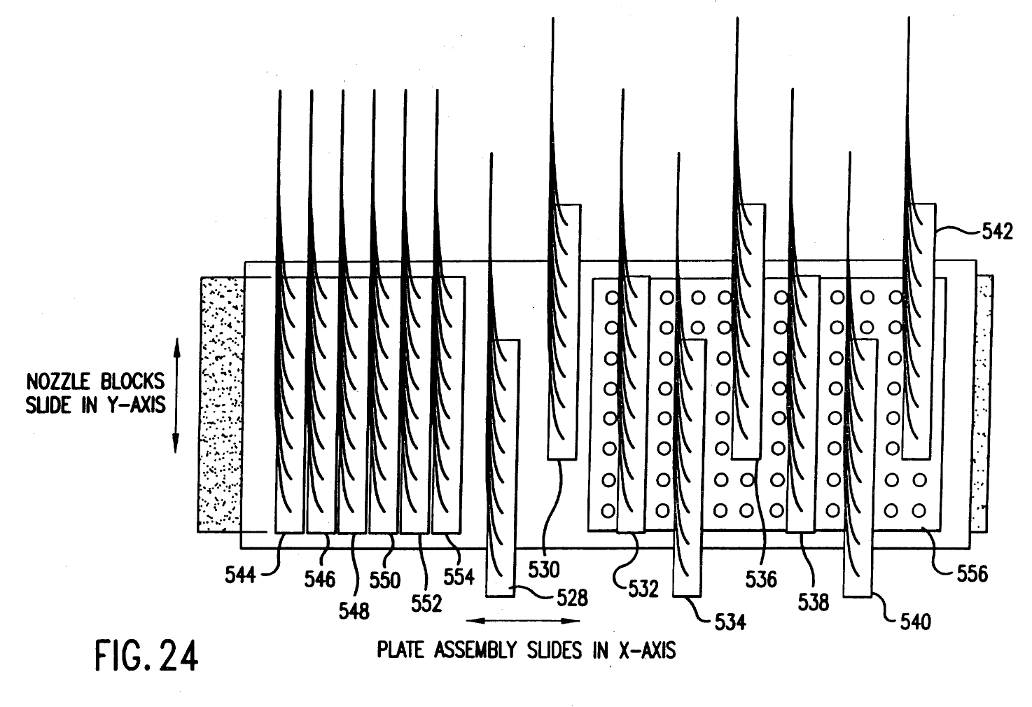

The automated, multi-well parallel array synthesizer employs a reagent array delivery format, in which each reagent utilized has a dedicated plumbing system. As seen in FIGS. 23 and 24, an inert atmosphere 522 is maintained during all phases of a synthesis. Temperature is controlled via a thermal transfer plate 524, which holds an injection molded reaction block 526. The reaction plate assembly slides in the X-axis direction, while for example eight nozzle blocks (528, 530, 532, 534, 536, 538, 540 and 542) holding the reagent lines slide in the Y-axis direction, allowing for the extremely rapid delivery of any of 64 reagents to 96 wells. In addition, there are for example, six banks of fixed nozzle blocks (544, 546, 548, 550, 552 and 554) which deliver the same reagent or solvent to eight wells at once, for a total of 72 possible reagents.

In synthesizing oligonucleotides for screening, the target reaction vessels, a 96 well plate 556 (a 2-dimensional array), moves in one direction along the X axis, while the series of independently controlled reagent delivery nozzles (528, 530, 532, 534, 536, 538, 540 and 542) move along the Y-axis relative to the reaction vessel 558. As the reaction plate 556 and reagent nozzles (528, 530, 532, 534, 536, 538, 540 and 542) can be moved independently at the same time, this arrangement facilitates the extremely rapid delivery of up to 72 reagents independently to each of the 96 reaction vessel wells.

The system software allows the straightforward programming of the synthesis of a large number of compounds by supplying the general synthetic procedure in the form of the command file to call upon certain reagents to be added to specific wells via lookup in the sequence file with the bottle position, flow rate, and concentration of each reagent being stored in the separate reagent table file. Compounds can be synthesized on various scales. For oligonucleotides, a 200 nmole scale is typically selected while for other compounds larger scales, as for example a 10 μmole scale (3-5 mg), might be utilized. The resulting crude compounds are generally >80% pure, and are utilized directly for high throughput screening assays. Alternatively, prior to use the plates can be subjected to quality control (see general procedure 600 and Example 8) to ascertain their exact purity. Use of the synthesizer results in a very efficient means for the parallel synthesis of compounds for screening.

The software inputs accept tab delimited text files (as discussed above for file 504 and 512) from any text editor. A typical command file, a .cmd file, is shown in Example 3 at Table 2. Typical sequence files, .seq files, are shown in Example 3 at Tables 3 and 4 (.SEQ file), and a typical reagent file, a .tab file, is shown in Example 3 at Table 5. Table 3 illustrates the sequence file for an oligonucleotide having 2′-deoxy nucleotides at each position with a phosphorothioate backbone throughout. Table 4 illustrates the sequence file for an oligonucleotide, again having a phosphorothioate backbone throughout, however, certain modified nucleoside are utilized in portions of the oligonucleotide. As shown in this table, 2′-O-(2-methoxyethyl) modified nucleosides are utilized in a first region (a wing) of the oligonucleotide, followed by a second region (a gap) of 2′-deoxy nucleotides and finally a third region (a further wing) that has the same chemistry as the first region. Typically some of the wells of the 96 well plate 556 may be left empty (depending on the number of oligonucleotides to be made during an individual synthesis) or some of the wells may have oligonucleotides that will serve as standards for comparison or analytical purposes.

Prior to loading reagents, moisture sensitive reagent lines are purged with argon at 522 for 20 minutes. Reagents are dissolved to appropriate concentrations and installed on the synthesizer. Large bottles, collectively identified as 558 in FIG. 23 (containing 8 delivery lines) are used for wash solvents and the delivery of general activators, trityl group cleaving reagents and other reagents that may be used in multiple wells during any particular synthesis. Small septa bottles, collectively identified as 560 in FIG. 23, are utilized to contain individual nucleotide amidite precursor compounds. This allows for anhydrous preparation and efficient installation of multiple reagents by using needles to pressurize the bottle, and as a delivery path. After all reagents are installed, the lines are primed with reagent, flow rates measured, then entered into the reagent table (.tab file). A dry resin loaded plate is removed from vacuum and installed in the machine for the synthesis.

The modified 96 well polypropylene plate 556 is utilized as the reaction vessel. The working volume in each well is approximately 700 μl. The bottom of each well is provided with a pressed-fit 20 μm polypropylene frit and a long capillary exit into a lower collection chamber as is illustrated in FIG. 5 of the above referenced U.S. Pat. No. 5,372,672. The solid support for use in holding the growing oligonucleotide during synthesis is loaded into the wells of the synthesis plate 556 by pipetting the desired volume of a balanced density slurry of the support suspended in an appropriate solvent, typically an acetonitrile-methylene chloride mixture. Reactions can be run on various scales as for instance the above noted 200 nmole and 10 μmol scales. For oligonucleotide synthesis a CPG support is preferred, however other medium loading polystyrene-PEG supports such as TENTAGEL™ or ARGOGEL™ can also be used.

As seen in FIG. 24, the synthesis plate is transported back and forth in the X-direction under an array of 8 moveable banks (530, 532, 534, 536, 538, 540, 542 and 544) of 8 nozzles (64 total) in the Y-direction, and 6 banks (544, 546, 548, 550, 552 and 554) of 48 fixed nozzles, so that each well can receive the appropriate amounts of reagents and/or solvents from any reservoir (large bottle or smaller septa bottle). A sliding balloon-type seal 562 surrounds this nozzle array and joins it to the reaction plate headspace 564. A slow sweep of nitrogen or argon 522 at ambient pressure across the plate headspace is used to preserve an anhydrous environment.

The liquid contents in each well do not drip out until the headspace pressure exceeds the capillary forces on the liquid in the exit nozzle. A slight positive pressure in the lower collection chamber can be added to eliminate residual slow leakage from filled wells, or to effect agitation by bubbling inert gas through the suspension. In order to empty the wells, the headspace gas outlet valve is closed and the internal pressure raised to about 2 psi. Normally, liquid contents are blown directly to waste 566. However, a 96 well microtiter plate can be inserted into the lower chamber beneath the synthesis plate in order to collect the individual well eluents for spectrophotometric monitoring (trityl, etc.) of reaction progress and yield.

The basic plumbing scheme for the machine is the gas-pressurized delivery of reagents. Each reagent is delivered to the synthesis plate through a dedicated supply line, collectively identified at 568, solenoid valve collectively identified at 570 and nozzle, collectively identified at 572. Reagents never cross paths until they reach the reaction well. Thus, no line needs to be washed or flushed prior to its next use and there is no possibility of cross-contamination of reagents. The liquid delivery velocity is sufficiently energetic to thoroughly mix the contents within a well to form a homogeneous solution, even when employing solutions having drastically different densities. With this mixing, once reactants are in homogeneous solution, diffusion carries the individual components into and out of the solid support matrix where the desired reaction takes place. Each reagent reservoir can be plumbed to either a single nozzle or any combination of up to 8 nozzles. Each nozzle is also provided with a concentric nozzle washer to wash the outside of the delivery nozzles in order to eliminate problems of crystallized reactant buildup due to slow evaporation of solvent at the tips of the nozzles. The nozzles and supply lines can be primed into a set of dummy wells directly to waste at any time.

The entire plumbing system is fabricated with teflon tubing, and reagent reservoirs are accessed via syringe needle/septa or direct connection into the higher capacity bottles. The septum vials 560 are held in removable 8-bottle racks to facilitate easy setup and cleaning. The priming volume for each line is about 350 μl. The minimum delivery volume is about 2 μl, and flow rate accuracy is ±5%. The actual amount of material delivered depends on a timed flow of liquid. The flow rate for a particular solvent will depend on its viscosity and wetting characteristics of the teflon tubing. The flow rate (typically 200-350 μl per sec) is experimentally determined, and this information is contained in the reagent table setup file.

Heating and cooling of the reaction block 526 is effected utilizing a recirculating heat exchanger plate 524, similar to that found in PCR thermocyclers, that nests with the polypropylene synthesis plate 556 to provide good thermal contact. The liquid contents in a well can be heated or cooled at about 10° C. per minute over a range of +5 to +80° C., as polypropylene begins to soften and deform at about 80° C. For temperatures greater than this, a non-disposable synthesis plate machined from stainless steel or monel with replaceable frits can be utilized.

The hardware controller can be any of a wide variety, but conveniently can be designed around a set of three 1 MHz 86332 chips. This controller is used to drive the single X-axis and 8 Y-axis stepper motors as well as provide the timing functions for a total of 154 solenoid valves. Each chip has 16 bidirectional timer I/O and 8 interrupt channels in its timer processing unit (TPU). These are used to provide the step and direction signals, and to read 3 encoder inputs and 2 limit switches for controlling up to three motors per chip. Each 86332 chip also drives a serial chain of 8 UNC5891A darlington array chips to provide power to 64 valves with msec resolution. The controller communicates with the Windows software interface program running on a PC via a 19200 Hz serial channel, and uses an elementary instruction set to communicate valve_number, time_open, motor_number and position_data.

The three components of the software program that run the array synthesizer are the generalized procedure or command (.cmd) file which specifies the synthesis instructions to be performed, the sequence (.seq) file which specifies the scale of the reaction and the order in which variable groups will be added to the core synthon, and the reagent table (.tab) file which specifies the name of a chemical, its location (bottle number), flow rate, and concentration are utilized in conjunction with a basic set of command instructions.

One basic set of command instructions can be:

| ADD | | |
|---|---|---|
| IF | {block of instructions} | END_IF |
| REPEAT | {block of instructions} | END_REPEAT |

PRIME, NOZZLE_WASH
WAIT, DRAIN
LOAD, REMOVE
NEXT_SEQUENCE
LOOP_BEGIN, LOOP_END

The ADD instruction has two forms, and is intended to have the look and feel of a standard chemical equation. Reagents are specified to be added by a molar amount if the number proceeds the name identifier, or by an absolute volume in microliters if the number follows the identifier. The number of reagents to be added is a parsed list, separated by the “+” sign. For variable reagent identifiers, the key word, <seq>, means look in the sequence table for the identity of the reagent to be added, while the key word, <act>, means add the reagent which is associated with that particular <seq>. Reagents are delivered in the order specified in the list.

Thus:

ADD ACN 300

means: Add 300 μl of the named reagent acetonitrile; ACN to each well of active synthesis

ADD <seq>300

means: If the sequence pointer in the .seq file is to a reagent in the list of reagents, independent of scale, add 300 μl of that particular reagent specified for that well.

ADD 1.1 PYR+1.0<seq>+1.1<act1>

means: If the sequence pointer in the .seq file is to a reagent in the list of acids in the Class ACIDS_{—}1, and PYR is the name of pyridine, and ethyl chloroformate is defined in the .tab file to activate the class, ACIDS_{—}1, then this instruction means:

ADD 1.1 equiv. pyridine

1.0 equiv. of the acid specified for that well and

1.1 equiv. of the activator, ethyl chloroformate

The IF command allows one to test what type of reagent is specified in the <seq>variable and process the succeeding block of commands accordingly.

| Thus: | |
|---|---|
| ACYLATION | {the procedure name} |

BEGIN

IF CLASS = ACIDS_1

ADD 1.0 <seq> + 1.1 <act1> + 1.1 PYR
WAIT 60

ENDIF
IF CLASS = ACIDS_2

ADD 1.0 <seq> + 1.2 <act1> + 1.2 TEA

ENDIF
WAIT 60
DRAIN 10

END

means: Operate on those wells for which reagents contained in the Acid_{—}1 class are specified, WAIT 60 sec, then operate on those wells for which reagents contained in the Acid_{—}2 class are specified, then WAIT 60 sec longer, then DRAIN the whole plate. Note that the Acid_{—}1 group has reacted for a total of 120 sec, while the Acid_{—}2 group has reacted for only 60 sec.

The REPEAT command is a simple way to execute the same block of commands multiple times.

| Thus: | |
|---|---|
| WASH_1 | {the procedure name} |

BEGIN

REPEAT 3

ADD ACN 300
DRAIN 15

END_REPEAT

END

means: repeats the add acetonitrile and drain sequence for each well three times.

The PRIME command will operate either on specific named reagents or on nozzles which will be used in the next associated <seq>operation. The μl amount dispensed into a prime port is a constant that can be specified in a config.dat file.

The NOZZLE_WASH command for washing the outside of reaction nozzles free from residue due to evaporation of reagent solvent will operate either on specific named reagents or on nozzles which have been used in the preceding associated <seq>operation. The machine is plumbed such that if any nozzle in a block has been used, all the nozzles in that block will be washed into the prime port.

The WAIT and DRAIN commands are by seconds, with the drain command applying a gas pressure over the top surface of the plate in order to drain the wells.

The LOAD and REMOVE commands are instructions for the machine to pause for operator action.

The NEXT_SEQUENCE command increments the sequence pointer to the next group of substituents to be added in the sequence file. The general form of a .seq file entry is the definition:

Well_No Well_ID Scale Sequence

The sequence information is conveyed by a series of columns, each of which represents a variable reagent to be added at a particular position. The scale (μmole) variable is included so that reactions of different scale can be run at the same time if desired. The reagents are defined in a lookup table (the .tab file), which specifies the name of the reagent as referred to in the sequence and command files, its location (bottle number), flow rate, and concentration. This information is then used by the controller software and hardware to determine both the appropriate slider motion to position the plate and slider arms for delivery of a specific reagent, as well as the specific valve and time required to deliver the appropriate reagents. The adept classification of reagents allows the use of conditional IF loops from within a command file to perform addition of different reagents differently during a “single step” performed across 96 wells simultaneously. The special class ACTIVATORS defines certain reagents that always get added with a particular class of reagents (for example tetrazole during a phosphitylation reaction in adding the next nucleotide to a growing oligonucleotide).

The general form of the .tab file is the definition:

Class Bottle Reagent Name Flow_rate Conc.

The LOOP_BEGIN and LOOP_END commands define the block of commands which will continue to operate until a NEXT_SEQUENCE command points past the end of the longest list of reactants in any well.

Not included in the command set is a MOVE command. For all of the above commands, if any plate or nozzle movement is required, this is automatically executed in order to perform the desired solvent or reagent delivery operation. This is accomplished by the controller software and hardware, which determines the correct nozzle(s) and well(s) required for a particular reagent addition, then synchronizes the position of the requisite nozzle and well prior to adding the reagent.

A MANUAL mode can also be utilized in which the synthesis plate and nozzle blocks can be “homed” or moved to any position by the operator, the nozzles primed or washed, the various reagent bottles depressurized or washed with solvent, the chamber pressurized, etc. The automatic COMMAND mode can be interrupted at any point, MANUAL commands executed, and then operation resumed at the appropriate location. The sequence pointer can be incremented to restart a synthesis anywhere within a command file.

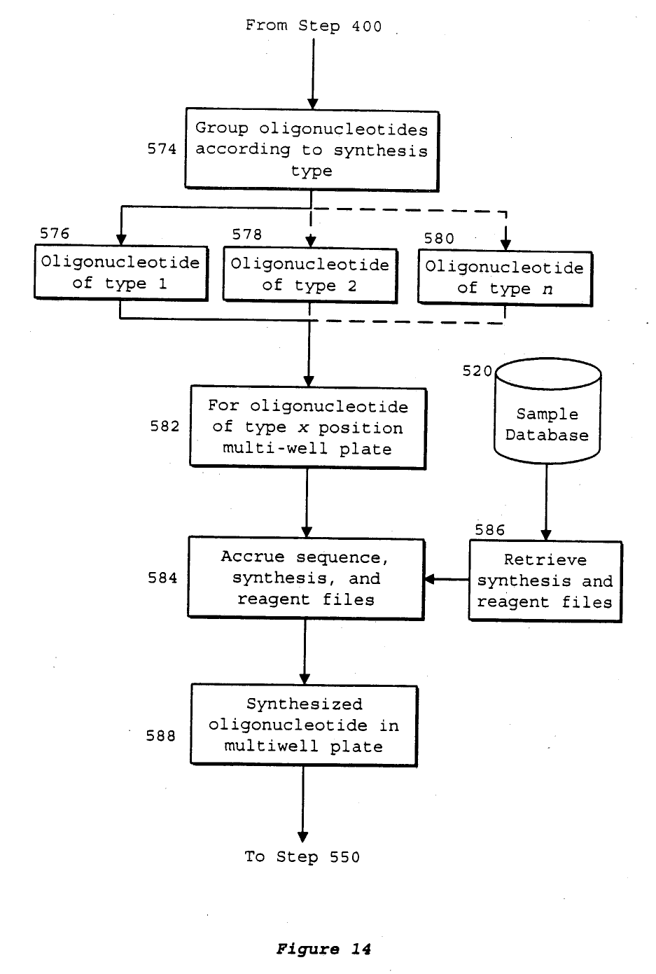

In reference to FIG. 14, the list of oligonucleotides for synthesis can be rearranged or grouped for optimization of synthesis. Thus at process step 574, the oligonucleotides are grouped according to a factor on which to base the optimization of synthesis. As illustrated in the Examples below, one such factor is the 3′ most nucleoside of the oligonucleotide. Using the amidite approach for oligonucleotide synthesis, a nucleotide bearing a 3′ phosphoramite is added to the 5′ hydroxyl group of a growing nucleotide chain. The first nucleotide (at the 3′ terminus of the oligonucleotide—the 3′ most nucleoside) is first connected to a solid support. This is normally done batchwise on a large scale as is standard practice during oligonucleotide synthesis.

Such solid supports pre-loaded with a nucleoside are commercially available. In utilizing the multi well format for oligonucleotide synthesis, for each oligonucleotide to be synthesized, an aliquot of a solid support bearing the proper nucleoside thereon is added to the well for synthesis. Prior to loading the sequence of oligonucleotides to be synthesized in the .seq file, they are sorted by the 3′ terminal nucleotide. Based on that sorting, all of the oligonucleotide sequences having an “A” nucleoside at their 3′ end are grouped together, those with a “C” nucleoside are grouped together as are those with “G” or “T” nucleosides. Thus in loading the nucleoside-bearing solid support into the synthesis wells, machine movements are conserved.

The oligonucleotides can be grouped by the above described parameter or other parameters that facilitate the synthesis of the oligonucleotides. Thus in FIG. 14, sorting is noted as being effected by some parameter of type 1, as for instance the above described 3′ most nucleoside, or other types of parameters from type 2 to type n at process steps 576, 578 and 580. Since synthesis will be from the 3′ end of the oligonucleotides to the 5′ end, the oligonucleotide sequences are reverse sorted to read 3′ to 5′. The oligonucleotides are entered in the .seq file in this form, i.e., reading 3′ to 5′.

Once sorted into types, the position of the oligonucleotides on the synthesis plates is specified at process step 582 by the creation of a .seq file as described above. The .seq file is associated with the respective .cmd and .tab files needed for synthesis of the particular chemistries specified for the oligonucleotides at process step 584 by retrieval of the .cmd and .tab files at process step 586 from the sample database 520. These files are then input into the multi well synthesizer at process step 588 for oligonucleotide synthesis. Once physically synthesized, the list of oligonucleotides again enters the general procedure flow as indicated in FIG. 1. For shipping, storage or other handling purposes, the plates can be lyophilized at this point if desired. Upon lyophilization, each well contains the oligonucleotides located therein as a dry compound.

14. Quality Control.

In an optional step, quality control is performed on the oligonucleotides at process step 600 after a decision is made (decision step 550) to perform quality control. Although optional, quality control may be desired when there is some reason to think that some aspect of the synthetic process step 500 has been compromised. Alternatively, samples of the oligonucleotides may be taken and stored in the event that the results of assays conducted using the oligonucleotides (process step 700) yield confusing results or suboptimal data. In the latter event, for example, quality control might be performed after decision step 800 if no oligonucleotides with sufficient activity are identified. In either event, decision step 650 follows quality control step process 600. If one or more of the oligonucleotides do not pass quality control, process step 500 can be repeated, i.e., the oligonucleotides are synthesized for a second time.

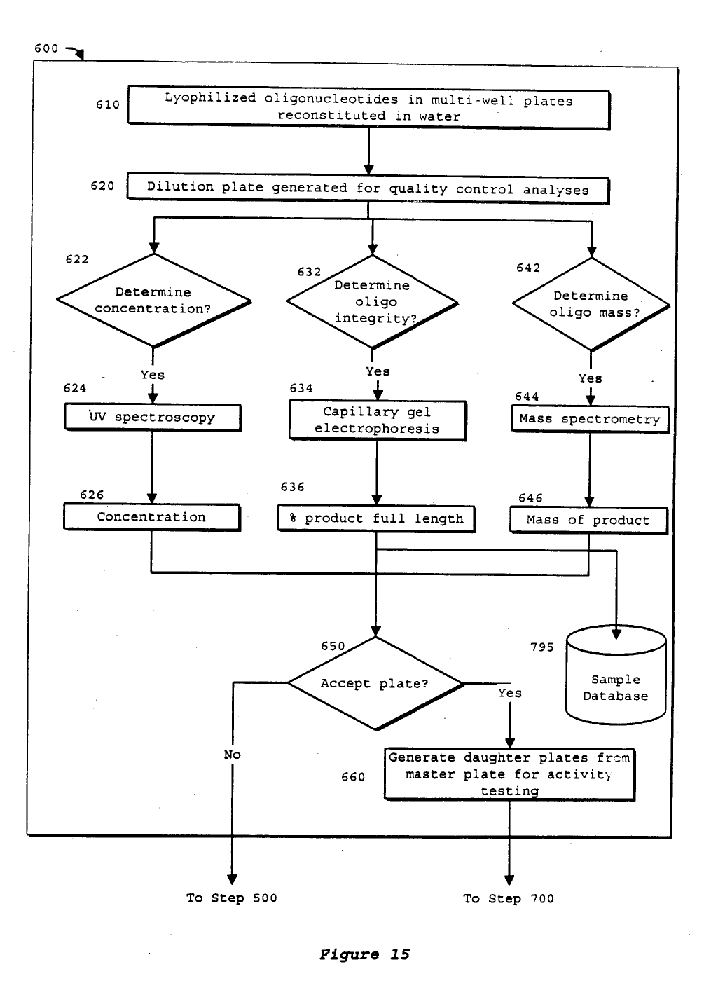

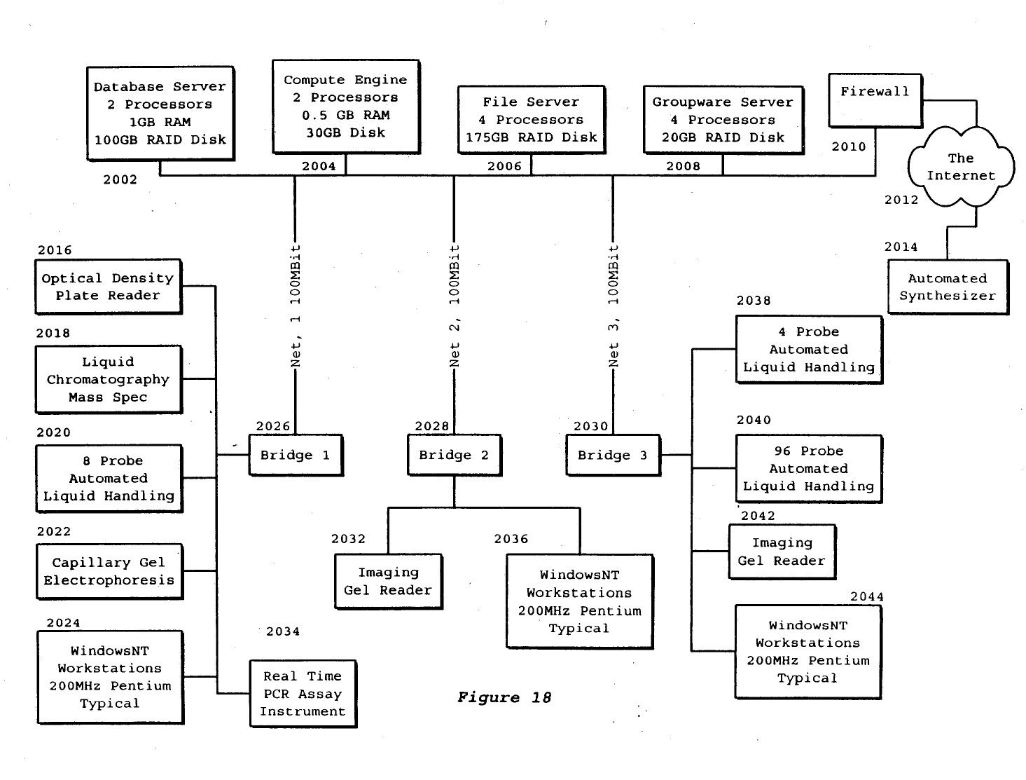

The operation of the quality control system general procedure 600 is detailed in steps 610-660 of FIG. 15. Also referenced in the following discussion are the robotics and associated analytical instrumentation as shown in FIG. 18.

During step 610 (FIG. 15), sterile, double-distilled water is transferred by an automated liquid handler (2040 of FIG. 18) to each well of a multi-well plate containing a set of lyophilized antisense oligonucleotides. The automated liquid handler (2040 of FIG. 18) reads the barcode sticker on the multi-well plate to obtain the plate's identification number. Automated liquid handler 2040 then queries Sample Database 520 (which resides in Database Server 2002 of FIG. 18) for the quality control assay instruction set for that plate and executes the appropriate steps. Three quality control processes are illustrated, however, it is understood that other quality control processes or steps maybe practiced in addition to or in place of the processes illustrated.

The first illustrative quality control process (steps 622 to 626) quantitates the concentration of oligonucleotide in each well. If this quality control step is performed, an automated liquid handler (2040 of FIG. 18) is instructed to remove an aliquot from each well of the master plate and generate a replicate daughter plate for transfer to the UV spectrophotometer (2016 of FIG. 18). The UV spectrophotometer (2016 of FIG. 18) then measures the optical density of each well at a wavelength of 260 nanometers. Using standardized conversion factors, a microprocessor within UV spectrophotometer (2016 of FIG. 18) then calculates a concentration value from the measured absorbance value for each well and output the results to Sample Database 520.

The second illustrative quality control process steps 632 to 636) quantitates the percent of total oligonucleotide in each well that is full length. If this quality control step is performed, an automated liquid handler (2040 of FIG. 18) is instructed to remove an aliquot from each well of the master plate and generate a replicate daughter plate for transfer to the multichannel capillary gel electrophoresis apparatus (2022 of FIG. 18). The apparatus electrophoretically resolves in capillary tube gels the oligonucleotide product in each well. As the product reaches the distal end of the tube gel during electrophoresis, a detection window dynamically measures the optical density of the product that passes by it. Following electrophoresis, the value of percent product that passed by the detection window with respect to time is utilized by a built in microprocessor to calculate the relative size distribution of oligonucleotide product in each well. These results are then output to the Sample Database (520.

The third illustrative quality control process steps 632 to 636) quantitates the mass of the oligonucleotide in each well that is full length. If this quality control step is performed, an automated liquid handler (2040 of FIG. 18) is instructed to remove an aliquot from each well of the master plate and generate a replicate daughter plate for transfer to the multichannel liquid electrospray mass spectrometer (2018 of FIG. 18). The apparatus then uses electrospray technology to inject the oligonucleotide product into the mass spectrometer. A built in microprocessor calculates the mass-to-charge ratio to arrive at the mass of oligonucleotide product in each well. The results are then output to Sample Database 520.

Following completion of the selected quality control processes, the output data is manually examined or is examined using an appropriate algorithm and a decision is made as to whether or not the plate receives “Pass” or “Fail” status. The current criteria for acceptance, for 18 mer oligonucleotides, is that at least 85% of the oligonucleotides in a multi-well plate must be 85% or greater full length product as measured by both capillary gel electrophoresis and mass spectrometry. An input (manual or automated) is then made into Sample Database 520 as to the pass/fail status of the plate. If a plate fails, the process cycles back to step 500, and a new plate of the same oligonucleotides is automatically placed in the plate synthesis request queue (process 554 of FIG. 15). If a plate receives “Pass” status, an automated liquid handler (2040 of FIG. 18) is instructed to remove appropriate aliquots from each well of the master plate and generate two replicate daughter plates in which the oligonucleotide in each well is at a concentration of 30 micromolar. The plate then moves on to process 700 for oligonucleotide activity evaluation.

15. Cell Lines for Assaying Oligonucleotide Activity.

The effect of antisense compounds on target nucleic acid expression can be tested in any of a variety of cell types provided that the target nucleic acid, or its gene product, is present at measurable levels. This can be routinely determined using, for example, PCR or Northern blot analysis. The following four cell types are provided for illustrative purposes, but other cell types can be routinely used.

T-24 cells: The transitional cell bladder carcinoma cell line T-24 is obtained from the American Type Culture Collection (ATCC) (Manassas, Va.). T-24 cells were routinely cultured in complete McCoy's 5A basal media (Life Technologies, Gaithersburg, Md.) supplemented with 10% fetal calf serum, penicillin 100 units per milliliter, and streptomycin 100 micrograms per milliliter (all from Life Technologies). Cells are routinely passaged by trypsinization and dilution when they reach 90% confluence. Cells are routinely seeded into 96-well plates (Falcon-Primaria #3872) at a density of 7000 cells/well for use in RT-PCR analysis. For Northern blotting or other analysis, cells are seeded onto 100 mm or other standard tissue culture plates and treated similarly, using appropriate volumes of medium and oligonucleotide.

A549 cells: The human lung carcinoma cell line A549 is obtained from the ATCC (Manassas, Va.). A549 cells were routinely cultured in DMEM basal media (Life Technologies) supplemented with 10% fetal calf serum, penicillin 100 units per milliliter, and streptomycin 100 micrograms per milliliter (all from Life Technologies). Cells are routinely passaged by trypsinization and dilution when they reach 90% confluence.

NHDF cells: Human neonatal dermal fibroblast (NHDF) were obtained from the Clonetics Corporation (Walkersville, Md.). NHDFs were routinely maintained in Fibroblast Growth Medium (Clonetics Corp.) as provided by the supplier. Cells are maintained for up to 10 passages as recommended by the supplier.

HEK cells: Human embryonic keratinocytes (HEK) were obtained from the Clonetics Corp. HEKs were routinely maintained in Keratinocyte Growth Medium (Clonetics Corp.) as provided by the supplier. Cell are routinely maintained for up to 10 passages as recommended by the supplier.

16. Treatment of Cells with Candidate Compounds:

When cells reach about 80% confluency, they are treated with oligonucleotide. For cells grown in 96-well plates, wells are washed once with 200 μl OPTI-MEM-1™ reduced-serum medium (Life Technologies) and then treated with 130 μl of OPTI-MEM-1™ containing 3.75 μg/ml LIPOFECTIN™ (Life Technologies) and the desired oligonucleotide at a final concentration of 150 nM. After 4 hours of treatment, the medium was replaced with fresh medium. Cells were harvested 16 hours after oligonucleotide treatment.

Alternatively, for cells resistant to cationic mediated transfection, oligonucleotides can be introduced by electroporation. Electroporation conditions must be optimized for every cell type. In general, oligonucleotide is added directly to complete growth media to a final concentration between 1 and 20 micromolar. An electronic pulse is delivered to the cells using a BTX T820 ELECTRO SQUARE PORATOR™ using a Multi-coaxial 96-well electrode (BT840) (BTX Corporation, San Diego, Calif.). Following electroporation, the cells are returned to the incubator for 16 hours.

17. Assaying Oligonucleotide Activity:

Oligonucleotide-mediated modulation of expression of a target nucleic acid can be assayed in a variety of ways known in the art. For example, target RNA levels can be quantitated by, e.g., Northern blot analysis, competitive PCR, or reverse transcriptase polymerase chain reaction (RT-PCR). RNA analysis can be performed on total cellular RNA or, preferably in the case of polypeptide-encoding nucleic acids, poly(A)+ mRNA. For RT-PCR, poly(A)+ mRNA is preferred. Methods of RNA isolation are taught in, for example, Ausubel et al. (Short Protocols in Molecular Biology, 2nd Ed., pp. 4-1 to 4-13, Greene Publishing Associates and John Wiley & Sons, New York, 1992). Northern blot analysis is routine in the art (Id., pp. 4-14 to 4-29).

Alternatively, total RNA can be prepared from cultured cells or tissue using the QIAGEN RNeasy®-96 kit for the high throughput preparation of RNA (QIAGEN, Inc., Valencia, Calif.). Essentially, protocols are carried out according to the manufacturer's directions. Optionally, a DNase step is included to remove residual DNA prior to RT-PCR.

To improve efficiency and accuracy the repetitive pipeting steps and elution step have been automated using a QIAGEN Bio-Robot 9604. Essentially after lysing of the oligonucleotide treated cell cultures in situ, the plate is transferred to the robot deck where the pipeting, DNase treatment, and elution steps are carried out.

Reverse transcriptase polymerase chain reaction (RT-PCR) can be conveniently accomplished using the commercially available ABI PRISM® 7700 Sequence Detection System (PE-Applied Biosystems, Foster City, Calif.) according to the manufacturer's instructions. Other methods of PCR are also known in the art.

Target protein levels can be quantitated in a variety of ways well known in the art, such as immunoprecipitation, Western blot analysis (immunoblotting), Enzyme-linked immunosorbent assay (ELISA) or fluorescence-activated cell sorting (FACS). Antibodies directed to a protein encoded by a target nucleic acid can be identified and obtained from a variety of sources, such as the MSRS catalog of antibodies, (Aerie Corporation, Birmingham, Mich. or via the world wide web of the interact at ANTIBODIES-PROBES.com/), or can be prepared via conventional antibody generation methods. Methods for preparation of polyclonal, monospecific (“antipeptide”) and monoclonal antisera are taught by, for example, Ausubel et al. (Short Protocols in Molecular Biology, 2nd Ed., pp. 11-3 to 11-54, Greene Publishing Associates and John Wiley & Sons, New York, 1992).

Immunoprecipitation methods are standard in the art and are described by, for example, Ausubel et al. (Id., pp. 10-57 to 10-63). Western blot (immunoblot) analysis is standard in the art (Id., pp. 10-32 to 10-10-35). Enzyme-linked immunosorbent assays (ELISA) are standard in the art (Id., pp. 11-5 to 11-17).

Because it is preferred to assay the compounds of the invention in a batchwise fashion, i.e., in parallel to the automated synthesis process described above, preferred means of assaying are suitable for use in 96-well plates and with robotic means. Accordingly, automated RT-PCR is preferred for assaying target nucleic acid levels, and automated ELISA is preferred for assaying target protein levels.

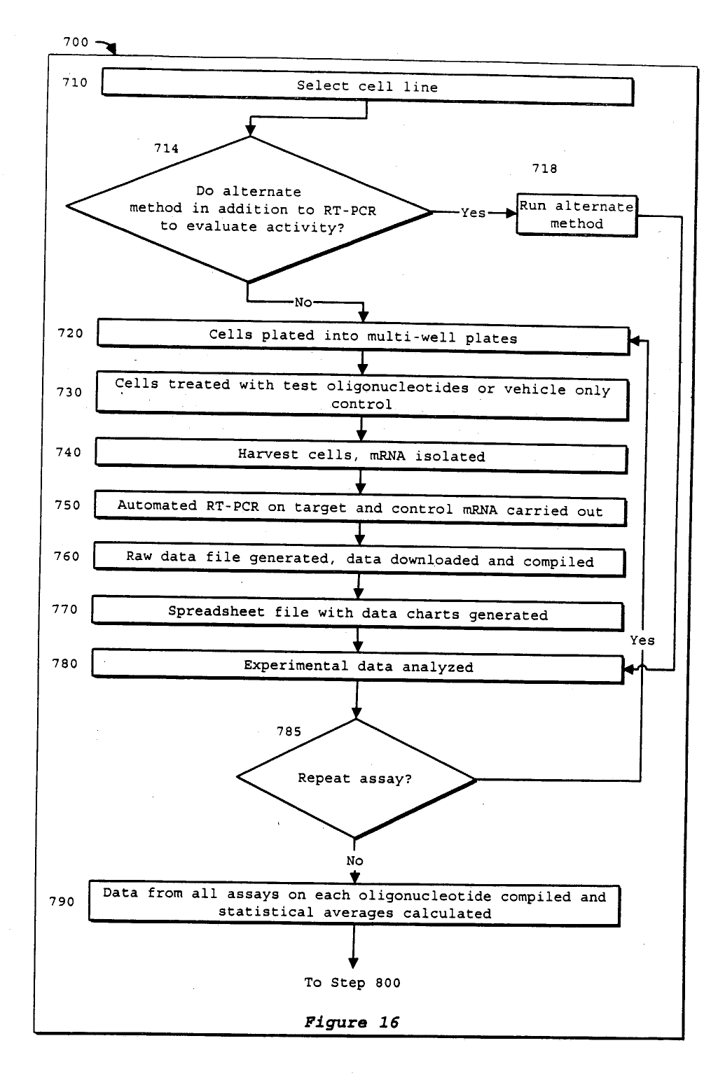

The assaying step, general procedure step 700, is described in detail in FIG. 16. After an appropriate cell line is selected at process step 710, a decision is made at decision step 714 as to whether RT-PCR will be the only method by which the activity of the compounds is evaluated. In some instances, it is desirable to run alternative assay methods at process step 718; for example, when it is desired to assess target polypeptide levels as well as target RNA levels, an immunoassay such as an ELISA is run in parallel with the RT-PCR assays. Preferably, such assays are tractable to semi-automated or robotic means.

When RT-PCR is used to evaluate the activities of the compounds, cells are plated into multi-well plates (typically, 96-well plates) in process step 720 and treated with test or control oligonucleotides in process step 730. Then, the cells are harvested and lysed in process step 740 and the lysates are introduced into an apparatus where RT-PCR is carried out in process step 750. A raw data file is generated, and the data is downloaded and compiled at step 760. Spreadsheet files with data charts are generated at process step 770, and the experimental data is analyzed at process step 780. Based on the results, a decision is made at process step 785 as to whether it is necessary to repeat the assays and, if so, the process begins again with step 720. In any event, data from all the assays on each oligonucleotide are compiled and statistical parameters are automatically determined at process step 790.

18. Classification of Compounds Based on their Activity:

Following assaying, general procedure step 700, oligonucleotide compounds are classified according to one or more desired properties. Typically, three classes of compounds are used: active compounds, marginally active (or “marginal”) compounds and inactive compounds. To some degree, the selection criteria for these classes vary from target to target, and members of one or more classes may not be present for a given set of oligonucleotides.

However, some criteria are constant. For example, inactive compounds will typically comprise those compounds having 5% or less inhibition of target expression (relative to basal levels). Active compounds will typically cause at least 30% inhibition of target expression, although lower levels of inhibition are acceptable in some instances. Marginal compounds will have activities intermediate between active and inactive compounds, with preferred marginal compounds having activities more like those of active compounds.

19. Optimization of Lead Compounds by Sequence.

One means by which oligonucleotide compounds are optimized for activity is by varying their nucleobase sequences so that different regions of the target nucleic acid are targeted. Some such regions will be more accessible to oligonucleotide compounds than others, and “sliding” a nucleobase sequence along a target nucleic acid only a few bases can have significant effects on activity. Accordingly, varying or adjusting the nucleobase sequences of the compounds of the invention is one means by which suboptimal compounds can be made optimal, or by which new active compounds can be generated.

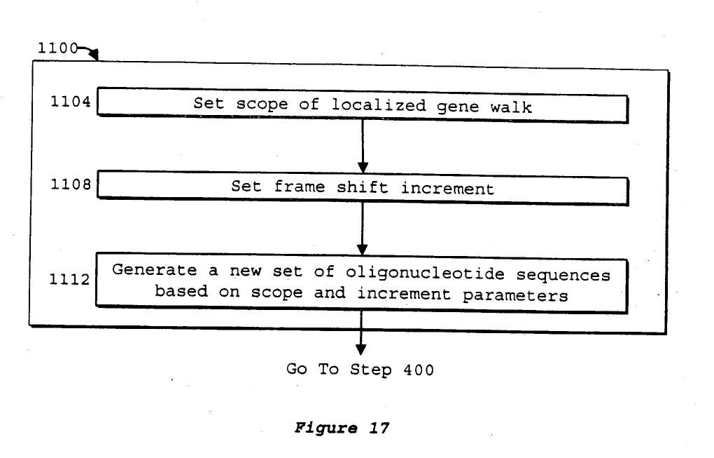

The operation of the gene walk process 1100 detailed in steps 1104-1112 of FIG. 17 is detailed as follows. As used herein, the term “gene walk” is defined as the process by which a specified oligonucleotide sequence x that binds to a specified nucleic acid target y is used as a frame of reference around which a series of new oligonucleotides sequences capable of hybridizing to nucleic acid target y are generated that are sequence shifted increments of oligonucleotide sequence x. Gene walking can be done “downstream”, “upstream” or in both directions from a specified oligonucleotide.

During step 1104 the user manually enters the identification number of the oligonucleotide sequence around which it is desired to execute gene walk process 1100 and the name of the corresponding target nucleic acid. The user then enters the scope of the gene walk at step 1104, by which is meant the number of oligonucleotide sequences that it is desired to generate. The user then enters in step 1108 a positive integer value for the sequence shift increment. Once this data is generated, the gene walk is effected. This causes a subroutine to be executed that automatically generates the desired list of sequences by walking along the target sequence. At that point, the user proceeds to process 400 to assign chemistries to the selected oligonucleotides.

Example 18 below, details a gene walk. In subsequent steps, this new set of nucleobase sequences generated by the gene walk is used to direct the automated synthesis at general procedure step 500 of a second set of candidate oligonucleotides. These compounds are then taken through subsequent process steps to yield active compounds or reiterated as necessary to optimize activity of the compounds.

20. Optimization of Lead Compounds by Chemistry.

Another means by which oligonucleotide compounds of the invention are optimized is by reiterating portions of the process of the invention using marginal or active compounds from the first iteration and selecting additional chemistries to the nucleobase sequences thereof.

Thus, for example, an oligonucleotide chemistry different from that of the first set of oligonucleotides is assigned at general procedure step 400. The nucleobase sequences of marginal compounds are used to direct the synthesis at general procedure step 500 of a second set of oligonucleotides having the second assigned chemistry. The resulting second set of oligonucleotide compounds is assayed in the same manner as the first set at procedure process step 700 and the results are examined to determine if compounds having sufficient activity have been generated at decision step 800.

21. Identification of Sites Amenable to Antisense Technologies.

In a related process, a second oligonucleotide chemistry is assigned at procedure step 400 to the nucleobase sequences of all of the oligonucleotides (or, at least, all of the active and marginal compounds) and a second set of oligonucleotides is synthesized at procedure step 500 having the same nucleobase sequences as the first set of compounds. The resulting second set of oligonucleotide compounds is assayed in the same manner as the first set at procedure step 700 and active and marginal compounds are identified at procedure steps 800 and 1000.

In order to identify sites on the target nucleic acid that are amenable to a variety of antisense technologies, the following mathematically simple steps are taken. The sequences of active and marginal compounds from two or more such automated syntheses/assays are compared and a set of nucleobase sequences that are active, or marginally so, in both sets of compounds is identified. The reverse complements of these nucleobase sequences corresponds to sequences of the target nucleic acid that are tractable to a variety of antisense and other sequence-based technologies. These antisense-sensitive sites are assembled into contiguous sequences (contigs) using the procedures described for assembling target nucleotide sequences (at procedure step 200).

22. Systems for Executing Preferred Methods of the Invention.

An embodiment of computer, network and instrument resources for effecting the methods of the invention is shown in FIG. 18. In this embodiment, four computer servers are provided. First, a large database server 2002 stores all chemical structure, sample tracking and genomic, assay, quality control, and program status data. Further, this database server serves as the platform for a document management system. Second, a compute engine 2004 runs computational programs including RNA folding, oligonucleotide walking, and genomic searching. Third, a file server 2006 allows raw instrument output storage and sharing of robot instructions. Fourth, a groupware server 2008 enhances staff communication and process scheduling.

A redundant high-speed network system is provided between the main servers and the bridges 2026, 2028 and 2030. These bridges provide reliable network access to the many workstations and instruments deployed for this process. The instruments selected to support this embodiment are all designed to sample directly from standard 96 well microtiter plates, and include an optical density reader 2016, a combined liquid chromatography and mass spectroscopy instrument 2018, a gel fluorescence and scintillation imaging system 2032 and 2042, a capillary gel electrophoreses system 2022 and a real-time PCR system 2034.

Most liquid handling is accomplished automatically using robots with individually controllable robotic pipetters 2038 and 2020 as well as a 96-well pipette system 2040 for duplicating plates. Windows NT or Macintosh workstations 2044, 2024, and 2036 are deployed for instrument control, analysis and productivity support.

23. Relational Database.

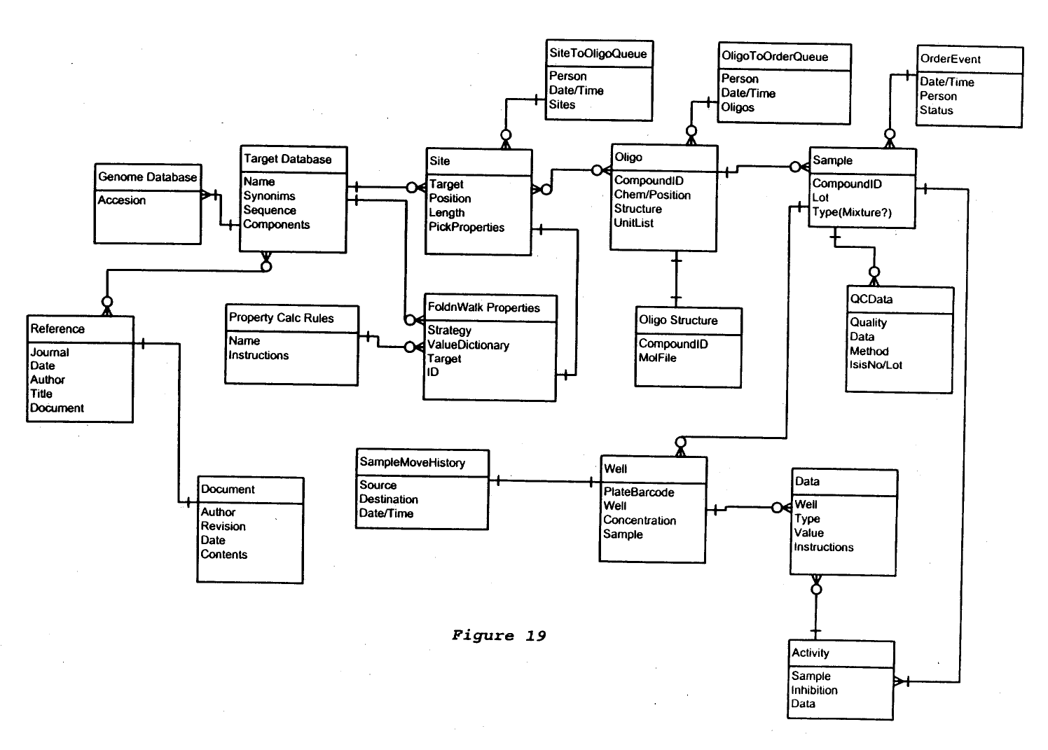

Data is stored in an appropriate database. For use with the methods of the invention, a relational database is preferred. FIG. 19 illustrates the data structure of a sample relational database. Various elements of data are segregated among linked storage elements of the database.

### EXAMPLES

The following examples illustrate the invention and are not intended to limit the same. Those skilled in the art will recognize, or be able to ascertain through routine experimentation, numerous equivalents to the specific procedures, materials and devices described herein. Such equivalents are considered to be within the scope of the present invention.

### Example 1

Selection of CD40 as a Target

Cell-cell interactions are a feature of a variety of biological processes. In the activation of the immune response, for example, one of the earliest detectable events in a normal inflammatory response is adhesion of leukocytes to the vascular endothelium, followed by migration of leukocytes out of the vasculature to the site of infection or injury. The adhesion of leukocytes to vascular endothelium is an obligate step in their migration out of the vasculature (for a review, see Albelda et al., FASEB J., 1994, 8, 504). As is well known in the art, cell-cell interactions are also critical for propagation of both B-lymphocytes and T-lymphocytes resulting in enhanced humoral and cellular immune responses, respectively (for a reviews, see Makgoba et al., Immunol. Today, 1989, 10, 417; Janeway, Sci. Amer., 1993, 269, 72).

CD40 was first characterized as a receptor expressed on B-lymphocytes. It was later found that engagement of B-cell CD40 with CD40L expressed on activated T-cells is essential for T-cell dependent B-cell activation (i.e. proliferation, immunoglobulin secretion, and class switching) (for a review, see Gruss et al. Leuk. Lymphoma, 1997, 24, 393). A full cDNA sequence for CD40 is available (GenBank accession number X60592, incorporated herein by reference as SEQ ID NO: 85).

As interest in CD40 mounted, it was subsequently revealed that functional CD40 is expressed on a variety of cell types other than B-cells, including macrophages, dendritic cells, thymic epithelial cells, Langerhans cells, and endothelial cells (Ibid.). These studies have led to the current belief that CD40 plays a much broader role in immune regulation by mediating interactions of T-cells with cell types other than B-cells. In support of this notion, it has been shown that stimulation of CD40 in macrophages and dendritic results is required for T-cell activation during antigen presentation (Id.). Recent evidence points to a role for CD40 in tissue inflammation as well. Production of the inflammatory mediators IL-12 and nitric oxide by macrophages has been shown to be CD40 dependent (Buhlmann et al., J. Clin. Immunol., 1996, 16, 83). In endothelial cells, stimulation of CD40 by CD40L has been found to induce surface expression of E-selectin, ICAM-1, and VCAM-1, promoting adhesion of leukocytes to sites of inflammation (Buhlmann et al., J. Clin. Immunol, 1996, 16, 83; Gruss et al., Leuk Lymphoma, 1997, 24, 393). Finally, a number of reports have documented overexpression of CD40 in epithelial and hematopoietic tumors as well as tumor infiltrating endothelial cells, indicating that CD40 may play a role in tumor growth and/or angiogenesis as well (Gruss et al., Leuk Lymphoma, 1997, 24, 393-422; Kluth et al. Cancer Res, 1997, 57, 891).

Due to the pivotal role that CD40 plays in humoral immunity, the potential exists that therapeutic strategies aimed at downregulating CD40 may provide a novel class of agents useful in treating a number of immune associated disorders, including but not limited to graft versus host disease, graft rejection, and autoimmune diseases such as multiple sclerosis, systemic lupus erythematosus, and certain forms of arthritis. Inhibitors of CD40 may also prove useful as an anti-inflammatory compound, and could therefore be useful as treatment for a variety of diseases with an inflammatory component such as asthma, rheumatoid arthritis, allograft rejections, inflammatory bowel disease, and various dermatological conditions, including psoriasis. Finally, as more is learned about the association between CD40 overexpression and tumor growth, inhibitors of CD40 may prove useful as anti-tumor agents as well.

Currently, there are no known therapeutic agents which effectively inhibit the synthesis of CD40. To date, strategies aimed at inhibiting CD40 function have involved the use of a variety of agents that disrupt CD40/CD40L binding. These include monoclonal antibodies directed against either CD40 or CD40L, soluble forms of CD40, and synthetic peptides derived from a second CD40 binding protein, A20. The use of neutralizing antibodies against CD40 and/or CD40L in animal models has provided evidence that inhibition of CD40 stimulation would have therapeutic benefit for GVHD, allograft rejection, rheumatoid arthritis, SLE, MS, and B-cell lymphoma (Buhlmann et al., J. Clin. Immunol, 1996, 16, 83). However, due to the expense, short half-life, and bioavailability problems associated with the use of large proteins as therapeutic agents, there is a long felt need for additional agents capable of effectively inhibiting CD40 function. Oligonucleotides compounds avoid many of the pitfalls of current agents used to block CD40/CD40L interactions and may therefore prove to be uniquely useful in a number of therapeutic applications.

### Example 2

Generation of Virtual Oligonucleotides Targeted to CD40

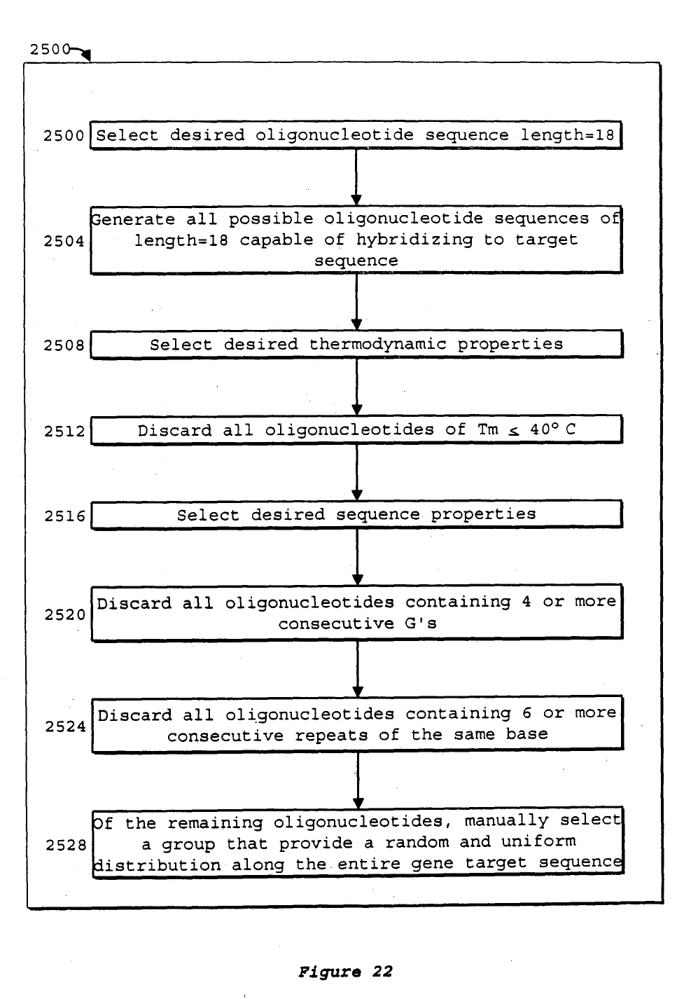

The process of the invention was used to select oligonucleotides targeted to CD40, generating the list of oligonucleotide sequences with desired properties as shown in FIG. 22. From the assembled CD40 sequence, the process began with determining the desired oligonucleotide length to be eighteen nucleotides, as represented in step 2500. All possible oligonucleotides of this length were generated by Oligo 5.0™, as represented in step 2504. Desired thermodynamic properties were selected in step 2508. The single parameter used was oligonucleotides of melting temperature less than or equal to 40° C. were discarded. In step 2512, oligonucleotide melting temperatures were calculated by Oligo 5.0™. Oligonucleotide sequences possessing an undesirable score were discarded. It is believed that oligonucleotides with melting temperatures near or below physiological and cell culture temperatures will bind poorly to target sequences. All oligonucleotide sequences remaining were exported into a spreadsheet. In step 2516, desired sequence properties are selected. These include discarding oligonucleotides with at least one stretch of four guanosines in a row and stretches of six of any other nucleotide in a row. In step 2520, a spreadsheet macro removed all oligonucleotides containing the text string “GGGG.” In step 2524, another spreadsheet macro removed all oligonucleotides containing the text strings “AAAAAA” or “CCCCCC” or “TTTTTT.” From the remaining oligonucleotide sequences, 84 sequences were selected manually with the criteria of having an uniform distribution of oligonucleotide sequences throughout the target sequence, as represented in step 2528. These oligonucleotide sequences were then passed to the next step in the process, assigning actual oligonucleotide chemistries to the sequences.

### Example 3

Input Files For Automated Oligonucleotide Synthesis Command File (.cmd File)

Table 2 is a command file for synthesis of an oligonucleotide having regions of 2′-O-(2-methoxyethyl) nucleosides and a central region of 2′-deoxy nucleosides each linked by phosphorothioate internucleotide linkages.

TABLE 2

SOLID_SUPPORT_SKIP

BEGIN

Next_Sequence

END

INITIAL-WASH

BEGIN

Add ACN 300
Drain 10

END

LOOP-BEGIN
DEBLOCK

BEGIN

Prime TCA
Load Tray
Repeat 2

Add TCA 150
Wait 10
Drain 8

End_Repeat
Remove Tray

Add TCA 125
Wait 10
Drain 8

END

WASH_AFTER_DEBLOCK

BEGIN

Repeat 3

Add ACN 250 To_All
Drain 10

End_Repeat

END

COUPLING

BEGIN

if class = DEOXY_THIOATE

Nozzle wash <act1>
prime <act1>
prime <seq>
Add <act1> 70 + <seq> 70

Wait 40

Drain 5

end-if

if class = MOE_THIOATE

Nozzle wash <act1>

Prime <act1>
prime <seq>
Add <act1> 120 + <seq> 120
Wait 230
Drain 5

End_if

END

WASH_AFTER_COUPLING

BEGIN

Add ACN 200 To_All
Drain 10

END

OXIDIZE

BEGIN

if class = DEOXY_THIOATE

Add BEAU 180
Wait 40

Drain 7

end_if
if class = MOE_THIOATE
Add BEAU 200

Wait 120
Drain 7

end_if

END

CAP

BEGIN

Add CAP_B 80 + CAP_A 80
Wait 20
Drain 7

END

WASH_AFTER_CAP

BEGIN

Add ACN 150 To_All
Drain 5
Add ACN 250 To_All
Drain 11

END

BASE_COUNTER

BEGIN

Next_Sequence

END

LOOP_END
DEBLOCK_FINAL

BEGIN

Prime TCA
Load Tray
Repeat 2

Add TCA 150 To_All
Wait 10
Drain 8

End_Repeat
Remove Tray
Add TCA 125 To_All
Wait 10
Drain 10

END

FINAL_WASH

BEGIN

Repeat 4

Add ACN 300 to_All
Drain_12

End_Repeat

END

ENDALL

BEGIN

Wait 3

END

Sequence Files (.seq Files)

Table 3 is a .seq file for oligonucleotides having 2′-deoxy nucleosides linked by phosphorothioate internucleotide linkages.

TABLE 3

Identity of columns:
Syn #, Well, Scale, Nucleotide at particular
position (identified using base identifier
followed by backbone identifier where “s” is
phosphorothioate). Note the columns wrap around
to next line when longer than one line.

| | | | | | | | | | | | | | |
|---|---|---|---|---|---|---|---|---|---|---|---|---|---|
| 1 | A01 | 200 | As | Cs | Cs | As | Gs | Gs | As | Cs | Gs | Gs | Cs |
| 2 | A02 | 200 | As | Cs | Gs | Gs | Cs | Gs | Gs | As | Cs | Cs | As |
| 3 | A03 | 200 | As | Cs | Cs | As | As | Gs | Cs | As | Gs | As | Cs |
| 4 | A04 | 200 | As | Gs | Gs | As | Gs | As | Cs | Cs | Cs | Cs | Gs |
| 5 | A05 | 200 | As | Cs | Cs | Cs | Cs | Gs | As | Cs | Gs | As | As |
| 6 | A06 | 200 | As | Cs | Gs | As | As | Cs | Gs | As | Cs | Ts | Gs |
| 7 | A07 | 200 | As | Cs | Gs | As | Cs | Ts | Gs | Gs | Cs | Gs | As |
| 8 | A08 | 200 | As | Cs | As | Gs | Gs | Ts | As | Gs | Gs | Ts | Cs |
| 9 | A09 | 200 | As | Gs | Gs | Ts | Cs | Ts | Ts | Gs | Gs | Ts | Gs |
| 10 | A10 | 200 | As | Cs | Ts | Cs | As | Cs | Cs | As | Cs | As | As |
| 11 | A11 | 200 | As | Cs | Gs | As | Cs | As | As | Gs | As | As | As |
| 12 | A12 | 200 | As | Cs | As | As | As | Cs | As | Cs | Gs | Gs | Ts |
| 13 | B01 | 200 | As | As | Cs | As | Cs | Gs | Gs | Ts | Cs | Gs | Gs |
| 14 | B02 | 200 | As | Cs | Ts | Cs | As | Cs | Ts | Gs | As | Cs | Gs |
| 15 | B03 | 200 | As | Cs | Gs | Gs | As | As | Gs | Gs | As | As | Cs |
| 16 | B04 | 200 | As | Ts | Cs | Ts | Gs | Ts | Gs | Gs | As | Cs | Cs |
| 17 | B05 | 200 | As | Cs | As | Cs | Ts | Ts | Cs | Ts | Ts | Cs | Cs |
| 18 | B06 | 200 | As | Cs | Ts | Cs | Ts | Cs | Gs | As | Cs | As | Cs |
| 19 | B07 | 200 | As | As | As | Cs | Cs | Cs | Cs | As | Gs | Ts | Ts |
| 20 | B08 | 200 | As | Ts | Gs | Ts | Cs | Cs | Cs | Cs | As | As | As |
| 21 | B09 | 200 | As | Cs | Gs | Cs | Ts | Cs | Gs | Gs | Gs | As | Cs |
| 22 | B10 | 200 | As | Gs | Cs | Cs | Gs | As | As | Gs | As | As | Gs |
| 23 | B11 | 200 | As | Cs | As | Cs | As | Gs | Ts | As | Gs | As | Cs |
| 24 | B12 | 200 | As | Cs | As | Cs | Ts | Cs | Ts | Gs | Gs | Ts | Ts |
| 25 | C01 | 200 | As | Cs | Gs | As | Cs | Cs | As | Gs | As | As | As |
| 26 | C02 | 200 | As | Gs | Ts | Ts | As | As | As | As | Gs | Gs | Gs |
| 27 | C03 | 200 | As | Gs | Gs | Ts | Ts | Gs | Ts | Gs | As | Cs | Gs |
| 28 | C04 | 200 | As | As | Ts | Gs | Ts | As | Cs | Cs | Ts | As | Cs |
| 29 | C05 | 200 | As | Gs | Ts | Cs | As | Cs | Gs | Ts | Cs | Cs | Ts |
| 30 | C06 | 200 | Cs | Ts | Gs | Gs | Cs | Gs | As | Cs | As | Gs | Gs |
| 31 | C07 | 200 | Cs | Ts | Cs | Ts | Gs | Ts | Gs | Ts | Gs | As | Cs |
| 32 | C08 | 200 | Cs | As | Gs | Gs | Ts | Cs | Gs | Ts | Cs | Ts | Ts |
| 33 | C09 | 200 | Cs | Ts | Gs | Ts | Gs | Gs | Ts | As | Gs | As | Cs |
| 34 | C10 | 200 | Cs | Ts | As | As | Cs | Gs | As | Ts | Gs | Ts | Cs |
| 35 | C11 | 200 | Cs | Ts | Gs | Ts | Ts | Cs | Gs | As | Cs | As | Cs |
| 36 | C12 | 200 | Cs | Ts | Gs | Gs | As | Cs | Cs | As | As | Cs | As |
| 37 | D01 | 200 | Cs | Cs | Gs | Ts | Cs | Cs | Gs | Ts | Gs | Ts | Ts |
| 38 | D02 | 200 | Cs | Ts | Gs | As | Cs | Ts | As | Cs | As | As | Cs |
| 39 | D03 | 200 | Cs | As | As | Cs | As | Gs | As | Cs | As | Cs | Cs |
| 40 | D04 | 200 | Cs | As | Gs | Gs | Gs | Gs | Ts | Cs | Cs | Ts | As |
| 41 | D05 | 200 | Cs | Ts | Cs | Ts | As | Gs | Ts | Ts | As | As | As |
| 42 | D06 | 200 | Cs | Ts | Gs | Cs | Ts | As | Gs | As | As | Gs | Gs |
| 43 | D07 | 200 | Cs | Ts | Gs | As | As | As | Ts | Gs | Ts | As | Cs |
| 44 | D08 | 200 | Cs | As | Cs | Cs | Cs | Gs | Ts | Ts | Ts | Gs | Ts |
| 45 | D09 | 200 | Cs | Ts | Cs | Gs | As | Ts | As | Cs | Gs | Gs | Gs |
| 46 | D10 | 200 | Gs | Gs | Ts | As | Gs | Gs | Ts | Cs | Ts | Ts | Gs |
| 47 | D11 | 200 | Gs | As | Cs | Ts | Ts | Ts | Gs | Cs | Cs | Ts | Ts |
| 48 | D12 | 200 | Gs | Ts | Gs | Gs | As | Gs | Ts | Cs | Ts | Ts | Ts |
| 49 | E01 | 200 | Gs | Gs | As | Gs | Ts | Cs | Ts | Ts | Ts | Gs | Ts |
| 50 | E02 | 200 | Gs | Gs | As | Cs | As | Cs | Ts | Cs | Ts | Cs | Gs |
| 51 | E03 | 200 | Gs | As | Cs | As | Cs | As | Gs | Gs | As | Cs | Gs |
| 52 | E04 | 200 | Gs | As | Gs | Ts | As | Cs | Gs | As | Gs | Cs | Gs |
| 53 | E05 | 200 | Gs | As | Cs | Ts | As | Ts | Gs | Gs | Ts | As | Gs |
| 54 | E06 | 200 | Gs | As | As | Gs | As | Gs | Gs | Ts | Ts | As | Cs |
| 55 | E07 | 200 | Gs | As | Gs | Gs | Ts | Ts | As | Cs | As | Cs | As |
| 56 | E08 | 200 | Gs | Ts | Ts | Gs | Ts | Cs | Cs | Gs | Ts | Cs | Cs |
| 57 | E09 | 200 | Gs | As | Cs | Ts | Cs | Ts | Cs | Gs | Gs | Gs | As |
| 58 | E10 | 200 | Gs | Ts | As | Gs | Gs | As | Gs | As | As | Cs | Cs |
| 59 | E11 | 200 | Gs | Gs | Ts | Ts | Cs | Ts | Ts | Cs | Gs | Gs | Ts |
| 60 | E12 | 200 | Gs | Ts | Gs | Gs | Gs | Gs | Ts | Ts | Cs | Gs | Ts |
| 61 | F01 | 200 | Gs | Ts | Cs | As | Cs | Gs | Ts | Cs | Cs | Ts | Cs |
| 62 | F02 | 200 | Gs | Ts | Cs | Cs | Ts | Cs | Cs | Ts | As | Cs | Cs |
| 63 | F03 | 200 | Gs | Ts | Cs | Cs | Cs | Cs | As | Cs | Gs | Ts | Cs |
| 64 | F04 | 200 | Ts | Cs | As | Cs | Cs | As | Gs | Cs | As | Gs | Cs |
| 65 | F05 | 200 | Ts | As | Cs | Cs | As | As | Gs | Cs | As | Gs | As |
| 66 | F06 | 200 | Ts | Cs | Cs | Ts | Gs | Ts | Cs | Ts | Ts | Ts | Gs |
| 67 | F07 | 200 | Ts | Gs | Ts | Cs | Ts | Ts | Ts | Gs | As | Cs | Cs |
| 68 | F08 | 200 | Ts | Gs | As | Cs | Cs | As | Cs | Ts | Cs | As | Cs |
| 69 | F09 | 200 | Ts | Gs | As | Cs | Gs | Ts | Gs | Ts | Cs | Ts | Cs |
| 70 | F10 | 200 | Ts | Cs | As | As | Gs | Ts | Gs | As | Cs | Ts | Ts |
| 71 | F11 | 200 | Ts | Gs | Ts | Ts | Ts | As | Ts | Gs | As | Cs | Gs |
| 72 | F12 | 200 | Ts | Ts | As | Ts | Gs | As | Cs | Gs | Cs | Ts | Gs |
| 73 | G01 | 200 | Ts | Gs | As | Cs | Gs | Cs | Ts | Gs | Gs | Gs | Gs |
| 74 | G02 | 200 | Ts | Cs | Gs | Ts | Cs | Ts | Ts | Cs | Cs | Cs | Gs |
| 75 | G03 | 200 | Ts | Gs | Gs | Ts | As | Gs | As | Cs | Gs | Ts | Gs |
| 76 | G04 | 200 | Ts | Ts | Cs | Ts | Ts | Cs | Cs | Gs | As | Cs | Cs |
| 77 | G05 | 200 | Ts | Gs | Gs | Ts | As | Gs | As | Cs | Gs | Cs | Ts |
| 78 | G06 | 200 | Ts | As | Gs | As | Cs | Gs | Cs | Ts | Cs | Gs | Gs |
| 79 | G07 | 200 | Ts | Ts | Ts | Ts | As | Cs | As | Gs | Ts | Gs | Gs |
| 80 | G08 | 200 | Ts | Gs | Gs | Gs | As | As | Cs | Cs | Ts | Gs | Ts |
| 81 | G09 | 200 | Ts | Cs | Gs | Gs | Gs | As | Cs | Cs | As | Cs | Cs |
| 82 | G10 | 200 | Ts | As | Gs | Gs | As | Cs | As | As | As | Cs | Gs |
| 83 | G11 | 200 | Ts | Gs | Cs | Ts | As | Gs | As | As | Gs | Gs | As |
| 84 | G12 | 200 | Ts | Cs | Ts | Gs | Ts | Cs | As | Cs | Ts | Cs | Cs |

Table 4 is a .seq file for oligonucleotides having regions of 2′-O-(2-methoxyethyl)nucleosides and a central region of 2′-deoxy nucleosides each linked by phosphorothioate internucleotide linkages.

TABLE 4

Identity of columns:
Syn #, Well, Scale, Nucleotide at particular position
(identified using base identifier followed by backbone
identifier where “s” is phosphorothioate and “moe”
indicated a 2′-O-(2-methoxyethyl) substituted nucleoside).
The columns wrap around to next line
when longer than one line.

```
  1 A01 200 moeAs moeCs moeCs moeAs Gs Gs As Cs Gs Gs Cs Gs Gs As
moeCs moeCs moeAs moeG
  2 A02 200 moeAs moeCs moeCs moeGs Cs Gs Gs As Cs Cs As Gs As Gs
moeTs moeGs moeGs moeA
  3 A03 200 moeAs moeCs moeCs moeAs As Gs Cs As Gs As Cs Gs Gs As
moeGs moeAs moeCs moeG
  4 A04 200 moeAs moeGs moeGs moeAs Gs As Cs Cs Cs Cs Gs As Cs Gs
moeAs moeAs moeCs moeG
  5 A05 200 moeAs moeCs moeCs moeCs Cs Gs As Cs Gs As As Cs Gs As
moeCs moeTs moeGs moeG
  6 A06 200 moeAs moeCs moeGs moeAs As Cs Gs As Cs Ts Gs Gs Cs Gs
moeAs moeCs moeAs moeG
  7 A07 200 moeAs moeCs moeGs moeAs Cs Ts Gs Gs Cs Gs As Cs As Gs
moeGs moeTs moeAs moeG
  8 A08 200 moeAs moeCs moeAs moeGs Gs Ts As Gs Gs Ts Cs Ts Ts Gs
moeGs moeTs moeGs moeG
  9 A09 200 moeAs moeGs moeGs moels Cs Ts Ts Gs Gs Ts Gs Gs Gs Ts
moeGs moeAs moeCs moeG
10 A10 200 moeAs moeGs moeTs moeCs As Cs Gs As Cs As As Gs As As
moeAs moeCs moeAs moeC
11 A11 200 moeAs moeCs moeGs moeAs Cs As As Gs As As As Cs As Cs
moeGs moeGs moeTs moeC
12 A12 200 moeAs moeGs moeAs moeAs As Cs As Cs Gs Gs Ts Cs Gs Gs
moeTs moeCs moeCs moeT
13 B01 200 moeAs moeAs moeCs moeAs Cs Gs Gs Ts Cs Gs Gs Ts Cs Cs
moeTs moeGs moeTs moeC
14 B02 200 moeAs moeCs moeTs moeCs As Cs Ts Gs As Cs Gs Ts Gs Ts
moeCs moeTs moeCs moeA
15 B03 200 mocAs moeCs moeGs moeGs As As Gs Gs As As Cs Gs Cs Cs
moeAs moeCs moeTs moeT
16 B04 200 moeAs moeTs moeCs moeTs Gs Ts Gs Gs As Cs Cs Ts Ts Gs
moeTs moeCs moeTs moeC
17 B05 200 moeAs moeCs moeAs moeCs Ts Ts Cs Ts Ts Cs Cs Gs As Cs
moeCs moeGs moeTs moeG
18 B06 200 moeAs moeCs moeTs moeCs Ts Cs Gs As Cs As Cs As Gs Gs
moeAs moeCs moeGs moeT
19 B07 200 moeAs moeAs moeAs moeCs Cs Cs Cs As Gs Ts Ts Cs Gs Ts
moeCs moeTs moeAs moeA
20 B08 200 moeAs moeTs moeGs moeTs Cs Cs Cs Cs As As As Gs As Cs
moeTs moeAs moeTs moeG
21 B09 200 moeAs moeCs moeGs moeCs Ts Cs Gs Gs Gs As Cs Gs Gs Gs
moeTs moeCs moeAs moeG
22 B10 200 moeAs moeGs moeCs moeCs Gs As As Gs As As Gs As Gs Gs
moeTs moeTs moeAs moeC
23 B11 200 moeAs moeCs moeAs moeCs As Gs Ts As Gs As Cs Gs As As
moeAs moeGs moeCs moeT
24 B12 200 moeAs moeCs moeAs moeCs Ts Cs Ts Gs Gs Ts Ts Ts Cs Ts
moeGs moeGs moeAs moeC
25 C01 200 moeAs moeCs moeGs moeAs Cs Cs As Hs As As As Ts As Gs
moeTs moeTs moeTs moeT
26 C02 200 moeAs moeGs moeTs moeTs As As As As Gs Gs Gs Cs Ts Gs
moeCs moeTs moeAs moeG
27 C03 200 moeAs moeGs moeGs moeTs Ts Gs Ts Gs As Cs Gs As Cs Gs
moeAs moeGs moeGs moeT
28 C04 200 moeAs moeAs moeTs moeGs Ts As Cs Cs Ts As Cs Gs Gs Ts
moeTs moeGs moeGs moeC
29 CO5 200 moeAs moeGs moeTs moeCs As Cs Gs Ts Cs Cs Ts Cs Ts Cs
moeTs moeGs moeTs moeC
30 C06 200 moeCs moeTs moeGs moeGs Cs Gs As Cs As Gs Gs Ts As Gs
moeGs moeTs moeCs moeT
31 C07 200 moeCs moeTs moeGs moeTs Gs Ts Gs Ts Gs As Cs Gs Gs Ts
moeGs moeGs moeTs moeC
32 C08 200 moeCs moeAs moeGs moeGs Ts Cs Gs Ts Cs Ts Ts Cs Cs Cs
moeGs moeTs moeGs moeG
33 C09 200 moeCs moeTs moeGs moeTs Gs Gs Ts As Gs As Cs Gs Ts Gs
moeGs moeAs moeCs moeA
34 C10 200 moeCs moeTs moeAs moeAs Cs Gs As Ts Gs Ts Cs Cs Cs Cs
moeAs rnoeAs moeAs moeG
35 C11 200 moeCs moeTs moeGs moeTs Ts Cs Gs As Cs As Cs Ts Cs Ts
moeGs moeGs moeTs moeT
36 C12 200 moeCs moeTs moeGs moeGs As Cs Cs As As Cs As Cs Gs Ts
moeTs macGs moeTs moeC
37 D01 200 moeCs moeCs moeGs moeTs Cs Cs Gs Ts Gs Ts Ts Ts Gs Ts
moeTs moeCs moeTs moeG
38 D02 200 moeCs moeTs moeGs mocAs Cs Ts As Cs As As Cs As Gs As
moeCs moeAs moeCs moeC
39 D03 200 moeCs moeAs moeAs moeCs As Gs As Cs As Cs Cs As Gs Gs
moeGs moeGs moels moeC
40 D04 200 moeCs moeAs moeGs moeGs Gs Gs Ts Cs Cs Ts As Gs Cs Cs
moeGs moeAs moeCs moeT
41 D05 200 moeCs moeTs moeCs moeTs As Gs Ts Ts As As As As Gs Gs
moeGs moeCs moeTs moeG
42 D06 200 moeCs moeTs moeGs moeCs Ts As Gs As As Gs Gs As Cs Cs
moeGs moeAs moeGs moeG
43 D07 200 moeCs moeTs moeGs moeAs As As Ts Gs Ts As Cs Cs Ts As
moeCs moeGs moeGs moeT
44 D08 200 moeCs moeAs moeCs moeCs Cs Gs Ts Ts Ts Cs Ts Cs Cs Gs
moeTs moeCs moeAs moeA
45 D09 200 moeCs moeTs moeCs moeGs As Ts As Cs Gs Gs Gs Ts Cs As
moeGs moeTs moeCs moeA
46 D10 200 moeGs moeGs moeTs moeAs Gs Gs Ts Cs Ts Ts Gs Gs Ts Gs
moeGs moeGs moeTs moeG
47 D11 200 moeGs moeAs moeCs moeTs Ts Ts Gs Cs Cs Ts Ts As Cs Gs
moeGs moeAs moeAs moeG
48 D12 200 moeGs moeTs moeGs moeGs As Gs Ts Cs Ts Ts Ts Gs Ts Cs
moeTs moeGs moeTs moeG
49 E01 200 moeGs moeGs moeAs moeGs Ts Cs Ts Ts Ts Gs Ts Cs Ts Gs
moeTs moeGs moeGs moeT
50 E02 200 moeGs moeGs moeAs moeCs As Cs Ts Cs Ts Cs Gs As Cs As
moeCs moeAs moeGs moeG
51 E03 200 moeGs moeAs moeCs moeAs Cs As Gs Gs As Cs Gs Ts Gs Gs
moeCs moeGs moeAs moeG
52 E04 200 moeGs moeAs moeGs moeTs As Cs Gs As Gs Cs Gs Gs Gs Cs
moeCs moeGs moeAs moeA
53 E05 200 moeGs moeAs moeCs moeTs As Ts Gs Gs Ts As Gs As Cs Gs
moeCs moeTs moeCs moeG
54 E06 200 moeGs moeAs moeAs moeGs As Gs Gs Ts Ts As Cs As Cs As
moeGs moeTs moeAs moeG
55 E07 200 moeGs moeAs moeGs moeGs Ts Ts As Cs As Cs As Gs Ts As
moeGs moeAs moeCs moeG
56 E08 200 moeGs moeTs moeTs moeGs Ts Cs Cs Gs Ts Cs Cs Gs Ts Gs
moeTs moels moeTs moeG
57 E09 200 moeGs moeAs moeCs moeTs Cs Ts Cs Gs Gs Gs As Cs Cs As
moeCs moeCs moeAs moeC
58 E10 200 moeGs moeTs moeAs moeGs Gs As Gs As As Cs Cs As Cs Gs
moeAs moeCs moeCs moeA
59 E11 200 moeGs moeGs moeTs moels Cs Ts Ts Cs Gs Gs Ts Ts Gs Gs
moeTs moeTs moeAs moeT
60 E12 200 moeGs moeTs moeGs moeGs Gs Gs Ts Ts Cs Gs Ts Cs Cs Ts
moeTs moeGs moeGs moeG
61 F01 200 moeGs moeTs moeCs moeAs Cs Gs Ts Cs Cs Ts Cs Ts Gs As
moeAs moeAs moeTs moeG
62 F02 200 moeGs moeTs moeCs moeCs Ts Cs Cs Ts As Cs Cs Gs Ts Ts
moeTs moeCs moeTs moeC
63 F03 200 moeGs moeTs moeCs moeCs Cs Cs As Cs Gs Ts Cs Cs Gs Ts
moeCs moeTs moeTs moeC
64 F04 200 moeTs moeCs moeAs moeCs Cs As Gs Gs As Cs Gs Gs Cs Gs
moeGs moeAs moeCs moeC
65 F05 200 moeTs mocAs moeCs moeCs As As Gs Cs As Gs As Cs Gs Gs
moeAs moeGs moeAs moeC
66 F06 200 moeTs moeCs moeCs moeTs Gs Ts Cs Ts Ts Ts Gs As Cs Cs
moeAs moeCs moeTs moeC
67 F07 200 moeTs moeGs moeTs moeCs Ts Ts Ts Gs As Cs Cs As Gs Ts
moeCs moeAs moeCs moeT
68 F08 200 moeTs moeGs moeAs moeCs Cs As Cs Ts Cs As Cs Ts Gs As
moeCs moeGs moeTs moeG
69 F09 200 moeTs moeGs moeAs moeCs Gs Ts Gs Ts Cs Ts Cs As As Gs
moeTs moeGs moeAs moeC
70 F10 200 moeTs moeCs moeAs moeAs Gs Ts Gs As Cs Ts Ts Ts Gs Cs
moeCs moeTs moeTs moeA
71 F11 200 moeTs moeGs moeTs moeTs Ts As Ts Cs As Cs Gs Cs Ts Gs
moeGs moeGs moeGs moeT
72 F12 200 moeTs moeTs moeAs moeTs Gs As Cs Gs Cs Ts Gs Gs Gs Gs
moeTs moeTs moeGs moeG
73 G01 200 moeTs moeGs moeAs moeCs Gs Cs Ts Gs Gs Gs Gs Ts Ts Gs
moeGs moeAs moeTs moeC
74 G02 200 moeTs moeCs moeGs moeTs Cs Ts Ts Cs Cs Cs Gs Ts Gs Gs
moeAs moeGs moeTs moeC
75 G03 200 moeTs moeGs moeGs moeTs As Gs As Cs Gs Ts Gs Gs As Cs
moeAs moeCs moeTs moeT
76 G04 200 moeTs moeTs moeCs moeTs Ts Cs Cs Gs As Cs Cs Gs Ts Gs
moeAs moeCs moeAs moeT
77 G05 200 moeTs moeCs moeCs moeTs As Gs As Cs Gs Cs Ts Cs Gs Gs
moeGs moeAs moeCs moeG
78 G06 200 moeTs moeAs moeGs moeAs Cs Gs Cs Ts Cs Gs Gs Gs As Cs
moeGs moeGs moeGs moeT
79 G07 200 moeTs moeTs moeTs moeTs As Cs As Gs Ts Gs Gs Gs As As
moeCs moeCs moeTs moeG
80 G08 200 moeTs moeGs moeGs moeGs As As Cs Cs Ts Gs Ts Ts Cs Gs
moeAs moeCs moeAs moeC
81 G09 200 moeTs moeCs moeGs moeGs Gs As Cs Cs As Cs Cs As Cs Ts
moeAs moeGs moeGs moeG
82 G10 200 moeTs moeAs moeGs moeGs As Cs As As As Cs Gs Gs Ts As
moeGs moeGs moeAs moeG
83 G11 200 moeTs moeGs moeCs moeTs As Gs As As Gs Gs As Cs Cs Gs
moeAs moeGs moeGs moeT
84 G12 200 moeTs moeCs moeTs moeGs Ts Cs As Cs Ts Cs Cs Gs As Cs
moeGs moeTs moeGs moeG
```

Reagent File (.tab File)

Table 5 is a .tab file for reagents necessary for synthesizing an oligonucleotides having both 2′-O-(2-methoxyethyl)nucleosides and 2′-deoxy nucleosides located therein.

TABLE 5
Identity of columns: GroupName, Bottle ID, ReagentName,
FlowRate, Concentration. Wherein reagent name is
identified using base identifier, “moe” indicated
a 2′-O-(2-methoxyethyl) substituted nucleoside
and “cpg” indicates a control pore glass solid support
medium. The columns wrap around to next line when longer
than one line.

SUPPORT

| BEGIN | | | | | |
|---|---|---|---|---|---|
| 0 | moeG | moeG | cpg | 100 | 1 |
| 0 | moe5meC | moe5meC | cpg | 100 | 1 |
| 0 | moeA | moeA | cpg | 100 | 1 |
| 0 | moeT | moeT | cpg | 100 | 1 |
| END | | | | | |

DEBLOCK

| | | | | |
|---|---|---|---|---|
| BEGIN | | | | |
| 70 | TCA | TCA | 100 | 1 |
| END | | | | |

WASH

| | | | | |
|---|---|---|---|---|
| BEGIN | | | | |
| 65 | ACN | ACN | 190 | 1 |
| END | | | | |

OXIDIZERS

| | | | | |
|---|---|---|---|---|
| BEGIN | | | | |
| 68 | BEAU | BEAUCAGE | 320 | 1 |
| END | | | | |

CAPPING

| BEGIN | | | |
|---|---|---|---|
| 66 | CAP_BCAP_B | 220 | 1 |

| | | | | |
|---|---|---|---|---|
| 67 | CAP_A | CAP_A | 230 | 1 |
| END | | | | |

DEOXY THIOATE

| BEGIN | | | | |
|---|---|---|---|---|
| 31,32 | Gs | deoxyG | 270 | 1 |

| | | | |
|---|---|---|---|
| 39,40 | 5meCs 5methyldeoxyC | 270 | 1 |

| | | | | |
|---|---|---|---|---|
| 37,38 | As | deoxyA | 270 | 1 |

| | | | | |
|---|---|---|---|---|
| 29,30 | Ts | deoxyT | 270 | 1 |
| END | | | | |

MOE-THIOATE

| BEGIN | | | |
|---|---|---|---|
| 15,16 | moeGs methoxyethoxyG | 240 | 1 |

| | | | | |
|---|---|---|---|---|
| 23,24 | moe5meCs | methoxyethoxyC | 240 | 1 |
| 21,22 | moeAs | methoxyethoxyA | 240 | 1 |
| 13,14 | moeTs | methoxyethoxyT | 240 | 1 |
| END | | | | |

ACTIVATORS

BEGIN
5,6,7,8 SET s-ethyl-tet 280

Activates
DEOXY_THIOATE
MOE_THIOATE

END

### Example 4

Synthesis of Nucleoside Phosphoramidites

The following compounds, including amidites and their intermediates were prepared as described in U.S. Pat. No. 6,426,220 and published PCT WO 02/36743; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5′-O-Dimethoxytrityl-2′-deoxy-N4-benzoyl-5-methylcytidine penultimate intermediate for 5-methyl dC amidite, [5′-O-(4,4′-Dimethoxytriphenylmethyl)-2′-deoxy-N⁴-benzoyl-5-methylcytidin-3′-O-yl]-2-cyanoethyl-N,N-diisopropylphosphoramidite (5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, [5′-O-(4,4′-Dimethoxytriphenylmethyl)-2′-O-(2-methoxyethyl)-5-methyluridin-3′-O-yl]-2-cyanoethyl-N,N-diisopropylphosphoramidite (MOE T amidite), 5′-O-Dimethoxytrityl-2′-O-(2-methoxyethyl)-5-methylcytidine intermediate, 5′-O-dimethoxytrityl-2′-O-(2-methoxyethyl)-N⁴-benzoyl-5-methyl-cytidine penultimate intermediate, [5′-O-(4,4′-Dimethoxytriphenylmethyl)-2′-O-(2-methoxyethyl)-N4-benzoyl-5-methylcytidin-3′-O-yl]-2-cyanoethyl-N,N-diisopropylphosphoramidite (MOE 5-Me-C amidite), [5′-O-(4,4′-Dimethoxytriphenylmethyl)-2′-O-(2-methoxyethyl)-N⁶-benzoyladenosin-3′-O-yl]-2-cyanoethyl-N,N-diisopropylphosphoramidite (MOE A amdite), [5′-O-(4,4′-Dimethoxytriphenylmethyl)-2′-O-(2-methoxyethyl)-N⁴-isobutyrylguanosin-3′-O-yl]-2-cyanoethyl-N,N-diisopropylphosphoramidite (MOE G amidite), 2′-O-(Aminooxyethyl) nucleoside amidites and 2′-O-(dimethylaminooxyethyl) nucleoside amidites, 2′-(Dimethylaminooxyethoxy) nucleoside amidites, 5′-O-tert-Butyldiphenylsilyl-O²-2′-anhydro-5-methyluridine, 5′-O-tert-Butyldiphenylsilyl-2′-O-(2-hydroxyethyl)-5-methyluridine, 2′-O-([2-phthalimidoxy)ethyl]-5′-t-butyldiphenylsilyl-5-methyluridine, 5′-O-tert-butyldiphenylsilyl-2′-O-[(2-formadoximinooxy)ethyl]-5-methyluridine, 5′-O-tert-Butyldiphenylsilyl-2′-O-[N,N dimethylaminooxyethyl]-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-[(2-cyanoethyl)-N,N-diisopropylphosphoramidite], 2′-(Aminooxyethoxy) nucleoside amidites, N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite], 2′-dimethylaminoethoxyethoxy (2′-DMAEOE) nucleoside amidites, 2′-O-[2(2-N,N-dimethylaminoethoxy)ethyl]-5-methyl uridine, 5′-O-dimethoxytrityl-2′-O-[2(2-N,N-dimethylaminoethoxy)-ethyl)]-5-methyl uridine and 5′-O-Dimethoxytrityl-2′-O-[2(2-N,N-dimethylaminoethoxy)-ethyl)]-5-methyl uridine-3′-O-(cyanoethyl-N,N-diisopropyl)phosphoramidite.

### Example 5

Oligonucleotide and Oligonucleoside Synthesis

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH4OAc solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3+-Deoxy-3′-methylene phosphonate oligonucleotides are prepared as described in U.S. Pat. Nos. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

Oligonucleosides: Methylenemethylimino linked oligonucleosides, also identified as MMI linked oligonucleosides, methylenedi-methyl-hydrazo linked oligonucleosides, also identified as MDH linked oligonucleosides, and methylenecarbonylamino linked oligonucleosides, also identified as amide-3 linked oligonucleosides, and methyleneaminocarbonyl linked oligo-nucleosides, also identified as amide-4 linked oligo-nucleo-sides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligo-nucleo-sides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 6

Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH4OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (±32±48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 7

Chimeric Oligonucleotide Synthesis

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers.”

A. [2′-O-Me]—[2′-deoxy]—[2′-O-Me] Chimeric Phosphorothioate Oligonucleotides

Chimeric oligonucleotides having 2′-O-alkyl phosphorothioate and 2′-deoxy phosphorothioate oligonucleotide segments are synthesized using 2′-deoxy-5′-dimethoxytrityl-3′-O-phosphoramidites for the DNA portion and 5′-dimethoxytrityl-2′-O-methyl-3′-O-phosphoramidites for 5′ and 3′ wings. The standard synthesis cycle is modified by increasing the wait step after the delivery of tetrazole and base to 600 s repeated four times for DNA and twice for 2′-O-methyl. The fully protected oligonucleotide was cleaved from the support and the phosphate group is deprotected in 3:1 Ammonia/Ethanol at room temperature overnight then lyophilized to dryness. Treatment in methanolic ammonia for 24 hrs at room temperature is done to deprotect all bases and the samples are again lyophilized to dryness.

B. [2′-O-(2-Methoxyethyl)]—[2′-deoxy]—[2′-O-(2-Methoxyethyl)] Chimeric Phosphorothioate Oligonucleotides

2′-O-(2-methoxyethyl)]—[2′-deoxy]—[-2′-O-(2-methoxyethyl)] chimeric phosphorothioate oligonucleotides are prepared as per the procedure above for the 2′-O-methyl chimeric oligonucleotide, with the substitution of 2′-O-(2-methoxyethyl) amidites for the 2′-O-methyl amidites.

C. [2′-O-(2-Methoxyethyl)Phosphodiester]—[2′-deoxy Phosphoro-thioate]—[2′-O-(2-Methoxyethyl) Phosphodiester] Chimeric Oligo-Nucleotide

2′-O-(2-methoxyethyl phosphodiester]—[2′-deoxy phosphorothioate]—[2′-O-(2-methoxyethyl) phosphodiester] chimeric oligonucleotides are prepared as per the above procedure for the 2′-O-methyl chimeric oligonucleotide with the substitution of 2′-O-(2-methoxyethyl) amidites for the 2′-O-methyl amidites in the wing portions. Sulfurization utilizing 3,H-1,2 benzodithiole-3-one 1,1 dioxide (Beaucage Reagent) is used to generate the phosphorothioate internucleotide linkages within the wing portions of the chimeric structures. Oxidization with iodine is used to generate the phosphodiester inter-nucleotide linkages for the center gap.

Other chimeric oligonucleotides, chimeric oligonucleosides and mixed chimeric oligonucleotides/oligonucleosides are synthesized according to U.S. Pat. No. 5,623,065, which is incorporated herein by reference in its entirety.

### Example 8

Oligonucleotide Analysis

Oligonucleotides are analyzed by mass spectrometry as follows: for a typical oligonucleotide, a 0.01 OD aliquot dissolved in 10 uL of water is mixed with 90 uL of a 1:1 mixture of acetonitrile and water containing 20 mM imidazole and 20 mM piperidine (Greig M. J., Griffey R. H.: Rapid Commun. Mass Spec., 9:97-102, 1995). The sample is transferred to a 96 or 384-well plate, and each well on the plate is sampled systematically using an Agilent 1100 liquid handler. Other types of robotic liquid handlers such as a Leap Pal or Gilson 215 could be used to introduce sample to the mass spectrometer under computer control. The sample is infused to an Agilent MSD VX quadrupole mass spectrometer at a rate of 3 uL/min. The electrospray ionization is produced with 60 psi of nitrogen gas and a 4 kV potential between the source needle and the inlet capillary. The capillary is heated to 250° C. to effect desolvation of the ions. Typically, a total of 32 accumulations are averaged over a mass/charge range of 500-1500 m/z. The resulting data is saved into a file containing the sample ID information, and deconvoluted using the Agilent algorithm to calculate the neutral masses and abundances of the oligonucleotides and associated impurities. In a preferred embodiment, the neutral masses and abundances of compounds present in the sample obtained from the ESI-MS spectrum are written to a relational database. A logical algorithm then compares the measured mass of the oligonucleotide to the mass calculated from the base sequence and expected chemical structure of the oligonucleotide stored in a relational database. If the calculated and observed masses for the most abundant species agree within ±1.5 Da, the oligonucleotide is deemed to have “passed”. If the measured mass of the most abundant species differs by more than 1.5 Da, or the integrated ion abundance is <50% of the sample, the oligonucleotide “fails” and a new synthesis is requested.

Oligonucleotides that pass the ESI-MS analysis are transferred to 96-well master plates for storage at 10 mM concentrations in aqueous solution using multichannel robotics, such as a Beckman FX or Packard MultiProbe. The plates are given sequential identifying bar codes and this information on sample location is stored for later retrieval in a relational database.

### Example 9

PNA Synthesis

Peptide nucleic acids (PNAs) are prepared in accordance with any of the various procedures referred to in Peptide Nucleic Acids (PNA): Synthesis, Properties and Potential Applications, Bioorganic & Medicinal Chemistry, 1996, 4, 5. They may also be prepared in accordance with U.S. Pat. Nos. 5,539,082; 5,700,922, and 5,719,262, each of which is incorporated herein by reference in its entirety.

### Example 10

RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′-hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand,. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

RNA antisense compounds (RNA oligonucleotides) of the present invention can be synthesized by the methods herein or purchased from Dharmacon Research, Inc (Lafayette, Colo.). Once synthesized, complementary RNA antisense compounds can then be annealed by methods known in the art to form double stranded (duplexed) antisense compounds. For example, duplexes can be formed by combining 30 μl of each of the complementary strands of RNA oligonucleotides (50 uM RNA oligonucleotide solution) and 15 μl of 5X annealing buffer (100 mM potassium acetate, 30 mM HEPES-KOH pH 7.4, 2 mM magnesium acetate) followed by heating for 1 minute at 90° C., then 1 hour at 37° C. The resulting duplexed antisense compounds can be used in kits, assays, screens, or other methods to investigate the role of a target nucleic acid.

### Example 11

Output Oligonucleotides from Automated Oligonucleotide Synthesis

Using the .seq files, the .cmd files and .tab file of Example 3, oligonucleotides were prepared as per the protocol of the 96 well format of Example 5. The oligonucleotides were prepared utilizing phosphorothioate chemistry to give in one instance a first library of phosphorothioate oligodeoxynucleotides. The oligonucleotides were prepared in a second instance as a second library of hybrid oligonucleotides having phosphorothioate backbones with a first and third “wing” region of 2′-O-(2-methoxyethyl)nucleotides on either side of a center gap region of 2′-deoxy nucleotides. The two libraries contained the same set of oligonucleotide sequences. Thus the two libraries are redundant with respect to sequence but are unique with respect to the combination of sequence and chemistry. Because the sequences of the second library of compounds is the same as the first (however the chemistry is different), for brevity sake, the second library is not shown.

For illustrative purposes Tables 6-a and 6-b show the sequences of an initial first library, i.e., a library of phosphorothioate oligonucleotides targeted to a CD40 target. The compounds of Table 6-a shows the members of this library listed in compliance with the established rule for listing SEQ ID NO:, i.e., in numerical SEQ ID NO: order.

TABLE 6-a

Sequences of Oligonucleotides
Targeted to CD40 by SEQ ID NO.:

| | |
|---|---|
| NUCLEOBASE SEQUENCE | SEQ ID NO. |

| | |
|---|---|
| CCAGGCGGCAGGACCACT | 1 |
| GACCAGGCGGCAGGACCA | 2 |
| AGGTGAGACCAGGCGGCA | 3 |
| CAGAGGCAGACGAACCAT | 4 |
| GCAGAGGCAGACGAACCA | 5 |
| GCAAGCAGCCCCAGAGGA | 6 |
| GGTCAGCAAGCAGCCCCA | 7 |
| GACAGCGGTCAGCAAGCA | 8 |
| GATGGACAGCGGTCAGCA | 9 |
| TCTGGATGGACAGCGGTC | 10 |
| GGTGGTTCTGGATGGACA | 11 |
| GTGGGTGGTTCTGGATGG | 12 |
| GCAGTGGGTGGTTCTGGA | 13 |
| CACAAAGAACAGCACTGA | 14 |
| CTGGCACAAAGAACAGCA | 15 |
| TCCTGGCTGGCACAAAGA | 16 |
| CTGTCCTGGCTGGCACAA | 17 |
| CTCACCAGTTTCTGTCCT | 18 |
| TCACTCACCAGTTTCTGT | 19 |
| GTGCAGTCACTCACCAGT | 20 |
| ACTCTGTGCAGTCACTCA | 21 |
| CAGTGAACTCTGTGCAGT | 22 |
| ATTCCGTTTCAGTGAACT | 23 |
| GAAGGCATTCCGTTTCAG | 24 |
| TTCACCGCAAGGAAGGCA | 25 |
| CTCTGTTCCAGGTGTCTA | 26 |
| CTGGTGGCAGTGTGTCTC | 27 |
| TGGGGTCGCAGTATTTGT | 28 |
| GGTTGGGGTCGCAGTATT | 29 |
| CTAGGTTGGGGTCGCAGT | 30 |
| GGTGCCCTTCTGCTGGAC | 31 |
| CTGAGGTGCCCTTCTGCT | 32 |
| GTGTCTGTTTCTGAGGTG | 33 |
| TGGTGTCTGTTTCTGAGG | 34 |
| ACAGGTGCAGATGGTGTC | 35 |
| TTCACAGGTGCAGATGGT | 36 |
| GTGCCAGCCTTCTTCACA | 37 |
| TACAGTGCCAGCCTTCTT | 38 |
| GGACACAGCTCTCACAGG | 39 |
| TGCAGGACACAGCTCTCA | 40 |
| GAGCGGTGCAGGACACAG | 41 |
| AAGCCGGGCGAGCATGAG | 42 |
| AATCTGCTTGACCCCAAA | 43 |
| GAAACCCCTGTAGCAATC | 44 |
| GTATCAGAAACCCCTGTA | 45 |
| GCTCGCAGATGGTATCAG | 46 |
| GCAGGGCTCGCAGATGGT | 47 |
| TGGGCAGGGCTCGCAGAT | 48 |
| GACTGGGCAGGGCTCGCA | 49 |
| CATTGGAGAAGAAGCCGA | 50 |
| GATGACACATTGGAGAAG | 51 |
| GCAGATGACACATTGGAG | 52 |
| TCGAAAGCAGATGACACA | 53 |
| GTCCAAGGGTGACATTTT | 54 |
| CACAGCTTGTCCAAGGGT | 55 |
| TTGGTCTCACAGCTTGTC | 56 |
| CAGGTCTTTGGTCTCACA | 57 |
| CTGTTGCACAACCAGGTC | 58 |
| GTTTGTGCCTGCCTGTTG | 59 |
| GTCTTGTTTGTGCCTGCC | 60 |
| CCACAGACAACATCAGTC | 61 |
| CTGGGGACCACAGACAAC | 62 |
| TCAGCCGATCCTGGGGAC | 63 |
| CACCACCAGGGCTCTCAG | 64 |
| GGGATCACCACCAGGGCT | 65 |
| GAGGATGGCAAACAGGAT | 66 |
| ACCAGCACCAAGAGGATG | 67 |
| TTTTGATAAAGACCAGCA | 68 |
| TATTGGTTGGCTTCTTGG | 69 |
| GGGTTCCTGCTTGGGGTG | 70 |
| GTCGGGAAAATTGATCTC | 71 |
| GATCGTCGGGAAAATTGA | 72 |
| GGAGCCAGGAAGATCGTC | 73 |
| TGGAGCCAGGAAGATCGT | 74 |
| TGGAGCAGCAGTGTTGGA | 75 |
| GTAAAGTCTCCTGCACTG | 76 |
| TGGCATCCATGTAAAGTC | 77 |
| CGGTTGGCATCCATGTAA | 78 |
| CTCTTTGCCATCCTCCTG | 79 |
| CTGTCTCTCCTGCACTGA | 80 |
| GGTGCAGCCTCACTGTCT | 81 |
| AACTGCCTGTTTGCCCAC | 82 |
| CTTCTGCCTGCACCCCTG | 83 |
| ACTGACTGGGCATAGCTC | 84 |

The sequences shown in Table 6-a, above, and Table 6-b, below, are in a 5′ to 3′ direction. This is reversed with respect to 3′ to 5′ direction shown in the .seq files of Example 3. For synthesis purposes, the .seq files are generated reading from 3′ to 5′. This allows for aligning all of the 3′ most “A” nucleosides together, all of the 3′ most “G” nucleosides together, all of the 3′ most “C” nucleosides together and all of the 3′ most “T” nucleosides together. Thus when the first nucleoside of each particular oligonucleotide (attached to the solid support) is added to the wells on the plates, machine movement is reduced since an automatic pipette can move in a linear manner down one row and up another on the 96 well plate.

The location of the well holding each particular oligonucleotides is indicated by row and column. There are eight rows designated A to G and twelve columns designated 1 to 12 in a typical 96 well format plate. Any particular well location is indicated by its “Well No.” which is indicated by the combination of the row and the column, e.g. A08 is the well at row A, column 8.

In Table 6-b below, the oligonucleotides of Table 6-a are shown reordered according to the Well No. on their synthesis plate. The order shown in Table 6-b is the actually order as synthesized on an automated synthesizer taking advantage of the preferred placement of the first nucleoside according to the above alignment criteria.

TABLE 6-b

Sequences of Oligonucleotides
Targeted to CD40 Order by Synthesis Well No.

| | |
|---|---|
| Well No. | SEQ ID NO: |

| | | |
|---|---|---|
| A01 | GACCAGGCGGCAGGACCA | 2 |
| A02 | AGGTGAGACCAGGCGGCA | 3 |
| A03 | GCAGAGGCAGACGAACCA | 5 |
| A04 | GCAAGCAGCCCCAGAGGA | 6 |
| A05 | GGTCAGCAAGCAGCCCCA | 7 |
| A06 | GACAGCGGTCAGCAAGCA | 8 |
| A07 | GATGGACAGCGGTCAGCA | 9 |
| A08 | GGTGGTTCTGGATGGACA | 11 |
| A09 | GCAGTGGGTGGTTCTGGA | 13 |
| A10 | CACAAAGAACAGCACTGA | 14 |
| A11 | CTGGCACAAAGAACAGCA | 15 |
| A12 | TCCTGGCTGGCACAAAGA | 16 |
| B01 | CTGTCCTGGCTGGCACAA | 17 |
| B02 | ACTCTGTGCAGTCACTCA | 21 |
| B03 | TTCACCGCAAGGAAGGCA | 25 |
| B04 | CTCTGTTCCAGGTGTCTA | 26 |
| B05 | GTGCCAGCCTTCTTCACA | 37 |
| B06 | TGCAGGACACAGCTCTCA | 40 |
| B07 | AATCTGCTTGACCCCAAA | 43 |
| B08 | GTATCAGAAACCCCTGTA | 45 |
| B09 | GACTGGGCAGGGCTCGCA | 49 |
| B10 | CATTGGAGAAGAAGCCGA | 50 |
| B11 | TCGAAAGCAGATGACACA | 53 |
| B12 | CAGGTCTTTGGTCTCACA | 57 |
| C01 | TTTTGATAAAGACCAGCA | 68 |
| C02 | GATCGTCGGGAAAATTGA | 72 |
| C03 | TGGAGCAGCAGTGTTGGA | 75 |
| C04 | CGGTTGGCATCCATGTAA | 78 |
| C05 | CTGTCTCTCCTGCACTGA | 80 |
| C06 | TCTGGATGGACAGCGGTC | 10 |
| C07 | CTGGTGGCAGTGTGTCTC | 27 |
| C08 | GGTGCCCTTCTGCTGGAC | 31 |
| C09 | ACAGGTGCAGATGGTGTC | 35 |
| C10 | GAAACCCCTGTAGCAATC | 44 |
| C11 | TTGGTCTCACAGCTTGTC | 56 |
| C12 | CTGTTGCACAACCAGGTC | 58 |
| D01 | GTCTTGTTTGTGCCTGCC | 60 |
| D02 | CCACAGACAACATCAGTC | 61 |
| D03 | CTGGGGACCACAGACAAC | 62 |
| D04 | TCAGCCGATCCTGGGGAC | 63 |
| D05 | GTCGGGAAAATTGATCTC | 71 |
| D06 | GGAGCCAGGAAGATCGTC | 73 |
| D07 | TGGCATCCATGTAAAGTC | 77 |
| D08 | AACTGCCTGTTTGCCCAC | 82 |
| D09 | ACTGACTGGGCATAGCTC | 84 |
| D10 | GTGGGTGGTTCTGGATGG | 12 |
| D11 | GAAGGCATTCCGTTTCAG | 24 |
| D12 | GTGTCTGTTTCTGAGGTG | 33 |
| E01 | TGGTGTCTGTTTCTGAGG | 34 |
| E02 | GGACACAGCTCTCACAGG | 39 |
| E03 | GAGCGGTGCAGGACACAG | 41 |
| E04 | AAGCCGGGCGAGCATGAG | 42 |
| E05 | GCTCGCAGATGGTATCAG | 46 |
| E06 | GATGACACATTGGAGAAG | 51 |
| E07 | GCAGATGACACATTGGAG | 52 |
| E08 | GTTTGTGCCTGCCTGTTG | 59 |
| E09 | CACCACCAGGGCTCTCAG | 64 |
| E10 | ACCAGCACCAAGAGGATG | 67 |
| E11 | TATTGGTTGGCTTCTTGG | 69 |
| E12 | GGGTTCCTGCTTGGGGTG | 70 |
| F01 | GTAAAGTCTCCTGCACTG | 76 |
| F02 | CTCTTTGCCATCCTCCTG | 79 |
| F03 | CTTCTGCCTGCACCCCTG | 83 |
| F04 | CCAGGCGGCAGGACCACT | 1 |
| F05 | CAGAGGCAGACGAACCAT | 4 |
| F06 | CTCACCAGTTTCTGTCCT | 18 |
| F07 | TCACTCACCAGTTTCTGT | 19 |
| F08 | GTGCAGTCACTCACCAGT | 20 |
| F09 | CAGTGAACTCTGTGCAGT | 22 |
| F10 | ATTCCGTTTCAGTGAACT | 23 |
| F11 | TGGGGTCGCAGTATTTGT | 28 |
| F12 | GGTTGGGGTCGCAGTATT | 29 |
| G01 | CTAGGTTGGGGTCGCAGT | 30 |
| G02 | CTGAGGTGCCCTTCTGCT | 32 |
| G03 | TTCACAGGTGCAGATGGT | 36 |
| G04 | TACAGTGCCAGCCTTCTT | 38 |
| G05 | GCAGGGCTCGCAGATGGT | 47 |
| G06 | TGGGCAGGGCTCGCAGAT | 48 |
| G07 | GTCCAAGGGTGACATTTT | 54 |
| G08 | CACAGCTTGTCCAAGGGT | 55 |
| G09 | GGGATCACCACCAGGGCT | 65 |
| G10 | GAGGATGGCAAACAGGAT | 66 |
| G11 | TGGAGCCAGGAAGATCGT | 74 |
| G12 | GGTGCAGCCTCACTGTCT | 81 |

### Example 12

Automated Assay of CD40 Oligonucleotide Activity

A. Poly(A)+ mRNA Isolation.

Poly(A)+ mRNA was isolated according to Miura et al. (Clin. Chem., 1996, 42, 1758). Briefly, for cells grown on 96-well plates, growth medium was removed from the cells and each well was washed with 200 μl cold PBS. 60 μi lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μl of lysate was transferred to Oligo d(T) coated 96 well plates (AGCT Inc., Irvine, Calif.). Plates were incubated for 60 minutes at room temperature, washed 3 times with 200 ml of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 ml of elution buffer (5 mM Tris-HCl pH 7.6), preheated to 70° C. was added to each well, the plate was incubated on a 90° C. plate for 5 minutes, and the eluate then transferred to a fresh 96-well plate. Cells grown on 100 mm or other standard plates may be treated similarly, using appropriate volumes of all solutions.

B. Total RNA Isolation

Total mRNA was isolated using an RNEASY 96™ kit and buffers purchased from Qiagen Inc. (Valencia Calif.) following the manufacturer's recommended procedures. Briefly, for cells grown on 96-well plates, growth medium was removed from the cells and each well was washed with 200 mL cold PBS. 100 mL Buffer RLT was added to each well and the plate vigorously agitated for 20 seconds. 100 mL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 15 seconds. 1 mL of Buffer RW1 was added to each well of the RNEASY 96™ plate and the vacuum again applied for 15 seconds. 1 mL of Buffer RPE was then added to each well of the RNEASY 96™ plate and the vacuum applied for a period of 15 seconds. The Buffer RPE wash was then repeated and the vacuum was applied for an additional 10 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 60 mL water into each well, incubating 1 minute, and then applying the vacuum for 30 seconds. The elution step was repeated with an additional 60 mL water.

C. RT-PCR Analysis of CD40 mRNA Levels

Quantitation of CD40 mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5× PCR buffer minus MgCl₂; 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5× ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

For human GAPDH the PCR primers were:

forward primer: GAAGGTGAAGGTCGGAGTC(SEQ ID NO: 89)

reverse primer: GAAGATGGTGATGGGATTTC (SEQ ID NO: 90) and the PCR probe

was: 5′ JOE-CAAGCTTCCCGTTCTCAGCC-TAMRA 3′ (SEQ ID NO: 91) where JOE is the fluorescent reporter dye and TAMRA is the quencher dye.

### Example 13

Inhibition of CD40 Expression by Phosphorothioate Oligodeoxynucleotides

In accordance with the present invention, a series of oligonucleotides complementary to mRNA were designed to target different regions of the human CD40 mRNA, using published sequences (GenBank accession number X60592, incorporated herein by reference as SEQ ID NO: 85). The oligonucleotides are shown in Table 7. Target sites are indicated by the beginning nucleotide numbers, as given in the sequence source reference (X60592), to which the oligonucleotide binds. All compounds in Table 7 are oligodeoxynucleotides with phosphorothioate backbones (internucleoside linkages) throughout. Data are averages from three experiments.

TABLE 7

Inhibition of CD40 mRNA Levels
by Phosphorothioate Oligodeoxynucleotides

| | | | | |
|---|---|---|---|---|
| | TARGET | | % | SEQ ID |
| ISIS# | SITE | SEQUENCE | INHIB. | NO. |

| | | | | |
|---|---|---|---|---|
| 18623 | 18 | CCAGGCGGCAGGACCAC | 30.71 | 1 |
| 18624 | 20 | GACCAGGCGGCAGGAC | 28.09 | 2 |
| 18625 | 26 | AGGTGAGACCAGGCGG | 21.89 | 3 |
| 18626 | 48 | CAGAGGCAGACGAACC | 0.00 | 4 |
| 18627 | 49 | GCAGAGGCAGACGAAC | 0.00 | 5 |
| 18628 | 73 | GCAAGCAGCCCCAGAG | 0.00 | 6 |
| 18629 | 78 | GGTCAGCAAGCAGCCCC | 29.96 | 7 |
| 18630 | 84 | GACAGCGGTCAGCAAGC | 0.00 | 8 |
| 18631 | 88 | GATGGACAGCGGTCAGC | 0.00 | 9 |
| 18632 | 92 | TCTGGATGGACAGCGGT | 0.00 | 10 |
| 18633 | 98 | GGTGGTTCTGGATGGAC | 0.00 | 11 |
| 18634 | 101 | GTGGGTGGTTCTGGATG | 0.00 | 12 |
| 18635 | 104 | GCAGTGGGTGGTTCTGG | 0.00 | 13 |
| 18636 | 152 | CACAAAGAACAGCACTG | 0.00 | 14 |
| 18637 | 156 | CTGGCACAAAGAACAGC | 0.00 | 15 |
| 18638 | 162 | TCCTGGCTGGCACAAAG | 0.00 | 16 |
| 18639 | 165 | CTGTCCTGGCTGGCACA | 4.99 | 17 |
| 18640 | 176 | CTCACCAGTTTCTGTCCT | 0.00 | 18 |
| 18641 | 179 | TCACTCACCAGTTTCTG | 0.00 | 19 |
| 18642 | 185 | GTGCAGTCACTCACCAG | 0.00 | 20 |
| 18643 | 190 | ACTCTGTGCAGTCACTC | 0.00 | 21 |
| 18644 | 196 | CAGTGAACTCTGTGCAG | 5.30 | 22 |
| 18645 | 205 | ATTCCGTTTCAGTGAAC | 0.00 | 23 |
| 18646 | 211 | GAAGGCATTCCGTTTCA | 9.00 | 24 |
| 18647 | 222 | TTCACCGCAAGGAAGGC | 0.00 | 25 |
| 18648 | 250 | CTCTGTTCCAGGTGTCT | 0.00 | 26 |
| 18649 | 267 | CTGGTGGCAGTGTGTCT | 0.00 | 27 |
| 18650 | 286 | TGGGGTCGCAGTATTTG | 0.00 | 28 |
| 18651 | 289 | GGTTGGGGTCGCAGTAT | 0.00 | 29 |
| 18652 | 292 | CTAGGTTGGGGTCGCAG | 0.00 | 30 |
| 18653 | 318 | GGTGCCCTTCTGCTGGA | 19.67 | 31 |
| 18654 | 322 | CTGAGGTGCCCTTCTGC | 15.63 | 32 |
| 18655 | 332 | GTGTCTGTTTCTGAGGT | 0.00 | 33 |
| 18656 | 334 | TGGTGTCTGTTTCTGAG | 0.00 | 34 |
| 18657 | 345 | ACAGGTGCAGATGGTGT | 0.00 | 35 |
| 18658 | 348 | TTCACAGGTGCAGATGG | 0.00 | 36 |
| 18659 | 360 | GTGCCAGCCTTCTTCAC | 5.67 | 37 |
| 18660 | 364 | TACAGTGCCAGCCTTCT | 7.80 | 38 |
| 18661 | 391 | GGACACAGCTCTCACAG | 0.00 | 39 |
| 18662 | 395 | TGCAGGACACAGCTCTC | 0.00 | 40 |
| 18663 | 401 | GAGCGGTGCAGGACAC | 0.00 | 41 |
| 18664 | 416 | AAGCCGGGCGAGCATG | 0.00 | 42 |
| 18665 | 432 | AATCTGCTTGACCCCAA | 5.59 | 43 |
| 18666 | 446 | GAAACCCCTGTAGCAAT | 0.10 | 44 |
| 18667 | 452 | GTATCAGAAACCCCTGT | 0.00 | 45 |
| 18668 | 463 | GCTCGCAGATGGTATCA | 0.00 | 46 |
| 18669 | 468 | GCAGGGCTCGCAGATGG | 34.05 | 47 |
| 18670 | 471 | TGGGCAGGGCTCGCAGA | 0.00 | 48 |
| 18671 | 474 | GACTGGGCAGGGCTCGC | 2.71 | 49 |
| 18672 | 490 | CATTGGAGAAGAAGCCG | 0.00 | 50 |
| 18673 | 497 | GATGACACATTGGAGAA | 0.00 | 51 |
| 18674 | 500 | GCAGATGACACATTGGA | 0.00 | 52 |
| 18675 | 506 | TCGAAAGCAGATGACAC | 0.00 | 53 |
| 18676 | 524 | GTCCAAGGGTGACATTT | 8.01 | 54 |
| 18677 | 532 | CACAGCTTGTCCAAGGG | 0.00 | 55 |
| 18678 | 539 | TTGGTCTCACAGCTTGT | 0.00 | 56 |
| 18679 | 546 | CAGGTCTTTGGTCTCAC | 6.98 | 57 |
| 18680 | 558 | CTGTTGCACAACCAGGT | 18.76 | 58 |
| 18681 | 570 | GTTTGTGCCTGCCTGTT | 2.43 | 59 |
| 18682 | 575 | GTCTTGTTTGTGCCTGCC | 0.00 | 60 |
| 18683 | 590 | CCACAGACAACATCAGT | 0.00 | 61 |
| 18684 | 597 | CTGGGGACCACAGACAA | 0.00 | 62 |
| 18685 | 607 | TCAGCCGATCCTGGGGA | 0.00 | 63 |
| 18686 | 621 | CACCACCAGGGCTCTCA | 23.31 | 64 |
| 18687 | 626 | GGGATCACCACCAGGGC | 0.00 | 65 |
| 18688 | 657 | GAGGATGGCAAACAGG | 0.00 | 66 |
| 18689 | 668 | ACCAGCACCAAGAGGAT | 0.00 | 67 |
| 18690 | 679 | TTTTGATAAAGACCAGC | 0.00 | 68 |
| 18691 | 703 | TATTGGTTGGCTTCTTG | 0.00 | 69 |
| 18692 | 729 | GGGTTCCTGCTTGGGGT | 0.00 | 70 |
| 18693 | 750 | GTCGGGAAAATTGATCT | 0.00 | 71 |
| 18694 | 754 | GATCGTCGGGAAAATTG | 0.00 | 72 |
| 18695 | 765 | GGAGCCAGGAAGATCGT | 0.00 | 73 |
| 18696 | 766 | TGGAGCCAGGAAGATCG | 0.00 | 74 |
| 18697 | 780 | TGGAGCAGCAGTGTTGG | 0.00 | 75 |
| 18698 | 796 | GTAAAGTCTCCTGCACT | 0.00 | 76 |
| 18699 | 806 | TGGCATCCATGTAAAGT | 0.00 | 77 |
| 18700 | 810 | CGGTTGGCATCCATGTA | 0.00 | 78 |
| 18701 | 834 | CTCTTTGCCATCCTCCTG | 4.38 | 79 |
| 18702 | 861 | CTGTCTCTCCTGCACTG | 0.00 | 80 |
| 18703 | 873 | GGTGCAGCCTCACTGTC | 0.00 | 81 |
| 18704 | 910 | AACTGCCTGTTTGCCCA | 33.89 | 82 |
| 18705 | 954 | CTTCTGCCTGCACCCCT | 0.00 | 83 |
| 18706 | 976 | ACTGACTGGGCATAGCT | 0.00 | 84 |

As shown in Table 7, SEQ ID NOS: 1, 2, 7, 47 and 82 demonstrated at least 25% inhibition of CD40 expression and are therefore preferred compounds of the invention.

### Example 14

Inhibition of CD40 Expression by Phosphorothioate 2′-MOE Gapmer Oligonucleotides

In accordance with the present invention, a second series of oligonucleotides complementary to mRNA were designed to target different regions of the human CD40 mRNA, using published sequence X60592. The oligonucleotides are shown in Table 8. Target sites are indicated by the beginning or initial nucleotide numbers, as given in the sequence source reference (X60592), to which the oligonucleotide binds.

All compounds in Table 8 are chimeric oligonucleotides (“gapmers”) 18 nucleotides in length, composed of a central “gap” region consisting of ten 2′-deoxynucleotides, which is flanked on both sides (5′ and 3′ directions) by four-nucleotide “wings.” The wings are composed of 2′-O-(2-methoxyethyl) (2′-MOE) nucleotides. The intersugar (backbone) linkages are phosphorothioate (P═S) throughout the oligonucleotide. Cytidine residues in the 2′-MOE wings are 5-methylcytidines. Data are averaged from three experiments.

TABLE 8

Inhibition of CD40 mRNA Levels
by Chimeric Phosphorothioate Oligonucleotides

| | | | | |
|---|---|---|---|---|
| | TARGET | | % | |
| ISIS# | SITE | SEQUENCE | Inhibition | SEQ ID |

| | | | | |
|---|---|---|---|---|
| 19211 | 18 | CCAGGCGGCAGGACCA | 75.71 | 1 |
| 19212 | 20 | GACCAGGCGGCAGGAC | 77.23 | 2 |
| 19213 | 26 | AGGTGAGACCAGGCGG | 80.82 | 3 |
| 19214 | 48 | CAGAGGCAGACGAACC | 23.68 | 4 |
| 19215 | 49 | GCAGAGGCAGACGAAC | 45.97 | 5 |
| 19216 | 73 | GCAAGCAGCCCCAGAG | 65.80 | 6 |
| 19217 | 78 | GGTCAGCAAGCAGCCC | 74.73 | 7 |
| 19218 | 84 | GACAGCGGTCAGCAAG | 67.21 | 8 |
| 19219 | 88 | GATGGACAGCGGTCAG | 65.14 | 9 |
| 19220 | 92 | TCTGGATGGACAGCGGT | 78.71 | 10 |
| 19221 | 98 | GGTGGTTCTGGATGGAC | 81.33 | 11 |
| 19222 | 101 | GTGGGTGGTTCTGGATG | 57.79 | 12 |
| 19223 | 104 | GCAGTGGGTGGTTCTGG | 73.70 | 13 |
| 19224 | 152 | CACAAAGAACAGCACT | 40.25 | 14 |
| 19225 | 156 | CTGGCACAAAGAACAG | 60.11 | 15 |
| 19226 | 162 | TCCTGGCTGGCACAAAG | 10.18 | 16 |
| 19227 | 165 | CTGTCCTGGCTGGCACA | 24.37 | 17 |
| 19228 | 176 | CTCACCAGTTTCTGTCC | 22.30 | 18 |
| 19229 | 179 | TCACTCACCAGTTTCTG | 40.64 | 19 |
| 19230 | 185 | GTGCAGTCACTCACCAG | 82.04 | 20 |
| 19231 | 190 | ACTCTGTGCAGTCACTC | 37.59 | 21 |
| 19232 | 196 | CAGTGAACTCTGTGCAG | 40.26 | 22 |
| 19233 | 205 | ATTCCGTTTCAGTGAAC | 56.03 | 23 |
| 19234 | 211 | GAAGGCATTCCGTTTCA | 32.21 | 24 |
| 19235 | 222 | TTCACCGCAAGGAAGG | 61.03 | 25 |
| 19236 | 250 | CTCTGTTCCAGGTGTcT | 62.19 | 26 |
| 19237 | 267 | CTGGTGGCAGTGTGTCT | 70.32 | 27 |
| 19238 | 286 | TGGGGTCGCAGTATTTG | 0.00 | 28 |
| 19239 | 289 | GGTTGGGGTCGCAGTAT | 19.40 | 29 |
| 19240 | 292 | CTAGGTTGGGGTCGCAG | 36.32 | 30 |
| 19241 | 318 | GGTGCCCTTCTGCTGGA | 78.91 | 31 |
| 19242 | 322 | CTGAGGTGCCCTTCTGC | 69.84 | 32 |
| 19243 | 332 | GTGTCTGTTTCTGAGGT | 63.32 | 33 |
| 19244 | 334 | TGGTGTCTGTTTCTGAG | 42.83 | 34 |
| 19245 | 345 | ACAGGTGCAGATGGTGT | 73.31 | 35 |
| 19246 | 348 | TTCACAGGTGCAGATGG | 47.72 | 36 |
| 19247 | 360 | GTGCCAGCCTTCTTCAC | 61.32 | 37 |
| 19248 | 364 | TACAGTGCCAGCCTTCT | 46.82 | 38 |
| 19249 | 391 | GGACACAGCTCTCACAG | 0.00 | 39 |
| 19250 | 395 | TGCAGGACACAGCTCTC | 52.05 | 40 |
| 19251 | 401 | GAGCGGTGCAGGACAC | 50.15 | 41 |
| 19252 | 416 | AAGCCGGGCGAGCATG | 32.36 | 42 |
| 19253 | 432 | AATCTGCTTGACCCCAA | 0.00 | 43 |
| 19254 | 446 | GAAACCCCTGTAGCAAT | 0.00 | 44 |
| 19255 | 452 | GTATCAGAAACCCCTGT | 36.13 | 45 |
| 19256 | 463 | GCTCGCAGATGGTATCA | 64.65 | 46 |
| 19257 | 468 | GCAGGGCTCGCAGATG | 74.95 | 47 |
| 19258 | 471 | TGGGCAGGGCTCGCAG | 0.00 | 48 |
| 19259 | 474 | GACTGGGCAGGGCTCG | 82.00 | 49 |
| 19260 | 490 | CATTGGAGAAGAAGCC | 41.31 | 50 |
| 19261 | 497 | GATGACACATTGGAGA | 13.81 | 51 |
| 19262 | 500 | GCAGATGACACATTGG | 78.48 | 52 |
| 19263 | 506 | TCGAAAGCAGATGAcA | 59.28 | 53 |
| 19264 | 524 | GTCCAAGGGTGACATTT | 70.99 | 54 |
| 19265 | 532 | CACAGCTTGTCCAAGGG | 0.00 | 55 |
| 19266 | 539 | TTGGTCTCACAGCTTGT | 45.92 | 56 |
| 19267 | 546 | CAGGTCTTTGGTCTCAc | 63.95 | 57 |
| 19268 | 558 | CTGTTGCACAACCAGGT | 82.32 | 58 |
| 19269 | 570 | GTTTGTGCCTGCCTGTT | 70.10 | 59 |
| 19270 | 575 | GTCTTGTTTGTGCCTGC | 68.95 | 60 |
| 19271 | 590 | CCACAGACAACATCAGT | 11.22 | 61 |
| 19272 | 597 | CTGGGGACCACAGACA | 9.04 | 62 |
| 19273 | 607 | TCAGCCGATCCTGGGGA | 0.00 | 63 |
| 19274 | 621 | CACCACCAGGGCTCTCA | 23.08 | 64 |
| 19275 | 626 | GGGATCACCACCAGGG | 57.94 | 65 |
| 19276 | 657 | GAGGATGGCAAACAGG | 49.14 | 66 |
| 19277 | 668 | ACCAGCACCAAGAGGA | 3.48 | 67 |
| 19278 | 679 | TTTTGATAAAGACCAGC | 30.58 | 68 |
| 19279 | 703 | TATTGGTTGGCTTCTTG | 49.26 | 69 |
| 19280 | 729 | GGGTTCCTGCTTGGGGT | 13.95 | 70 |
| 19281 | 750 | GTCGGGAAAATTGATcT | 54.78 | 71 |
| 19282 | 754 | GATCGTCGGGAAAATTG | 0.00 | 72 |
| 19283 | 765 | GGAGCCAGGAAGATCG | 69.47 | 73 |
| 19284 | 766 | TGGAGCCAGGAAGATC | 54.48 | 74 |
| 19285 | 780 | TGGAGCAGCAGTGTTGG | 15.17 | 75 |
| 19286 | 796 | GTAAAGTCTCCTGCACT | 30.62 | 76 |
| 19287 | 806 | TGGCATCCATGTAAAGT | 65.03 | 77 |
| 19288 | 810 | CGGTTGGCATCCATGTA | 34.49 | 78 |
| 19289 | 834 | CTCTTTGCCATCCTCCT | 41.84 | 79 |
| 19290 | 861 | CTGTCTCTCCTGCACTG | 25.68 | 80 |
| 19291 | 873 | GGTGCAGCCTCACTGTC | 76.27 | 81 |
| 19292 | 910 | AACTGCCTGTTTGCCCA | 63.34 | 82 |
| 19293 | 954 | CTTCTGCCTGCACCCCT | 0.00 | 83 |
| 19294 | 976 | ACTGACTGGGCATAGCT | 11.55 | 84 |

As shown in Table 8, SEQ ID NOS: 1, 2, 3, 6, 7, 8, 9, 10, 11, 12, 13, 15, 20, 23, 25, 26, 27, 31, 32, 33, 35, 37, 40, 41, 46, 47, 49, 52, 53, 54, 57, 58, 59, 60, 65, 71, 73, 74, 77, 81 and 82 demonstrated at least 50% inhibition of CD40 expression and are therefore preferred compounds of the invention.

### Example 15

Oligonucleotide-Sensitive Sites of the CD40 Target Nucleic Acid

As the data presented in the preceding two examples shows, several sequences were present in preferred compounds of two distinct oligonucleotide chemistries. Specifically, compounds having SEQ ID NOS: 1, 2, 7, 47 and 82 are preferred in both instances. These compounds map to different regions of the CD40 transcript but nevertheless define accessible sites of the target nucleic acid.

For example, SEQ ID NOS: 1 and 2 overlap each other and both map to the 5-untranslated region (5′-UTR) of CD40. Accordingly, this region of CD40 is particularly preferred for modulation via sequence-based technologies. Similarly, SEQ ID NOS: 7 and 47 map to the open reading frame of CD40, whereas SEQ ID NO: 82 maps to the 3′-untranslated region (3′-UTR). Thus, the ORF and 3′-UTR of CD40 may be targeted by sequence-based technologies as well.

The reverse complements of the active CD40 compounds are easily determined by those skilled in the art and may be assembled to yield nucleotide sequences corresponding to accessible sites on the target nucleic acid. For example, the assembled reverse complement of SEQ ID NOS: 1 and 2 is represented below as SEQ ID NO: 92:

5′-AGTGGTCCTGCCGCCTGGTC-3′ SEQ ID NO: 92

TCACCAGGACGGCGGACC-5′ SEQ ID NO: 1

ACCAGGACGGCGGACCAG-5′ SEQ ID NO: 2

Through multiple iterations of the process of the invention, more extensive “footprints” are generated. A library of this information is compiled and may be used by those skilled in the art in a variety of sequence-based technologies to study the molecular and biological functions of CD40 and to investigate or confirm its role in various diseases and disorders.

### Example 16

Site Selection Program

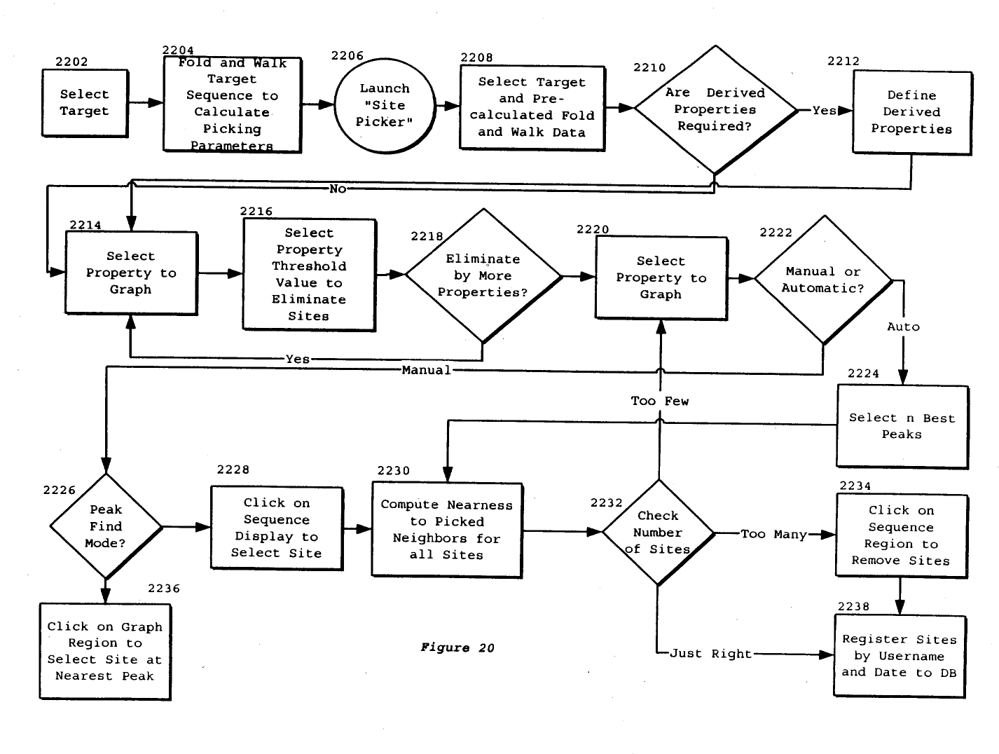

In a preferred embodiment of the invention, illustrated in FIG. 20, an application is deployed which facilitates the selection process for determining the target positions of the oligos to be synthesized, or “sites.” This program is written using a three-tiered object-oriented approach. All aspects of the software described, therefore, are tightly integrated with the relational database. For this reason, explicit database read and write steps are not shown. It should be assumed that each step described includes database access. The description below illustrates one way the program can be used. The actual interface allows users to skip from process to process at will, in any order.

Before running the site picking program, the target must have all relevant properties computed as described previously and indicated in process step 2204. When the site picking program is launched at process step 2206 the user is presented with a panel showing targets which have previously been selected and had their properties calculated. The user selects one target to work with at process step 2208 and proceeds to decide if any derived properties will be needed at process step 2210. Derived properties are calculated by performing mathematical operations on combinations of pre-calculated properties as defined by the user at process step 2212.

The derived properties are made available as peers with all the pre-calculated properties. The user selects one of the properties to view plotted versus target position at process step 2214. This graph is shown above a linear representation of the target. The horizontal or position axis of both the graph and target are linked and scalable by the user. The zoom range goes from showing the full target length to showing individual target bases as letters and individual property points. The user next selects a threshold value below or above which all sites will be eliminated from future consideration at process step 2216. The user decides whether to eliminate more sites based on any other properties at process step 2218. If they choose to eliminate more, they return to pick another property to display at process step 2214 and threshold at process step 2216.

After eliminating sites, the user selects from the remaining list by choosing any property at process step 2220 and then choosing a manual or automatic selection technique at process step 2222. In the automatic technique, the user decides whether they want to pick from maxima or minima and the number of maxima or minima to be selected as sites at process step 2224. The software automatically finds and picks the points. When picking manually the user must decide if they wish to use automatic peak finding at process step 2226. If the user selects automatic peak finding, then user must click on the graphed property with the mouse at process step 2236. The nearest maxima or minima, depending on the modifier key held down, to the selected point will be picked as the site. Without the peak finding option, the user must pick a site at process step 2238 by clicking on its position on the linear representation of target.

Each time a site, or group of sites, is picked, a dynamic property is calculated for all possible sites (not yet eliminated) at process step 2230. This property indicates the nearness of the site to a picked site allowing the user to pick sites in subsequent iterations based on target coverage. After new sites are picked, the user determines if the desired number of sites has been picked. If too few sites have been picked the user returns to pick more 2220. If too many sites have been picked, the user may eliminate them by selecting and deleting them on the target display at process step 2234. If the correct number of sites is picked, and the user is satisfied with the set of picked sites, the user registers these sites to the database along with their name, notebook number, and page number at process step 2238. The database time stamps this registration event.

### Example 17

Site Selection Program

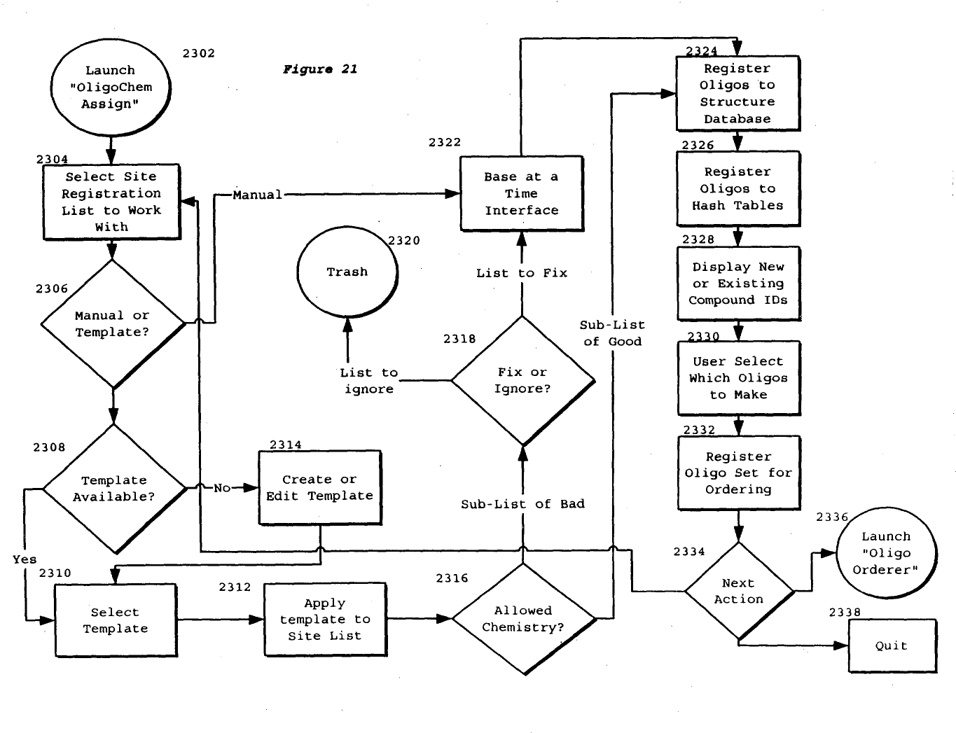

In a preferred embodiment of the invention, illustrated in FIG. 21, an application is deployed which facilitates the assignment of specific chemical structure to the complement of the sequence of the sites previously picked and facilitates the registration and ordering of these now fully defined antisense compounds. This program is written using a three-tiered object-oriented approach. All aspects of the software described, therefore, are tightly integrated with the relational database. For this reason, explicit database read and write steps are not shown, it being understood that each step described also includes appropriate database read/write access.

To begin using the oligonucleotide chemistry assignment program, the user launches it at process step 2302. The user then selects from the previously selected sets of oligonucleotides at process step 2304, registered to the database in site picker's process step 2238. Next, the user must decide whether to manually assign the chemistry a base at a time, or run the sites through a template at process step 2306. If the user chooses to use a template, they must determine if a desired template is available at process step 2308. If a template is not available with the desired chemistry modifications and the correct length, the user can define one at process step 2314.

To define a template, the user must select the length of the oligonucleotide the template is to define. This oligonucleotide is then represented as a bar with selectable regions. The user sets the number of regions on the oligonucleotide, and the positions and lengths of these regions by dragging them back and forth on the bar. Each region is represented by a different color.

For each region, the user defines the chemistry modifications for the sugars, the linkers, and the heterocycles at each base position in the region. At least four heterocycle chemistries must be given, one for each of the four possible base types (A, G, C or T or U) in the site sequence the template will be applied to. A user interface is provided to select these chemistries which show the molecular structure of each component selected and its modification name. By pushing on a pop-up list next to each of the pictures, the user may choose from a list of structures and names, those possible to put in this place. For example, the heterocycle that represents the base type G is shown as a two dimensional structure diagram. If the user clicks on the pop-up list, a row of other possible structures and names is shown. The user drags the mouse to the desired chemistry and releases the mouse. Now the newly selected molecule is displayed as the choice for G type heterocycle modifications.

Once the user has created a template, or selected an existing one, the software applies the template at process step 2312 to each of the complements of the sites in the list. When the templates are applied, it is possible that chemistries will be defined which are impossible to make with the chemical precursors-presently used on the automatic synthesizer. To check this, a database is maintained of all precursors previously designed, and their availability for automated synthesis. When the templates are applied, the resulting molecules are tested at process step 2316 against this database to see if they are readily synthesized.

If a molecule is not readily synthesized, it is added to a list that the user inspects. At process step 2318, the user decides whether to modify the chemistry to make it compatible with the currently recognized list of available chemistries or to ignore it. To modify a chemistry, the user must use the base at a time interface at process step 2322. The user can also choose to go directly to this step, bypassing templates all together at process step 2306.

The base at a time interface at process step 2322 is very similar to the template editor at process step 2314 except that instead of specifying chemistries for regions, they are defined one base at a time. This interface also differs in that it dynamically checks to see if the design is readily synthesized as the user makes selections. In other words, each choice made limits the choices the software makes available on the pop-up selection lists. To accommodate this function, an additional choice is made available on each pop-up of “not defined.” For example, this allows the user to inhibit linker choice from restricting the sugar choices by first setting the linker to “not defined.” The user would then pick the sugar, and then pick from the remaining linker choices available.

Once all of the sites on the list are assigned chemistries or dropped, they are registered at process step 2324 to a commercial chemical structure database. Registering to this database makes sure the structure is unique, assigns it a new identifier if it is unique, and allows future structure and substructure searching by creating various hash-tables. The compound definition is also stored at process step 2326 to various hash tables referred to as chemistry/position tables. These allow antisense compound searching and categorization based on oligonucleotide chemistry modification sequences and equivalent base sequences.

The results of the registration are displayed at process step 2328 with the new IDs if they are new compounds and with the old IDs if they have been previously registered. The user next selects which of the compounds processed they wish to order for synthesis at process step 2330 and registers an order list at process step 2332 by including scientist name, notebook number and page number. The database time-stamps this entry. The user may then choose at process step 2334, to quit the program at process step 2338, go back to the beginning and choose a new site list to work with process step 2304, or start the oligonucleotide ordering interface at process step 2336.

### Example 18

Gene Walk to Optimize Oligonucleotide Sequence

A gene walk is executed using a CD40 antisense oligonucleotide having SEQ IS NO: 5′-CTGGCACAAAGAACAGCA-3′). In effecting this gene walk, the following parameters are used:

| Gene Walk Parameter | Entered value |
|---|---|
| Oligonucleotide Sequence ID: | 15 |
| Name of Gene Target: | CD40 |
| Scope of Gene Walk: | 20 |
| Sequence Shift Increment: | 1 |

Entering these values and effecting the gene walk centered on SEQ ID NO: 15 automatically generates the following new oligonucleotides:

TABLE 9
Oligonucleotide Generated By Gene Walk

| SEQ ID | Sequence |
|---|---|
| 93 | GAACAGCACTGACTGT |
| 94 | AGAACAGCACTGACTG |
| 95 | AAGAACAGCACTGACT |
| 96 | AAAGAACAGCACTGAC |
| 97 | CAAAGAACAGCACTGA |
| 98 | ACAAAGAACAGCACTG |
| 99 | CACAAAGAACAGCACT |
| 100 | GCACAAAGAACAGCAC |
| 101 | GGCACAAAGAACAGCA |
| 102 | TGGCACAAAGAACAGC |
| 15 | CTGGCACAAAGAACAG |
| 103 | GCTGGCACAAAGAACA |
| 104 | GGCTGGCACAAAGAAC |
| 105 | TGGCTGGCACAAAGAA |
| 106 | CTGGCTGGCACAAAGA |
| 107 | CCTGGCTGGCACAAAG |
| 108 | TCCTGGCTGGCACAAA |
| 109 | GTCCTGGCTGGCACAA |
| 110 | TGTCCTGGCTGGCACA |
| 111 | CTGTCCTGGCTGGCAC |
| 112 | TCTGTCCTGGCTGGCAC |

The list shown above contains 20 oligonucleotide sequences directed against the CD40 nucleic acid sequence. They are ordered by the position along the CD40 sequence at which the 5′ terminus of each oligonucleotide hybridizes. Thus, the first ten oligonucleotides are single-base frame shift sequences directed against the CD40 sequence upstream of compound SEQ ID NO: 15 and the latter ten are single-base frame shift sequences directed against the CD40 sequence downstream of compound SEQ ID NO: 15.

### Example 19

Automated Assay of RhoC Oligonucleotide Activity

RhoC, a member of the Rho subfamily of small GTPases, is a protein that has been shown to be involved in a diverse set of signaling pathways including the ultimate regulation of the dynamic organization of the cytoskeleton.

Oligonucleotides targeting RhoC were designed, synthesized, analyzed and assayed according to procedures outlined in U.S. Ser. No. 09/067,638. Alternatively, oligonucleotides targeting RhoC can be designed as described in Example 2, synthesized as described in Examples 3-7, analyzed as described in Example 8 and assayed by RT-PCR as described in Example 12.

RhoC probes and primers were designed to hybridize to the human RhoC sequence, using published sequence information (GenBank accession number L25081, incorporated herein by reference as SEQ ID NO: 113).

For RhoC the PCR primers were:

forward primer TGATGTCATCCTCATGTGCTTCT (SEQ ID NO: 114)

reverse primer CCAGGATGATGGGCACGTT (SEQ ID NO: 115) and the PCR probe

was: FAM-CGACAGCCCTGACAGCCTGGAAA-TAMRA (SEQ ID NO: 116) where FAM (PE-Applied Biosystems, Foster City, Calif.) is the fluorescent reporter dye) and TAMRA (PE-Applied Biosystems, Foster City, Calif.) is the quencher dye.

### Example 20

Antisense Inhibition of RhoC Expression-Phosphorothioate Oligodeoxynucleotides

In accordance with the present invention, a series of oligonucleotides were designed to target different regions of the human RhoC RNA, using published sequences (GenBank accession number L25081, incorporated herein by reference as SEQ ID NO: 113). The oligonucleotides are shown in Table 10. Target sites are indicated by nucleotide numbers, as given in the sequence source reference (Genbank accession no. L25081), to which the oligonucleotide binds. All compounds in Table 10 are oligodeoxynucleotides with phosphorothioate backbones (internucleoside linkages) throughout. The compounds were analyzed for effect on RhoC mRNA levels by quantitative real-time PCR as described in other examples herein. Data are averages from three experiments. If present, “N.D.” indicates “no data”.

TABLE 10

Inhibition of RhoC mRNA levels
by phosphorothioate oligodeoxynucleotides

| | | | | | |
|---|---|---|---|---|---|
| | | | | % | |
| | | | | Inhi- | |
| | | TARGET | | bi- | SEQ ID |
| ISIS# | REGION | SITE | SEQUENCE | tion | NO. |

| | | | | | |
|---|---|---|---|---|---|
| 25304 | 5′ UTR | 4 | gagctgagatgaagtcaa | 29 | 117 |
| 25305 | 5′ UTR | 44 | gctgaagttcccaggctg | 25 | 118 |
| 25306 | 5′ UTR | 47 | ccggctgaagttcccagg | 42 | 119 |
| 25307 | Coding | 104 | ggcaccatccccaacgat | 81 | 120 |
| 25308 | Coding | 105 | aggcaccatccccaacga | 81 | 121 |
| 25309 | Coding | 111 | tcccacaggcaccatccc | 70 | 122 |
| 25310 | Coding | 117 | aggtcttcccacaggcac | 40 | 123 |
| 25311 | Coding | 127 | atgaggaggcaggtcttc | 41 | 124 |
| 25312 | Coding | 139 | ttgctgaagacgatgagg | 23 | 125 |
| 25313 | Coding | 178 | tcaaagacagtagggacg | 0 | 126 |
| 25314 | Coding | 181 | ttctcaaagacagtaggg | 2 | 127 |
| 25315 | Coding | 183 | agttctcaaagacagtag | 38 | 128 |
| 25316 | Coding | 342 | tgttttccaggctgtcag | 59 | 129 |
| 25317 | Coding | 433 | tcgtcttgcctcaggtcc | 79 | 130 |
| 25318 | Coding | 439 | gtgtgctcgtcttgcctc | 67 | 131 |
| 25319 | Coding | 445 | ctcctggtgtgctcgtct | 67 | 132 |
| 25320 | Coding | 483 | cagaccgaacgggctcct | 65 | 133 |
| 25321 | Coding | 488 | ttcctcagaccgaacggg | 57 | 134 |
| 25322 | Coding | 534 | actcaaggtagccaaagg | 33 | 135 |
| 25323 | Coding | 566 | ctcccgcactccctcctt | 91 | 136 |
| 25324 | Coding | 575 | ctcaaacacctcccgcac | 34 | 137 |
| 25325 | Coding | 581 | ggccatctcaaacacctc | 64 | 138 |
| 25326 | Coding | 614 | cttgttcttgcggacctg | 72 | 139 |
| 25327 | Coding | 625 | cccctccgacgcttgttc | 66 | 140 |
| 25328 | 3′ UTR | 737 | gtatggagccctcaggag | 60 | 141 |
| 25329 | 3′ UTR | 746 | gagccttcagtatggagc | 63 | 142 |
| 25330 | 3′ UTR | 753 | gaaaatggagccttcagt | 24 | 143 |
| 25331 | 3′ UTR | 759 | ggaactgaaaatggagcc | 2 | 144 |
| 25332 | 3′ UTR | 763 | ggagggaactgaaaatgg | 13 | 145 |
| 25333 | 3′ UTR | 766 | gcaggagggaactgaaaa | 27 | 146 |
| 25334 | 3′ UTR | 851 | agggcagggcataggcgt | 31 | 147 |
| 25335 | 3′ UTR | 854 | ggaagggcagggcatagg | 21 | 148 |
| 25336 | 3′ UTR | 859 | catgaggaagggcagggc | 0 | 149 |
| 25337 | 3′ UTR | 920 | taaagtgctggtgtgtga | 39 | 150 |
| 25338 | 3′ UTR | 939 | cctgtgagccagaagtgt | 69 | 151 |
| 25339 | 3′ UTR | 941 | ttcctgtgagccagaagt | 69 | 152 |
| 25340 | 3′ UTR | 945 | cactttcctgtgagccag | 82 | 153 |
| 25341 | 3′ UTR | 948 | agacactttcctgtgagc | 69 | 154 |
| 25342 | 3′ UTR | 966 | actctgggtccctactgc | 20 | 155 |
| 25343 | 3′ UTR | 992 | tgcagaaacaactccagg | 0 | 156 |

### Example 21

Antisense Inhibition of RhoC Expression-Phosphorothioate 2′-MOE Gapmer Oligonucleotides

In accordance with the present invention, a second series of oligonucleotides targeted to human RhoC were synthesized. The oligonucleotide sequences are shown in Table 11. Target sites are indicated by nucleotide numbers, as given in the sequence source reference (Genbank accession No. L25081), to which the oligonucleotide binds.

All compounds in Table 11 are chimeric oligonucleotides (“gapmers”) 18 nucleotides in length, composed of a central “gap” region consisting of ten 2′-deoxynucleotides, which is flanked on both sides (5′ and 3′ directions) by four-nucleotide “wings.” The wings are composed of 2′-methoxyethyl (2′-MOE) nucleotides. The internucleoside (backbone) linkages are phosphorothioate (P═S) throughout the oligonucleotide. Cytidine residues in the 2′-MOE wings are 5-methylcytidines.

Data were obtained by real-time quantitative PCR as described in other examples herein and are averaged from three experiments. If present, “N.D.” indicates “no data”.

TABLE 11

Inhibition of RhoC mRNA levels
by chimeric phosphorothioate oligonucleotides
having 2′-MOE wings and a deoxy gap

| | | | | | |
|---|---|---|---|---|---|
| | | | | % | |
| | | | | Inhi- | |
| | | TARGET | | bi- | SEQ ID |
| ISIS# | REGION | SITE | SEQUENCE | tion | NO. |

| | | | | | |
|---|---|---|---|---|---|
| 25344 | 5′ UTR | 4 | gagctgagatgaagtcaa | 0 | 117 |
| 25345 | 5′ UTR | 44 | gctgaagttcccaggctg | 35 | 118 |
| 25346 | 5′ UTR | 47 | ccggctgaagttcccagg | 53 | 119 |
| 25347 | Coding | 104 | ggcaccatccccaacgat | 50 | 120 |
| 25348 | Coding | 105 | aggcaccatccccaacga | 56 | 121 |
| 25349 | Coding | 111 | tcccacaggcaccatccc | 4 | 122 |
| 25350 | Coding | 117 | aggtcttcccacaggcac | 11 | 123 |
| 25351 | Coding | 127 | atgaggaggcaggtcttc | 6 | 124 |
| 25352 | Coding | 139 | ttgctgaagacgatgagg | 15 | 125 |
| 25353 | Coding | 178 | tcaaagacagtagggacg | 32 | 126 |
| 25354 | Coding | 181 | ttctcaaagacagtaggg | 7 | 127 |
| 25355 | Coding | 183 | agttctcaaagacagtag | 39 | 128 |
| 25356 | Coding | 342 | tgttttccaggctgtcag | 59 | 129 |
| 25357 | Coding | 433 | tcgtcttgcctcaggtcc | 48 | 130 |
| 25358 | Coding | 439 | gtgtgctcgtcttgcctc | 36 | 131 |
| 25359 | Coding | 445 | ctcctggtgtgctcgtct | 61 | 132 |
| 25360 | Coding | 483 | cagaccgaacgggctcct | 50 | 133 |
| 25361 | Coding | 488 | ttcctcagaccgaacggg | 14 | 134 |
| 25362 | Coding | 534 | actcaaggtagccaaagg | 32 | 135 |
| 25363 | Coding | 566 | ctcccgcactccctcctt | 21 | 136 |
| 25364 | Coding | 575 | ctcaaacacctcccgcac | 9 | 137 |
| 25365 | Coding | 581 | ggccatctcaaacacctc | 66 | 138 |
| 25366 | Coding | 614 | cttgttcttgcggacctg | 61 | 139 |
| 25367 | Coding | 625 | cccctccgacgcttgttc | 0 | 140 |
| 25368 | 3′ UTR | 737 | gtatggagccctcaggag | 28 | 141 |
| 25369 | 3′ UTR | 746 | gagccttcagtatggagc | 32 | 142 |
| 25370 | 3′ UTR | 753 | gaaaatggagccttcagt | 0 | 143 |
| 25371 | 3′ UTR | 759 | ggaactgaaaatggagcc | 40 | 144 |
| 25372 | 3′ UTR | 763 | ggagggaactgaaaatgg | 45 | 145 |
| 25373 | 3′ UTR | 766 | gcaggagggaactgaaaa | 35 | 146 |
| 25374 | 3′ UTR | 851 | agggcagggcataggcgt | 5 | 147 |
| 25375 | 3′ UTR | 854 | ggaagggcagggcatagg | 0 | 148 |
| 25376 | 3′ UTR | 859 | catgaggaagggcagggc | 0 | 149 |
| 25377 | 3′ UTR | 920 | taaagtgctggtgtgtga | 20 | 150 |
| 25378 | 3′ UTR | 939 | cctgtgagccagaagtgt | 67 | 151 |
| 25379 | 3′ UTR | 941 | ttcctgtgagccagaagt | 61 | 152 |
| 25380 | 3′ UTR | 945 | cactttcctgtgagccag | 80 | 153 |
| 25381 | 3′ UTR | 948 | agacactttcctgtgagc | 0 | 154 |
| 25382 | 3′ UTR | 966 | actctgggtccctactgc | 0 | 155 |
| 25383 | 3′ UTR | 992 | tgcagaaacaactccagg | 0 | 156 |

### Example 22

Automated Assay of Cellular Inhibitor of Apoptosis-2 Expression Oligonucleotide Activity

Cellular Inhibitor of Apoptosis-2 (also known as c-IAP-2, apoptosis inhibitor 2, API-2, hIAP-1, and MIHC) is a member of the inhibitor of apoptosis (IAP) family of anti-apoptotic proteins which interfere with the transmission of intracellular death signals.

Oligonucleotides targeting Cellular Inhibitor of Apoptosis-2 were designed, synthesized, analyzed and assayed according to procedures outlined in U.S. Ser. No. 09/067,638. Alternatively, Oligonucleotides targeting Cellular Inhibitor of Apoptosis-2 can be designed as described in Example 2, synthesized as described in Examples 3-7, analyzed as described in Example 8 and assayed by RT-PCR as described in Example 12.

Cellular Inhibitor of Apoptosis-2 probes and primers were designed to hybridize to the human Cellular Inhibitor of Apoptosis-2 sequence, using published sequences information (GenBank accession number U37546, incorporated herein by reference as SEQ ID NO: 157).

For Cellular Inhibitor of Apoptosis-2-the PCR primers were:

forward primer: GGACTCAGGTGTTGGGAATCTG (SEQ ID NO: 158)

reverse primer: CAAGTACTCACACCTTGGAAACCA (SEQ ID NO: 159) and the PCR

probe was: FAM-AGATGATCCATGGGTTCAACATGCCAA-TAMRA (SEQ ID NO: 160) where FAM (PE-Applied Biosystems, Foster City, Calif.) is the fluorescent reporter dye) and TAMRA (PE-Applied Biosystems, Foster City, Calif.) is the quencher dye.

### Example 23

Antisense Inhibition of Cellular Inhibitor of Apoptosis-2 Expression-Phosphorothioate Oligodeoxynucleotides

In accordance with the present invention, a series of oligonucleotides were designed to target different regions of the human Cellular Inhibitor of Apoptosis-2 RNA, using published sequences (GenBank accession number U37546, incorporated herein by reference as SEQ ID NO: 157). The oligonucleotides are shown in Table 12. Target sites are indicated by nucleotide numbers, as given in the sequence source reference (Genbank accession no. U37546), to which the oligonucleotide binds. All compounds in Table 12 are oligodeoxynucleotides with phosphorothioate backbones (internucleoside linkages) throughout. The compounds were analyzed for effect on Cellular Inhibitor of Apoptosis-2 mRNA levels by quantitative real-time PCR as described in other examples herein. Data are averages from three experiments. If present, “N.D.” indicates “no data”.

TABLE 12

Inhibition of Cellular Inhibitor
of Apoptosis-2 mRNA levels
by phosphorotbioate oligodeoxynucleotides

| | | | | | |
|---|---|---|---|---|---|
| | | | | % | |
| | | | | Inhi- | |
| | | TARGET | | bi- | SEQ ID |
| ISIS# | REGION | SITE | SEQUENCE | tion | NO. |

| | | | | | |
|---|---|---|---|---|---|
| 23412 | 5′ UTR | 3 | actgaagacattttgaat | 62 | 161 |
| 23413 | 5′ UTR | 37 | cttagaggtacgtaaaat | 29 | 162 |
| 23414 | 5′ UTR | 49 | gcacttuatttcttaga | 70 | 163 |
| 23415 | 5′ UTR | 62 | attttaattagaagcact | 0 | 164 |
| 23416 | 5′ UTR | 139 | accatatttcactgattc | 70 | 165 |
| 23417 | 5′ UTR | 167 | ctaactcaaaggaggaaa | 0 | 166 |
| 23418 | 5′ UTR | 175 | cacaagacctaactcaaa | 27 | 167 |
| 23419 | 5′ UTR | 268 | gctctgctgtcaagtgtt | 57 | 168 |
| 23420 | 5′ UTR | 303 | tgtgtgactcatgaagct | 23 | 169 |
| 23421 | 5′ UTR | 335 | ttcagtggcattcaatca | 23 | 170 |
| 23422 | 5′ UTR | 357 | cttctccaggctactaga | 50 | 171 |
| 23423 | 5′ UTR | 363 | ggtcaacttctccaggct | 65 | 172 |
| 23424 | 5′ UTR | 437 | taaaacccttcacagaag | 0 | 173 |
| 23425 | 5′ UTR | 525 | ttaaggaagaaatacaca | 0 | 174 |
| 23426 | 5′ UTR | 651 | gcatggctttgcttttat | 0 | 175 |
| 23427 | Coding | 768 | caaacgtgttggcgcttt | 35 | 176 |
| 23428 | Coding | 830 | agcaggaaaagtggaata | 0 | 177 |
| 23429 | Coding | 1015 | ttaacggaatttagactc | 0 | 178 |
| 23430 | Coding | 1064 | atttgttactgaagaagg | 0 | 179 |
| 23431 | Coding | 1118 | agagccacggaaatatcc | 9 | 180 |
| 23432 | Coding | 1168 | aaatcttgatttgctctg | 7 | 181 |
| 23433 | Coding | 1231 | gtaagtaatctggcattt | 0 | 182 |
| 23434 | Coding | 1323 | agcaagccactctgtctc | 50 | 183 |
| 23435 | Coding | 1436 | tgaagtgtcttgaagctg | 0 | 184 |
| 23436 | Coding | 1580 | tttgacatcatcactgtt | 0 | 185 |
| 23437 | Coding | 1716 | tggcttgaacttgacgga | 0 | 186 |
| 23438 | Coding | 1771 | tcatctcctgggctgtct | 40 | 187 |
| 23439 | Coding | 1861 | gcagcattaatcacagga | 0 | 188 |
| 23440 | Coding | 2007 | tttctctctcctcttccc | 10 | 189 |
| 23441 | Coding | 2150 | aacatcatgttcttgttc | 9 | 190 |
| 23442 | Coding | 2273 | atataacacagcttcagc | 0 | 191 |
| 23443 | Coding | 2350 | aattgftcttccactggt | 0 | 192 |
| 23444 | Coding | 2460 | aagaaggagcacaatctt | 70 | 193 |
| 23445 | 3′ UTR | 2604 | gaaaccaaattaggataa | 12 | 194 |
| 23446 | 3′ UTR | 2753 | tgtagtgctacctcttu | 69 | 195 |
| 23447 | 3′ UTR | 2779 | ctgaaatutgattgaat | 14 | 196 |
| 23448 | 3′ UTR | 2795 | tacaatttcaataatgct | 38 | 197 |
| 23449 | 3′ UTR | 2920 | gggtctcagtatgctgcc | 21 | 198 |
| 23450 | 3′ UTR | 3005 | ccttcgatgtataggaca | 0 | 199 |
| 23451 | 3′ UTR | 3040 | catgtccctaaaatgtca | 0 | 200 |

### Example 24

Antisense Inhibition of Cellular Inhibitor of Apoptosis-2 Expression-Phosphorothioate 2′-MOE Gapmer Oligonucleotides

In accordance with the present invention, a second series of oligonucleotides targeted to human Cellular Inhibitor of Apoptosis-2 were synthesized. The oligonucleotide sequences are shown in Table 13. Target sites are indicated by nucleotide numbers, as given in the sequence source reference (Genbank accession no. U37546), to which the oligonucleotide binds.

All compounds in Table 13 are chimeric oligonucleotides (“gapmers”) 18 nucleotides in length, composed of a central “gap” region consisting of ten 2′-deoxynucleotides, which is flanked on both sides (5′ and 3′ directions) by four-nucleotide “wings.” The wings are composed of 2′-methoxyethyl (2′-MOE)nucleotides. The internucleoside (backbone) linkages are phosphorothioate (P═S) throughout the oligonucleotide. Cytidine residues in the 2′-MOE wings are 5-methylcytidines.

Data were obtained by real-time quantitative PCR as described in other examples herein and are averaged from three experiments. If present, “N.D.” indicates “no data”.

TABLE 13

Inhibition of Cellular Inhibitor
of Apoptosis-2 mRNA levels
by chimeric phosphorothioate oligonucleotides
having 2′-MOE wings and a deoxy gap

| | | | | | |
|---|---|---|---|---|---|
| | | | | % | |
| | | | | Inhi- | |
| | | TARGET | | bi- | SEQ ID |
| ISIS# | REGION | SITE | SEQUENCE | tion | NO. |

| | | | | | |
|---|---|---|---|---|---|
| 23452 | 5′ UTR | 3 | actgaagacattttgaat | 35 | 161 |
| 23453 | 5′ UTR | 37 | cttagaggtacgtaaaat | 26 | 162 |
| 23454 | 5′ UTR | 49 | gcacttttatttcttaga | 76 | 163 |
| 23455 | 5′ UTR | 62 | attttaattagaagcact | 0 | 164 |
| 23456 | 5′ UTR | 139 | accatatttcactgauc | 0 | 165 |
| 23457 | 5′ UTR | 167 | ctaactcaaaggaggaaa | 5 | 166 |
| 23458 | 5′ UTR | 175 | cacaagacctaactcaaa | 0 | 167 |
| 23459 | 5′ UTR | 268 | gctctgctgtcaagtgtt | 57 | 168 |
| 23460 | 5′ UTR | 303 | tgtgtgactcatgaagct | 67 | 169 |
| 23461 | 5′ UTR | 335 | ttcagtggcattcaatca | 59 | 170 |
| 23462 | 5′ UTR | 357 | cttctccaggctactaga | 0 | 171 |
| 23463 | 5′ UTR | 363 | ggtcaacttctccaggct | 75 | 172 |
| 23464 | 5′ UTR | 437 | taaaacccttcacagaag | 11 | 173 |
| 23465 | 5′ UTR | 525 | ttaaggaagaaatacaca | 0 | 174 |
| 23466 | 5′ UTR | 651 | gcatggctttgcttttat | 46 | 175 |
| 23467 | Coding | 768 | caaacgtgttggcgcttt | 47 | 176 |
| 23468 | Coding | 830 | agcaggaaaagtggaata | 39 | 177 |
| 23469 | Coding | 1015 | ttaacggaatttagactc | 12 | 178 |
| 23470 | Coding | 1064 | atttgttactgaagaagg | 34 | 179 |
| 23471 | Coding | 1118 | agagccacggaaatatcc | 54 | 180 |
| 23472 | Coding | 1168 | aaatcttgatttgctctg | 34 | 181 |
| 23473 | Coding | 1231 | gtaagtaatctggcattt | 0 | 182 |
| 23474 | Coding | 1323 | agcaagccactctgtctc | 42 | 183 |
| 23475 | Coding | 1436 | tgaagtgtcttgaagctg | 0 | 184 |
| 23476 | Coding | 1580 | tttgacatcatcactgtt | 57 | 185 |
| 23477 | Coding | 1716 | tggcttgaacttgacgga | 23 | 186 |
| 23478 | Coding | 1771 | tcatctcctgggctgtct | 66 | 187 |
| 23479 | Coding | 1861 | gcagcattaatcacagga | 65 | 188 |
| 23480 | Coding | 2007 | tttctctctcctcttccc | 0 | 189 |
| 23481 | Coding | 2150 | aacatcatgttcttgttc | 13 | 190 |
| 23482 | Coding | 2273 | atataacacagcttcagc | 0 | 191 |
| 23483 | Coding | 2350 | aattgttcttccactggt | 60 | 192 |
| 23484 | Coding | 2460 | aagaaggagcacaatctt | 65 | 193 |
| 23485 | 3′ UTR | 2604 | gaaaccaaattaggataa | 0 | 194 |
| 23486 | 3′ UTR | 2753 | tgtagtgctacctctttt | 73 | 195 |
| 23487 | 3′ UTR | 2779 | ctgaaattttgattgaat | 4 | 196 |
| 23488 | 3′ UTR | 2795 | tacaatttcaataatgct | 0 | 197 |
| 23489 | 3′ UTR | 2920 | gggtctcagtatgctgcc | 42 | 198 |
| 23490 | 3′ UTR | 3005 | ccttcgatgtataggaca | 71 | 199 |
| 23491 | 3′ UTR | 3040 | catgtccctaaaatgtca | 45 | 200 |

### Example 25

Automated Assay of ELK-1 Oligonucleotide Activity

ELK-1 (also known as p62TCF) is a member of the ternary complex factor (TCF) subfamily of Ets domain proteins and utilizes a bipartite recognition mechanism mediated by both protein-DNA and protein-protein interactions. This results in gene regulation not only by direct DNA binding but also by indirect DNA binding through recruitment by other factors (Rao et al., Science, 1989, 244, 66-70). The formation of ternary complexes with an array of proteins allows the differential regulation of many genes. The mechanism by which ELK-1 controls various signal transduction pathways involves regulating the activity of the Egr-1, pip92, nur77 and c-fos promoters by binding to the serum response element (SRE) in these promoters in response to extracellular stimuli such as growth factors, mitogens and oncogene products (Sharrocks et al., Int. J. Biochem. Cell Biol., 1997, 29, 1371-1387). ELK-1 has also been shown to mediate other functions within the cell including apoptosis.

Oligonucleotides targeting ELK-1 were designed, synthesized, analyzed and assayed according to procedures outlined in U.S. Ser. No. 09/067,638. Alternatively, Oligonucleotides targeting ELK-1 can be designed as described in Example 2, synthesized as described in Examples 3-7, analyzed as described in Example 8 and assayed by RT-PCR as described in Example 12.

ELK-1 probes and primers were designed to hybridize to the human ELK-1 sequence, using published sequence information (GenBank accession number M25269, incorporated herein by reference as SEQ ID NO: 201).

For ELK-1 the PCR primers were:

forward primer: GCAAGGCAATGGCCACAT (SEQ ID NO: 202)

reverse primer: CTCCTCTGCATCCACCAGCTT (SEQ ID NO: 203) and the PCR probe

was: FAM-TCTCCTGGACTTCACGGGATGGTGGT-TAMRA (SEQ ID NO: 204) where FAM (PE-Applied Biosystems, Foster City, Calif.) is the fluorescent reporter dye) and TAMRA (PE-Applied Biosystems, Foster City, Calif.) is the quencher dye.

### Example 26

Antisense Inhibition of ELK-1 Expression-Phosphorothioate Oligodeoxynucleotides

In accordance with the present invention, a series of oligonucleotides were designed to target different regions of the human ELK-1 RNA, using published sequences (GenBank accession number M25269, incorporated herein by reference as SEQ ID NO: 201). The oligonucleotides are shown in Table 14. Target sites are indicated by nucleotide numbers, as given in the sequence source reference (Genbank accession no. M25269), to which the oligonucleotide binds. All compounds in Table 14 are oligodeoxynucleotides with phosphorothioate backbones (internucleoside linkages) throughout. The compounds were analyzed for effect on ELK-1 mRNA levels by quantitative real-time PCR as described in other examples herein. Data are averages from three experiments. If present, “N.D.” indicates “no data”.

TABLE 14

Inhibition of ELK-1 mRNA levels
by phosphorothioate oligodeoxynucleotides

| | | | | | |
|---|---|---|---|---|---|
| | | | | % | |
| | | | | Inhi- | |
| | | TARGET | | bi- | SEQ ID |
| ISIS# | REGION | SITE | SEQUENCE | tion | NO. |

| | | | | | |
|---|---|---|---|---|---|
| 24752 | 5′ UTR | 11 | cccctgcgtttccctaca | 15 | 205 |
| 24753 | 5′ UTR | 50 | ggtggtggtggcggtggc | 29 | 206 |
| 24754 | 5′ UTR | 139 | ggcgttggcaatgttggc | 82 | 207 |
| 24755 | 5′ UTR | 167 | aagttgaggctgtgtgta | 0 | 208 |
| 24756 | 5′ UTR | 189 | aggccacggacgggtctc | 92 | 209 |
| 24757 | 5′ UTR | 229 | gattgattcgctacgatg | 71 | 210 |
| 24758 | 5′ UTR | 255 | gggatgcggaggagtgcg | 74 | 211 |
| 24759 | 5′ UTR | 289 | agtgctcacgccatccca | 22 | 212 |
| 24760 | Coding | 328 | aaactgccacagcgtcac | 64 | 213 |
| 24761 | Coding | 381 | gaagtccaggagatgatg | 62 | 214 |
| 24762 | Coding | 395 | caccaccatcccgtgaag | 88 | 215 |
| 24763 | Coding | 455 | tcttgttcttgcgtagtc | 62 | 216 |
| 24764 | Coding | 512 | tgttcttgtcatagtagt | 52 | 217 |
| 24765 | Coding | 527 | tcaccttgcggatgatgt | 57 | 218 |
| 24766 | Coding | 582 | gagcaccctgcgacctca | 72 | 219 |
| 24767 | Coding | 600 | ggcgggcagtcctcagtg | 82 | 220 |
| 24768 | Coding | 787 | ggtgaaggtggaatagag | 58 | 221 |
| 24769 | Coding | 993 | tccgatttcaggtuggg | 55 | 222 |
| 24770 | Coding | 1110 | ttggtggtuctggcaca | 67 | 223 |
| 24771 | Coding | 1132 | tggagggacttctggctc | 69 | 224 |
| 24772 | Coding | 1376 | gcgtaggaagcagggatg | 34 | 225 |
| 24773 | Coding | 1440 | gtgctccagaagtgaatg | 64 | 226 |
| 24774 | Coding | 1498 | actggatggaaactggaa | 34 | 227 |
| 24775 | Coding | 1541 | ggccatccacgctgatag | 74 | 228 |
| 24776 | 3′ UTR | 1701 | ccaccacaatcagagcat | 74 | 229 |
| 24777 | 3′ UTR | 1711 | gatccccaccccaccaca | 16 | 230 |
| 24778 | 3′ UTR | 1765 | tgttttctgtggaggaga | 48 | 231 |
| 24779 | 3′ UTR | 1790 | aaacagagaagttgtgga | 11 | 232 |
| 24780 | 3′ UTR | 1802 | gggactgacagaaaacag | 0 | 233 |
| 24781 | 3′ UTR | 1860 | ataaataaataaaccgcc | 18 | 234 |
| 24782 | 3′ UTR | 1894 | gttaggtcaggctcatcc | 56 | 235 |
| 24783 | 3′ UTR | 1974 | gttctcaagccagacctc | 52 | 236 |
| 24784 | 3′ UTR | 1992 | aataaagaaagaaaggtc | 41 | 237 |
| 24785 | 3′ UTR | 2006 | agggcaggctgagaaata | 29 | 238 |
| 24786 | 3′ UTR | 2053 | cttctactcacatccaaa | 54 | 239 |
| 24787 | 3′ UTR | 2068 | caaaacaaactaactctt | 24 | 240 |
| 24788 | 3′ UTR | 2080 | ggaataataaaacaaaac | 40 | 241 |
| 24789 | 3′ UTR | 2107 | ttcttcctggacccctga | 93 | 242 |
| 24790 | 3′ UTR | 2161 | ccaagggtgtgattcttc | 81 | 243 |
| 24791 | 3′ UTR | 2200 | tgtctgagagaaaggttg | 55 | 244 |

### Example 27

Antisense Inhibition of ELK-1 Expression-Phosphorothioate 2′-MOE Gapmer Oligonucleotides

In accordance with the present invention, a second series of oligonucleotides targeted to human ELK-1 were synthesized. The oligonucleotide sequences are shown in Table 15. Target sites are indicated by nucleotide numbers, as given in the sequence source reference (Genbank accession no. M25269), to which the oligonucleotide binds.

All compounds in Table 15 are chimeric oligonucleotides (“gapmers”) 18 nucleotides in length, composed of a central “gap” region consisting of ten 2′-deoxynucleotides, which is flanked on both sides (5′ and 3′ directions) by four-nucleotide “wings.” The wings are composed of 2′-methoxyethyl (2′-MOE)nucleotides. The internucleoside (backbone) linkages are phosphorothioate (P═S) throughout the oligonucleotide. Cytidine residues in the 2′-MOE wings are 5-methylcytidines.

Data were obtained by real-time quantitative PCR as described in other examples herein and are averaged from three experiments. If present, “N.D.” indicates “no data”.

TABLE 15

Inhibition of ELK-1 mRNA levels
by chimeric phosphorothioate oligonucleotides
having 2′-MOE wings and a deoxy gap

| | | | | | |
|---|---|---|---|---|---|
| | | | | % | |
| | | | | Inhi- | |
| | | TARGET | | bi- | SEQ ID |
| ISIS# | REGION | SITE | SEQUENCE | tion | NO. |

| | | | | | |
|---|---|---|---|---|---|
| 24792 | 5′ UTR | 11 | cccctgcgtttccctaca | 23 | 205 |
| 24793 | 5′ UTR | 50 | ggtggtggtggcggtggc | 80 | 206 |
| 24794 | 5′ UTR | 139 | ggcgttggcaatgttggc | 91 | 207 |
| 24795 | 5′ UTR | 167 | aagttgaggctgtgtgta | 27 | 208 |
| 24796 | 5′ UTR | 189 | aggccacggacgggtctc | 79 | 209 |
| 24797 | 5′ UTR | 229 | gattgattcgctacgatg | 69 | 210 |
| 24798 | 5′ UTR | 255 | gggatgcggaggagtgcg | 42 | 211 |
| 24799 | 5′ UTR | 289 | agtgctcacgccatccca | 45 | 212 |
| 24800 | Coding | 328 | aaactgccacagcgtcac | 57 | 213 |
| 24801 | Coding | 381 | gaagtccaggagatgatg | 55 | 214 |
| 24802 | Coding | 395 | caccaccatcccgtgaag | 41 | 215 |
| 24803 | Coding | 455 | tcttgttcttgcgtagtc | 80 | 216 |
| 24804 | Coding | 512 | tgttcttgtcatagtagt | 65 | 217 |
| 24805 | Coding | 527 | tcaccttgcggatgatgt | 70 | 218 |
| 24806 | Coding | 582 | gagcaccctgcgacctca | 64 | 219 |
| 24807 | Coding | 600 | ggcgggcagtcctcagtg | 67 | 220 |
| 24808 | Coding | 787 | ggtgaaggtggaatagag | 45 | 221 |
| 24809 | Coding | 993 | tccgatttcaggtttggg | 75 | 222 |
| 24810 | Coding | 1110 | ttggtggtttctggcaca | 82 | 223 |
| 24811 | Coding | 1132 | tggagggacttctggctc | 60 | 224 |
| 24812 | Coding | 1376 | gcgtaggaagcagggatg | 49 | 225 |
| 24813 | Coding | 1440 | gtgctccagaagtgaatg | 71 | 226 |
| 24814 | Coding | 1498 | actggatggaaactggaa | 62 | 227 |
| 24815 | Coding | 1541 | ggccatccacgctgatag | 78 | 228 |
| 24816 | 3′ UTR | 1701 | ccaccacaatcagagcat | 54 | 229 |
| 24817 | 3′ UTR | 1711 | gatccccaccccaccaca | 44 | 230 |
| 24818 | 3′ UTR | 1765 | tgttttctgtggaggaga | 74 | 231 |
| 24819 | 3′ UTR | 1790 | aaacagagaagttgtgga | 64 | 232 |
| 24820 | 3′ UTR | 1802 | gggactgacagaaaacag | 16 | 233 |
| 24821 | 3′ UTR | 1860 | ataaataaataaaccgcc | 38 | 234 |
| 24822 | 3′ UTR | 1894 | gttaggtcaggctcatcc | 59 | 235 |
| 24823 | 3′ UTR | 1974 | gttctcaagccagacctc | 62 | 236 |
| 24824 | 3′ UTR | 1992 | aataaagaaagaaaggtc | 35 | 237 |
| 24825 | 3′ UTR | 2006 | agggcaggctgagaaata | 0 | 238 |
| 24826 | 3′ UTR | 2053 | cttctactcacatccaaa | 46 | 239 |
| 24827 | 3′ UTR | 2068 | caaaacaaactaactctt | 38 | 240 |
| 24828 | 3′ UTR | 2080 | ggaataataaaacaaaac | 37 | 241 |
| 24829 | 3′ UTR | 2107 | ttcttcctggacccctga | 71 | 242 |
| 24830 | 3′ UTR | 2161 | ccaagggtgtgattcttc | 88 | 243 |
| 24831 | 3′ UTR | 2200 | tgtctgagagaaaggttg | 65 | 244 |

### Example 28

Automated Assay of Gi alpha-11 Oligonucleotide Activity

G-alpha-11 is a member of the Gq subfamily of G proteins whose primary function is to activate PLC-b isoforms producing second messengers and affecting intracellular calcium stores.

Oligonucleotides targeting Gi alpha-11 were designed, synthesized, analyzed and assayed according to procedures outlined in U.S. Ser. No. 09/067,638. Alternatively, oligonucleotides targeting Gi alpha-11 can be designed as described in Example 2, synthesized as described in Examples 3-7, analyzed as described in Example 8 and assayed by RT-PCR as described in Example 12.

G-alpha-11 probes and primers were designed to hybridize to the human G-alpha-11 sequence, using published sequence information (GenBank accession number AF011497, incorporated herein by reference as SEQ ID NO: 245). For G-alpha-11 the PCR primers were:

forward primer: TGACCACCTTCGAGCATCAG (SEQ ID NO: 246)

reverse primer: CGGTCGTAGCATTCCTGGAT (SEQ ID NO: 247) and the PCR probe

was: FAM-TCAGTGCCATCAAGACCCTGTGGGAG-TAMRA (SEQ ID NO: 248) where FAM (PE-Applied Biosystems, Foster City, Calif.) is the fluorescent reporter dye) and TAMRA (PE-Applied Biosystems, Foster City, Calif.) is the quencher dye.

### Example 29

Antisense Inhibition of G-alpha-11 Expression-Phosphorothioate Oligodeoxynucleotides

In accordance with the present invention, a series of oligonucleotides were designed to target different regions of the human G-alpha-11 RNA, using published sequences (GenBank accession number AF011497, incorporated herein by reference as SEQ ID NO: 245). The oligonucleotides are shown in Table 16. Target sites are indicated by nucleotide numbers, as given in the sequence source reference (Genbank accession no. AF011497), to which the oligonucleotide binds. All compounds in Table 16 are oligodeoxynucleotides with phosphorothioate backbones (internucleoside linkages) throughout. The compounds were analyzed for effect on G-alpha-11 mRNA levels by quantitative real-time PCR as described in other examples herein. Data are averages from three experiments. If present, “N.D.” indicates “no data”.

TABLE 16

Inhibition of G-alpha-11 mRNA levels
by phosphorothioate oligodeoxynucleotides

| | | | | | |
|---|---|---|---|---|---|
| | | | | % | |
| | | | | Inhi- | |
| | | TARGET | | bi- | SEQ ID |
| ISIS# | REGION | SITE | SEQUENCE | tion | NO. |

| | | | | | |
|---|---|---|---|---|---|
| 20576 | Coding | 1 | gatggactccagagtcat | 0 | 249 |
| 20577 | Coding | 6 | gccatgatggactccaga | 75 | 250 |
| 20578 | Coding | 9 | cacgccatgatggactcc | 0 | 251 |
| 20579 | Coding | 25 | ctcatcgctcaggcaaca | 61 | 252 |
| 20580 | Coding | 31 | cttcacctcatcgctcag | 20 | 253 |
| 20581 | Coding | 36 | gactccttcacctcatcg | 15 | 254 |
| 20582 | Coding | 45 | atccgcttggactccttc | 17 | 255 |
| 20583 | Coding | 50 | cgttgatccgcttggact | 0 | 256 |
| 20584 | Coding | 61 | ctcgatctcggcgttgat | 0 | 257 |
| 20585 | Coding | 77 | cccgccgcagctgcttct | 58 | 258 |
| 20586 | Coding | 106 | cttgagctcgcgccgggc | 31 | 259 |
| 20587 | Coding | 116 | gcagcagcagcttgagct | 0 | 260 |
| 20588 | Coding | 127 | gcccgtgccgagcagcag | 0 | 261 |
| 20589 | Coding | 146 | acgtgctcttcccgctct | 28 | 262 |
| 20590 | Coding | 159 | atctgcttgatgaacgtg | 0 | 263 |
| 20591 | Coding | 162 | cgcatctgcttgatgaac | 0 | 264 |
| 20592 | Coding | 184 | gtagccggcgccgtggat | 1 | 265 |
| 20593 | Coding | 197 | tgtcctcctccgagtagc | 0 | 266 |
| 20594 | Coding | 199 | cttgtcctcctccgagta | 79 | 267 |
| 20595 | Coding | 207 | aagccgcgcttgtcctcc | 56 | 268 |
| 20596 | Coding | 222 | tagacgagcttggtgaag | 0 | 269 |
| 20597 | Coding | 230 | tgttctggtagacgagct | 0 | 270 |
| 20598 | Coding | 242 | tggcggtgaagatgttct | 0 | 271 |
| 20599 | Coding | 258 | cggatcatggcctgcatg | 1 | 272 |
| 20600 | Coding | 271 | cgtctccatggcccggat | 49 | 273 |
| 20601 | Coding | 285 | tagaggatcttgagcgtc | 0 | 274 |
| 20602 | Coding | 287 | tgtagaggatcttgagcg | 0 | 275 |
| 20603 | Coding | 297 | tgctcgtacttgtagagg | 7 | 276 |
| 20604 | Coding | 306 | gccttgttctgctcgtac | 25 | 277 |
| 20605 | Coding | 309 | ttggccttgttctgctcg | 0 | 278 |
| 20606 | Coding | 319 | caggagcgcattggcctt | 0 | 279 |
| 20607 | Coding | 340 | ctccacgtccacctcccg | 69 | 280 |
| 20608 | Coding | 349 | ggtcaccttctccacgtc | 27 | 281 |
| 20609 | Coding | 362 | gatgctcgaaggtggtca | 33 | 282 |
| 20610 | Coding | 373 | actgacgtactgatgctc | 36 | 283 |
| 20611 | Coding | 382 | cttgatggcactgacgta | 78 | 284 |
| 20612 | Coding | 388 | cagggtcttgatggcact | 0 | 285 |
| 20613 | Coding | 409 | ctggatgcccgggtcctc | 0 | 286 |
| 20614 | Coding | 411 | tcctggatgcccgggtcc | 30 | 287 |
| 20615 | Coding | 429 | cgcctgcggtcgtagcat | 0 | 288 |
| 20616 | Coding | 440 | gctggtactcgcgcctgc | 41 | 289 |
| 20617 | Coding | 459 | tacttggcagagtcggag | 34 | 290 |
| 20618 | Coding | 468 | gtcaggtagtacttggca | 76 | 291 |
| 20619 | Coding | 479 | ggtcaacgtcggtcaggt | 18 | 292 |
| 20620 | Coding | 489 | gtggcgatgcggtcaacg | 1 | 293 |
| 20621 | Coding | 503 | gcaggtagcccaaggtgg | 20 | 294 |
| 20622 | Coding | 518 | cgtcctgctgggtgggca | 40 | 295 |
| 20623 | Coding | 544 | ggtggtgggcacgcggac | 0 | 296 |
| 20624 | Coding | 555 | tcgatgatgccggtggtg | 0 | 297 |
| 20625 | Coding | 572 | ccaggtcgaaagggtact | 0 | 298 |
| 20626 | Coding | 578 | tgttctccaggtcgaaag | 33 | 299 |
| 20627 | Coding | 584 | agatgatgttctccaggt | 0 | 300 |
| 20628 | Coding | 591 | atccggaagatgatgttc | 0 | 301 |
| 20629 | Coding | 624 | ctccgctccgaccgctgg | 56 | 302 |
| 20630 | Coding | 634 | gatccacttcctccgctc | 59 | 303 |
| 20631 | Coding | 655 | tgtcacguctcaaagca | 0 | 304 |
| 20632 | Coding | 663 | atgatggatgtcacgttc | 0 | 305 |
| 20633 | Coding | 671 | cgagaaacatgatggatg | 0 | 306 |
| 20634 | Coding | 682 | gctgagggcgacgagaaa | 75 | 307 |
| 20635 | Coding | 709 | cgactccaccaggacttg | 40 | 308 |
| 20636 | Coding | 726 | atccggttctcgugtcc | 22 | 309 |
| 20637 | Coding | 728 | ccatccggttctcgttgt | 19 | 310 |
| 20638 | Coding | 744 | agggctttgctctcctcc | 77 | 311 |
| 20639 | Coding | 754 | ggtccggaacagggcttt | 26 | 312 |
| 20640 | Coding | 766 | gtaggtgatgatggtccg | 0 | 313 |
| 20641 | Coding | 787 | ggaggagttctggaacca | 64 | 314 |
| 20642 | Coding | 803 | tgaggaagaggatgacgg | 0 | 315 |
| 20643 | Coding | 818 | gcaggtccttcttgttga | 6 | 316 |
| 20644 | Coding | 831 | atcttgtcctccagcagg | 4 | 317 |
| 20645 | Coding | 842 | gcgagtacaggatcttgt | 17 | 318 |
| 20646 | Coding | 858 | aagtagtccaccaggtgc | 0 | 319 |
| 20647 | Coding | 910 | gatgaactcccgcgccgc | 52 | 320 |
| 20648 | Coding | 935 | ggttcaggtccacgaaca | 71 | 321 |
| 20649 | Coding | 958 | gtagatgatcttgtcgct | 0 | 322 |
| 20650 | Coding | 972 | cacgtgaagtgtgagtag | 0 | 323 |
| 20651 | Coding | 993 | atgttctccgtgtcggtg | 0 | 324 |
| 20652 | Coding | 1014 | acggccgcgaacacgaag | 6 | 325 |
| 20653 | Coding | 1027 | gatggtgtccttcacggc | 0 | 326 |
| 20654 | Coding | 1043 | tcaggttcagctgcagga | 3 | 327 |
| 20655 | Coding | 1059 | accagattgtactccttc | 0 | 328 |

### Example 30

Antisense Inhibition of G-alpha-11 Expression-Phosphorothioate 2′-MOE Gapmer Oligonucleotides

In accordance with the present invention, a second series of oligonucleotides targeted to human G-alpha-11 were synthesized. The oligonucleotide sequences are shown in Table 16. Target sites are indicated by nucleotide numbers, as given in the sequence source reference (Genbank accession no. AF011497), to which the oligonucleotide binds.

All compounds in Table 17 are chimeric oligonucleotides (“gapmers”) 18 nucleosides in length, composed of a central “gap” region consisting of ten 2′-deoxynucleotides, which is flanked on both sides (5′ and 3′ directions) by four-nucleotide “wings.” The wings are composed of 2′-methoxyethyl (2′-MOE)nucleotides. The internucleoside (backbone) linkages are phosphorothioate (P═S) throughout the oligonucleotide. Cytidine residues in the 2′-MOE wings are 5-methylcytidines.

Data were obtained by real-time quantitative PCR as described in other examples herein and are averaged from three experiments. If present, “N.D.” indicates “no data”.

TABLE 17

Inhibition of G-alpha-11 mRNA levels
by chimeric phosphorothioate oligonucleotides
having 2′-MOE wings and a deoxy gap

| | | | | | |
|---|---|---|---|---|---|
| | | | | % | |
| | | | | Inhi- | |
| | | TARGET | | bi- | SEQ ID |
| ISIS# | REGION | SITE | SEQUENCE | tion | NO. |

| | | | | | |
|---|---|---|---|---|---|
| 20981 | Coding | 1 | gatggactccagagtcat | 0 | 249 |
| 20982 | Coding | 6 | gccatgatggactccaga | 0 | 250 |
| 20983 | Coding | 9 | cacgccatgatggactcc | 0 | 251 |
| 20984 | Coding | 25 | ctcatcgctcaggcaaca | 0 | 252 |
| 20985 | Coding | 31 | cttcacctcatcgctcag | 2 | 253 |
| 20986 | Coding | 36 | gactccttcacctcatcg | 0 | 254 |
| 20987 | Coding | 45 | atccgcttggactccttc | 19 | 255 |
| 20988 | Coding | 50 | cgttgatccgcttggact | 15 | 256 |
| 20989 | Coding | 61 | ctcgatctcggcgttgat | 0 | 257 |
| 20990 | Coding | 77 | cccgccgcagctgcttct | 41 | 258 |
| 20991 | Coding | 106 | cttgagctcgcgccgggc | 19 | 259 |
| 20992 | Coding | 116 | gcagcagcagcttgagct | 23 | 260 |
| 20993 | Coding | 127 | gcccgtgccgagcagcag | 38 | 261 |
| 20994 | Coding | 146 | acgtgctcttcccgctct | 34 | 262 |
| 20995 | Coding | 159 | atctgcttgatgaacgtg | 56 | 263 |
| 20996 | Coding | 162 | cgcatctgcttgatgaac | 31 | 264 |
| 20997 | Coding | 184 | gtagccggcgccgtggat | 0 | 265 |
| 20998 | Coding | 197 | tgtcctcctccgagtagc | 42 | 266 |
| 20999 | Coding | 199 | cttgtcctcctccgagta | 0 | 267 |
| 21000 | Coding | 207 | aagccgcgcttgtcctcc | 73 | 268 |
| 21001 | Coding | 222 | tagacgagcttggtgaag | 0 | 269 |
| 21002 | Coding | 230 | tgttctggtagacgagct | 61 | 270 |
| 21003 | Coding | 242 | tggcggtgaagatguct | 14 | 271 |
| 21004 | Coding | 258 | cggatcatggcctgcatg | 84 | 272 |
| 21005 | Coding | 271 | cgtctccatggcccggat | 70 | 273 |
| 21006 | Coding | 285 | tagaggatcttgagcgtc | 39 | 274 |
| 21007 | Coding | 287 | tgtagaggatcttgagcg | 28 | 275 |
| 21008 | Coding | 297 | tgctcgtacttgtagagg | 70 | 276 |
| 21009 | Coding | 306 | gccttgttctgctcgtac | 76 | 277 |
| 21010 | Coding | 309 | ttggccttgttctgctcg | 0 | 278 |
| 21011 | Coding | 319 | caggagcgcattggcctt | 87 | 279 |
| 21012 | Coding | 340 | ctccacgtccacctcccg | 0 | 280 |
| 21013 | Coding | 349 | ggtcaccttctccacgtc | 69 | 281 |
| 21014 | Coding | 362 | gatgctcgaaggtggtca | 0 | 282 |
| 21015 | Coding | 373 | actgacgtactgatgctc | 69 | 283 |
| 21016 | Coding | 382 | cttgatggcactgacgta | 32 | 284 |
| 21017 | Coding | 388 | cagggtcttgatggcact | 19 | 285 |
| 21018 | Coding | 409 | ctggatgcccgggtcctc | 63 | 286 |
| 21019 | Coding | 411 | tcctggatgcccgggtcc | 56 | 287 |
| 21020 | Coding | 429 | cgcctgcggtcgtagcat | 73 | 288 |
| 21021 | Coding | 440 | gctggtactcgcgcctgc | 68 | 289 |
| 21022 | Coding | 459 | tacttggcagagtcggag | 50 | 290 |
| 21023 | Coding | 468 | gtcaggtagtacttggca | 13 | 291 |
| 21024 | Coding | 479 | ggtcaacgtcggtcaggt | 64 | 292 |
| 21025 | Coding | 489 | gtggcgatgcggtcaacg | 52 | 293 |
| 21026 | Coding | 503 | gcaggtagcccaaggtgg | 52 | 294 |
| 21027 | Coding | 518 | cgtcctgctgggtgggca | 0 | 295 |
| 21028 | Coding | 544 | ggtggtgggcacgcggac | 81 | 296 |
| 21029 | Coding | 555 | tcgatgatgccggtggtg | 48 | 297 |
| 21030 | Coding | 572 | ccaggtcgaaagggtaci | 61 | 298 |
| 21031 | Coding | 578 | tgttctccaggtcgaaag | 0 | 299 |
| 21032 | Coding | 584 | agatgatgttctccaggt | 0 | 300 |
| 21033 | Coding | 591 | atccggaagatgatguc | 0 | 301 |
| 21034 | Coding | 624 | ctccgctccgaccgctgg | 59 | 302 |
| 21035 | Coding | 634 | gatccacttcctccgctc | 17 | 303 |
| 21036 | Coding | 655 | tgtcacgttctcaaagca | 9 | 304 |
| 21037 | Coding | 663 | atgatggatgtcacgttc | 41 | 305 |
| 21038 | Coding | 671 | cgagaaacatgatggatg | 0 | 306 |
| 21039 | Coding | 682 | gctgagggcgacgagaaa | 11 | 307 |
| 21040 | Coding | 709 | cgactccaccaggacttg | 0 | 308 |
| 21041 | Coding | 726 | atccggttctcgttgtcc | 67 | 309 |
| 21042 | Coding | 728 | ccatccgguctcgttgt | 30 | 310 |
| 21043 | Coding | 744 | agggctttgctctcctcc | 61 | 311 |
| 21044 | Coding | 754 | ggtccggaacagggcttt | 72 | 312 |
| 21045 | Coding | 766 | gtaggtgatgatggtccg | 68 | 313 |
| 21046 | Coding | 787 | ggaggagttctggaacca | 54 | 314 |
| 21047 | Coding | 803 | tgaggaagaggatgacgg | 23 | 315 |
| 21048 | Coding | 818 | gcaggtccttcttgttga | 0 | 316 |
| 21049 | Coding | 831 | atcttgtcctccagcagg | 39 | 317 |
| 21050 | Coding | 842 | gcgagtacaggatcttgt | 74 | 318 |
| 21051 | Coding | 858 | aagtagtccaccaggtgc | 36 | 319 |
| 21052 | Coding | 910 | gatgaactcccgcgccgc | 67 | 320 |
| 21053 | Coding | 935 | ggttcaggtccacgaaca | 37 | 321 |
| 21054 | Coding | 958 | gtagatgatcttgtcgct | 64 | 322 |
| 21055 | Coding | 972 | cacgtgaagtgtgagtag | 37 | 323 |
| 21056 | Coding | 993 | atgttctccgtgtcggtg | 0 | 324 |
| 21057 | Coding | 1014 | acggccgcgaacacgaag | 0 | 325 |
| 21058 | Coding | 1027 | gatggtgtccttcacggc | 69 | 326 |
| 21059 | Coding | 1043 | tcaggttcagctgcagga | 0 | 327 |
| 21060 | Coding | 1059 | accagattgtactccttc | 0 | 328 |

### Example 31

Automated Assay of AKT-1 Oligonucleotide Activity

Akt-1 (also known as PKB alpha and RAC-PK alpha) is a member of the AKT/PKB family of serine/threonine kinases and has been shown to be involved in a diverse of signaling pathways.

Oligonucleotides targeting AKT-1 were designed, synthesized, analyzed and assayed according to procedures outlined in U.S. Ser. No. 09/067,638. Alternatively, oligonucleotides targeting AKT-1 can be designed as described in Example 2, synthesized as described in Examples 3-7, analyzed as described in Example 8 and assayed by RT-PCR as described in Example 12.

AKT-1 probes and primers were designed to hybridize to the human AKT-1 sequence, using published sequence information (GenBank accession number M63167, incorporated herein by reference as SEQ ID NO: 329). For Akt-1 the PCR primers were:

forward primer: CGTGACCATGAACGAGTTTGA (SEQ ID NO: 330)

reverse primer: CAGGATCACCTTGCCGAAA (SEQ ID NO: 331) and the PCR probe

was: FAM-CTGAAGCTGCTGGGCAAGGGCA-TAMRA (SEQ ID NO: 332) where FAM (PE-Applied Biosystems, Foster City, Calif.) is the fluorescent reporter dye) and TAMRA (PE-Applied Biosystems, Foster City, Calif.) is the quencher dye.

### Example 32

Antisense Inhibition of Akt-1 Expression-Phosphorothioate Oligodeoxynucleotides

In accordance with the present invention, a series of oligonucleotides were designed to target different regions of the human Akt-1 RNA, using published sequences (GenBank accession number M63167, incorporated herein by reference as SEQ ID NO: 329). The oligonucleotides are shown in Table 18. Target sites are indicated by nucleotide numbers, as given in the sequence source reference (Genbank accession no. M63167), to which the oligonucleotide binds. All compounds in Table 18 are oligodeoxynucleotides with phosphorothioate backbones (internucleoside linkages) throughout. The compounds were analyzed for effect on Akt-1 mRNA levels by quantitative real-time PCR as described in other examples herein. Data are averages from three experiments. If present, “N.D.” indicates “no data”.

TABLE 18

Inhibition of Akt-1 mRNA levels
by phosphorothioate oligodeoxynucleotides

| | | | | | |
|---|---|---|---|---|---|
| | | | | % | |
| | | | | Inhi- | |
| | | TARGET | | bi- | SEQ ID |
| ISIS# | REGION | SITE | SEQUENCE | tion | NO. |

| | | | | | |
|---|---|---|---|---|---|
| 28880 | 5′ UTR | 4 | ccctgtgccctgtcccag | 55 | 333 |
| 28881 | 5′ UTR | 27 | cctaagcccctggtgaca | 15 | 334 |
| 28882 | 5′ UTR | 62 | ctttgacttctttgaccc | 68 | 335 |
| 28883 | 5′ UTR | 70 | ggcagcccctttgacttc | 53 | 336 |
| 28884 | Coding | 213 | caaccctccttcacaata | 24 | 337 |
| 28885 | Coding | 234 | tactcccctcgtttgtgc | 0 | 338 |
| 28886 | Coding | 281 | tgccatcattcttgagga | 65 | 339 |
| 28887 | Coding | 293 | agccaatgaaggtgccat | 67 | 340 |
| 28888 | Coding | 352 | cacagagaagttgttgag | 22 | 341 |
| 28889 | Coding | 496 | agtctggatggcggttgt | 49 | 342 |
| 28890 | Coding | 531 | tcctcctcctcctgcttc | 9 | 343 |
| 28891 | Coding | 570 | cctgagttgtcactgggt | 49 | 344 |
| 28892 | Coding | 666 | ccgaaagtgcccttgccc | 56 | 345 |
| 28893 | Coding | 744 | gccacgatgacttccttc | 60 | 346 |
| 28894 | Coding | 927 | cggtcctcggagaacaca | 0 | 347 |
| 28895 | Coding | 990 | acgttcttctccgagtgc | 30 | 348 |
| 28896 | Coding | 1116 | gtgccgcaaaaggtcttc | 66 | 349 |
| 28897 | Coding | 1125 | tactcaggtgtgccgcaa | 66 | 350 |
| 28898 | Coding | 1461 | ggcttgaagggtgggctg | 41 | 351 |
| 28899 | Coding | 1497 | tcaaaatacctggtgtca | 51 | 352 |
| 28900 | Coding | 1512 | gccgtgaactcctcatca | 56 | 353 |
| 28901 | Coding | 1541 | ggtcaggtggtgtgatgg | 0 | 354 |
| 28902 | Coding | 1573 | ctcgctgtccacacactc | 61 | 355 |
| 28903 | 3′ UTR | 1671 | gcctctccatccctccaa | 76 | 356 |
| 28904 | 3′ UTR | 1739 | acagcgtggcttctctca | 12 | 357 |
| 28905 | 3′ UTR | 1814 | ttttcttccctaccccgc | 64 | 358 |
| 28906 | 3′ UTR | 1819 | gatagttttcttccctac | 0 | 359 |
| 28907 | 3′ UTR | 1831 | taaaacccgcaggatagt | 74 | 360 |
| 28908 | 3′ UTR | 1856 | ggagaacaaactggatga | 0 | 361 |
| 28909 | 3′ UTR | 1987 | ctggctgacagagtgagg | 59 | 362 |
| 28910 | 3′ UTR | 1991 | gcggctggctgacagagt | 61 | 363 |
| 28911 | 3′ UTR | 2031 | cccagagagatgacagat | 46 | 364 |
| 28912 | 3′ UTR | 2127 | gctgctgtgtgcctgcca | 38 | 365 |
| 28913 | 3′ UTR | 2264 | cataatacacaataacaa | 39 | 366 |
| 28914 | 3′ UTR | 2274 | atttgaacaacataatac | 11 | 367 |
| 28915 | 3′ UTR | 2397 | aagtgctaccgtggagag | 57 | 368 |
| 28916 | 3′ UTR | 2407 | cgaaaaggtcaagtgcta | 41 | 369 |
| 28917 | 3′ UTR | 2453 | cagggagtcagggagggc | 13 | 370 |
| 28918 | 3′ UTR | 2545 | aaagttgaatgttgtaaa | 10 | 371 |
| 28919 | 3′ UTR | 2553 | aaaatactaaagttgaat | 25 | 372 |

### Example 33

Antisense Inhibition of Akt-1 Expression-Phosphorothioate 2′-MOE Gapmer Oligonucleotides

In accordance with the present invention, a second series of oligonucleotides targeted to human Akt-1 were synthesized. The oligonucleotide sequences are shown in Table 19. Target sites are indicated by nucleotide numbers, as given in the sequence source reference (Genbank accession no. M63167), to which the oligonucleotide binds.

All compounds in Table 19 are chimeric oligonucleotides (“gapmers”) 18 nucleotides in length, composed of a central “gap” region consisting of ten 2′-deoxynucleotides, which is flanked on both sides (5′ and 3′ directions) by four-nucleotide “wings.” The wings are composed of 2′-methoxyethyl (2′-MOE)nucleotides. The internucleoside (backbone) linkages are phosphorothioate (P═S) throughout the oligonucleotide. Cytidine residues in the 2′-MOE wings are 5-methylcytidines.

Data were obtained by real-time quantitative PCR as described in other examples herein and are averaged from three experiments. If present, “N.D.” indicates “no data”.

TABLE 19

Inhibition of Akt-1 mRNA levels by chimeric
phosphorothioate oligonucleotides
having 2′-MOE wings and a deoxy gap

| | | | | | |
|---|---|---|---|---|---|
| | | | | % | |
| | | | | Inhi- | |
| | | TARGET | | bi- | SEQ ID |
| ISIS# | REGION | SITE | SEQUENCE | -tion | NO. |

| | | | | | |
|---|---|---|---|---|---|
| 28920 | 5′ UTR | 4 | ccctgtgccctgtcccag | 88 | 333 |
| 28921 | 5′ UTR | 27 | cctaagcccctggtgaca | 44 | 334 |
| 28922 | 5′ UTR | 62 | ctttgacttctttgaccc | 61 | 335 |
| 28923 | 5′ UTR | 70 | ggcagcccctttgacttc | 79 | 336 |
| 28924 | Coding | 213 | caaccctccttcacaata | 72 | 337 |
| 28925 | Coding | 234 | tactcccctcgtttgtgc | 39 | 338 |
| 28926 | Coding | 281 | tgccatcattcttgagga | 73 | 339 |
| 28927 | Coding | 293 | agccaatgaaggtgccat | 62 | 340 |
| 28928 | Coding | 352 | cacagagaagttgttgag | 48 | 341 |
| 28929 | Coding | 496 | agtctggatggcggttgt | 43 | 342 |
| 28930 | Coding | 531 | tcctcctcctcctgcttc | 49 | 343 |
| 28931 | Coding | 570 | cctgagttgtcactgggt | 71 | 344 |
| 28932 | Coding | 666 | ccgaaagtgcccttgccc | 64 | 345 |
| 28933 | Coding | 744 | gccacgatgacttccttc | 66 | 346 |
| 28934 | Coding | 927 | cggtcctcggagaacaca | 77 | 347 |
| 28935 | Coding | 990 | acgttcttctccgagtgc | 89 | 348 |
| 28936 | Coding | 1116 | gtgccgcaaaaggtcttc | 61 | 349 |
| 28937 | Coding | 1125 | tactcaggtgtgccgcaa | 74 | 350 |
| 28938 | Coding | 1461 | ggcttgaagggtgggctg | 54 | 351 |
| 28939 | Coding | 1497 | tcaaaatacctggtgtca | 78 | 352 |
| 28940 | Coding | 1512 | gccgtgaactcctcatca | 88 | 353 |
| 28941 | Coding | 1541 | ggtcaggtggtgtgatgg | 71 | 354 |
| 28942 | Coding | 1573 | ctcgctgtccacacactc | 83 | 355 |
| 28943 | 3′ UTR | 1671 | gcctctccatccctccaa | 86 | 356 |
| 28944 | 3′ UTR | 1739 | acagcgtggcttctctca | 73 | 357 |
| 28945 | 3′ UTR | 1814 | ttttcttccctaccccgc | 77 | 358 |
| 28946 | 3′ UTR | 1819 | gatagttttcttccctac | 43 | 359 |
| 28947 | 3′ UTR | 1831 | taaaacccgcaggatagt | 64 | 360 |
| 28948 | 3′ UTR | 1856 | ggagaacaaactggatga | 70 | 361 |
| 28949 | 3′ UTR | 1987 | ctggctgacagagtgagg | 90 | 362 |
| 28950 | 3′ UTR | 1991 | gcggctggctgacagagt | 82 | 363 |
| 28951 | 3′ UTR | 2031 | cccagagagatgacagat | 53 | 364 |
| 28952 | 3′ UTR | 2127 | gctgctgtgtgcctgcca | 80 | 365 |
| 28953 | 3′ UTR | 2264 | cataatacacaataacaa | 48 | 366 |
| 28954 | 3′ UTR | 2274 | atttgaacaacataatac | 39 | 367 |
| 28955 | 3′ UTR | 2397 | aagtgctaccgtggagag | 38 | 368 |
| 28956 | 3′ UTR | 2407 | cgaaaaggtcaagtgcta | 83 | 369 |
| 28957 | 3′ UTR | 2453 | cagggagtcagggagggc | 59 | 370 |
| 28958 | 3′ UTR | 2545 | aaagttgaatgttgtaaa | 25 | 371 |
| 28959 | 3′ UTR | 2553 | aaaatactaaagttgaat | 45 | 372 |

### Example 34

Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

HMVEC d Neo Cells:

The human microvascular endothelial cell line from neonatal dermis, HMVEC d Neo, was obtained from Cascade Biologics Inc., (Portland, Oreg.). Cells are cultured through multiple passages in Medium 131 supplemented with Microvascular Growth Supplement (MVGS) in the absence of antibiotics and antimycotics.

HuVEC Cells:

The human umbilical vein endothelial cell line HuVEC was obtained from the American Type Culture Collection (Manassas, Va.). HuVEC cells are routinely cultured in EBM (Clonetics Corporation Walkersville, Md.) supplemented with SingleQuots supplements (Clonetics Corporation, Walkersville, Md.). Cells are routinely passaged by trypsinization and dilution when they reach 90% confluence, are maintained for up to 15 passages. Cells are seeded into 96-well plates (Falcon-Primaria #3872) at a density of 10000 cells/ well for use in RT-PCR analysis.

For Northern blotting or other analyses, cells may be seeded onto 100 mm or other standard tissue culture plates and treated similarly, using appropriate volumes of medium and oligonucleotide.

HepG2 Cells:

The human hepatoblastoma cell line HepG2 was obtained from the American Type Culture Collection (Manassas, Va.). HepG2 cells are routinely cultured in Eagle's MEM supplemented with 10% fetal calf serum, non-essential amino acids, and 1 mM sodium pyruvate (Gibco/Life Technologies, Gaithersburg, Md.). Cells are routinely passaged by trypsinization and dilution when they reach 90% confluence. Cells were seeded into 96-well plates (Falcon-Primaria #3872) at a density of 7000 cells/well for use in RT-PCR analysis.

For Northern blotting or other analyses, cells may be seeded onto 100 mm or other standard tissue culture plates and treated similarly, using appropriate volumes of medium and oligonucleotide.

AML12 Cells:

The AML12 (alpha mouse liver 12) cell line was established from hepatocytes from a mouse (CD1 strain, line MT42) transgenic for human TGF alpha. Cells are cultured in a 1:1 mixture of Dulbecco's modified Eagle's medium and Ham's F12 medium with 0.005 mg/ml insulin, 0.005 mg/ml transferrin, 5 ng/ml selenium, and 40 ng/ml dexamethasone, and 90%; 10% fetal bovine serum. For subculturing, spent medium is removed and fresh media of 0.25% trypsin, 0.03% EDTA solution is added. Fresh trypsin solution (1 to 2 ml) is added and the culture is left to sit at room temperature until the cells detach.

Cells are routinely passaged by trypsinization and dilution when they reach 90% confluence. Cells are seeded into 96-well plates (Falcon-Primaria #3872) at a density of 7000 cells/well for use in RT-PCR analysis.

For Northern blotting or other analyses, cells may be seeded onto 100 mm or other standard tissue culture plates and treated similarly, using appropriate volumes of medium and oligonucleotide.

Primary Mouse Hepatocytes:

Primary mouse hepatocytes are prepared from CD-1 mice purchased from Charles River Labs (Wilmington, Mass.) and are routinely cultured in Hepatocyte Attachment Media (Gibco) supplemented with 10% Fetal Bovine Serum (Gibco/Life Technologies, Gaithersburg, Md.), 250 nM dexamethasone (Sigma), and 10 nM bovine insulin (Sigma). Cells are seeded into 96-well plates (Falcon-Primaria #3872) at a density of 10000 cells/well for use in RT-PCR analysis.

For Northern blotting or other analyses, cells are plated onto 100 mm or other standard tissue culture plates coated with rat tail collagen (200 ug/mL) (Becton Dickinson) and treated similarly using appropriate volumes of medium and oligonucleotide.

b.END Cells:

The mouse brain endothelial cell line b.END was obtained from Dr. Werner Risau at the Max Plank Institute (Bad Nauheim, Germany). b.END cells are routinely cultured in DMEM, high glucose (Gibco/Life Technologies, Gaithersburg, Md.) supplemented with 10% fetal calf serum (Gibco/Life Technologies, Gaithersburg, Md.). Cells are routinely passaged by trypsinization and dilution when they reach 90% confluence. Cells are seeded into 96-well plates (Falcon-Primaria #3872) at a density of 3000 cells/well for use in RT-PCR analysis.

For Northern blotting or other analyses, cells may be seeded onto 100 mm or other standard tissue culture plates and treated similarly, using appropriate volumes of medium and oligonucleotide.

Treatment with Antisense Compounds:

When cells reach 65-75% confluency, they are treated with oligonucleotide. For cells grown in 96-well plates, wells are washed once with 100 μL OPTI-MEM-1™ reduced-serum medium (Invitrogen Corporation, Carlsbad, Calif.) and then treated with 130 μL of OPTI-MEM-1™ containing 3.75 μg/mL LIPOFECTIN™ (Invitrogen Corporation, Carlsbad, Calif.) and the desired concentration of oligonucleotide. Cells are treated and data are obtained in triplicate. After 4-7 hours of treatment at 37° C., the medium is replaced with fresh medium. Cells are harvested 16-24 hours after oligonucleotide treatment. The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TCCGTCATCGCTCCTCAGGG, SEQ ID NO: 373) which is targeted to human H-ras, or ISIS 18078, (GTGCGCGCGAGCCCGAAATC, SEQ ID NO: 374) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, ATGCATTCTGCCCCCAAGGA, SEQ ID NO: 375, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM.

### Example 35

Design of Phenotypic Assays and in vivo Studies for Target Validation with Oligonucleotides

Phenotypic Assays:

Once target modulators have been identified by the methods disclosed herein, the compounds are further investigated in one or more phenotypic assays, each having measurable endpoints predictive of efficacy in the treatment of a particular disease state or condition.

Phenotypic assays, kits and reagents for their use are well known to those skilled in the art and are herein used to investigate the role and/or association of any given target in health and disease. Representative phenotypic assays, which can be purchased from any one of several commercial vendors, include those for determining cell viability, cytotoxicity, proliferation or cell survival (Molecular Probes, Eugene, Oreg.; Perkin Elmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with target modulators identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharrides or metals. Measurements of cellular status which include pH, stage of the cell cycle, intake or excretion of biological indicators by the cell, are also endpoints of interest.

Analysis of the geneotype of the cell (measurement of the expression of one or more of the genes of the cell) after treatment is also used as an indicator of the efficacy or potency of the target modulators. Hallmark genes, or those genes suspected to be associated with a specific disease state, condition, or phenotype, are measured in both treated and untreated cells.

In vivo Studies:

The individual subjects of the in vivo studies described herein are warm-blooded vertebrate animals, which includes humans.

The clinical trial is subjected to rigorous controls to ensure that individuals are not unnecessarily put at risk and that they are fully informed about their role in the study.

To account for the psychological effects of receiving treatments, volunteers are randomly given placebo or target modulator. Furthermore, to prevent the doctors from being biased in treatments, they are not informed as to whether the medication they are administering is a target modulator or a placebo. Using this randomization approach, each volunteer has the same chance of being given either the new treatment or the placebo.

Volunteers receive either the target modulator or placebo for eight week period with biological parameters associated with the indicated disease state or condition being measured at the beginning (baseline measurements before any treatment), end (after the final treatment), and at regular intervals during the study period. Such measurements include the levels of nucleic acid molecules encoding the target or target protein levels in body fluids, tissues or organs compared to pre-treatment levels. Other measurements include, but are not limited to, indices of the disease state or condition being treated, body weight, blood pressure, serum titers of pharmacologic indicators of disease or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are healthy adults (age 18 to 65 years) and roughly an equal number of males and females participate in the study. Volunteers with certain characteristics are equally distributed for placebo and target modulator treatment. In general, the volunteers treated with placebo have little or no response to treatment, whereas the volunteers treated with the target modulator show positive trends in their disease state or condition index at the conclusion of the study.

### Example 36

Northern Blot Analysis of Target mRNA Levels

Eighteen hours after antisense treatment, cell monolayers are washed twice with cold PBS and lysed in 1 mL RNAZOL™ (TEL-TEST “B” Inc., Friendswood, Tex.). Total RNA is prepared following manufacturer's recommended protocols. Twenty micrograms of total RNA is fractionated by electrophoresis through 1.2% agarose gels containing 1.1% formaldehyde using a MOPS buffer system (AMRESCO, Inc. Solon, Ohio). RNA is transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer is confirmed by UV visualization. Membranes are fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To normalize for variations in loading and transfer efficiency membranes were stripped and probed for human glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### Example 37

Western Blot Analysis of Target Protein Levels

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to target protein is used, with a radiolabeled or fluorescently labeled secondary antibody directed against the primary antibody species. Bands are visualized using a PHOSPHORIMAGER™ (Molecular Dynamics, Sunnyvale Calif.).

### Example 38

Antisense Inhibition of Human Jagged 2 Expression by Chimeric Phosphorothioate Oligonucleotides Having 2′-Moe Wings and a Deoxy Gap

Jagged 2 is a member of the Notch signaling pathway which plays an essential role in cellular differentiation. It has also been implicated in hyperproliferative disorders through its influences on apoptosis and proliferation.

Oligonucleotides targeting Jagged 2 were designed as described in Example 2, synthesized as described in Examples 3-7, analyzed as described in Example 8 and assayed by RT-PCR as described in Example 12.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents are obtained from Invitrogen Corporation, (Carlsbad, Calif.). RT-PCR reactions are carried out by adding 20 μL PCR cocktail (2.5× PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5× ROX dye) to 96-well plates containing 30 μL total RNA solution (20-200 ng). The RT reaction is carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

For human GAPDH the PCR primers were:

forward primer: GAAGGTGAAGGTCGGAGTC (SEQ ID NO: 89)

reverse primer: GAAGATGGTGATGGGATTTC (SEQ ID NO: 90) and the PCR probe

was: 5′ JOE-CAAGCTTCCCGTTCTCAGCC-TAMRA 3′ (SEQ ID NO: 91) where JOE is the fluorescent reporter dye and TAMRA is the quencher dye.

Probes and primers to human Jagged 2 were designed to hybridize to a human Jagged 2 sequence, using published sequence information (GenBank accession number NM_{—}002226.1, incorporated herein as SEQ ID NO: 376). For human Jagged 2 the PCR primers were: forward primer: CCCAGGGCTTCTCCGG (SEQ ID NO: 377) reverse primer: AATAGTCACCCTCCAGGTTATAGCAG (SEQ ID NO: 378) and the PCR probe was: FAM-TGGATGTCGACCTTTGTGAGCCAAGC-TAMRA (SEQ ID NO: 379) where FAM (PE-Applied Biosystems, Foster City, Calif.) is the fluorescent reporter dye) and TAMRA (PE-Applied Biosystems, Foster City, Calif.) is the quencher dye.

The series of oligonucleotides was designed to target different regions of the human Jagged 2 RNA, using published sequences (GenBank accession number NM_{—}002226.1, incorporated herein as SEQ ID NO: 376, GenBank accession number AF029778.1, incorporated herein as SEQ ID NO: 380, a genomic sequence of Jagged 2 represented by residues 104001-133000 of GenBank accession number AF111170.3, incorporated herein as SEQ ID NO: 381, and GenBank accession number BE674071.1, incorporated herein as SEQ ID NO: 382). The oligonucleotides are shown in Table 20. “Target site” indicates the first (5′-most) nucleotide number on the particular target sequence to which the oligonucleotide binds. All compounds in Table 20 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines. The compounds were analyzed for their effect on human Jagged 2 mRNA levels by quantitative real-time PCR as described in other examples herein. Data are averages from two experiments in which human neonatal dermal endothelial cells (HMVEC-d Neo cells) were cultured and treated with oligonucleotides ISIS 148702-148779 (SEQ ID NOs: 383-460 according to the protocol outlined in Example 34. If present, “N.D.” indicates “no data”.

TABLE 20

Inhibition Of Human Jagged 2 mRNA Levels By Chimeric Phosphorothioate
Oligonucleotides Having 2′-Moe Wings and a Deoxy Gap

| | | | | | | |
|---|---|---|---|---|---|---|
| | | TARGET | | | | |
| ISIS# | REGION | SEQ ID NO | TARGET SITE | SEQUENCE | % INHIB | SEQ ID NO |

| | | | | | | |
|---|---|---|---|---|---|---|
| 148702 | 3′ UTR | 376 | 4647 | tacaaaaatgcactttcacg | 79 | 383 |
| 148703 | 3′ UTR | 376 | 4698 | tggcattattcaatcaaata | 0 | 384 |
| 148704 | 5′ UTR | 380 | 2 gcgcacctgcatatgcatga | 10 | 385 | |
| 148705 | Coding | 380 | 475 | gaaatagcccatgggccgcg | 74 | 386 |
| 148706 | Coding | 380 | 487 | cagctgcagctcgaaatagc | 62 | 387 |
| 148707 | Coding | 380 | 497 | gcagcgcgctcagctgcagc | 63 | 388 |
| 148708 | Coding | 380 | 518 | gcagctccccgttcacgttc | 33 | 389 |
| 148709 | Coding | 380 | 523 | gctcagcagctccccgttca | 67 | 390 |
| 148710 | Coding | 380 | 621 | tggtactccttaaggcacac | 74 | 391 |
| 148711 | Coding | 380 | 631 | caccttggcctggtactcct | 72 | 392 |
| 148712 | Coding | 380 | 658 | gccgtagctgcagggccccg | 65 | 393 |
| 148713 | Coding | 380 | 702 | ggcaggtagaaggagttgcc | 49 | 394 |
| 148714 | Coding | 380 | 775 | gacgaggcccgggtcctggt | 64 | 395 |
| 148715 | Coding | 380 | 843 | ttgtcccagtcccaggcctc | 92 | 396 |
| 148716 | Coding | 380 | 927 | aggctcttccagcggtcctc | 63 | 397 |
| 148717 | Coding | 380 | 937 | gctgaagtgcaggctcttcc | 61 | 398 |
| 148718 | Coding | 380 | 947 | ccacgtggccgctgaagtgc | 54 | 399 |
| 148719 | Coding | 380 | 1023 | ggccggcagaacttgttgca | 30 | 400 |
| 148720 | Coding | 380 | 1068 | ttgccgtactggtcgcaggt | 79 | 401 |
| 148721 | Coding | 380 | 1078 | gcaggccttgttgccgtact | 63 | 402 |
| 148722 | Coding | 380 | 1093 | catccagccgtccatgcagg | 84 | 403 |
| 148723 | Coding | 380 | 1149 | cccccgtggagcaaattaca | 71 | 404 |
| 148724 | Coding | 380 | 1183 | gtagctgcacctgcactccc | 84 | 405 |
| 148725 | Coding | 380 | 1269 | cagttgcactgccagggctc | 85 | 406 |
| 148726 | Coding | 380 | 1279 | gttggtctcacagttgcact | 64 | 407 |
| 148727 | Coding | 380 | 1287 | ccgccccagttggtctcaca | 77 | 408 |
| 148728 | Coding | 380 | 1292 | gcaggccgccccagttggtc | 23 | 409 |
| 148729 | Coding | 380 | 1297 | acagagcaggccgccccagt | 72 | 410 |
| 148730 | Coding | 380 | 1302 | ttgtcacagagcaggccgcc | 81 | 411 |
| 148731 | Coding | 380 | 1311 | ttcaggtctttgtcacagag | 74 | 412 |
| 148732 | Coding | 380 | 1321 | gccacagtagttcaggtctt | 60 | 413 |
| 148733 | Coding | 380 | 1331 | ggtggtggctgccacagtag | 49 | 414 |
| 148734 | Coding | 380 | 1443 | gaggtgcaggcgtgctcagc | 63 | 415 |
| 148735 | Coding | 380 | 1672 | cccttcacactcattggcgt | 62 | 416 |
| 148736 | Coding | 380 | 1707 | aggtttttgcaagaaaaagc | 52 | 417 |
| 148737 | Coding | 380 | 1727 | cacagtaatagccgccaatc | 80 | 418 |
| 148738 | Coding | 380 | 1753 | gatgcccttccagcccggga | 75 | 419 |
| 148739 | Coding | 380 | 1810 | gcaggtgcccccatgctgac | 80 | 420 |
| 148740 | Coding | 380 | 1820 | ccaggtccttgcaggtgccc | 88 | 421 |
| 148741 | Coding | 380 | 1845 | gggcacacacactggtaccc | 71 | 422 |
| 148742 | Coding | 380 | 1902 | gggctgctggcacacttgtc | 88 | 423 |
| 148743 | Coding | 380 | 2100 | gagcagttcttgccaccaaa | 85 | 424 |
| 148744 | Coding | 380 | 2154 | ccgcagccatcgatcactct | 93 | 425 |
| 148745 | Coding | 380 | 2334 | gtgcccccattgcggcaggg | 73 | 426 |
| 148746 | Coding | 380 | 2474 | agaagtcattgaccaggtcg | 77 | 427 |
| 148747 | Coding | 380 | 2480 | cacagtagaagtcattgacc | 79 | 428 |
| 148748 | Coding | 380 | 2520 | cgtgagtggcaggtcttgcc | 68 | 429 |
| 148749 | Coding | 380 | 2530 | ctggaactcgcgtgagtggc | 56 | 430 |
| 148750 | Coding | 380 | 2556 | ccgttgctgcaggtgtaggc | 72 | 431 |
| 148751 | Coding | 380 | 2565 | caggtgccaccgttgctgca | 75 | 432 |
| 148752 | Coding | 380 | 2570 | cgtagcaggtgccaccgttg | 80 | 433 |
| 148753 | Coding | 380 | 2658 | ttgggcaggcagctgctgtt | 64 | 434 |
| 148754 | Coding | 380 | 2770 | agggttgcagtcgttggtat | 50 | 435 |
| 148755 | Coding | 380 | 2824 | gcagcggaaccagttgacgc | 75 | 436 |
| 148756 | Coding | 380 | 2901 | ccgtaggcacagggcgagga | 78 | 437 |
| 148757 | Coding | 380 | 2925 | ttgatctcatccacacacgt | 80 | 438 |
| 148758 | Coding | 380 | 2949 | ggtgggcagctacagcgata | 75 | 439 |
| 148759 | Coding | 380 | 3061 | gcagctgttgcagtcttcca | 0 | 440 |
| 148760 | Coding | 380 | 3071 | ccaggcagcggcagctgttg | 71 | 441 |
| 148761 | Coding | 380 | 3504 | ctgctgtcaggcaggtccct | 48 | 442 |
| 148762 | Coding | 380 | 3514 | ctggatcaggctgctgtcag | 61 | 443 |
| 148763 | Coding | 380 | 3597 | tccaccttgacctcggtgac | 69 | 444 |
| 148764 | Coding | 380 | 4059 | gcgcggttgtccactttggg | 59 | 445 |
| 148765 | Stop Codon | 380 | 4104 | ccctactccttgccggcgta | 80 | 446 |
| 148766 | 3′ UTR | 380 | 4156 | gacggcatggctcccaccga | 75 | 447 |
| 148767 | 3′ UTR | 380 | 4274 | gaataatttatacaaggtta | 62 | 448 |
| 148768 | 3′ UTR | 380 | 4306 | aatactccattgttttcagc | 0 | 449 |
| 148769 | 3′ UTR | 380 | 4359 | tcatacagcgagtgccacgc | 74 | 450 |
| 148770 | 3′ UTR | 380 | 4378 | caccctttgctctctccttt | 67 | 451 |
| 148771 | 3′ UTR | 380 | 4492 | caccggcactttggcctgga | 64 | 452 |
| 148772 | 3′ UTR | 380 | 4538 | gggtcccaccaacagccatg | 83 | 453 |
| 148773 | 3′ UTR | 380 | 4845 | gaagggcacttctgaaagca | 56 | 454 |
| 148774 | 3′ UTR | 380 | 4928 | acagttccgagggttctgtg | 20 | 455 |
| 148775 | Intron 5 | 381 | 15219 | ctggctggatcccccacact | 83 | 456 |
| 148776 | Intron 5 | 381 | 17034 | gggagcactcctggctctgc | 38 | 457 |
| 148777 | Exon: Intron | 381 | 18740 | ccatactgactgatatggca | 78 | 458 |
| | Junction | | | | | |
| 148778 Intron: Exon | 381 | 20082 | cgacatccacctgcagggtg | 70 | 459 | |
| | Junction | | | | | |
| 148779 | 3′ UTR | 382 | 242 | tggcaggccccgactcaaca | 69 | 460 |

As shown in Table 20, SEQ ID NOs: 383, 386, 387, 388, 390, 391, 392, 393, 394, 395,396, 397, 398, 399, 401, 402,403, 404, 405, 406, 407, 408, 410, 411, 412, 413, 414, 415, 416, 417, 416, 417, 418, 419, 420, 421, 422, 423, 424, 425, 426, 427, 428, 429, 430, 431, 432, 433, 434, 435, 436, 437, 438, 439, 441, 442, 443, 444, 445, 446, 447, 448, 450, 451, 452, 453, 454, 456, 458, 459 and 460 demonstrated at least 40% inhibition of human Jagged 2 expression in this assay and are therefore preferred. The target sites to which these preferred sequences are complementary are herein referred to as “preferred target segments” and are therefore preferred sites for targeting by compounds of the present invention.

### Example 39

Gene Function Analysis—Caspase Assay to Determine the Effect of Modulating Jagged 2 on the Process of Apoptosis

With specific modulators of Jagged 2 now available, it is possible to examine the role that Jagged 2 plays in cancer.

Programmed cell death or apoptosis involves the activation of proteases, a family of intracellular proteases, through a cascade which leads to the cleavage of a select set of proteins. The caspase family contains at least 14 caspases, with differing substrate preferences. The caspase activity assay uses a DEVD peptide to detect activated caspases in cell culture samples. The peptide is labeled with a fluorescent molecule, 7-amino-4-trifluoromethyl coumarin (AFC). Activated caspases cleave the DEVD peptide resulting in a fluorescence shift of the AFC. Increased fluorescence is indicative of increased caspase activity. The chemotherapeutic drugs taxol, cisplatin, etoposide, gemcitabine, camptothecin, aphidicolin and 5-fluorouracil all have been shown to induce apoptosis in a caspase-dependent manner.

The effect of the Jagged 2 modulator ISIS 148715 (SEQ ID NO: 396) was examined in normal human mammary epithelial cells (HMECs) as well as in two breast carcinoma cell lines, MCF7 and T47D, obtained from the American Type Culture Collection (Manassas Va.). The latter two cell lines express similar genes but MCF7 cells express the tumor suppressor p53, while T47D cells are deficient in p53. MCF7 cells were routinely cultured in DMEM low glucose (Gibco/Life Technologies, Gaithersburg, Md.) supplemented with 10% fetal calf serum (Gibco/Life Technologies, Gaithersburg, Md.). Cells were routinely passaged by trypsinization and dilution when they reached 90% confluence. T47D cells were cultured in Gibco DMEM High glucose media supplemented with 10% FBS.

Cells were plated at 10,000 cells per well for HMEC cells or 20,000 cells per well for MCF-7 and T47D cells, and allowed to attach to wells overnight. Plates used were 96 well Costar plate 1603 (black sides, transparent bottom). DMEM high glucose medium, with and without phenol red, were obtained from Invitrogen (Carlsbad, Calif.). MEGM medium, with and without phenol red, were obtained from Biowhittaker (Walkersville Md.). The caspase-3 activity assay kit was obtained from Calbiochem (Cat. #HTS02).

Before adding to cells, the oligonucleotide cocktail was mixed thoroughly and incubated for 0.5 hrs. The Jagged 2 antisense oligonucleotide ISIS 148715 (SEQ ID NO: 396) or the mixed sequence 20-mer negative oligonucleotide control, ISIS 29848 (SEQ ID NO: 461) or the LIPOFECTIN™ only vehicle control was added (generally from a 3 μM stock of oligonucleotide) to a final concentration of 200 nM with 6 μg/ml LIPOFECTIN™. The medium was removed from the plates and the plates were tapped on sterile gauze. Each well was washed in 150 μl of PBS (150 μL HBSS for HMEC cells). The wash buffer in each well was replaced with 100 μL of the oligonucleotide/ OPTI-MEM™/LIPOFECTIN™ cocktail (this was T=0 for oligonucleotide treatment). The plates were incubated for 4 hours at 37° C., after which the medium was dumped and the plate was tapped on sterile gauze. 100 μl of full growth medium without phenol red was added to each well. After 48 hours, 50 μl of oncogene buffer (provided with Calbiochem kit) with 10 μM DTT was added to each well. 20 μl of oncogene substrate (DEVD-AFC) was added to each well. The plates were read at 400±25 nm excitation and 508±20 nm emission at t=0 and t=3 time points. The t=0×(0.8) time point was subtracted from the from the t=3 time point, and the data are shown as percent of LIPOFECTIN™-only treated cells.

It was thus demonstrated that the antisense modulator of Jagged 2 induces caspase activity in all three cell lines tested. The Jagged 2 oligonucleotide ISIS 148715 caused roughly a 78% reduction of Jagged 2 RNA and approximately a 5.5 fold increase in fluorescence (indicating apoptosis) when administered to HMEC cells at a 200 nM concentration. In MCF7 cells, this Jagged 2 antisense modulator reduced Jagged 2 RNA levels by approximately 50% and increased fluorescence (indicating apoptosis) by approximately 3.4 fold (200 nM concentration). Similarly, in T47D cells, Jagged 2 RNA was decreased by approximately 75% and increased fluorescence (indicating apoptosis) by 8 fold (200 nM dose of ISIS 148715). A second Jagged 2 modulator, ISIS 148744 (SEQ ID NO: 425), reduced Jagged 2 RNA to a slightly lesser extent (approx. 43% reduction) than did ISIS 148715, but also increased apoptosis by approximately 2.5 fold in MCF7 cells and 3.5 fold in T47D cells. Interestingly, ISIS 148744 did not inhibit apoptosis in the normal HMEC cells, but only in the two cancer cell lines.

### Example 40

Gene Function Analysis—Cell Cycle Analysis to Determine the Effect of Modulating Jagged 2 on the Process of Apoptosis

Cell cycle regulation is the basis for various cancer therapies. Under some circumstances normal cells undergo growth arrest, while transformed cells undergo apoptosis and this difference can be used to protect normal cells against death caused by chemotherapeutic drugs. Disruption of cell cycle checkpoints in cancer cells can increase sensitivity to chemotherapy while cells with normal checkpoints may take refuge in G1, thus increasing the therapeutic index. ISIS 148715, an antisense modulator of Jagged 2, was tested for effects on the cell cycle in normal HMEC cells and cancer cells, both with and without p53. 72 hours after treatment with ISIS 148715, cells were stained with propidium iodide to generate a cell cycle profile using a flow cytometer. The cell cycle profile was analyzed with the ModFit program (Verity Software House, Inc., Topsham Me.). Neither LIPOFECTIN™ alone nor a panel of negative antisense controls perturbed the cell cycle. However, it was found that ISIS 148715 induced apoptosis in all three cell lines, as measured by an increase in the percentage of sub-G1 cells. In T47D cells, the percent hypodiploid cells (indicative of apoptosis) was shown to increase from approximately 4.5% for LIPOFECTIN™ control-treated cells to approximately 16% for ISIS 148715-treated cells. In MCF7 cells, the percent hypodiploid cells increased from approximately 3% (LIPOFECTIN™ only) to approximately 12.5% (ISIS 148715). In normal HMEC cells the percent diploid cells increased from approximately 2% (LIPOFECTIN™ control) to approximately-8% for cells treated with ISIS 148715. This increase in apoptosis was dose-dependent. In MCF7 cells this increase went from approximately 4% at 200 nM oligonucleotide to 8% at 300 nM oligonucleotide.

### Example 41

Antisense Inhibition Of Human Transforming Growth Factor-Beta 3 Expression by Chimeric Phosphorothioate Oligonucleotides Having 2′-Moe Wings and a Deoxy Gap

The transforming growth factor-beta superfamily of cytokines regulates a diverse array of physiologic functions including cell proliferation and growth, cell migration, differentiation, development, production of extracellular matrix, and the immune response. Each subgroup of this superfamily initiates a unique intracellular signaling cascade activated by ligand-induced formation and activation of specific serine/threonine kinase receptor complexes. Transforming growth factor-beta 3 is believed to have a role in healing of wounds and bone fractures, and is not expressed in healthy skin.

Oligonucleotides targeting human transforming growth factor-beta 3 were designed as described in Example 2, synthesized as described in Examples 3-7, analyzed as described in Example 8 and assayed by RT-PCR as described in Example 12.

For human transforming growth factor-beta 3 the PCR primers were:

forward primer: ACCAATTACTGCTTCCGCAACT (SEQ ID NO: 463) reverse primer:

GATCCTGTCGGAAGTCAATGTAGA (SEQ ID NO: 464) and the PCR probe was:

FAM-AGGAGAACTGCTGTGTGCGCCCC-TAMRA (SEQ ID NO: 465) where FAM (PE-Applied Biosystems, Foster City, Calif.) is the fluorescent reporter dye) and TAMRA (PE-Applied Biosystems, Foster City, Calif.) is the quencher dye.

The series of oligonucleotides was designed to target different regions of the human transforming growth factor-beta 3 RNA, using published sequences (GenBank accession number NM_{—}003239.1, incorporated herein as SEQ ID NO: 462, and residues 138001-167000 of GenBank accession number AF107885, the complement of which is incorporated herein as SEQ ID NO: 466). The oligonucleotides are shown in Table 21. “Target site” indicates the first (5′-most) nucleotide number on the particular target sequence to which the oligonucleotide binds. All compounds in Table 21 are chimeric oligonucleotides (“gapmers”) 20 nucleotides in length, composed of a central “gap” region consisting of ten 2′-deoxynucleotides, which is flanked on both sides (5′ and 3′ directions) by five nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines. The compounds were analyzed for their effect on human transforming growth factor-beta 3 mRNA levels by quantitative real-time PCR as described in other examples herein. Data are averages from two experiments in which HuVEC cells were cultured and treated with oligonucleotides 155368-155715 (SEQ ID NOs: 467-544) according to the protocol outlined in Example 34. If present, “N.D.” indicates “no data”.

TABLE 21

Inhibition Of Human Transforming Growth Factor-Beta 3 mRNA Levels
by Chimeric Phosphorothioate Oligonucleotides
Having 2′-Moe Wings and a Deoxy Gap

| | | | | | | |
|---|---|---|---|---|---|---|
| | | TARGET | | | | |
| ISIS # | REGION | SEQ ID NO | TARGET SITE | SEQUENCE | % INHIB | SEQ ID NO. |

| | | | | | | | |
|---|---|---|---|---|---|---|---|
| 155638 | 5′ UTR | 462 | 5 | ttgttgtccatgtgtctaaa | 69 | 467 | |
| 155639 | 5′ UTR | 462 | 76 | ttcaggacttccaggaagcg | 62 | 468 | |
| 155640 | 5′ UTR | 462 | 106 | aggtgcatgaactcactgca | 75 | 469 | |
| 155641 | 5′ UTR | 462 | 205 | cggcaaggcctggagaggaa | 0 | 470 | |
| 155642 | Start Codon | 462 | 248 | aagtgcatcttcatgtgtga | 76 | 471 | |
| 155643 | Start Codon | 462 | 253 | tttgcaagtgcatcttcatg | 87 | 472 | |
| 155644 | Coding | 462 | 258 | agccctttgcaagtgcatct | 79 | 473 | |
| 155645 | Coding | 462 | 263 | accagagccctttgcaagtg | 70 | 474 | |
| 155646 | Coding | 462 | 284 | aagttcagcagggccaggac | 45 | 475 | |
| 155647 | Coding | 462 | 313 | aagtggacagagagaggctg | 64 | 476 | |
| 155648 | Coding | 462 | 316 | tgcaagtggacagagagagg | 47 | 477 | |
| 155649 | Coding | 462 | 320 | gtggtgcaagtggacagaga | 85 | 478 | |
| 155650 | Coding | 462 | 341 | ttgatgtggccgaagtccaa | 57 | 479 | |
| 155651 | Coding | 462 | 346 | tcttcttgatgtggccgaag | 69 | 480 | |
| 155652 | Coding | 462 | 351 | cctcttcttcttgatgtggc | 93 | 481 | |
| 155653 | Coding | 462 | 356 | tccaccctcttcttcttgat | 70 | 482 | |
| 155654 | Coding | 462 | 361 | tggcttccaccctcttcttc | 72 | 483 | |
| 155655 | Coding | 462 | 366 | cctaatggcttccaccctct | 87 | 484 | |
| 155656 | Coding | 462 | 371 | tgtcccctaatggcttccac | 73 | 485 | |
| 155657 | Coding | 462 | 376 | agatctgtcccctaatggct | 75 | 486 | |
| 155658 | Coding | 462 | 380 | ctcaagatctgtcccctaat | 72 | 487 | |
| 155659 | Coding | 462 | 383 | ttgctcaagatctgtcccct | 82 | 488 | |
| 155660 | Coding | 462 | 430 | ggacgtgggtcatcaccgtt | 85 | 489 | |
| 155661 | Coding | 462 | 566 | atcatgtcgaatttatggat | 43 | 490 | |
| 155662 | Coding | 462 | 572 | ccctggatcatgtcgaattt | 70 | 491 | |
| 155663 | Coding | 462 | 653 | tccactgaggacacattgaa | 90 | 492 | |
| 155664 | Coding | 462 | 656 | ttctccactgaggacacatt | 95 | 493 | |
| 155665 | Coding | 462 | 660 | atttttctccactgaggaca | 90 | 494 | |
| 155666 | Coding | 462 | 706 | tgggcacccgcaagacccgg | 90 | 495 | |
| 155667 | Coding | 462 | 812 | gtgggcagattcttgccacc | 0 | 496 | |
| 155668 | Coding | 462 | 860 | cgcacagtgtcagtgacatc | 0 | 497 | |
| 155669 | Coding | 462 | 929 | aaggtgtgacatggacagtg | 93 | 498 | |
| 155670 | Coding | 462 | 934 | gctgaaaggtgtgacatgga | 84 | 499 | |
| 155671 | Coding | 462 | 939 | attgggctgaaaggtgtgac | 0 | 500 | |
| 155672 | Coding | 462 | 944 | tctccattgggctgaaaggt | 69 | 501 | |
| 155673 | Coding | 462 | 983 | aatttgatttccatcacctc | 43 | 502 | |
| 155674 | Coding | 462 | 1022 | tctccacggccatggtcatc | 57 | 503 | |
| 155675 | Coding | 462 | 1163 | ttgcggaagcagtaattggt | 76 | 504 | |
| 155676 | Coding | 462 | 1269 | tgagcagaagttggcatagt | 69 | 505 | |
| 155677 | Coding | 462 | 1274 | gggcctgagcagaagttggc | 61 | 506 | |
| 155678 | Coding | 462 | 1279 | ggcaagggcctgagcagaag | 50 | 507 | |
| 155679 | Coding | 462 | 1295 | gcactgcggaggtatgggca | 50 | 508 | |
| 155680 | Coding | 462 | 1346 | tcagggttcagagtgttgta | 50 | 509 | |
| 155681 | Coding | 462 | 1457 | gacttcaccaccatgttgga | 37 | 510 | |
| 155682 | Stop Codon | 462 | 1478 | gggtctcagctacatttaca | 54 | 511 | |
| 155683 | 3′ UTR | 462 | 1562 | agtgaggtttgttgcttgtg | 72 | 512 | |
| 155684 | 3′ UTR | 462 | 1619 | gaaacctccatctcagccat | 59 | 513 | |
| 155685 | 3′ UTR | 462 | 1703 | agagttcagccttcctctaa | 92 | 514 | |
| 155686 | 3′ UTR | 462 | 1807 | ttagggtagcccaaatccca | 66 | 515 | |
| 155687 | 3′ UTR | 462 | 1834 | agccattctctgcccttcct | 90 | 516 | |
| 155688 | 3′ UTR | 462 | 1870 | tcagatctgaagtgtcttcc | 94 | 517 | |
| 155689 | 3′ UTR | 462 | 1918 | tccagattccctagagcaga | 72 | 518 | |
| 155690 | 3′ UTR | 462 | 1929 | gtataacataatccagattc | 0 | 519 | |
| 155691 | 3′ UTR | 462 | 1943 | aaaatgcttgccttgtataa | 79 | 520 | |
| 155692 | 3′ UTR | 462 | 1979 | ctgggactttgtcttcgtaa | 95 | 521 | |
| 155693 | 3′ UTR | 462 | 2030 | ttgcaaaagtaatagatttg | 0 | 522 | |
| 155694 | 3′ UTR | 462 | 2051 | ttaattgatgtagaggacag | 0 | 523 | |
| 155695 | 3′ UTR | 462 | 2082 | ctggattttctccctgtagt | 86 | 524 | |
| 155696 | 3′ UTR | 462 | 2093 | aactgcatgacctggatttt | 7 | 525 | |
| 155697 | 3′ UTR | 462 | 2112 | atacagttgatgggccagga | 74 | 526 | |
| 155698 | 3′ UTR | 462 | 2126 | atccaaaaggcccaatacag | 36 | 527 | |
| 155699 | 3′ UTR | 462 | 2151 | ccaccctttcttctgcgttc | 87 | 528 | |
| 155700 | 3′ UTR | 462 | 2235 | gtctaaccaagtgtccaagg | 92 | 529 | |
| 155701 | 3′ UTR | 462 | 2280 | tgcatggaaccacaatccag | 96 | 530 | |
| 155702 | 3′ UTR | 462 | 2292 | atgccccaaggctgcatgga | 75 | 531 | 3 |
| 155703 | 3′ UTR | 462 | 2335 | aatgaacacagggtcttgga | 87 | 532 | |
| 155704 | 3′ UTR | 462 | 2356 | cacctgcttccaggaacacc | 87 | 533 | |
| 155705 | 3′ UTR | 462 | 2361 | tgtagcacctgcttccagga | 28 | 534 | |
| 155706 | 3′ UTR | 462 | 2407 | agtcactgtgtggcacatgt | 4 | 535 | |
| 155707 | 3′ UTR | 462 | 2456 | agtaatattcatacttgtct | 23 | 536 | |
| 155708 | 3′ UTR | 462 | 2482 | atatttatttatacaaagat | 0 | 537 | |
| 155709 | 3′ UTR | 462 | 2534 | ctgttctagaaacaatattc | 62 | 538 | |
| 155710 | Intron | 466 | 11878 | ctgctggaagcaaaggcagg | 0 | 539 | |
| 155711 | Intron: | 466 | 12956 | gaggagttacctggaagagc | 0 | 540 | |
| | Exon | | | | | | |
| | Junction | | | | | | |
| 155712 | Intron: | 466 | 13385 | gtccacctacctcttctcaa | 33 | 541 | |
| | Exon | | | | | | |
| | Junction | | | | | | |
| 155713 | Intron | 466 | 18442 | atgccatctacatggttttt | 9 | 542 | |
| 155714 | Intron: | 466 | 21023 | ttgtccacgcctgaagaagg | 56 | 543 | |
| | Exon | | | | | | |
| | Junction | | | | | | |
| 155715 | Intron: | 466 | 21195 | ccagtctcaccggaagcagt | 1 | 544 | |
| | Exon | | | | | | |
| | Junction | | | | | | |

As shown in Table 21, SEQ ID NOs: 467, 468, 469, 471, 472, 473, 474, 475, 476, 477, 478, 479, 480, 481, 482, 483, 484, 485, 486, 487, 488, 489, 490, 491, 492, 493, 494, 495, 498, 499, 501, 502, 503, 504, 505, 506, 507, 508, 509, 511, 512, 513, 514, 515, 516, 517, 518, 520, 521, 524, 526, 528, 529, 530, 531, 532, 533, 538 and 543 demonstrated at least 40% inhibition of human transforming growth factor-beta 3 expression in this assay and are therefore preferred. The target sites to which these preferred sequences are complementary are herein referred to as “preferred target segments” and are therefore preferred sites for targeting by compounds of the present invention.

### Example 42

Treatment Of Angiogenic Disease: Breast Cancer

Breast carcinoma accounts for the most common type of tumors in women over 40 years age and is a leading cause of death. The beneficial effect on patients with breast cancer with the transforming growth factor beta 3 modulator may be shown in the following clinical trials: In a first clinical trial, 5 patients suffering from metastatic breast carcinoma are studied, who have no previous systemic treatment of metastasis (adjuvant treatment is ignored) and have facile vein access. The patients have PS 0 or 1 and may be post-menopausal. The transforming growth factor beta 3 modulator may be continuously administered parenterally, e.g. s.c. by means of a pump at the rate of e.g. 0.5 to 2 mg per 24 hours, over at least 3 days. The growth factor IGF profile is determined and the levels are found to be reduced.

A second clinical trial may be effected as follows: In a second trial the transforming growth factor beta 3 modulators are administered to at least 14 patients having breast cancer and the extent and duration of the response are determined.

Patients are included who have breast cancer as evidenced by histological biopsy (glandular analysis—EOA). They present a metastatic illness and/or loco-regional localization which is measurable and evaluable. If desired, patients are included who are resistant to other treatment to conventional therapy such as surgery, radiotherapy, other chemotherapy and/or hormone therapy.

The patients present at least one target (identifier), on X-ray analysis, which is measurable or evaluable such as a primitive metastatic tumor which is cutaneous or sub-cutaneous. It may be gangliar or visceral. Preferably the patients have lesions which have progressed within the month preceding the trial and have an estimated survival time of at least 3 month.

Preferably the trial excludes: patients in which the sole criteria for diagnosing breast cancer are biological modifications; patients administered with an embroynic carcinoma antigen pathology; patients with ascitis, a pleural effusion, a pulmonary carcinoma lymphangitis, or an osseous localization as sole metastatic manifestation; patients treated on a unique tumoral target by radiotherapy less than eight weeks before inclusion in the study (they are eligible however if evidence of progression during this time); patients with a unique cerebral localization; patients presenting another malignant tumor with the exception of a carcinoma in situ in the cervix uteri or a spino- or basocellular skin cancer; and patients not able to attend regular consultations.

With these exclusions the efficacy of the transforming growth factor beta 3 modulators may be followed more clearly. The transforming growth factor beta 3 modulators may be used in the method of treatment at the invention, however, in treating patients falling in the above exclusion.

The transforming growth factor beta 3 modulators may be administered at the same dosage as or at a lower dosage than in the first trial, but preferably in two doses, one in the morning and one in the evening. The treatment is for at least 3 months or until complete remission. The response may be followed by conventional methodology, e.g. according to IUCC response criteria, e.g. progression, stabilization, partial or complete remission. The evaluation is effected e.g. on day 0, 15, 45, 60 and 90.

A third clinical trial may be effected as follows: Patients with advanced breast cancer are included. The patients have progressive disease and measurable and/or evaluable parameters according to criteria of the IUCC (i.e. appearance of new lesions or growth of existing metastatic lesions) not responding to primary hormonal and/or cytotoxic therapy. As in the above indicated second clinical trial, the third trial preferably also excludes patients with previous or concurrent malignancies at other sites, with the exception of cone biopsied in situ carcinoma of the cervix uteri and adequately treated basal or squamous cell carcinoma of the skin.

The transforming growth factor beta 3 modulators may be administered at the same dosage as or at a lower dosage than in the second trial. Preferably the modulators are administered parenterally, e.g. subcutaneous, particularly in a continuous subcutaneous way by means of a portable syringe pump (infusion pump). Treatment is for at least 2 months or until complete remission. The response may be followed by conventional methodology e.g. according to IUCC response criteria. The evaluation is effected e.g. on day 0, 30 and 60. All lesions are measured at each assessment or when multiple lesions are present, a representative number of 5 lesions may be selected for measurement. Regression of the lesions is the sum of the products of the diameters of each individual lesion or those selected for study, which decreases by 50% or more.

### Example 43

Treatment Of Angiogenic Disease: Melanoma

In an in vivo test, Meth-A sarcoma and melanoma cells (1.times. 10.sup.6) are inoculated subcutaneously in 0.1 ml saline in the same position of the dorsal skin of C3H mice (n=20). On the same day, the mice receive orally either transforming growth factor beta 3 modulators, at 100 mg per kg in body weight, suspended in 300 uL of olive oil (n=10) or 300 uL olive oil alone (n=10). This treatment is carried out every day and the diameter of the tumors is monitored every second day. On day 12 the mice are sacrificed and the tumor weights are measured.

Meth-A sarcoma tumor growth in mice treated with transforming growth factor beta 3 modulators is slower than in control mice. The weight (grams) of both the Meth-A sarcoma and melanoma tumors on day 12 is measured, and the mice treated with transforming growth factor beta 3 modulators have lower tumor mass. In a small number of control and 2-methoxyestradiol mice, the dorsal skin, together with the tumor, are excised and the angiogenesis within the subcutaneous fascia in the control and treated mice is visualized with Indian ink. Apart from their marginally lower weight, the treated mice exhibit no apparent signs of toxicity and are all alive after 12 days of daily treatment. Transforming growth factor beta 3 modulators thus has potent pharmacological properties which may be applied in the treatment angiogenic diseases, including solid tumors.

### Example 44

Methods Of Inhibiting Angiogenesis

Angiogenesis is the growth of new blood vessels (veins & arteries) by endothelial cells. This process is important in the development of a number of human diseases, and is believed to be particularly important in regulating the growth of solid tumors. Without new vessel formation it is believed that tumors will not grow beyond a few millimeters in size. In addition to their use as anti-cancer agents, modulators of angiogenesis have potential for the treatment of diabetic retinopathy, cardiovascular disease, rheumatoid arthritis and psoriasis.

During the process of angiogenesis, endothelial cells perform several distinct functions, including the degradation of the extracellular matrix (ECM), migration, proliferation and the formation of tube-like structures. Various genes may regulate some of these processes in primary human umbilical vein endothelial cells (HUVECs). The below experiments employed an antisense compound as a transforming growth factor beta 3 modulators. The antisense compound comprises ISIS NO. 155701 (SEQ ID NO: 530).

### Example 45

Gene Function Analysis—Matrix Metalloproteinase Assay to Determine the Effect of Modulating Transforming Growth Factor-Beta 3 on the Process of Angiogenesis

During angiogenesis, endothelial cells need to be able to degrade the extracellular matrix so they can migrate and form new vessels. Endothelial cells secrete matrix metalloproteinases (MMPs) in order to accomplish this degradation. MMPs are a family of zinc-dependent endopeptidases that fall into eight distinct classes: five are secreted and three are membrane-type MMPs (MT-MMPs). MMPs exert these effects by cleaving a diverse group of substrates, which include not only structural components of the extracellular matrix, but also growth-factor-binding proteins, growth-factor precursors, receptor tyrosine kinases, cell-adhesion molecules and other proteinases. In this assay the activity of MMPs secreted into the media above antisense oligonucleotide-treated HUVECs is measured.

MMP activity in the media above HUVECs is measured using the EnzChek Gelatinase/Collagenase Assay Kit (Molecular Probes, Eugene, Oreg.). HUVECs are plated at 3000 cells/well in 96-well plates. One day later, cells are transfected with antisense oligonucleotides according to standard published procedures (Monia et al., (1 993) J Biol Chem. Jul. 5, 1993;268(19):14514-22) with 75 nM oligonucleotide in LIPOFECTIN™ (Gibco, Grand Island, N.Y.). Antisense oligonucleotides are tested in triplicate on each 96-well plate, except for positive and negative antisense controls, which are measured up to six times per plate. Twenty hours post-transfection, MMP production is stimulated by the addition of recombinant human vascular endothelial growth factor (VEGF). Fifty hours post-transfection, a p-aminophenylmercuric acetate (APMA; Sigma-Aldrich, St. Louis, Mo.) solution is added to each well of a Corning-Costar 96-well clear bottom plate (VWR International, Brisbane, Calif.). The APMA solution is used to promote cleavage of inactive MMP precursor proteins (Nagase et al., (1991) Biomed Biochim Acta, 50(4-6):749-54). Medium above the HUVECs is then transferred to the wells. After 30 minutes, the quenched, fluorogenic MMP cleavage substrate is added, and baseline fluorescence is read immediately at 485 nm exitation/530 nm emission. Following an overnight incubation at 37° C. in the dark, plates are read again to determine the amount of fluorescence, which corresponds to MMP activity. Total protein from HUVEC lysates is used to normalize the readings, and MMP activities±standard deviation are expressed relative to transfectant-only controls.

The modulators caused a 52% reduction of MMP activity, as compared to MMP activity in lipid-treated cells. Thus, it is shown that transforming growth factor beta 3 modulators can prevent angiogenesis.

### Example 46

Gene Function Analysis—Endothelial Tube Formation Assay to Determine the Effect of Modulating Transforming Growth Factor-Beta 3 on the Process of Angiogenesis

Angiogenesis is stimulated by numerous factors that promote interaction of endothelial cells with each other and with extracellular matrix molecules, resulting in the formation of capillary tubes. This morphogenic process is necessary for the delivery of oxygen to nearby tissues and plays an essential role in embryonic development, wound healing, and tumor growth. Moreover, this process can be reproduced in tissue culture by the formation of tube-like structures by endothelial cells. There are several different variations of the assay that use different matrices, such as collagen I (Kanayasu, 1991), Matrigel (Yamagishi, 1997) and fibrin (Bach, 1998) as growth substrates for the cells. In this assay, HUVECs are plated on a matrix derived from the Engelbreth-Holm-Swarm mouse tumor, which is very similar to Matrigel (Kleinman, 1986; Madri, 1986). Untreated HUVECs form tube-like structures when grown on this substrate. Loss of tube formation in-vitro has been correlated with the inhibition of angiogenesis in-vivo (Carmeliet et al., (2000) Nature 407:249-257; and Zhang et al., (2002) Cancer Research 62:2034-42), which supports the use of in-vitro tube formation as an endpoint for angiogenesis.

The Tube Formation Assay is performed using an In-vitro Angiogenesis Assay Kit (Chemicon International, Temecula, Calif.), or growth factor reduced Mortigel (BD Biosciences, Bedford, Mass.). Cells are plated and transfected with transforming growth factor beta 3 modulators (antisense oligonucleotides) as described for the MMP activity assay, except cells are plated at 4000 cells/well. Fifty hours post-transfection, cells are transferred to 96-well plates coated with ECMatrix™ (Chemicon International) or growth factor depleted matrigel. Under these conditions, untreated HUVECs form tube-like structures. After an overnight incubation at 37° C., treated and untreated cells are inspected by light microscopy. Individual wells are assigned discrete scores from 1 to 5 depending on the extent of tube formation. A score of 1 refers to a well with no tube formation while a score of 5 is given to wells where all cells are forming an extensive tubular network.

As calculated from the assigned discreet scores, cells treated with transforming growth factor beta 3 modulators had a tube formation score reduction of about 60% as compared to lipid-treated cells. Thus, it is shown that transforming growth factor beta 3 modulators can inhibit angiogenesis.

### Example 47

Gene Function Analysis—Measurement of RNA Expression Levels of Angiogenic Genes to Determine the Effect of Modulating Transforming Growth Factor-Beta 3 on the Process of Angiogenesis

Endothelial cells must regulate the expression of many genes in order to perform the functions necessary for angiogenesis. This gene regulation has been the subject of intense scrutiny, and many genes have been identified as being important for the angiogenic phenotype. The expression levels of four genes, previously identified as being highly expressed in angiogenic endothelial cells, is measured here (Integrin beta 3, endoglin/CD105, TEM5 and MMP-14/MT-MMP1).

Integrin beta 3 is part of a family of heterodimeric transmembrane receptors that consist of alpha and beta subunits. Each subunit recognizes a unique set of ECM ligands, thereby allowing cells to transmit angiogenic signals from the extracellular matrix. Integrin beta 3 is prominently expressed on proliferating vascular endothelial cells, and it plays roles in allowing new blood vessels to form at tumor sites as well as allowing the epithelial cells of breast tumors to spread. Blockage of Integrin alpha 3 with monoclonal antibodies or low molecular weight antagonists inhibits blood vessel formation in a variety of in-vivo models, including tumor angiogenesis and neovascularization during oxygen-induced retinopathy.

Endoglin is a Transforming Growth Factor receptor-associated protein highly expressed on endothelial cells, and present on some leukemia cells and minor subsets of bone marrow cells. Its expression is upregulated in endothelial cells of angiogenic tissues and is therefore used as a prognostic indicator in various tumors. Endoglin functions as an ancillary receptor influencing binding of the Transforming Growth Factor beta (TGF-beta) family of ligands to signaling receptors, thus mediating cell survival. Mutations of the endoglin gene result in a genetic disease of the vasculature-Hereditary Haemorrhagic Telangiectasia (HHT), which is characterized by bleeding from malformed blood vessels. Defective signaling by different TGF-beta ligands and receptors is thought to be involved.

Tumor endothelial marker 5 (TEM5) is a putative 7-pass transmembrane protein (GPCR) for which EST sequence but no other information is available. The mRNA transcript, designated KIAA1531, encodes one of many tumor endothelium markers (TEMs) that display elevated expression (greater than 10-fold) during tumor angiogenesis. TEM5 is coordinately expressed with other TEMs on tumor endothelium in humans and mice.

MMP-14, a membrane-type MMP (MT-MMP) covalently linked to the cell membrane, is involved in matrix detachment and migration. MMP-14 is thought to promote tumor angiogenesis; antibodies directed against the catalytic domain of MMP-14 block endothelial-cell migration, invasion and capillary tube formation in-vitro. MMP-14 can degrade the fibrin matrix that surrounds newly formed vessels potentially allowing the endothelial cells to invade further into the tumor tissue. MMP-14 null mice have impaired angiogenesis during development, further demonstrating the role of MMP-14 in angiogenesis.

Cells are plated and transfected as described for the MMP activity assay. Twenty hours post-transfection, cells are stimulated with recombinant human VEGF. Total RNA is harvested 52 hours post-transfection, and the amount of total RNA from each sample is determined using a Ribogreen Assay (Molecular Probes, Eugene, Oreg.). Real-time PCR is performed on the total RNA using primer/probe sets for four Angiogenic Hallmark Genes: integrin beta 3, endoglin, Tumor endothelial marker 5 (TEM5) and Matrix Metalloproteinase 14 (MMP14/MTI-MMP). Expression levels for each gene are normalized to total RNA, and values are expressed relative to controls.

Cells treated with transforming growth factor beta 3 modulators had the following mRNAs reduced as compared to mRNAs of the controls: Integrin beta 3 mRNA was 88% of the control, endoglin mRNA was 74% of the control, TEM5 mRNA was 91% of the control, and MMP14/MT1-MMP was 86% of the control.

### Example 48

Antisense Inhibition Of Human Apolipoprotein B mRNA Expression by Chimeric Phosphorothioate Oligonucleotides having 2′-Moe Wings and a Deoxy Gap

Lipoproteins are globular, micelle-like particles that consist of a non-polar core of acylglycerols and cholesteryl esters surrounded by an amphiphilic coating of protein, phospholipid and cholesterol. Apolipoprotein B is a large glycoprotein that serves an indispensable role in the assembly and secretion of lipids and in the transport and receptor-mediated uptake and delivery of distinct classes of lipoproteins. Elevated plasma levels of the ApoB-100-containing lipoprotein Lp(a) are associated with increased risk for atherosclerosis and its manifestations, which may include hypercholesterolemia, myocardial infarction, and thrombosis.

Oligonucleotides targeting human apolipoprotein B were designed as described in Example 2, synthesized as described in Examples 3-7, analyzed as described in Example 8 and assayed by RT-PCR as described in Example 12.

Probes and primers to human apolipoprotein B were designed to hybridize to a human apolipoprotein B sequence, using published sequence information (GenBank accession number NM_{—}000384.1, incorporated herein as SEQ ID NO: 545). For human apolipoprotein B the PCR primers were: forward primer:

TGCTAAAGGCACATATGGCCT (SEQ ID NO: 546) reverse primer:

CTCAGGTTGGACTCTCCATTGAG (SEQ ID NO: 547) and the PCR probe was: FAM-CTTGTCAGAGGGATCCTAACACTGGCCG-TAMRA (SEQ ID NO: 548) where FAM (PE-Applied Biosystems, Foster City, Calif.) is the fluorescent reporter dye) and TAMRA (PE-Applied Biosystems, Foster City, Calif.) is the quencher dye.

The series of oligonucleotides was designed to target different regions of the human apolipoprotein B RNA, using published sequence information (GenBank accession number NM_{—}000384.1, incorporated herein as SEQ ID NO: 545). The oligonucleotides are shown in Table 22. “Target site” indicates the first (5′-most) nucleotide number on the particular target sequence to which the oligonucleotide binds. All compounds in Table 22 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines. The compounds were analyzed for their effect on human apolipoprotein B mRNA levels in HepG2 cells by quantitative real-time PCR as described on other examples herein. Data are averages from two experiments in which HepG2 cells were cultured and treated with oligonucleotides 147780-147833 (SEQ ID NOs: 549-602) according to the protocol outlined in Example 34. If present, “N.D.” indicates “no data”.

TABLE 22

Inhibition Of Human Apolipoprotein B mRNA Levels
By Chimeric Phosphorothioate Oligonucleotides
Having 2′-Moe Wings and a Deoxy Gap

| | | | | | | |
|---|---|---|---|---|---|---|
| | | TARGET | | | | |
| ISIS # | REGION | SEQ ID NO | TARGET SITE | SEQUENCE | % INHIB | SEQ ID NO |

| | | | | | | |
|---|---|---|---|---|---|---|
| 147780 | 5′ UTR | 545 | 1 | ccgcaggtcccggtgggaat | 40 | 549 |
| 147781 | 5′ UTR | 545 | 21 | accgagaagggcactcagcc | 35 | 550 |
| 147782 | 5′ UTR | 545 | 71 | gcctcggcctcgcggccctg | 67 | 551 |
| 147783 | Start Codon | 545 | 114 | tccatcgccagctgcggtgg | N.D. | 552 |
| 147784 | Coding | 545 | 151 | cagcgccagcagcgccagca | 70 | 553 |
| 147785 | Coding | 545 | 181 | gcccgccagcagcagcagca | 29 | 554 |
| 147786 | Coding | 545 | 321 | cttgaatcagcagtcccagg | 34 | 555 |
| 147787 | Coding | 545 | 451 | cttcagcaaggctttgccct | N.D. | 556 |
| 147788 | Coding | 545 | 716 | tttctgttgccacattgccc | 95 | 557 |
| 147789 | Coding | 545 | 911 | ggaagaggtgttgctccttg | 24 | 558 |
| 147790 | Coding | 545 | 951 | tgtgctaccatcccatactt | 33 | 559 |
| 147791 | Coding | 545 | 1041 | tcaaatgcgaggcccatctt | N.D. | 560 |
| 147792 | Coding | 545 | 1231 | ggacacctcaatcagctgtg | 26 | 561 |
| 147793 | Coding | 545 | 1361 | tcagggccaccaggtaggtg | N.D. | 562 |
| 147794 | Coding | 545 | 1561 | gtaatcttcatccccagtgc | 47 | 563 |
| 147795 | Coding | 545 | 1611 | tgctccatggtttggcccat | N.D. | 564 |
| 147796 | Coding | 545 | 1791 | gcagccagtcgcttatctcc | 8 | 565 |
| 147797 | Coding | 545 | 2331 | gtatagccaaagtggtccac | N.D. | 566 |
| 147798 | Coding | 545 | 2496 | cccaggagctggaggtcatg | N.D. | 567 |
| 147799 | Coding | 545 | 2573 | ttgagcccttcctgatgacc | N.D. | 568 |
| 147800 | Coding | 545 | 2811 | atctggaccccactcctagc | N.D. | 569 |
| 147801 | Coding | 545 | 2842 | cagacccgactcgtggaaga | 38 | 570 |
| 147802 | Coding | 545 | 3367 | gccctcagtagattcatcat | N.D. | 571 |
| 147803 | Coding | 545 | 3611 | gccatgccaccctcttggaa | N.D. | 572 |
| 147804 | Coding | 545 | 3791 | aacccacgtgccggaaagtc | N.D. | 573 |
| 147805 | Coding | 545 | 3841 | actcccagatgccttctgaa | N.D. | 574 |
| 147806 | Coding | 545 | 4281 | atgtggtaacgagcccgaag | 100 | 575 |
| 147807 | Coding | 545 | 4391 | ggcgtagagacccatcacat | 25 | 576 |
| 147808 | Coding | 545 | 4641 | gtgttaggatccctctgaca | N.D. | 577 |
| 147809 | Coding | 545 | 5241 | cccagtgatagctctgtgag | 60 | 578 |
| 147810 | Coding | 545 | 5355 | atttcagcatatgagcccat | 0 | 579 |
| 147811 | Coding | 545 | 5691 | ccctgaaccttagcaacagt | N.D. | 580 |
| 147812 | Coding | 545 | 5742 | gctgaagccagcccagcgat | N.D. | 581 |
| 147813 | Coding | 545 | 5891 | acagctgcccagtatgttct | N.D. | 582 |
| 147814 | Coding | 545 | 7087 | cccaataagatttataacaa | 34 | 583 |
| 147815 | Coding | 545 | 7731 | tggcctaccagagacaggta | 45 | 584 |
| 147816 | Coding | 545 | 7841 | tcatacgtttagcccaatct | 100 | 585 |
| 147817 | Coding | 545 | 7901 | gcatggtcccaaggatggtc | 0 | 586 |
| 147818 | Coding | 545 | 8491 | agtgatggaagctgcgatac | 30 | 587 |
| 147819 | Coding | 545 | 9181 | atgagcatcatgcctcccag | N.D. | 588 |
| 147820 | Coding | 545 | 9931 | gaacacatagccgaatgccg | 100 | 589 |
| 147821 | Coding | 545 | 10263 | gtggtgccctctaatttgta | N.D. | 590 |
| 147822 | Coding | 545 | 10631 | cccgagaaagaaccgaaccc | N.D. | 591 |
| 147823 | Coding | 545 | 10712 | tgccctgcagcttcactgaa | 19 | 592 |
| 147824 | Coding | 545 | 11170 | gaaatcccataagctcttgt | N.D. | 593 |
| 147825 | Coding | 545 | 12301 | agaagctgcctcttcttccc | 72 | 594 |
| 147826 | Coding | 545 | 12401 | tcagggtgagccctgtgtgt | 80 | 595 |
| 147827 | Coding | 545 | 12471 | ctaatggccccttgataaac | 13 | 596 |
| 147828 | Coding | 545 | 12621 | acgttatccttgagtccctg | 12 | 597 |
| 147829 | Coding | 545 | 12741 | tatatcccaggtttccccgg | 64 | 598 |
| 147830 | Coding | 545 | 12801 | acctgggacagtaccgtccc | N.D. | 599 |
| 147831 | 3′ UTR | 545 | 13921 | ctgcctactgcaaggctggc | 0 | 600 |
| 147832 | 3′ UTR | 545 | 13991 | agagaccttccgagccctgg | N.D. | 601 |
| 147833 | 3′ UTR | 545 | 14101 | atgatacacaataaagactc | 25 | 602 |

As shown in Table 22, SEQ ID NOs: 549, 550, 551, 553, 555, 557, 559, 563, 570, 575, 578, 583, 584, 585, 587, 589, 594, 595 and 598 demonstrated at least 30% inhibition of human apolipoprotein B expression in this assay and are therefore preferred. The target sites to which these preferred sequences are complementary are herein referred to as “preferred target segments” and are therefore preferred sites for targeting by compounds of the present invention. As apolipoprotein B exists in two forms in mammals (ApoB-48 and ApoB-100) which are colinear at the amino terminus, antisense oligonucleotides targeting nucleotides 1-6530 hybridize to both forms, while those targeting nucleotides 6531-14121 are specific to the long form of apolipoprotein B.

### Example 49

Antisense Inhibition of Human Apolipoprotein B mRNA Expression by Chimeric Phosphorothioate Oligonucleotides having 2′-Moe Wings and a Deoxy Gap-Dose Response Study

In accordance with the present invention, a subset of the antisense oligonucleotides in Example 48 were further investigated in dose-response studies. Treatment doses were 50, 150 and 250 nM. The compounds were analyzed for their effect on human apolipoprotein B mRNA levels in HepG2 cells by quantitative real-time PCR as described in other examples herein. Data are averages from two-experiments and are shown in Table 23.

TABLE 23
Inhibition of Human Apolipoprotein B mRNA
Levels By Chimeric Phosphorothioate
Oligonucleotides Having 2′-Moe Wings and a Deoxy Gap

| | Percent Inhibition | | |
|---|---|---|---|
| ISIS # | 50 nM | 150 nM | 250 nM |
| 147788 | 54 | 63 | 72 |
| 147806 | 23 | 45 | 28 |
| 147816 | 25 | 81 | 65 |
| 147820 | 10 | 0 | 73 |

### Example 50

Antisense Inhibition of Mouse Apolipoprotein B Expression by Chimeric Phosphorothioate Oligonucleotides having 2′-Moe Wings and a Deoxy Gap

Oligonucleotides targeting mouse apolipoprotein B were designed as described in Example 2, synthesized as described in Examples 3-7, analyzed as described in Example 8 and assayed by RT-PCR as described in Example 12.

Probes and primers to mouse apolipoprotein B were designed to hybridize to a mouse apolipoprotein B sequence, using published sequence information (GenBank accession number M35186.1, incorporated herein as SEQ ID NO: 603). For mouse apolipoprotein B the PCR primers were: forward primer: CGTGGGCTCCAGCATTCTA (SEQ ID NO: 604) reverse primer: AGTCATTTCTGCCTTTGCGTC (SEQ ID NO: 605) and the PCR probe was: FAM-CCAATGGTCGGGCACTGCTCAA-TAMRA (SEQ ID NO: 606) where FAM (PE-Applied Biosystems, Foster City, Calif.) is the fluorescent reporter dye) and TAMRA (PE-Applied Biosystems, Foster City, Calif.) is the quencher dye.

The series of oligonucleotides was designed to target different regions of the mouse apolipoprotein B RNA, using published sequence information (GenBank accession number M35186.1, incorporated herein as SEQ ID NO: 603). The oligonucleotides are shown in Table 24. “Target site” indicates the first (5′-most) nucleotide number on the particular target sequence to which the oligonucleotide binds. All compounds in Table 24 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines. The compounds were analyzed for their effect on mouse apolipoprotein B mRNA levels in primary hepatocytes by quantitative real-time PCR as described in other examples herein. Data are averages from two experiments in which AML12 cells were cultured and treated with oligonucleotides 147475-147778 (SEQ ID NOs: 607-659) according to the protocol outlined in Example 34. If present, “N.D.” “no data”.

TABLE 24

Inhibition of Mouse Apolipoprotein B mRNA Levels
by Chimeric Phosphorothioate Oligonucleotides
Having 2′-Moe Wings and a Deoxy Gap

| | | | | | | |
|---|---|---|---|---|---|---|
| | | TARGET | | | | |
| ISIS # | REGION | SEQ ID NO | TARGET SITE | SEQUENCE | % INHIB | SEQ ID NO |

| | | | | | | |
|---|---|---|---|---|---|---|
| 147475 | Coding | 603 | 13 | attgtatgtgagaggtgagg | 79 | 607 |
| 147476 | Coding | 603 | 66 | gaggagattggatcttaagg | 13 | 608 |
| 147477 | Coding | 603 | 171 | cttcaaattgggactctcct | N.D. | 609 |
| 147478 | Coding | 603 | 211 | tccaggaattgagcttgtgc | 78 | 610 |
| 147479 | Coding | 603 | 238 | ttcaggactggaggatgagg | N.D. | 611 |
| 147480 | Coding | 603 | 291 | tctcaccctcatgctccatt | 54 | 612 |
| 147481 | Coding | 603 | 421 | tgactgtcaagggtgagctg | 24 | 613 |
| 147482 | Coding | 603 | 461 | gtccagcctaggaacactca | 59 | 614 |
| 147483 | Coding | 603 | 531 | atgtcaatgccacatgtcca | N.D. | 615 |
| 147484 | Coding | 603 | 581 | ttcatccgagaagttgggac | 49 | 616 |
| 147485 | Coding | 603 | 601 | atttgggacgaatgtatgcc | 64 | 617 |
| 147486 | Coding | 603 | 711 | agttgaggaagccagattca | N.D. | 618 |
| 147487 | Coding | 603 | 964 | ttcccagtcagctttagtgg | 73 | 619 |
| 147488 | Coding | 603 | 1023 | agcttgcttgttgggcacgg | 72 | 620 |
| 147489 | Coding | 603 | 1111 | cctatactggcttctatgtt | 5 | 621 |
| 147490 | Coding | 603 | 1191 | tgaactccgtgtaaggcaag | N.D. | 622 |
| 147491 | Coding | 603 | 1216 | gagaaatccttcagtaaggg | 71 | 623 |
| 147492 | Coding | 603 | 1323 | caatggaatgcttgtcactg | 68 | 624 |
| 147493 | Coding | 603 | 1441 | gcttcattataggaggtggt | 41 | 625 |
| 147494 | Coding | 603 | 1531 | acaactgggatagtgtagcc | 84 | 626 |
| 147495 | Coding | 603 | 1631 | gttaggaccagggattgtga | 0 | 627 |
| 147496 | Coding | 603 | 1691 | accatggaaaactggcaact | 19 | 628 |
| 147497 | Coding | 603 | 1721 | tgggaggaaaaacttgaata | N.D. | 629 |
| 147498 | Coding | 603 | 1861 | tgggcaacgatatctgattg | 0 | 630 |
| 147499 | Coding | 603 | 1901 | ctgcagggcgtcagtgacaa | 29 | 631 |
| 147500 | Coding | 603 | 1932 | gcatcagacgtgatgttccc | N.D. | 632 |
| 147501 | Coding | 603 | 2021 | cttggttaaactaatggtgc | 18 | 633 |
| 147502 | Coding | 603 | 2071 | atgggagcatggaggttggc | 16 | 634 |
| 147503 | Coding | 603 | 2141 | aatggatgatgaaacagtgg | 26 | 635 |
| 147504 | Coding | 603 | 2201 | atcaatgcctcctgttgcag | N.D. | 636 |
| 147505 | Coding | 603 | 2231 | ggaagtgagactttctaagc | 76 | 637 |
| 147506 | Coding | 603 | 2281 | aggaaggaactcttgatatt | 58 | 638 |
| 147507 | Coding | 603 | 2321 | attggcttcattggcaacac | 81 | 639 |
| 147759 | Coding | 603 | 1 | aggtgaggaagttggaattc | 19 | 640 |
| 147760 | Coding | 603 | 121 | ttgttccctgaagttgttac | N.D. | 641 |
| 147761 | Coding | 603 | 251 | gttcatggattccttcagga | 45 | 642 |
| 147762 | Coding | 603 | 281 | atgctccattctcacatgct | 46 | 643 |
| 147763 | Coding | 603 | 338 | tgcgactgtgtctgatttcc | 34 | 644 |
| 147764 | Coding | 603 | 541 | gtccctgaagatgtcaatgc | 97 | 645 |
| 147765 | Coding | 603 | 561 | aggcccagttccatgaccct | 59 | 646 |
| 147766 | Coding | 603 | 761 | ggagcccacgtgctgagatt | 59 | 647 |
| 147767 | Coding | 603 | 801 | cgtccttgagcagtgcccga | 5 | 648 |
| 147768 | Coding | 603 | 1224 | cccatatggagaaatccttc | 24 | 649 |
| 147769 | Coding | 603 | 1581 | catgcctggaagccagtgtc | 89 | 650 |
| 147770 | Coding | 603 | 1741 | gtgttgaatcccttgaaatc | 67 | 651 |
| 147771 | Coding | 603 | 1781 | ggtaaagttgcccatggctg | 68 | 652 |
| 147772 | Coding | 603 | 1841 | gttataaagtccagcattgg | 78 | 653 |
| 147773 | Coding | 603 | 1931 | catcagacgtgatgttccct | 85 | 654 |
| 147774 | Coding | 603 | 1956 | tggctagtttcaatcccctt | 84 | 655 |
| 147775 | Coding | 603 | 2002 | ctgtcatgactgccctttac | 52 | 656 |
| 147776 | Coding | 603 | 2091 | gcttgaagttcattgagaat | 92 | 657 |
| 147777 | Coding | 603 | 2291 | ttcctgagaaaggaaggaac | N.D. | 658 |
| 147778 | Coding | 603 | 2331 | tcagatatacattggcttca | 14 | 659 |

As shown in Table 24, SEQ ID NOs: 607, 610, 612, 614, 617, 619, 620, 623, 624, 626, 637, 638, 639, 645, 646, 647, 650, 651, 652, 653, 654, 655, 656 and 657 demonstrated at least 50% inhibition of mouse apolipoprotein B expression in this assay and are therefore preferred. The target sites to which these preferred sequences are complementary are herein referred to as “preferred target segments” and are therefore preferred sites for targeting by compounds of the present invention.

### Example 51

Antisense Inhibition of Mouse Apolipoprotein B mRNA Expression by Chimeric Phosphorothioate Oligonucleotides having 2′-Moe Wings and a Deoxy Gap-Dose Response Study

In accordance with the present invention, a subset of the antisense oligonucleotides in Example 50 were further investigated in dose-response studies. Treatment doses were 50, 150 and 300 nM. The compounds were analyzed for their effect on mouse apolipoprotein B mRNA levels in mouse primary hepatocytes by quantitative real-time PCR as described in other examples herein. Data are averages from two experiments and are shown in Table 25.

TABLE 25
Inhibition of Mouse Apolipoprotein B mRNA
Levels by Chimeric Phosphorothioate
Oligonucleotides Having 2′-Moe Wings and a Deoxy Gap

| | Percent Inhibition | | |
|---|---|---|---|
| ISIS # | 50 nM | 150 nM | 300 nM |
| 147483 | 56 | 88 | 89 |
| 147764 | 48 | 84 | 90 |
| 147769 | 3 | 14 | 28 |
| 147776 | 0 | 17 | 44 |

### Example 52

Target Validation—Effects of Antisense Inhibition of Apolipoprotein B (ISIS 147764) in C57BL/6 Mice: Lean Animals vs. High Fat Fed Animals

C57BL/6 mice, a strain reported to be susceptible to hyperlipidemia-induced atherosclerotic plaque formation were used in the following studies to evaluate antisense oligonucleotides as potential lipid lowering compounds in lean versus high fat fed mice.

Male C57BL/6 mice were divided into two matched groups; (1) wild-type control animals (lean animals) and (2) animals receiving a high fat diet (60% kcal fat). Control animals received saline treatment and were maintained on a normal rodent diet. After overnight fasting, mice from each group were dosed intraperitoneally every three days with saline or 50 mg/kg ISIS 147764 (SEQ ID NO: 645) for six weeks. At study termination and forty eight hours after the final injections, animals were sacrificed and evaluated for target mRNA levels in liver, cholesterol and triglyceride levels, liver enzyme levels and serum glucose levels. The results of the comparative studies are shown in Table 26.

TABLE 26
Effects of ISIS 147764 Treatment On Apolipoprotein
B mRNA, Cholesterol, Lipid, Triglyceride, Liver Enzyme
And Glucose Levels in Lean and High Fat Mice.

| | Percent Change | | | | | | | | |
|---|---|---|---|---|---|---|---|---|---|
| Treatment | | | Lipoproteins | | | | Liver Enzymes | | |
| Group | MRNA | CHOL | VLDL | LDL | HDL | TRIG | AST | ALT | GLUC |
| Lean-control | −73 | −63 | No | −64 | −44 | −34 | Slight | No change | No change |
| | | | change | | | | decrease | | |
| High Fat | −87 | −67 | No | −87 | −65 | No change | Slight | Slight | −28 |
| Group | | | change | | | | decrease | increase | |

It is evident from these data that treatment with ISIS 147764 lowered cholesterol as well as LDL and HDL lipoproteins and serum glucose in both lean and high fat mice and that the effects demonstrated are, in fact, due to the inhibition of apolipoprotein B expression as supported by the decrease in mRNA levels. No significant changes in liver enzyme levels were observed, indicating that the antisense oligonucleotide was not toxic to either treatment group.

### Example 53

Target Validation—Effects Of Antisense Inhibition Of Apolipoprotein B (ISIS 147764) on High Fat Fed Mice; 6 Week Timecourse Study

A 6-week timecourse study was performed to further investigate the effects of ISIS 147764 on lipid and glucose metabolism in high fat fed mice.

Male C57BL/6 mice (n=8) receiving a high fat diet (60% kcal fat) were evaluated over the course of 6 weeks for the effects of treatment with the antisense oligonucleotide, ISIS 147764. Control animals received saline treatment (50 mg/kg). A subset of animals received a daily oral dose (20 mg/kg) atorvastatin calcium (Lipitor®, Pfizer Inc.). All mice, except atorvastatin-treated animals, were dosed intraperitoneally every three days (twice a week), after fasting overnight, with 5, 25, 50 mg/kg ISIS 147764 (SEQ ID NO: 645) or saline (50 mg/kg) for six weeks. Serum cholesterol and lipoproteins were analyzed at 0, 2 and 6 week interim timepoints. At study termination, animals were sacrificed 48 hours after the final injections and evaluated for levels of target mRNA levels in liver, cholesterol, lipoprotein, triglyceride, liver enzyme (AST and ALT) and serum glucose levels as well as body, liver, spleen and fat pad weights.

### Example 54

Target Validation—Effects Of Antisense Inhibition of Apolipoprotein B (ISIS 147764) In High Fat Fed Mice-mRNA Expression in Liver

Male C57BL/6 mice (n=8) receiving a high fat diet (60% kcal fat) were evaluated over the course of 6 weeks for the effects of ISIS 147764 on mRNA expression. Control animals received saline treatment (50 mg/kg). Mice were dosed intraperitoneally every three days (twice a week), after fasting overnight, with 5, 25, 50 mg/kg ISIS 147764 (SEQ ID NO: 645) or saline (50 mg/kg) for six weeks. At study termination, animals were sacrificed 48 hours after the final injections and evaluated for levels of target mRNA levels in liver. ISIS 147764 showed a dose-response effect, reducing mRNA levels by 15, 75 and 88% at doses of 5, 25 and 50 mg/kg, respectively.

### Example 55

Target Validation—Effects Of Antisense Inhibition of Apolipoprotein B (ISIS 147764) on Serum Cholesterol and Triglyceride Levels

Male C57BL/6 mice (n=8) receiving a high fat diet (60% kcal fat) were evaluated over the course of 6 weeks for the effects of ISIS 147764 on serum cholesterol and triglyceride levels. Control animals received saline treatment (50 mg/kg). Mice were dosed intraperitoneally every three days (twice a week), after fasting overnight, with 5, 25, 50 mg/kg ISIS 147764 (SEQ ID NO: 645) or saline (50 mg/kg) for six weeks.

Serum cholesterol levels were measured at 0, 2 and 6 weeks and this data is shown in Table 27. Values in the table are expressed as percent inhibition and are normalized to the saline control.

In addition to serum cholesterol, at study termination, animals were sacrificed 48 hours after the final injections and evaluated for triglyceride levels.

Mice treated with ISIS 147764 showed a reduction in both serum cholesterol (240 mg/dL for control animals and 225, 125 and 110 mg/dL for doses of 5, 25, and 50 mg/kg, respectively) and triglycerides (115 mg/dL for control animals and 125, 150 and 85 mg/dL for doses of 5, 25, and 50 mg/kg, respectively) to normal levels by study end.

These data were also compared to the effects of atorvastatin calcium at an oral dose of 20 mg/kg which showed only a minimal decrease in serum cholesterol of 20 percent at study termination.

TABLE 27
Percent Inhibition of Mouse Apolipoprotein B
Cholesterol Levels by ISIS 147764

Percent Inhibition

| time | Saline | 5 mg/kg | 25 mg/kg | 50 mg/kg |
|---|---|---|---|---|
| 0 weeks | 0 | 0 | 0 | 0 |
| 2 weeks | 0 | 5 | 12 | 20 |
| 6 weeks | 0 | 10 | 45 | 55 |

### Example 56

Target Validation—Effects Of Antisense Inhibition of Apolipoprotein B (ISIS 147764) on Lipoprotein Levels

Male C57BL/6 mice (n=8) receiving a high fat diet (60% kcal fat) were evaluated over the course of 6 weeks for the effects of ISIS 147764 on lipoprotein (VLDL, LDL and HDL) levels. Control animals received saline treatment (50 mg/kg). Mice were dosed intraperitoneally every three days (twice a week), after fasting overnight, with 5, 25, 50 mg/kg ISIS 147764 (SEQ ID NO: 645) or saline (50 mg/kg) for six weeks.

Lipoprotein levels were measured at 0, 2 and 6 weeks and this data is shown in Table 28. Values in the table are expressed as percent inhibition and are normalized to the saline control. Negative values indicate an observed increase in lipoprotein levels.

These data were also compared to the effects of atorvastatin calcium at a daily oral dose of 20 mg/kg at 0, 2 and 6 weeks.

These data demonstrate that at a dose of 50 mg/kg, ISIS 147764 is capable of lowering all categories of serum lipoproteins investigated to a greater extent than atorvastatin.

TABLE 28
Percent Inhibition of Mouse Apolipoprotein B
Lipoprotein Levels by ISIS 147764 as
Compared to Atorvastatin

| | | Percent Inhibition | | | | |
|---|---|---|---|---|---|---|
| | | Dose | | | | |
| Lipo- | Time | | 5 | 25 | 50 | atorvastatin |
| protein | (weeks) | Saline | mg/kg | mg/kg | mg/kg | (20 mg/kg) |
| VLDL | 0 | 0 | 0 | 0 | 0 | 0 |
| | 2 | 0 | 25 | 30 | 40 | 15 |
| | 6 | 0 | 10 | −30 | 15 | −5 |
| LDL | 0 | 0 | 0 | 0 | 0 | 0 |
| | 2 | 0 | −30 | 10 | 40 | 10 |
| | 6 | 0 | −10 | 55 | 90 | −10 |
| HDL | 0 | 0 | 0 | 0 | 0 | 0 |
| | 2 | 0 | 5 | 10 | 10 | 15 |
| | 6 | 0 | 10 | 45 | 50 | 20 |

### Example 57

Target Validation—Effects of Antisense Inhibition of Apolipoprotein B (ISIS 147764) on Serum AST and ALT Levels

Male C57BL/6 mice (n=8) receiving a high fat diet (60% kcal fat) were evaluated over the course of 6 weeks for the effects of ISIS 147764 on liver enzyme (AST and ALT) levels. Control animals received saline treatment (50 mg/kg). Mice were dosed intraperitoneally every three days (twice a week), after fasting overnight, with 5, 25, 50 mg/kg ISIS 147764 (SEQ ID NO: 645) or saline (50 mg/kg) for six weeks.

AST and ALT levels were measured at 6 weeks and this data is shown in Table 29. Values in the table are expressed as IU/L. Increased levels of the liver enzymes ALT and AST indicate toxicity and liver damage.

Mice treated with ISIS 147764 showed no significant change in AST levels over the duration of the study compared to saline controls (105, 70 and 80 IU/L for doses of 5, 25 and 50 mg/kg, respectively compared to 65 IU/L for saline control). Mice treated with atorvastatin at a daily oral dose of 20 mg/kg had AST levels of 85 IU/L.

ALT levels were increased by all treatments over the duration of the study compared to saline controls (50, 70 and 100 IU/L for doses of 5, 25 and 50 mg/kg, respectively compared to 25 IU/L for saline control). Mice treated with atorvastatin at a daily oral dose of 20 mg/kg had AST levels of 40 IU/L.

### Example 58

Target Validation—Effects of Antisense Inhibition of Apolipoprotein B (ISIS 147764) on Serum Glucose Levels

Male C57BL/6 mice (n=8) receiving a high fat diet (60% kcal fat) were evaluated over the course of 6 weeks for the effects of ISIS 147764 on serum glucose levels. Control animals received saline treatment (50 mg/kg). Mice were dosed intraperitoneally every three days (twice a week), after fasting overnight, with 5, 25, 50 mg/kg ISIS 147764 (SEQ ID NO: 645) or saline (50 mg/kg) for six weeks.

At study termination, animals were sacrificed 48 hours after the final injections and evaluated for serum glucose levels. ISIS 147764 showed a dose-response effect, reducing serum glucose levels to 225, 190 and 180 mg/dL at doses of 5, 25 and 50 mg/kg, respectively compared to the saline control of 300 mg/dL. Mice treated with atorvastatin at a daily oral dose of 20 mg/kg had serum glucose levels of 215 mg/dL. These data demonstrate that ISIS 147764 is capable of reducing serum glucose levels in high fat fed mice.

### Example 59

Target Validation—Effects of Antisense Inhibition of Apolipoprotein B (ISIS 147764) on Body, Spleen, Liver and Fat Pad Weight

Male C57BL/6 mice (n=8) receiving a high fat diet (60% kcal fat) were evaluated over the course of 6 weeks for the effects of ISIS 147764 on body, spleen, liver and fat pad weight. Control animals received saline treatment (50 mg/kg). Mice were dosed intraperitoneally every three days (twice a week), after fasting overnight, with 5, 25, 50 mg/kg ISIS 147764 (SEQ ID NO: 645) or saline (50 mg/kg) for six weeks.

At study termination, animals were sacrificed 48 hours after the final injections and body, spleen, liver and fat pad weights were measured. These data are shown in Table 29. Values are expressed as percent change in body weight or organ weight compared to the saline-treated control animals. Data from mice treated with atorvastatin at a daily dose of 20 mg/kg are also shown in the table. Negative values indicated a decrease in weight.

TABLE 29
Effects of Antisense Inhibition of Mouse Apolipoprotein
B on Body and Organ Weight

| | Percent Change | | | |
|---|---|---|---|---|
| | Dose | | | Atorvastatin |
| Tissue | 5 mg/kg | 25 mg/kg | 50 mg/kg | 20 mg/kg |
| Total Body | 5 | 5 | −4 | 1 |
| Wt. | | | | |
| Spleen | 10 | 10 | 46 | 10 |
| Liver | 18 | 70 | 80 | 15 |
| Fat | 10 | 6 | −47 | 7 |

These data show a decrease in fat over the dosage range of ISIS 147764 counterbalanced by an increase in both spleen and liver weight with increased dose to give an overall decrease in total body weight.

### Example 60

Target Validation—Effects of Antisense Inhibition of Apolipoprotein B (ISIS 147764) in b6.129p-apoe^{tm1unc} Knockout Mice: Lean Animals vs. High Fat-Fed Animals

B6.129P-ApoE^{tm1Unc} knockout mice (herein referred to as ApoE knockout mice) obtained from The Jackson Laboratory (Bar Harbor, Me.), are homozygous for the Apoe^{tm1Unc} mutation and show a marked increase in total plasma cholesterol levels that are unaffected by age or sex. These animals present with fatty streaks in the proximal aorta at 3 months of age. These lesions increase with age and progress to lesions with less lipid but more elongated cells, typical of a more advanced stage of pre-atherosclerotic lesion.

The mutation in these mice resides in the apolipoprotein E (ApoE) gene. The primary role of the ApoE protein is to transport cholesterol and triglycerides throughout the body. It stabilizes lipoprotein structure, binds to the low density lipoprotein receptor (LDLR) and related proteins, and is present in a subclass of HDLs, providing them the ability to bind to LDLR. ApoE is expressed most abundantly in the liver and brain.

Female B6.129P-Apoe^{tm1Unc} knockout mice (ApoE knockout mice) were used in the following studies to evaluate antisense oligonucleotides as potential lipid lowering compounds.

Female ApoE knockout mice ranged in age from 5 to 7 weeks and were placed on a normal diet for 2 weeks before study initiation. ApoE knockout mice were then fed ad libitum a 60% fat diet, with 0.15% added cholesterol to induce dyslipidemia and obesity. Control animals were maintained on a high-fat diet with no added cholesterol. After overnight fasting, mice from each group were dosed intraperitoneally every three days with saline, 50 mg/kg of a control antisense oligonucleotide (ISIS 29837 TCGATCTCCTTTTATGCCCG; SEQ ID NO. 660) or 5, 25 or 50 mg/kg ISIS 147764 (SEQ ID NO: 645) for six weeks.

The control oligonucleotide is a chimeric oligonucleotide (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines.

At study termination and forty eight hours after the final injections, animals were sacrificed and evaluated for target mRNA levels in liver by RT-PCR methods verified by Northern Blot analysis, glucose levels, cholesterol and lipid levels by HPLC separation methods and triglyceride and liver enzyme levels (performed by LabCorp Preclinical Services; San Diego, Calif.). Data from ApoE knockout mice treated with atorvastatin at a daily dose of 20 mg/kg are also shown in the table for comparison.

The results of the comparative studies are shown in Table 29. Data are normalized to saline controls.

TABLE 30
Effects of ISIS 147764 Treatment on Apolipoprotein
B mRNA, Cholesterol, Glucose, Lipid, Triglyceride
and Liver Enzyme Levels in Apoe Knockout Mice.

| | | Percent Inhibition | | | | |
|---|---|---|---|---|---|---|
| | | Dose | | | | atorvastatin (20 |
| | | Control | 5 mg/kg | 25 mg/kg | 50 mg/kg | mg/kg) |
| mRNA | | 0 | 2 | 42 | 70 | 10 |
| | | Glucose Levels (mg/dL) | | | | |
| Glucose | | 225 | 195 | 209 | 191 | 162 |
| | | Cholesterol Levels (mg/dL) | | | | |
| Cholesterol | | 1750 | 1630 | 1750 | 1490 | 938 |
| | | Lipoprotein Levels (mg/dL) | | | | |
| Lipoprotein | HDL | 51 | 49 | 62 | 61 | 42 |
| | LDL | 525 | 475 | 500 | 325 | 250 |
| | VLDL | 1190 | 1111 | 1194 | 1113 | 653 |
| | | Liver Enzyme Levels (IU/L) | | | | |
| Liver Enzymes | AST | 55 | 50 | 60 | 85 | 75 |
| | ALT | 56 | 48 | 59 | 87 | 76 |

It is evident from these data that treatment with ISIS 147764 lowered glucose and cholesterol as well as all lipoproteins investigated (HDL, LDL and VLDL) in ApoE knockout mice. Further, these decreases correlated with a decrease in both protein and RNA levels of apolipoprotein B, demonstrating an antisense mechanism of action. No significant changes in liver enzyme levels were observed, indicating that the antisense oligonucleotide was not toxic to either treatment group.

### Example 61

Antisense Inhibition of Human BH3 Interacting Domain Death Agonist mRNA Expression by Chimeric Phosphorothioate Oligonucleotides Having 2′-Moe Wings and a Deoxy Gap

The Bcl-2 family of proteins, which includes both positive and negative regulators of apoptosis, act as checkpoints upstream of activated protease cascades orchestrated by caspases and are required for all aspects of cell death. BH3 interacting domain death agonist is a member of the Bcl-2 family and has been shown to dimerize with either Bcl-2, a cell death antagonist, or Bax, a cell death agonist. Due to the integral role played by BH3 interacting domain death agonist in apoptosis, the pharmacological modulation of BH3 interacting domain death agonist activity and/or expression may therefore be an appropriate point of therapeutic intervention in pathological conditions involving deregulated cell death.

Oligonucleotides targeting human BH3 interacting domain death agonist were designed as described in Example 2, synthesized as described in Examples 3-7, analyzed as described in Example 8 and assayed by RT-PCR as described in Example 12.

Probes and primers to human BH3 Interacting domain death agonist were designed to hybridize to a human BH3 Interacting domain death agonist sequence, using published sequence information (GenBank accession number NM_{—}001196.1, incorporated herein as SEQ ID NO: 661). For human BH3 Interacting domain death agonist the PCR primers were: forward primer: AGAAGACATCATCCGGAATATTGC (SEQ ID NO: 662) reverse primer: GGAGGGATGCTACGGTCCAT (SEQ ID NO: 663) and the PCR probe was: FAM-AGGCACCTCGCCCAGGTCGG-TAMRA (SEQ ID NO: 664) where FAM (PE-Applied Biosystems, Foster City, Calif.) is the fluorescent reporter dye) and TAMRA (PE-Applied Biosystems, Foster City, Calif.) is the quencher dye.

The series of oligonucleotides was designed to target different regions of the human BH3 Interacting domain death agonist RNA, using published sequences (GenBank accession number NM_{—}001196.1, incorporated herein as SEQ ID NO: 661, and residues 12001-28000 of GenBank accession number AC006285, incorporated herein as SEQ ID NO: 665). The oligonucleotides are shown in Table 31. “Target site” indicates the first (5=-most) nucleotide number on the particular target sequence to which the oligonucleotide binds. All compounds in Table 31 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human BH3 Interacting domain death agonist mRNA levels by quantitative real-time PCR as described in other examples herein. Data are averages from two experiments in which T-24 cells were cultured as described in Section 15 (15. Cell Lines for Assaying Oligonucleotide Activity) and treated with oligonucleotides 119845-119922 (SEQ ID NOs: 666-743) according to the protocol outlined in Example 34. If present, “N.D.” indicates “no data”.

TABLE 31

Inhibition of Human BH3 Interacting Domain Death Agonist mRNA Levels
by Chimeric Phosphorothioate Oligonucleotides
Having 2′-Moe Wings and a Deoxy Gap

| | | | | | | |
|---|---|---|---|---|---|---|
| | | TARGET | | | | |
| ISIS # | REGION | SEQ ID NO | TARGET SITE | SEQUENCE | % INHIB | SEQ ID NO |

| | | | | | | |
|---|---|---|---|---|---|---|
| 119845 | Coding | 661 | 354 | ctttcagaatctgcctctat | 67 | 666 |
| 119846 | Coding | 661 | 707 | agtccatcccatttctggct | 74 | 667 |
| 119847 | 5′ UTR | 665 | 60 | actgtggtgagtctcccacc | 88 | 668 |
| 119848 | 5′ UTR | 665 | 2083 | agtgtcccagtggcgacctg | 90 | 669 |
| 119849 | Coding | 665 | 2134 | cacagtccatggcctgggca | 98 | 670 |
| 119850 | Intron | 665 | 3582 | ctccgcttcctcactccgaa | 84 | 671 |
| 119851 | Intron | 665 | 3845 | tactcgggaggctgaggcag | 88 | 672 |
| 119852 | Intron | 665 | 3906 | ccgtctttactaagatacaa | 90 | 673 |
| 119853 | Intron | 665 | 4540 | tcaagacagtaaatcctgca | 93 | 674 |
| 119854 | Intron | 665 | 4580 | ctttttagatcacaggaaaa | 89 | 675 |
| 119855 | Intron | 665 | 4987 | gccatttaattccaagaata | 92 | 676 |
| 119856 | Intron | 665 | 5092 | ggcccactgagtggacagct | 93 | 677 |
| 119857 | Intron | 665 | 5373 | gcatctgttgtttaaagcca | 81 | 678 |
| 119858 | Intron | 665 | 5778 | acggagcagccgcatggcac | 85 | 679 |
| 119859 | Intron | 665 | 6999 | ggtttcaccatgttggtcag | 85 | 680 |
| 119860 | Intron | 665 | 7125 | tctcggctcactacaacctc | 75 | 681 |
| 119861 | Intron | 665 | 7369 | agggacgctgagatctgcgc | 92 | 682 |
| 119862 | Intron | 665 | 8083 | ggtctcaacaggcagaggca | 83 | 683 |
| 119863 | Coding | 665 | 8254 | atccctgaggctggaaccgt | 96 | 684 |
| 119864 | Coding | 665 | 8282 | caaacaccagtaggtttgtg | 92 | 685 |
| 119865 | Coding | 665 | 8287 | gaagccaaacaccagtaggt | 86 | 686 |
| 119866 | Coding | 665 | 8318 | tgcggaagctgttgtcagaa | 81 | 687 |
| 119867 | Coding | 665 | 8362 | gggagccagcactggcagct | 79 | 688 |
| 119868 | Coding | 665 | 8418 | cgggagtggctgctgcggtt | 88 | 689 |
| 119869 | Intron | 665 | 9135 | gctggacctgggtttcctca | 86 | 690 |
| 119870 | Intron | 665 | 9353 | aagcagccccttggcaaagg | 94 | 691 |
| 119871 | Intron | 665 | 9424 | agggctggatctggaagtgg | 74 | 692 |
| 119872 | Intron | 665 | 9797 | agaaggcagagacattctca | 93 | 693 |
| 119873 | Intron | 665 | 9875 | gcccttcctggaccttccca | 95 | 694 |
| 119874 | Intron | 665 | 9992 | ctcagtctagaggcaaaggc | 90 | 695 |
| 119875 | Intron | 665 | 10172 | ctgatccgtctgtgtccagc | 96 | 696 |
| 119876 | Intron | 665 | 10643 | aagtagctgggattacaggc | 83 | 697 |
| 119877 | Intron | 665 | 11311 | ggccctgtacctagctccca | 94 | 698 |
| 119878 | Intron | 665 | 11394 | atcataccactacactccag | 18 | 699 |
| 119879 | Intron | 665 | 11641 | ttgtattttaagtagagacg | 85 | 700 |
| 119880 | Intron | 665 | 12649 | acaaggccagcccccactgg | 74 | 701 |
| 119881 | Intron | 665 | 12734 | ggcagagacagagcagactc | 77 | 702 |
| 119882 | Coding | 665 | 12795 | tgcctggcaatattccggat | 95 | 703 |
| 119883 | Coding | 665 | 12811 | cccgacctgggcgaggtgcc | 99 | 704 |
| 119884 | Coding | 665 | 12832 | gatgctacggtccatgctgt | 97 | 705 |
| 119885 | Coding | 665 | 12894 | acctcctccgaccggctggt | 98 | 706 |
| 119886 | Coding | 665 | 14042 | ccagggcagtggccaggtcc | 95 | 707 |
| 119887 | Coding | 665 | 14067 | ctagggtaggcctgcagcag | 94 | 708 |
| 119888 | Coding | 665 | 14072 | tgtctctagggtaggcctgc | 94 | 709 |
| 119889 | Coding | 665 | 14151 | cggagcaaggacggcgtgtg | 97 | 710 |
| 119890 | Coding | 665 | 14178 | aaattcactgttgtgtgaaa | 96 | 711 |
| 119891 | Coding | 665 | 14198 | tgcgtaggttctggttaata | 98 | 712 |
| 119892 | Intron | 665 | 14635 | agagcagtgggatcacaggc | 80 | 713 |
| 119893 | Intron | 665 | 14694 | tgttggccagggtggtctgg | 77 | 714 |
| 119894 | Intron | 665 | 16361 | agctgtccatacagactgct | 90 | 715 |
| 119895 | Coding | 665 | 16678 | cttctggaactgtccgttca | 96 | 716 |
| 119896 | 3′ UTR | 665 | 16753 | gttgacatgccagggctccg | 98 | 717 |
| 119897 | 3′ UTR | 665 | 16798 | atagaagtcacagctatctt | 95 | 718 |
| 119898 | 3′ UTR | 665 | 16933 | tgtagatttacagatgtgca | 68 | 719 |
| 119899 | 3′ UTR | 665 | 17176 | ttaagatagatagtccctat | 89 | 720 |
| 119900 | 3′ UTR | 665 | 17185 | tccttagtattaagatagat | 84 | 721 |
| 119901 | 3′ UTR | 665 | 17236 | tagttcagaatctctgtgcc | 62 | 722 |
| 119902 | 3′ UTR | 665 | 17267 | ccggacttcccatcatttga | 86 | 723 |
| 119903 | 3′ UTR | 665 | 17293 | aaaagtcaagcccctgtgta | 77 | 724 |
| 119904 | 3′ UTR | 665 | 17300 | aagttgaaaaagtcaagccc | 59 | 725 |
| 119905 | 3′ UTR | 665 | 17391 | gtaaacaaacagtggctgac | 82 | 726 |
| 119906 | 3′ UTR | 665 | 17415 | gtatgcagttagttacctga | 86 | 727 |
| 119907 | 3′ UTR | 665 | 17439 | tgatgtcatggaaagagaaa | 80 | 728 |
| 119908 | 3′ UTR | 665 | 17452 | tttagcaaagtcttgatgtc | 72 | 729 |
| 119909 | 3′ UTR | 665 | 17456 | tgtctttagcaaagtcttga | 89 | 730 |
| 119910 | 3′ UTR | 665 | 17588 | aacctgttctctccagatgc | 80 | 731 |
| 119911 | 3′ UTR | 665 | 17592 | tagaaacctgttctctccag | 85 | 732 |
| 119912 | 3′ UTR | 665 | 17596 | tgcttagaaacctgttctct | 90 | 733 |
| 119913 | 3′ UTR | 665 | 17632 | aatttttaaaaagtccaact | 24 | 734 |
| 119914 | 3′ UTR | 665 | 17731 | tgttgcactgtttctaaagc | 85 | 735 |
| 119915 | 3′ UTR | 665 | 17757 | agcttaccactggaacagca | 94 | 736 |
| 119916 | 3′ UTR | 665 | 17764 | gggacatagcttaccactgg | 70 | 737 |
| 119917 | 3′ UTR | 665 | 17779 | tttaaactgattcctgggac | 89 | 738 |
| 119918 | 3′ UTR | 665 | 17802 | gacccagcatccactgtcgt | 36 | 739 |
| 119919 | 3′ UTR | 665 | 17904 | gaagaaatcatgagtccgtc | 86 | 740 |
| 119920 | 3′ UTR | 665 | 17942 | gattttaaactcttaaagaa | 29 | 741 |
| 119921 | 3′ UTR | 665 | 17966 | tagagtttgtttttcctttc | 77 | 742 |
| 119922 | 3′ UTR | 665 | 17970 | aatatagagtttgtttttcc | 50 | 743 |

As shown in Table 31, SEQ ID NOs: 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 735, 736, 737, 738, 740, 742 and 743 demonstrated at least 50% inhibition of human BH3 Interacting domain death agonist expression in this assay and are therefore preferred. The target sites to which these preferred sequences are complementary are herein referred to as “preferred target segments” and are therefore preferred sites for targeting by compounds of the present invention.

### Example 62

Antisense Inhibition of Mouse BH3 Interacting Domain Death Agonist mRNA Expression by Chimeric Phosphorothioate Oligonucleotides having 2′-Moe Wings and a Deoxy Gap.

Oligonucleotides targeting mouse BH3 Interacting domain Death agonist were designed as described in Example 2, synthesized as described in Examples 3-7, analyzed as described in Example 8 and assayed by RT-PCR as described in Example 12.

For mouse BH3 Interacting domain Death agonist the PCR primers were: forward primer: TCGAAGACGAGCTGCAGACA (SEQ ID NO: 746) reverse primer: TGGCTCTATTCTTCCTTGGTTGA (SEQ ID NO: 747) and the PCR probe was: FAM-CAGCCAGGCCAGCCGCTCC-TAMRA (SEQ ID NO: 748) where FAM (PE-Applied Biosystems, Foster City, Calif.) is the fluorescent reporter dye) and TAMRA (PE-Applied Biosystems, Foster City, Calif.) is the quencher dye.

The series of oligonucleotides was designed to target different regions of the mouse BH3 Interacting domain Death agonist using published sequences (GenBank accession number U75506.1, incorporated herein as SEQ ID NO: 744, and residues 9000-120000 of GenBank accession number AC006945, incorporated herein as SEQ ID NO: 745). The oligonucleotides are shown in Table 32. “Target site” indicates the first (5=-most) nucleotide number on the particular target sequence to which the oligonucleotide binds. All compounds in Table 32 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines. The compounds were analyzed for their effect on mouse BH3 Interacting domain Death agonist mRNA levels by quantitative real-time PCR as described in other examples herein. Data are averages from two experiments in which b.END cells were cultured and treated with oligonucleotides 119925-120002 (SEQ ID NOs: 749-846) according to the protocol outlined in Example 34. If present, “N.D.” indicates “no data.”

TABLE 32

Inhibition of Mouse BH3 Interacting domain Death agonist mRNA Levels
by Chimeric Phosphorothioate Oligonucleotides
Having 2′-MOE Wings and a Deoxy Gap

| | | | | | | |
|---|---|---|---|---|---|---|
| | | TARGET | | | | |
| ISIS # | REGION | SEQ ID NO | TARGET SITE | SEQUENCE | % INHIB | SEQ ID NO |

| | | | | | | |
|---|---|---|---|---|---|---|
| 119925 | Start Codon | 744 | 21 | cgttgctgacctcagagtcc | 48 | 749 |
| 119926 | Coding | 744 | 232 | ctttcagaatctggctctat | 32 | 750 |
| 119927 | 5′ UTR | 742 | 4669 | ggcccggcgctctactccac | 39 | 751 |
| 119928 | 5′ UTR | 748 | 4699 | gctaaggcaaaggtttgcgg | 58 | 752 |
| 119929 | 5′ UTR | 748 | 5004 | cgggtccaccaggaggcctg | 42 | 753 |
| 119930 | 5′ UTR | 748 | 5693 | gccatggcaccaggcagtag | 71 | 754 |
| 119931 | 5′ UTR | 748 | 6758 | gccaggcagcgtgcccagaa | 74 | 755 |
| 119932 | 5′ UTR | 748 | 7548 | cttccccattcatacaccta | 61 | 756 |
| 119933 | 5′ UTR | 748 | 7977 | cacttgacaccaacagagac | 58 | 757 |
| 119934 | 5′ UTR | 748 | 8859 | gaagcctgtaatcctggcac | 73 | 758 |
| 119935 | 5′ UTR | 748 | 9373 | gaccatgtcctggccagaaa | 83 | 759 |
| 119936 | 5′ UTR | 748 | 9439 | gtcagtccagtaagggcttt | 61 | 760 |
| 119937 | 5′ UTR | 748 | 9698 | ttagcttagccacagaggga | 80 | 761 |
| 119938 | 5′ UTR | 748 | 9768 | cgcctgtgctctcttcctgc | 53 | 762 |
| 119939 | 5′ UTR | 748 | 10495 | cccatcttctggcctccttg | 35 | 763 |
| 119940 | 5′ UTR | 748 | 11230 | ctgaaactccaggctcagga | 76 | 764 |
| 119941 | 5′ UTR | 748 | 12652 | ctcatggcagctgcagcagt | 66 | 765 |
| 119942 | 5′ UTR | 748 | 14187 | cttgaaaaggaacaaagtgg | 44 | 766 |
| 119943 | 5′ UTR | 748 | 14566 | tctatacactactcataacc | 55 | 767 |
| 119944 | 5′ UTR | 748 | 17953 | ccatcacagaggccacttct | 41 | 768 |
| 119945 | 5′ UTR | 748 | 18196 | tccatccctggaacaatgtg | 58 | 769 |
| 119946 | 5′ UTR | 748 | 19488 | cagagctcagctttcttccc | 68 | 770 |
| 119947 | 5′ UTR | 748 | 19741 | agctcacagagtccagggaa | 55 | 771 |
| 119948 | 5′ UTR | 748 | 19752 | caagcactgccagctcacag | 59 | 772 |
| 119949 | Coding | 748 | 19782 | tcagagtccatggcacaagc | 61 | 773 |
| 119950 | Intron | 748 | 20989 | ttgccaaacagaagacacca | 3 | 774 |
| 119951 | Intron | 748 | 21013 | gcagagaaacaggctgtggt | 42 | 775 |
| 119952 | Coding | 748 | 21182 | gtctgtgatgtgcttggccc | 63 | 776 |
| 119953 | Coding | 748 | 21205 | tggagaaagccgaacaccag | 57 | 777 |
| 119954 | Coding | 748 | 21259 | acaggcagttcccgacccag | 71 | 778 |
| 119955 | Coding | 748 | 21282 | ggtctgcctcccagtaagct | 27 | 779 |
| 119956 | Coding | 748 | 21306 | cgtctgtctgcagctcgtct | 89 | 780 |
| 119957 | Intron | 748 | 21950 | cttttctgaatgacttgata | 39 | 781 |
| 119958 | Intron | 748 | 22293 | cactgataggaagtgtgtcc | 54 | 782 |
| 119959 | Intron | 748 | 22835 | ctcagttgctgtaaacacag | 57 | 783 |
| 119960 | Intron | 748 | 22883 | ccacagcgctctgagcactc | 73 | 784 |
| 119961 | Intron | 748 | 23125 | gtcctgaagtatcctgacct | 72 | 785 |
| 119962 | Intron | 748 | 23239 | gaaataaactagccagaggg | 26 | 786 |
| 119963 | Coding | 748 | 24169 | tttcttcctgactttcagaa | 33 | 787 |
| 119964 | Coding | 748 | 24201 | ttgggcgagatgtctggcaa | 55 | 788 |
| 119965 | Coding | 748 | 24208 | cgcctatttgggcgagatgt | 51 | 789 |
| 119966 | Coding | 748 | 24264 | gaactgtgcggctagctgtc | 62 | 790 |
| 119967 | Intron | 748 | 24515 | cgccacaagagaagactgag | 54 | 791 |
| 119968 | Intron | 748 | 24877 | aatgtgtgtgtctttgacag | 53 | 792 |
| 119969 | Intron | 748 | 25363 | ctacatgttatcttcccttc | 37 | 793 |
| 119970 | Coding | 748 | 25705 | agggctttggccaggcagtt | 43 | 794 |
| 119971 | Coding | 748 | 25776 | acagcattgtcattatcagc | 67 | 795 |
| 119972 | Coding | 748 | 25814 | gagcaaagatggtgcgtgac | 54 | 796 |
| 119973 | Coding | 748 | 25830 | tgtggaagacatcacggagc | 78 | 797 |
| 119974 | Coding | 748 | 25838 | gacagtcgtgtggaagacat | 48 | 798 |
| 119975 | Coding | 748 | 25858 | aggttctggttaataaagtt | 34 | 799 |
| 119976 | Intron | 748 | 26838 | gtcattttccagcagtctca | 77 | 800 |
| 119977 | Coding | 748 | 27236 | gcgggctcctcagtccatct | 74 | 801 |
| 119978 | 3′ UTR | 748 | 27315 | gttctctgggacctgtcttc | 44 | 802 |
| 119979 | 3′ UTR | 748 | 27474 | tcattcccaagtgggaaccc | 49 | 803 |
| 119980 | 3′ UTR | 748 | 27577 | cagaagcccacctacatggt | 44 | 804 |
| 119981 | 3′ UTR | 748 | 27608 | atgcacctctcctaatgctg | 58 | 805 |
| 119982 | 3′ UTR | 748 | 27612 | gccgatgcacctctcctaat | 67 | 806 |
| 119983 | 3′ UTR | 748 | 27657 | gagcacttcagtgtccacta | 56 | 807 |
| 119984 | 3′ UTR | 748 | 27700 | agatcagccattcggctttt | 58 | 808 |
| 119985 | 3′ UTR | 748 | 27711 | cccatggtttgagatcagcc | 75 | 809 |
| 119986 | 3′ UTR | 748 | 27788 | gatagaaatcttgagataat | 11 | 810 |
| 119987 | 3′ UTR | 748 | 27834 | caccacacagataagtcgtg | 65 | 811 |
| 119988 | 3′ UTR | 748 | 27842 | gtaactgacaccacacagat | 60 | 812 |
| 119989 | 3′ UTR | 748 | 27851 | agcctgagtgtaactgacac | 54 | 813 |
| 119990 | 3′ UTR | 748 | 27859 | gtagcaagagcctgagtgta | 48 | 814 |
| 119991 | 3′ UTR | 748 | 27868 | ttgcattccgtagcaagagc | 51 | 815 |
| 119992 | 3′ UTR | 748 | 27934 | agtgacctgctgctgtttta | 37 | 816 |
| 119993 | 3′ UTR | 748 | 28042 | cttttgatatggaatcttct | 50 | 817 |
| 119994 | 3′ UTR | 748 | 28067 | aatacagaagcggagggaac | 32 | 818 |
| 119995 | 3′ UTR | 748 | 28083 | gaggccttgtctctgaaata | 78 | 819 |
| 119996 | 3′ UTR | 748 | 28107 | cgtaacaacgcttgaggata | 63 | 820 |
| 119997 | 3′ UTR | 748 | 28145 | gctgacgatcccagctttaa | 38 | 821 |
| 119998 | 3′ UTR | 748 | 28167 | cttgcaggctgtggcggctc | 65 | 822 |
| 119999 | 3′ UTR | 748 | 28170 | atacttgcaggctgtggcgg | 52 | 823 |
| 120000 | 3′ UTR | 748 | 28192 | ctgggatgagttcagaacta | 73 | 824 |
| 120001 | 3′ UTR | 748 | 28332 | cacatatttttagaacagaa | 38 | 825 |
| 120002 | 3′ UTR | 748 | 28378 | gagccttttattttgaagaa | 60 | 826 |

As shown in Table 32, SEQ ID NOs 749, 750, 751, 752, 753, 754, 755, 756, 757, 758, 759, 760, 761, 762, 763, 764,765, 766, 767, 768, 769, 770, 771, 772, 773, 775, 776, 777, 778, 780, 781, 782, 783, 784, 785, 787, 788, 789, 790, 791, 792, 793, 794, 795, 796, 797, 798, 799, 800, 801, 802, 803, 804, 805, 806, 807, 808, 809, 811, 812, 813, 814, 815, 816, 817, 818, 819, 820, 821, 822, 823, 824, 825 and 826 demonstrated at least 30% inhibition of mouse BH3 Interacting domain death agonist expression in this experiment and are therefore preferred. The target sites to which these preferred sequences are complementary are herein referred to as “preferred target segments” and are therefore preferred sites for targeting by compounds of the present invention.

### Example 63

Target Validation—Effect of BH3 Interacting Death Domain Antisense Oligonucleotides in a Fas Cross-Linking Antibody Murine Model for Hepatitis

Injection of agonistic Fas-specific antibody into mice can induce massive hepatocyte apoptosis and liver hemorrhage, and death from acute hepatic failure (Ogasawara, J., et al., Nature, 1993, 364, 806-809). Apoptosis-mediated aberrant cell death has been shown to play an important role in a number of human diseases. For example, in hepatitis, Fas and Fas ligand up-regulated expression are correlated with liver damage and apoptosis. It is thought that apoptosis in the livers of patients with fulminant hepatitis, acute and chronic viral hepatitis or autoimmune hepatitis, as well as chemical or drug induced liver intoxication may result from Fas activation on hepatocytes. There are various indices of liver damage and/or apoptosis that are commonly used. These include measurement of the liver enzymes, AST and ALT.

Eight to ten week-old female Balb/c mice were intraperitoneally injected with oligonucleotide 119935 (SEQ ID NO. 759) at 24 mg/kg, daily for 4 days or with saline at a dose of 7 ug. Four hours after the last dose, 7.5 ug of mouse Fas antibody (Pharmingen, San Diego, Calif.) was injected into the mice. Mortality of the mice was measured for 48 hours following antibody treatment. Results are shown in Table 33. Mortality is expressed as percent survival.

TABLE 33
Protective Effects of BH3 Interacting Death Domain
Antisense Chimeric (deoxy gapped) Phosphorothioate
Oligonucleotides in Fas Antibody Cross-linking Induced
Death in Balb/c Mice

| | SEQ ID | Percent Survival | | | | | |
|---|---|---|---|---|---|---|---|
| ISIS # | NO: | 4 Hr | 6 Hr | 8 Hr | 12 Hr | 24 Hr | 48 |
| Saline | — | 100 | 90 | 20 | 0 | 0 | 0 |
| 119935 | 107 | 100 | 100 | 100 | 100 | 100 | 100 |

Oligonucleotide 119935 (SEQ ID NO. 759) completely protected the Fas-antibody treated mice from death. Injection with saline alone did not confer any protective effect.

After challenge with a higher dose of Fas antibody (15 ug), protection from fulminant death by the BH3 interacting death domain antisense oligonucleotides was lost with survival rates dropping to 1 percent at 5 hours post-treatment. An increase in antisense oligonucleotide dosage to 50 mg/kg given 6 times every 3 days also failed to produce protection from fulminant death at the higher dose of Fas antibody.

The BH3 interacting death domain antisense oligonucleotide was also shown to override sensitization to Fas antibody-induced death by Bcl-xL antisense oligonucleotide in the same model.

In these experiments, 8-10 week-old female Balb/c mice were intraperitoneally injected with oligonucleotides ISIS 16009 (SEQ ID NO. 827, targeting murine Bcl-xL) alone or in combination with ISIS 119935 (SEQ ID NO. 756) at 50 mg/kg, 6 times a day for two days or with saline at a dose of 7 ug. ISIS 16009 is a chimeric oligonucleotide (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. Cytidine residues in the “wings” are 5-methylcytidines. Four hours after the last dose, 7 ug of mouse Fas antibody (Pharmingen, San Diego, Calif.) was injected into the mice. Mortality of the mice was measured for 48 hours following antibody treatment. Results are shown in Table 34. Mortality is expressed as percent survival. N.D. indicates no data for these timepoints.

TABLE 34
Protective Effects of BH3 Interacting Death
Domain Antisense Oligonucleotides in
Fas Antibody Cross-linking Induced Death in
Balb/c Mice sensitized by Bcl-xL
antisense oligonucleotide treatment.

| | | Percent Survival | | | | | |
|---|---|---|---|---|---|---|---|
| ISIS # | SEQ ID | 4 Hr | 6 Hr | 8 Hr | 12 Hr | 24 Hr | 48 Hr |
| saline | — | 90 | 60 | 20 | 0 | 0 | 0 |
| 16009 | 175 | 90 | 30 | 20 | 10 | N.D. | N.D. |
| 119935 + | 107 | 100 | 100 | 100 | 100 | 100 | 100 |
| 16009 | | | | | | | |

Western blot analysis of Bcl-xL and BH3 interacting death domain proteins revealed that the expression levels of both targets was reduced to greater than 90%.

### Example 64

Target Validation—Effect of BH3 Interacting Death Domain Antisense Oligonucleotides in an Endotoxin and D(+)-Galactosamine-induced Murine Model of Fulminant Hepatitis and Liver Injury

The lipopolysaccharide/D-galactosamine or LPS/GalN model is a well known experimental model of toxin-induced hepatitis. Injection of the endotoxin, lipopolysaccharide (LPS), induces septic shock death in the mouse, though with LPS alone, the mouse liver does not sustain major damage. Injection of D-Galactosamine (GalN), while metabolized in liver causing depletion of UTP, is not lethal to mice. It does, however, sensitize animals to TNF-α or LPS-induced endotoxic shock by over 1,000 fold. In the presence of GalN, LPS induces apoptotic cell death in liver, thymus, spleen, lymph nodes and the kidney and results in fulminant death in animals. The liver injury is known to be transferable via the serum, suggesting a mechanism of action under TNF-α control. Further support for this mechanism is provided by the finding that TNFR1 knockout mice are resistant to LPS/GalN-induced liver injury and death.

Eight-week-old female Balb/c mice were used to assess the activity of BH3 interacting death domain antisense oligonucleotides in the endotoxin and D(+)-Galactosamine-induced murine model of fulminant hepatitis and liver injury. Mice were intraperitoneally pretreated with 24 mg/kg of ISIS 119935 (SEQ ID NO. 759) four times a day for 2 days. Control mice were injected with saline. One day after the last dose of oligonucleotide, mice were injected intraperitoneally with 5 ng LPS (DIFCO laboratories) and 20 mg D-Galactosamine (Sigma) per animal in saline. At time intervals of 5.5, 7.5, 9.5, 21.5, 30, 45 and 53 hours after the final dose, animals were monitored for survival rates. Results are shown in Table 35.

TABLE 35
Protective Effects of BH3 Interacting Death
Domain Antisense Oligonucleotides in
Endotoxin-Mediated Death in Balb/c Mice

| | | Percent Survival | | | | | | |
|---|---|---|---|---|---|---|---|---|
| | | 5.5 | 7.5 | | | | | |
| ISIS # | SEQ ID | Hr | Hr | 9.5 Hr | 21.5 Hr | 30 Hr | 45 Hr | 53 Hr |
| Saline | — | 100 | 100 | 20 | 20 | 10 | 10 | 10 |
| 119935 | 107 | 100 | 100 | 100 | 100 | 100 | 100 | 100 |

BH3 interacting death domain antisense oligonucleotides were also shown to override sensitization to endotoxin-mediated death by Bcl-xL antisense oligonucleotides in the same model. In these experiments, 8-10 week old female Balb/c mice were intraperitoneally pretreated with 24 mg/kg of ISIS 16009 (SEQ ID NO. 827) alone or in combination with ISIS 119935 (SEQ ID NO. 756) four times a day for 2 days. Control mice were injected with saline. One day after the last dose of oligonucleotide, mice were injected intraperitoneally with 5 ng LPS (DIFCO laboratories) and 20 mg D-Galactosamine (Sigma) per animal in saline. At time intervals of 6, 6.5, 7, 7.5, 9, 9.5 and 22 hours after the final dose, animals were monitored for survival rates. Results are shown in Table 36. Mortality is expressed as percent survival.

TABLE 36
Protective Effects of BH3 Interacting Death
Domain Antisense Oligonucleotides in
Endotoxin-Mediated Death in Balb/c Mice
sensitized by Bcl-xL antisense
oligonucleotide treatment.
Percent Survival

| | SEQ | | | | | | 9.5 | |
|---|---|---|---|---|---|---|---|---|
| ISIS # | ID | 6 Hr | 6.5 Hr | 7 Hr | 7.5 Hr | 9 Hr | Hr | 22 Hr |
| Saline | — | 100 | 100 | 100 | 100 | 70 | 20 | 10 |
| 16009 | 175 | 100 | 80 | 30 | 0 | 0 | 0 | 0 |
| 119935 + | 107 | 100 | 100 | 100 | 100 | 100 | 100 | 100 |
| 16009 | | | | | | | | |

### Example 65

Antisense Inhibition of PTEN mRNA Expression by Chimeric Phosphorothioate Oligonucleotides Having 2′-Moe Wings and a Deoxy Gap

PTEN is a dual-specificity protein phosphatase recently implicated as a phosphoinositide phosphatase in the insulin-signaling pathway. The pharmacological modulation of PTEN activity and/or expression may be an appropriate point for therapeutic intervention in metabolic disorders such as diabetes which arise from degregulated insulin signaling.

Oligonucleotides targeting human PTEN were designed as described in Example 2, synthesized as described in Examples 3-7, analyzed as described in Example 8 and assayed by RT-PCR as described in Example 12.

Probes and primers to human PTEN were designed to hybridize to a human PTEN sequence, using published sequence information (GenBank accession number U92436.1, incorporated herein as SEQ ID NO: 828). For human PTEN the PCR primers were: forward primer: AATGGCTAAGTGAAGATGACAATCAT (SEQ ID NO: 829) reverse primer: TGCACATATCATTACACCAGTTCGT (SEQ ID NO: 830) and the PCR probe was: FAM-TTGCAGCAATTCACTGTAAAGCTGGAAAGG-TAMRA (SEQ ID NO: 831) where FAM (PE-Applied Biosystems, Foster City, Calif.) is the fluorescent reporter dye) and TAMRA (PE-Applied Biosystems, Foster City, Calif.) is the quencher dye.

The series of oligonucleotides was designed to target different regions of the human PTEN RNA, using published sequences (GenBank accession number U92436.1, incorporated herein as SEQ ID NO: 828). The oligonucleotides are shown in Table 37. “Target site” indicates the first (5=-most) nucleotide number on the particular target sequence to which the oligonucleotide binds. All compounds in Table 37 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines. The compounds were analyzed for their effect on human PTEN mRNA levels by quantitative real-time PCR as described in other examples herein. Data are averages from three experiments in which T-24 cells were cultured as described in Section 15 (15. Cell Lines for Assaying Oligonucleotide Activity) treated with oligonucleotides 29574-29613 (SEQ ID NOs: 832-867) according to the protocol outlined in Example 34. If present, “N.D.” indicates “no data”.

TABLE 37

Inhibition of Human PTEN mRNA levels
by chimeric phosphorothioate oligonucleotides
having 2′-MOE wings and a deoxy gap

| | | | | | | |
|---|---|---|---|---|---|---|
| | | TARGET | | | | |
| ISIS # | REGION | SEQ ID NO | TARGET SITE | SEQUENCE | % INHIB | SEQ ID NO. |

| | | | | | | |
|---|---|---|---|---|---|---|
| 29574 | 5′ UTR | 828 | 19 | cgagaggcggacgggacc | 29 | 832 |
| 29575 | 5′ UTR | 828 | 57 | cgggcgcctcggaagacc | 27 | 833 |
| 29576 | 5′ UTR | 828 | 197 | tggctgcagcttccgaga | 67 | 834 |
| 29577 | 5′ UTR | 828 | 314 | cccgcggctgctcacagg | 79 | 835 |
| 29578 | 5′ UTR | 828 | 421 | caggagaagccgaggaag | 51 | 836 |
| 29579 | 5′ UTR | 828 | 494 | gggaggtgccgccgccgc | 71 | 837 |
| 29581 | 5′ UTR | 828 | 671 | ccgggtccctggatgtgc | 88 | 838 |
| 29582 | 5′ UTR | 828 | 757 | cctccgaacggctgcctc | 66 | 839 |
| 29583 | 5′ UTR | 828 | 817 | tctcctcagcagccagag | 74 | 840 |
| 29584 | 5′ UTR | 828 | 891 | cgcttggctctggaccgc | 81 | 841 |
| 29585 | 5′ UTR | 828 | 952 | tcttctgcaggatggaaa | 63 | 842 |
| 29587 | Coding | 828 | 1106 | ggataaatataggtcaag | 50 | 843 |
| 29588 | Coding | 828 | 1169 | tcaatattgttcctgtat | 41 | 844 |
| 29589 | Coding | 828 | 1262 | ttaaatttggcggtgtca | 74 | 845 |
| 29590 | Coding | 828 | 1342 | caagatcttcacaaaagg | 64 | 846 |
| 29591 | Coding | 828 | 1418 | attacaccagttcgtccc | 55 | 847 |
| 29592 | Coding | 828 | 1504 | tgtctctggtccttactt | 64 | 848 |
| 29593 | Coding | 828 | 1541 | acatagcgcctctgactg | 73 | 849 |
| 29595 | Coding | 828 | 1694 | gaatatatcttcaccttt | 30 | 850 |
| 29596 | Coding | 828 | 1792 | ggaagaactctactttga | 61 | 851 |
| 29597 | Coding | 828 | 1855 | tgaagaatgtatttaccc | 31 | 852 |
| 29599 | Coding | 828 | 2020 | ggttggctttgtctttat | 56 | 853 |
| 29600 | Coding | 828 | 2098 | tgctagcctctggatttg | 80 | 854 |
| 29601 | Coding | 828 | 2180 | tctggatcagagtcagtg | 65 | 855 |
| 29602 | 3′ UTR | 828 | 2268 | tattttcatggtgtttta | 41 | 856 |
| 29603 | 3′ UTR | 828 | 2347 | tgttcctataactggtaa | 63 | 857 |
| 29604 | 3′ UTR | 828 | 2403 | gtgtcaaaaccctgtgga | 39 | 858 |
| 29605 | 3′ UTR | 828 | 2523 | actggaataaaacgggaa | 6 | 859 |
| 29606 | 3′ UTR | 828 | 2598 | acttcagttggtgacaga | 40 | 860 |
| 29607 | 3′ UTR | 828 | 2703 | tagcaaaacctttcggaa | 22 | 861 |
| 29608 | 3′ UTR | 828 | 2765 | aattatttcctttctgag | 23 | 862 |
| 29609 | 3′ UTR | 828 | 2806 | taaatagctggagatggt | 27 | 863 |
| 29610 | 3′ UTR | 828 | 2844 | cagattaataactgtagc | 42 | 864 |
| 29611 | 3′ UTR | 828 | 2950 | ccccaatacagattcact | 10 | 865 |
| 29612 | 3′ UTR | 828 | 3037 | attgttgctgtgtttctt | 61 | 866 |
| 29613 | 3′ UTR | 828 | 3088 | tgtttcaagcccattctt | 55 | 867 |

As shown in Table 37, SEQ ID NOs: 834, 835, 836, 837, 838, 839, 840, 841, 842, 843, 844, 845, 846, 847, 849, 850, 851, 852, 853, 854, 855, 856, 857, 858, 860, 864, 866 and 867 demonstrated at least 30% inhibition of PTEN expression in this experiment and are therefore preferred. The target sites to which these preferred sequences are complementary are herein referred to as “preferred target segments” and are therefore preferred sites for targeting by compounds of the present invention.

### Example 66

Target Validation—Inhibition of PTEN Expression-Dose Response in Human, Mouse and Rat Hepatocytes

In accordance with the present invention, two additional oligonucleotides targeted to human PTEN were designed and synthesized. ISIS 116847 (CTGCTAGCCTCTGGATTTGA, SEQ ID NO: 868) and ISIS 116845 (ACATAGCGCCTCTGACTGGG, SEQ ID NO: 869). The mismatch control for ISIS 116847 is ISIS 116848 (CTTCTGGCATCCGGTTTAGA, SEQ ID NO: 870), a six base pair mismatch of ISIS 116847, while the control used is the mixed sequence 20-mer negative oligonucleotide control ISIS 29848 (SEQ ID NO: 461). Both ISIS 116847 and ISIS 116845 target the coding region of Genbank accession no. U92436.1 (SEQ ID NO: 828), with ISIS 116847 starting at position 2097 and ISIS 116845 starting at position 1539.

These oligonucleotide sequences also target the mouse PTEN sequence with perfect complementarity, with ISIS 116845 targeting nucleotides 1453-1472 and ISIS 116847 targeting nucleotides 2012-2031 of GenBank accession number U92437 (SEQ ID NO: 871) (locus name MMU92437; Steck et al., Nature Genet., 1997, 15,356-362). For mouse PTEN the PCR primers were: forward primer:

ATGACAATCATGTTGCAGCAATTC (SEQ ID NO: 872) reverse primer:

CGATGCAATAAATATGCACAAATCA (SEQ ID NO: 873) and the PCR probe was:

FAM-CTGTAAAGCTGGAAAGGGACGGACTGGT-TAMRA (SEQ ID NO: 874) where FAM (PE-Applied Biosystems, Foster City, Calif.) is the fluorescent reporter dye) and TAMRA (PE-Applied Biosystems, Foster City, Calif.) is the quencher dye.

Similarly, these oligonucleotide sequences target the rat PTEN sequence with perfect complementarity, with ISIS 116845 targeting nucleotides 505-524 and ISIS 116847 targeting nucleotides 1063-1082 of GenBank accession number AF017185 (SEQ ID NO: 875). The mouse PTEN primers and probe listed above target the rat PTEN sequence with perfect complementarity and were used to determine the PTEN expression dose response in rat hepatocytes.

All compounds of this example are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotides. All cytidine residues are 5-methylcytidines.

Data were obtained by real-time quantitative PCR as described in other examples herein and are averaged from two experiments.

In a dose-response experiment, human hepatocyte cells (HEPG2; American Type Culture Collection, Manassas, Va.), mouse primary hepatocytes, and rat primary hepatocytes were treated with ISIS 116847 and its mismatch control, ISIS 116848 at doses of 10, 50, 100 and 200 nM oligonucleotide normalized to untreated controls. In all three species, the dose response was linear compared to vehicle treated controls.

In human HEPG2 cells, ISIS 116847 reduced PTEN mRNA levels to 55% of control at a dose of 10 nM, and to 5% of control at 200 nM while the PTEN mRNA levels in cells treated with the mismatch control oligonucleotide remained at greater than 90% of control across the entire dosing range.

In mouse primary hepatocytes the trend was the same with ISIS 116847 reducing PTEN mRNA levels to 85% of control at the lower dose of 10 nM, and down to 2% of control at the 200 nM dose. Again, the control oligonucleotide, ISIS 116848 failed to reduce PTEN mRNA levels and remained at or above 85% of control.

In rat primary hepatocytes, ISIS 116847 reduced PTEN mRNA levels to 55% of control at the lower dose of 10 nM and to 10% of control at the highest dose of 200 nM. PTEN mRNA levels in cells treated with the control oligonucleotide, ISIS 116848, remained at or above 95% of control across the entire dosing range.

### Example 67

Effects of Inhibition of PTEN on mRNA Expression in Fat and Liver

In the following examples, modulators of PTEN were tested in db/db mice (Jackson Laboratories, Bar Harbor, Me.). These mice are hyperglycemic, obese, hyperlipidemic, and insulin resistant, and are used as a standard animal model of diabetes.

Male db/db mice (age 14 weeks) were divided into matched groups (n=5) with the same average blood glucose levels and treated once a week for 4 weeks with ISIS 116847 at 10, 25, 50 or 100 mg/kg or ISIS 116845 at 50 mg/kg. Wild type mice were similarly treated. Controls included saline, ISIS 116848 (a mismatch control), ISIS 29848 (the mixed sequence 20-mer negative oligonucleotide control, SEQ ID NO: 461) and the sense control of ISIS 116847. As a comparison db/db mice were also treated with troglitazone, an oral antihyperglycemic agent which is used in the treatment of type II diabetes. It acts primarily to decrease insulin resistance, improve sensitivity to insulin in muscle and adipose tissue and inhibit hepatic gluconeogenesis. At day 28 mice were sacrificed and PTEN mRNA levels were measured.

Treatment of db/db mice with ISIS 116847 showed a dose-dependent decrease in PTEN mRNA levels in the liver to 10% of control at 50 mg/kg. ISIS 116845 showed a reduction in PTEN mRNA levels to 22% of control at a dose of 50 mg/kg.

In wild-type mice a level of 5% of control PTEN mRNA required a dose of 100 mg/kg of ISIS 116847. Neither troglitazone nor any of the controls had an effect on PTEN mRNA levels over saline control.

Similar results were seen in fat. Treatment of db/db mice with ISIS 116847 showed a dose-dependent decrease in PTEN mRNA levels in fat to 20% of control at 50 mg/kg. ISIS 116845 showed a reduction in PTEN mRNA levels to 35% of control at a dose of 50 mg/kg.

In wild-type mice a level of 18% of control required a dose of 100 mg/kg of ISIS 116847. Neither troglitazone nor any of the controls had an effect on PTEN mRNA levels over saline control.

In another experiment, male db/db mice (age 14 weeks) were divided into matched groups (n=5) with the same average blood glucose levels and treated intraperitoneally with saline or ISIS 116847 every other day (q2d) or twice a week (q4d) at a dose of 20 mg/kg. The control for both protocols was the mismatch control, ISIS 116848. Mice were exsanguinated on day 14 and PTEN mRNA levels in liver and fat were measured.

ISIS 116847 successfully reduced PTEN mRNA levels in both liver and fat of db/db mice at both the q2d and q4d dosing schedules in a dose-dependent manner, whereas the mismatch control and saline treated animals showed no reduction in PTEN mRNA.

There was no reduction of PTEN mRNA in skeletal muscle with any of the oligonucleotides used. This lack of an effect in muscle indicates that reduction of expression of PTEN in liver and fat alone is sufficient to lower hyperglycemia.

### Example 68

Target Validation—Effects of inhibition of PTEN on mRNA Expression in Kidney

Male db/db and wild-type mice were treated once a week for 4 weeks with ISIS 116847 at 10, 25, 50 or 100 mg/kg or ISIS 116845 at 50 mg/kg. Controls included saline, ISIS 116848 (a mismatch control), ISIS 29848 (the mixed sequence 20-mer negative oligonucleotide control, SEQ ID NO: 461) and the sense control of ISIS 116847. As a comparison db/db mice were also treated with troglitazone. At day 28 mice were sacrificed and PTEN mRNA levels were measured.

Treatment with ISIS 116847 showed a dose-dependent decrease in PTEN mRNA levels in kidney, being reduced to 70% of control at a dose of 50 mg/kg. ISIS 116845 reduced PTEN mRNA levels to 85% of control at the same dose.

In wild-type mice a level of 75% of control required a dose of 100 mg/kg of ISIS 116847. Neither troglitazone nor any of the controls had an effect on PTEN mRNA levels over saline control.

### Example 69

Target Validation—Effects of Inhibition of PTEN (ISIS 116847) on PTEN Protein Levels in Liver Extracts as a Function of Time and Dose

Male db/db and wild-type mice (age 14 weeks) were treated once a week for 4 weeks with saline, a control oligonucleotide, ISIS 29848 (the mixed sequence 20-mer negative oligonucleotide control, SEQ ID NO: 461) (50 mg/kg) or ISIS 116847 at 10, 25 or 50 mg/kg. Wild-type mice were treated with saline or ISIS 116847 at 100 mg/kg. Mice were sacrificed at day 28 and PTEN protein levels were measured by Western blotting as described in other examples herein.

In the db/db mice, treatment with ISIS 116847 caused a dose-dependent decrease in PTEN protein levels compared to saline controls or mismatch treated animals.

Protein levels in wild-type mice treated at 100 mg/kg were comparably reduced to the levels seen in db/db mice treated at the 50 mg/kg dose. There was no significant difference in the relative levels of PTEN protein in control lean versus db/db mice.

### Example 70

Target Validation—Effects of Inhibition of PTEN (ISIS 116847) on PTEN Protein Levels in Fat and Kidney as a Function of Time and Dose

Male db/db and wild-type mice (age 14 weeks) were treated once a week for 4 weeks with saline or ISIS 116847 at 50 mg/kg by intraperitoneal injection. Mice were sacrificed at day 28 and PTEN protein levels were measured by Western blotting described in other examples herein.

PTEN levels in fat were reduced in both db/db and wild-type mice by the PTEN oligomeric compounds as compared to control, and slight reduction of PTEN levels was seen in the kidney after treatment with oligomeric compounds.

### Example 71

Target Validation—Effects of Inhibition of PTEN on Blood Glucose Levels

Male db/db and wild type mice (age 14 weeks) were divided into matched groups (n=5) with the same average blood glucose levels and treated by intraperitoneal injection with saline or ISIS 116847 every other day (q2d) or twice a week (q4d) at a dose of 20 mg/kg. The control for both protocols was the mismatch control, ISIS 116848. Blood glucose levels were measured on day 7 and day 14.

By day 14 in db/db mice, blood glucose levels were reduced for both treatment schedules; from starting levels of 330 mg/dL to 175 mg/dL (q2d) and 170 mg/dL (q4d) which are levels within the range considered normal for wild-type mice. The mismatch control levels remained at 310 mg/dL throughout the study.

In wild-type mice, blood glucose levels remained constant throughout the study for all treatment groups (average 115 mg/dL).

In a similar experiment, male db/db and wild-type mice were treated once a week for 4 weeks with ISIS 116847 or ISIS 116845 at 50 mg/kg. Controls included saline, ISIS 116848 (a mismatch control) and ISIS 29848 (the mixed sequence 20-mer negative oligonucleotide control, SEQ ID NO: 461). At day 28 mice were sacrificed and serum glucose levels were measured.

In db/db mice, treatment with either ISIS 116847 or ISIS 116845 reduced serum glucose levels relative to saline control (480 mg/dL) to 240 and 280 mg/dL, respectively. This reduction was statistically significant (p<0.005). Neither the mismatch nor universal control had any effect on serum glucose levels. In wild-type animals, ISIS 116847 failed to reduce serum glucose levels from that of control (200 mg/dL).

### Example 72

Target Validation—Effects of Inhibition of PTEN (ISIS 116847) on Blood Glucose Levels of db/db Mice as a Function of Time and Dose

Male db/db mice (age 14 weeks) were treated once a week for 4 weeks with saline or ISIS 116847 at 10, 25 or 50 mg/kg intraperitoneally. Blood glucose levels were measured on day 7, 14, 21 and 28.

At the beginning of the study, all groups had blood glucose levels of 275 mg/dL which rose in the saline treated animals and those treated at the low dose of ISIS 116847 to 350 mg/dL and 320 mg/dL, respectively by day four. At the end of the first week, all three dosing schedules showed a reduction in blood glucose and continued to show linear dose response decreases throughout the study. At day 28, blood glucose levels in animals treated with oligomeric compounds were 275 mg/dL (10 mg/kg dose), 175 mg/dL (25 mg/kg dose) and 120 mg/dL (50 mg/kg dose) while saline treated levels remained at 350 mg/dL. The average glucose levels for oligonucleotide treated mice at the end of the four week study was 194 mg/dL as compared to 418 mg/dL for saline treated controls (p<0.0001).

### Example 73

Target Validation—Effects of Inhibition of PTEN (ISIS 116847) on Blood Glucose Levels of db/db Mice-Insulin Tolerance Test

Male db/db mice (age 14 weeks) were treated once with saline or ISIS 116847 50 mg/kg by intraperitoneal injection. The insulin tolerance test was performed after a four hour fast followed by an intraperitoneal injection of 1 U/kg human insulin (Lilly). On day 21, blood was withdrawn from the tail at 0, 30, 60 and 90 minutes and blood glucose levels were measured as a percentage of blood glucose at time zero. Statistical analysis was performed using ANOVA repeated measures followed by Bonferroni Dunn analysis, p<0.05.

Treatment with ISIS 116847 on day 21 resulted in a significant dose-dependent decrease in blood glucose (p<0.006) at the 90 minute post-treatment time point to 45% of control (55% decrease). Saline treatment resulted in a 30% reduction. These studies indicate that the PTEN oligonucleotide is capable of increasing sensitivity to insulin (decreasing insulin resistance) and that treatment does not cause hypoglycemia. Glucose levels in PTEN treated mice (both db/db and wild-type) fasted for 16 hours remained normal.

### Example 74

Target Validation—Effects of Inhibition of PTEN on Serum Triglyceride and Cholesterol Concentration

Male db/db and wild-type mice were treated once a week for 4 weeks with ISIS 116847 at 10, 25, 50 or 100 mg/kg or ISIS 116845 at 50 mg/kg. Controls included saline, ISIS 116848 (a mismatch control), ISIS 29848 (the mixed sequence 20-mer negative oligonucleotide control, SEQ ID NO: 461) and the sense control of ISIS 116847. As a comparison db/db mice were also treated with troglitazone. At day 28 mice were sacrificed and triglyceride and cholesterol levels were measured.

Treatment of db/db mice with ISIS 116847 resulted in a dose-dependent reduction of both triglycerides and cholesterol compared to saline controls (a reduction from 200 mg/dL to 100 mg/dL for triglycerides and from 130 mg/dL to 98 mg/dL for cholesterol). Treatment of db/db mice with ISIS 116845 at a dose of 50 mg/kg resulted in a decrease in both triglycerides and cholesterol levels to 130 mg/dL and 75 mg/dL, respectively. Troglitazone treatment of db/db mice reduced both triglyceride and cholesterol levels to 85 mg/dL each.

Wild-type animals did not respond to treatment with ISIS 116847 at a dose of 100 mg/kg as both triglyceride and cholesterol levels remained similar to control saline treated animals (between 85 and 105 mg/dL). The reductions seen in cholesterol and triglycerides were statistically significant at p<0.005.

### Example 75

Target Validation—Effects of Inhibition of PTEN on Body Weight

Male db/db and wild-type mice were treated once a week for 4 weeks with ISIS 116847 at 10, 25, 50 or 100 mg/kg or ISIS 116845 at 50 mg/kg. Controls included saline, ISIS 116848 (a mismatch control), ISIS 29848 (the mixed sequence 20-mer negative oligonucleotide control, SEQ ID NO: 461) and the sense control of ISIS 116847. As a comparison db/db mice were also treated with troglitazone. At day 28 mice were sacrificed and final body weights were measured.

Treatment with ISIS 116847 resulted in a dose-dependent increase in body weight over the dose range with animals treated at the high dose gaining an average of 8.7 grams while saline treated controls gained 2.8 grams. Animals treated with the mismatch or universal control oligonucleotide gained between 2.5 and 3.5 grams of body weight and troglitazone treated animals gained 5.0 grams.

Wild-type animals treated with 100 mg/kg of ISIS 116847 gained 2.0 grams of body weight compared to a gain of 1.3 grams for the wild-type saline or mismatch controls.

Weight gain in the PTEN oligomeric compound treated mice began to increase relative to that in saline or control groups at the same time that glucose levels began to drop.

### Example 76

Target Validation—Effects of Inhibition of PTEN on Liver Weight-Anterior Lobe

Male db/db and wild-type mice were treated once a week for 4 weeks with ISIS 116847 at 10, 25, 50 or 100 mg/kg or ISIS 116845 at 50 mg/kg. Controls included saline, ISIS 116848 (a mismatch control), ISIS 29848 (the mixed sequence 20-mer negative oligonucleotide control, SEQ ID NO: 461) and the sense control of ISIS 116847. As a comparison db/db mice were also treated with troglitazone. At day 28 mice were sacrificed and the weights of the anterior lobe of the liver were measured.

db/db animals treated at the high dose had liver weights of 1.2 grams while saline treated controls weighed 0.75 grams. db/db animals treated with ISIS 116845 at a dose of 50 mg/kg had comparable liver size to those treated with ISIS 116847 at a dose of 25 mg/kg (1.0 grams). Animals treated with the mismatch control, universal control or troglitazone had livers weighing an average of 1.0 gram.

Wild-type mouse livers treated with 100 mg/kg of ISIS 116847 weighed 0.7 grams compared to 0.5 grams for the wild-type saline treated controls.

BrdU (bromine deoxyuridine) staining of liver sections indicated that the increase in liver weight was not due to increased cell proliferation, and there was no increase in inflammatory infiltrates in the liver. Long-term studies show that the increases in liver weight are reversed.

### Example 77

Target Validation—Effects of Inhibition of PTEN (ISIS 116847) on PEPCK mRNA Expression in Liver of db/db Mice

PEPCK is the rate-limiting enzyme of gluconeogenesis and is expressed predominantly in liver where it acts in the gluconeogenic pathway (production of glucose from amino acids) and in kidney where it acts in the gluconeogenic pathway as well as being glyceroneogenic and ammoniagenic. In the liver, PEPCK is negatively regulated by insulin and has therefore been considered a potential contributing factor to hyperglycemia in diabetics (Sutherland et al., Philos. Trans. R. Soc. Lond. B. Biol. Sci., 1996, 351, 191-199).

Male db/db mice (age 14 weeks) with the same average blood glucose levels were divided into groups (n=5) and treated intraperitoneally with saline, ISIS 116847 or the mismatch control, ISIS 116848, every other day (q2d). Mice were exsanguinated on day 14 and PEPCK mRNA levels in liver were measured.

Mice treated with ISIS 116847 showed a reduction of PEPCK mRNA to 65% of saline treated controls. The mismatch control group remained at 98% of saline treated control.

### Example 78

Target Validation—Effects of Inhibition of PTEN (ISIS 116847) on Serum Insulin Levels of db/db Mice

Male db/db and wild type mice (age 14 weeks) were divided into matched groups (n=5) with the same average blood glucose levels and treated by intraperitoneal injection with saline or ISIS 116847 every other day (q2d) or twice a week (q4d) at a dose of 20 mg/kg. The control for both protocols was the mismatch control, ISIS 116848. Mice were exsanguinated on day 14 and serum insulin levels were measured.

On day 14 db/db mice treated on the q2d schedule had serum insulin levels of 7.8 ng/mL, compared to saline treated (9 ng/mL) and mismatch treated animals (12 ng/mL). In the q4d schedule there was a drop in the serum insulin levels of db/db mice treated with ISIS 116847 to 4 ng/mL while the mismatch control levels remained at 12 ng/mL. Wild-type mice had serum insulin levels of 1 ng/mL throughout the course of both treatment schedules.

### Example 79

Target Validation—Effects of Inhibition of PTEN on Liver Function-AST/ALT Levels

Male db/db and wild type mice (age 14 weeks) were divided into matched groups (n=5) with the same average blood glucose levels and treated by intraperitoneal injection with saline, troglitazone, or ISIS 116847 every other day (q2d) or twice a week (q4d) at a dose of 20 mg/kg. The control for both protocols was the mismatch control, ISIS 116848. Mice were exsanguinated on day 14 and liver enzyme levels were measured.

In the q2d treatment schedule there was an increase in ALT levels over saline treated animals from 125 IU/L (saline control) to 300 IU/L (both PTEN oligonucleotide, ISIS 116847, and mismatch control), whereas AST levels remained between 220 IU/L and 240 IU/L among the three treatment groups.

In the q4d treatment schedule, ALT levels increased from 125 IU/L (saline control) to 160 IU/L in animals treated with ISIS 116847 and 200 IU/L for mismatch control. AST levels decreased from saline control levels of 220 IU/L to 160 IU/L for ISIS 116847 treated animals, as well as in animals treated with the mismatch control (200 IU/L). As a comparison, AST and ALT levels were measured after treatment with troglitazone. Levels of both enzymes were found to be 260 IU/L.

In a similar experiment, male db/db and wild-type mice were treated once a week for 4 weeks with ISIS 116847 at 10, 25, 50 or 100 mg/kg or ISIS 116845 at 50 mg/kg. Controls included saline or ISIS 29848 (the mixed sequence 20-mer negative oligonucleotide control, SEQ ID NO: 461) As a comparison db/db mice were also treated with troglitazone. At day 28 mice were sacrificed and AST and ALT levels were measured.

Treatment of db/db mice with ISIS 116847 resulted in a dose-dependent increase in ALT levels over the dose range with animals treated at the high dose having ALT levels of 250 IU/L while AST levels remained constant at 165 IU/L. These levels represent an increase in ALT levels from saline treated controls of 110 IU/L and a decrease in AST levels from saline treated controls of 220 IU/L. db/db animals treated with ISIS 116845 at a dose of 50 mg/kg had comparable ALT and AST levels, 145 IU/L. Animals treated with the universal control had ALT and AST levels comparable to control levels and those treated with troglitazone showed an increase in ALT levels over control to 150 IU/L and a slight decrease in AST levels to 200 IU/L from control.

Wild-type mice treated with 100 mg/kg of ISIS 116847 had both increased ALT and AST levels (100 IU/L and 130 IU/L, respectively) compared to saline-treated control ALT and AST levels (50 IU/L and 95 IU/L, respectively).

Although ALT levels were slightly elevated in animals treated with PTEN oligomeric compounds, AST levels were reduced indicating that PTEN oligomeric compound effects on liver weight were not due to toxicity.

### Example 80

Design of Double Stranded Oligomeric Compounds Targeting PTEN

RNA interference (RNAi) and post-transcriptional gene silencing (PTGS) have become powerful and widely used tools for gene function analysis in invertebrates and plants (Fraser et al., Nature, 2000, 408, 325; Gönczy et al. Nature, 2000, 408, 331). Introduction of double-stranded RNA (dsRNA) into the cells of these organisms leads to the sequence-specific degradation of homologous gene transcripts.

A number of PCT applications have recently been published that relate to the RNAi phenomenon. These include: PCT publication WO 00/44895; PCT publication WO 00/49035; PCT publication WO 00/63364; PCT publication WO 01/36641; PCT publication WO 01/36646; PCT publication WO 99/32619; PCT publication WO 00/44914; PCT publication WO 01/29058; and PCT publication WO 01/75164.

U.S. Pat. Nos. 5,898,031 and 6,107,094, each of which is commonly owned with this application and each of which is herein incorporated by reference, describe certain oligonucleotides having RNA-like properties. When hybridized with RNA, these oligonucleotides serve as substrates for a dsRNase enzyme with resultant cleavage of the target RNA by the enzyme.

In accordance with the present invention, a series of 21 nucleotide oligomeric compounds, in this case duplex RNAs (also known as small interfering RNAs: siRNAs), were designed to target PTEN mRNA (Genbank accession no. U92436. 1; SEQ ID NO: 828). The nucleobase sequence of the antisense strand of the duplex is identical to the 18 nucleobase oligonucleotides in Table 37 with one additional complementary base on the 3′ end of the oligoribonucleotides followed by a two-nucleobase overhang of deoxythymidine (T), TT. The sequences of the antisense strands are listed in Table 38. The sense strand of the dsRNAs listed in Table 39 were designed and synthesized as the complement of the antisense strands and also contained the two-nucleobase overhang on the 3′ end making both strands of the dsRNA duplex complementary over the central 19 nucleobases and each having a two-base overhang on the 3′ end. For example, the dsRNA having ISIS 29574 (SEQ ID NO: 832) as the antisense strand is:

| | |
|---|---|
| cgagaggcggacgggaccgTT | ISIS 29574 |
| \|\|\|\|\|\|\|\|\|\|\|\|\|\|\|\|\|\|\| | |
| TTgctctccgcctgccctggc | Complement of ISIS 29574 |

Both strands of the dsRNAs were purchased from Dharmacon Research Inc. (Lafayette, Colo.), shipped lyophilized and annealed on-site using the manufacturer's protocol. Briefly, each RNA oligonucleotide was aliquoted and diluted to a concentration of 50 μM. Once diluted, 30 uL of each strand was combined with 1.5 μL of a 5× solution of annealing buffer. The final concentration of said buffer was 100 mM potassium acetate, 30 mM HEPES-KOH pH 7.4, and 2mM magnesium acetate. The final volume was 75 μL. This solution was incubated for 1 minute at 90° C. and then centrifuged for 15 seconds. The tube was allowed to sit for 1 hour at 37° C. at which time the dsRNA duplexes were used in experimentation. The final concentration of the dsRNA duplex was 20 μM. This solution can be stored frozen (−20° C.) and freeze-thawed up to 5 times.

### Example 81

Modulation of Human PTEN Expression by Double Stranded RNA (dsRNA)

In accordance with the present invention, a series of double stranded oligomeric compounds targeted to PTEN were evaluated for their ability to modulate PTEN expression in T-24 cells.

When cells reached 80% confluency, they were treated with dsRNA. For cells grown in 96-well plates, wells were washed once with 200 μL OPTI-MEM-1™ reduced-serum medium (Gibco BRL) and then treated with 130 μL of OPTI-MEM-1™ containing 12 μg/mL LIPOFECTIN™ (Gibco BRL) and the desired dsRNA at a final concentration of 200 nM. After 5 hours of treatment, the medium was replaced with fresh medium. Cells were harvested 16 hours after dsRNA or single-stranded oligonucleotide treatment, at which time RNA was isolated and target reduction measured by RT-PCR.

The sequences of the oligomeric compounds (antisense and sense to the PTEN target mRNA) of the dsRNAs are shown in Table 38 and 39, respectively. Prior to treatment of the T-24 cells, the dsRNA oligomers were generated by annealing the antisense and sense strands according to the method outlined in Example 80. Target sites are indicated by the first (5′ most) nucleotide number, as given in the sequence source reference (Genbank accession no. U92436.1), to which the antisense strand of the dsRNA oligonucleotide binds.

All compounds in Tables 38 and 39 are oligoribonucleotides, 21 nucleotides in length with the two nucleotides on the 3′ end being oligodeoxyribonucleotides, TT with phosphodiester backbones (internucleoside linkages) throughout. All oligoribonucleotides are depicted in the 5′→3′ direction.

Data were obtained by real-time quantitative PCR as described in other examples herein and are averaged from three experiments in which T-24 cells were cultured as described in Section 15 (15. Cell Lines for Assaying Oligonucleotide Activity) according treated with double stranded oligomeric compounds (composed of antisense strands SEQ ID NOs: 879-914 hybridized to sense strands: SEQ ID NOs: 915-950) targeting PTEN mRNA (SEQ ID NO: 828) according to the protocol outlined in Example 34. If present, “N.D.” indicates “no data”.

TABLE 38

Modulation of PTEN mRNA levels by dsRNA oligomers

| | | | | | | |
|---|---|---|---|---|---|---|
| dsRNA | | | | | | |
| CORRESPONDING | | TARGET | | SEQUENCE OF ANTISENSE | | |
| TO ISIS# | REGION | SEQ ID NO | TARGET SITE | STRAND OF dsRNA | % INHIB | SEQ ID NO. |

| | | | | | | |
|---|---|---|---|---|---|---|
| 29574 | 5′ UTR | 828 | 19 | cgagaggcggacgggaccgTT | 0 | 876 |
| 29575 | 5′ UTR | 828 | 57 | cgggcgcctcggaagaccgTT | 0 | 877 |
| 29576 | 5′ UTR | 828 | 197 | tggctgcagcttccgagagTT | 40 | 878 |
| 29577 | 5′ UTR | 828 | 314 | cccgcggctgctcacaggcTT | 25 | 879 |
| 29578 | 5′ UTR | 828 | 421 | caggagaagccgaggaagaTT | 64 | 880 |
| 29579 | 5′ UTR | 828 | 494 | gggaggtgccgccgccgccTT | 20 | 881 |
| 29581 | 5′ UTR | 828 | 671 | ccgggtccctggatgtgccTT | 35 | 882 |
| 29582 | 5′ UTR | 828 | 757 | cctccgaacggctgcctccTT | 59 | 883 |
| 29583 | 5′ UTR | 828 | 817 | tctcctcagcagccagaggTT | 50 | 884 |
| 29584 | 5′ UTR | 828 | 891 | cgcttggctctggaccgcaTT | 33 | 885 |
| 29585 | 5′ UTR | 828 | 952 | tcttctgcaggatggaaatTT | 27 | 886 |
| 29587 | Coding | 828 | 1106 | ggataaatataggtcaagtTT | 49 | 887 |
| 29588 | Coding | 828 | 1169 | tcaatattgttcctgtataTT | 50 | 888 |
| 29589 | Coding | 828 | 1262 | ttaaatttggcggtgtcatTT | 64 | 889 |
| 29590 | Coding | 828 | 1342 | caagatcttcacaaaagggTT | 75 | 890 |
| 29591 | Coding | 828 | 1418 | attacaccagttcgtccctTT | 77 | 891 |
| 29592 | Coding | 828 | 1504 | tgtctctggtccttacttcTT | 76 | 892 |
| 29593 | Coding | 828 | 1541 | acatagcgcctctgactggTT | 74 | 893 |
| 29595 | Coding | 828 | 1694 | gaatatatcttcacctttaTT | 10 | 894 |
| 29596 | Coding | 828 | 1792 | ggaagaactctactttgatTT | 29 | 895 |
| 29597 | Coding | 828 | 1855 | tgaagaatgtatttacccaTT | 72 | 896 |
| 29599 | Coding | 828 | 2020 | ggttggctttgtctttattTT | 0 | 897 |
| 29600 | Coding | 828 | 2098 | tgctagcctctggatttgaTT | 43 | 898 |
| 29601 | Coding | 828 | 2180 | tctggatcagagtcagtggTT | 19 | 899 |
| 29602 | 3′ UTR | 828 | 2268 | tattttcatggtgttttacTT | 59 | 900 |
| 29603 | 3′ UTR | 828 | 2347 | tgttcctataactggtaatTT | 40 | 901 |
| 29604 | 3′ UTR | 828 | 2403 | gtgtcaaaaccctgtggatTT | 45 | 902 |
| 29605 | 3′ UTR | 828 | 2523 | actggaataaaacgggaaaTT | 38 | 903 |
| 29606 | 3′ UTR | 828 | 2598 | acttcagttggtgacagaaTT | 25 | 904 |
| 29607 | 3′ UTR | 828 | 2703 | tagcaaaacctttcggaaaTT | 31 | 905 |
| 29608 | 3′ UTR | 828 | 2765 | aattatttcctttctgagcTT | 29 | 906 |
| 29609 | 3′ UTR | 828 | 2806 | taaatagctggagatggtcTT | 7 | 907 |
| 29610 | 3′ UTR | 828 | 2844 | cagattaataactgtagcaTT | 37 | 908 |
| 29611 | 3′ UTR | 828 | 2950 | ccccaatacagattcacttTT | 39 | 909 |
| 29612 | 3′ UTR | 828 | 3037 | attgttgctgtgtttcttaTT | 30 | 910 |
| 29613 | 3′ UTR | 828 | 3088 | tgtttcaagcccattctttTT | 40 | 911 |

TABLE 39

Modulation of PTEN mRNA levels by dsRNA oligomers

| | | | | | | |
|---|---|---|---|---|---|---|
| dsRNA | | | | | | |
| CORRESPONDING | | | | | | |
| TO COMPLEMENT | | TARGET | | SEQUENCE OF SENSE STRAND | | |
| OF ISIS# | REGION | SEQ ID NO | TARGET SITE | OF dsRNA | % INHIB | SEQ ID NO. |

| | | | | | | |
|---|---|---|---|---|---|---|
| 29574 | 5′ UTR | 828 | 19 | cggtcccgtccgcctctcgTT | 0 | 912 |
| 29575 | 5′ UTR | 828 | 57 | cggtcttccgaggcgcccgTT | 0 | 913 |
| 29576 | 5′ UTR | 828 | 197 | ctctcggaagctgcagccaTT | 40 | 914 |
| 29577 | 5′ UTR | 828 | 314 | gcctgtgagcagccgcgggTT | 25 | 915 |
| 29578 | 5′ UTR | 828 | 421 | tcttcctcggcttctcctgTT | 64 | 916 |
| 29579 | 5′ UTR | 828 | 494 | ggcggcggcggcacctcccTT | 20 | 917 |
| 29581 | 5′ UTR | 828 | 671 | ggcacatccagggacccggTT | 35 | 918 |
| 29582 | 5′ UTR | 828 | 757 | ggaggcagccgttcggaggTT | 59 | 919 |
| 29583 | 5′ UTR | 828 | 817 | cctctggctgctgaggagaTT | 50 | 920 |
| 29584 | 5′ UTR | 828 | 891 | tgcggtccagagccaagcgTT | 33 | 921 |
| 29585 | 5′ UTR | 828 | 952 | atttccatcctgcagaagaTT | 27 | 922 |
| 29587 | Coding | 828 | 1106 | acttgacctatatttatccTT | 49 | 923 |
| 29588 | Coding | 828 | 1169 | tatacaggaacaatattgaTT | 50 | 924 |
| 29589 | Coding | 828 | 1262 | atgacaccgccaaatttaaTT | 64 | 925 |
| 29590 | Coding | 828 | 1342 | cccttttgtgaagatcttgTT | 75 | 926 |
| 29591 | Coding | 828 | 1418 | agggacgaactggtgtaatTT | 77 | 927 |
| 29592 | Coding | 828 | 1504 | gaagtaaggaccagagacaTT | 76 | 928 |
| 29593 | Coding | 828 | 1541 | ccagtcagaggcgctatgtTT | 74 | 929 |
| 29595 | Coding | 828 | 1694 | taaaggtgaagatatattcTT | 10 | 930 |
| 29596 | Coding | 828 | 1792 | atcaaagtagagttcttccTT | 29 | 931 |
| 29597 | Coding | 828 | 1855 | tgggtaaatacattcttcaTT | 72 | 932 |
| 29599 | Coding | 828 | 2020 | aataaagacaaagccaaccTT | 0 | 933 |
| 29600 | Coding | 828 | 2098 | tcaaatccagaggctagcaTT | 43 | 934 |
| 29601 | Coding | 828 | 2180 | ccactgactctgatccagaTT | 19 | 935 |
| 29602 | 3′ UTR | 828 | 2268 | gtaaaacaccatgaaaataTT | 59 | 936 |
| 29603 | 3′ UTR | 828 | 2347 | attaccagttataggaacaTT | 40 | 937 |
| 29604 | 3′ UTR | 828 | 2403 | atccacagggttttgacacTT | 45 | 938 |
| 29605 | 3′ UTR | 828 | 2523 | tttcccgttttattccagtTT | 38 | 939 |
| 29606 | 3′ UTR | 828 | 2598 | ttctgtcaccaactgaagtTT | 25 | 940 |
| 29607 | 3′ UTR | 828 | 2703 | tttccgaaaggttttgctaTT | 31 | 941 |
| 29608 | 3′ UTR | 828 | 2765 | gctcagaaaggaaataattTT | 29 | 942 |
| 29609 | 3′ UTR | 828 | 2806 | gaccatctccagctatttaTT | 7 | 943 |
| 29610 | 3′ UTR | 828 | 2844 | tgctacagttattaatctgTT | 37 | 944 |
| 29611 | 3′ UTR | 828 | 2950 | aagtgaatctgtattggggTT | 39 | 945 |
| 29612 | 3′ UTR | 828 | 3037 | taagaaacacagcaacaatTT | 30 | 946 |
| 29613 | 3′ UTR | 828 | 3088 | aaagaatgggcttgaaacaTT | 40 | 947 |

The antisense strands represented by SEQ ID NOs: 879, 880, 882, 883, 884, 885, 887, 888, 889, 890, 891, 892, 893, 896, 998, 900, 901, 902, 903, 905, 908, 909, 910 and 911 (Table 38) are from the preferred dsRNAs which demonstrated at least 30% inhibition of PTEN expression in this experiment. The corresponding sense strands of the preferred dsRNA oligomers are represented by SEQ ID NOs: 914, 916, 918, 919, 920, 921, 923, 924, 925, 926, 927, 928, 929, 932, 934, 936, 937, 938, 939, 941, 944, 945, 946 and 947 (Table 39).

The target sites to which these preferred sequences are complementary are herein referred to as, “preferred target segments” and are therefore preferred sites for targeting by compounds of the present invention.

One having skill in the art will recognize that the methods of identification of preferred dsRNA oligomeric compounds for modulation of PTEN expression outlined in this example may be applied to any target for the purpose of target validation or gene function analysis. It will also be recognizable to one skilled in the art, that for any particular target, screening of antisense oligonucleotides need not be carried out prior to design of and screening of dsRNA compounds. Thus, a plurality of virtual dsRNA compounds targeted to functional regions of any target can be generated and subjected to a selection process, actual compounds corresponding to a subset of virtual compounds may be robotically synthesized, and modulators can be identified which may subsequently employed in the processes of gene function analysis or target validation via the methods herein described.

What is claimed is:

1. A method comprising:
(a) identifying a target;
(b) generating a plurality of virtual compounds targeted to said target;
(c) robotically synthesizing a plurality of real compounds corresponding to at least some of said virtual compounds;
(d) identifying a modulator of said target from said plurality of real compounds;
(e) contacting said modulator with said target in an assay of a biochemical or biological parameter indicative of a biological process to determine one of: an effect of modulation of said target on said parameter or a lack of an effect of modulation of said target on said parameter, thereby effecting gene function analysis.

2. The method of claim 1 wherein said plurality of real compounds corresponds to a subset of virtual compounds selected from said plurality of virtual compounds.

3. The method of claim 1 wherein said target is a gene.

4. The method of claim 3 wherein the best possible representation of the nucleotide sequence of said gene is obtained using computerized searches of available databases.

5. The method of claim 4 wherein said sequence represents a transcript isoform of said gene.

6. The method of claim 5 wherein the formation of said transcript isoform is directed by alternative splicing.

7. The method of claim 1 wherein said target is a polypeptide-encoding nucleic acid.

8. The method of claim 1 wherein said target is a non-polypeptide-encoding nucleic acid.

9. The method of claim 8 wherein said non-polypeptide-encoding nucleic acid is one of a structural RNA or an enzymatic RNA.

10. The method of claim 1 wherein said plurality of virtual compounds is targeted to functional regions of said target.

11. The method of claim 10 wherein said functional regions are selected from the group consisting of: the transcription start site, the 5′ cap, the 5′ untranslated region, the start codon, the coding region, the stop codon, the 3′ untranslated region, 5′ splice sites, 3′ splice sites, exons, introns, exon: intron junctions, intron: exon junctions, exon: exon junctions, mRNA destablization signals, mRNA destabilization signals, poly-A signals and 5′ sequences of pre-mRNA.

12. The method of claim 2 wherein said subset of virtual compounds is selected by evaluation of thermodynamic properties of said plurality of virtual compounds in silico.

13. The method of claim 1 wherein the accessibility of said target to said plurality of virtual compounds is evaluated in silico.

14. The method of claim 1 wherein said virtual compounds are 8 to 30 nucleobases in length and specifically hybridize with said target.

15. The method of claim 14 wherein said virtual compounds are antisense compounds.

16. The method of claim 15 wherein said antisense compounds are antisense oligonucleotides.

17. The method of claim 16 wherein said antisense oligonucleotides comprise at least one modified internucleoside linkage.

18. The method of claim 17 wherein said modified internucleoside linkage is a phosphorothioate linkage.

19. The method of claim 16 wherein said antisense oligonucleotides comprise at least one modified sugar moiety.

20. The method of claim 19 wherein said modified sugar moiety is a 2′-O-methoxyethyl sugar moiety.

21. The method of claim 16 wherein said antisense oligonucleotides comprise at least one modified nucleobase.

22. The method of claim 21 wherein said modified nucleobase is a 5-methylcytosine.

23. The method of claim 14 wherein said virtual compounds are double-stranded oligomeric compounds.

24. The method of claim 23 wherein said double-stranded oligomeric compounds are double-stranded RNA oligomeric compounds.

25. The method of claim 24 wherein said double-stranded RNA oligomeric compounds are siRNAs.

26. The method of claim 23 wherein said double-stranded oligomeric compounds comprise at least one two-nucleobase overhang of deoxythymidine.

27. The method of claim 23 wherein both strands of said double-stranded oligomeric compounds comprise at least one modified internucleoside linkage.

28. The method of claim 27 wherein said modified internucleoside linkage is a phosphorothioate linkage.

29. The method of claim 23 wherein both strands of said double-stranded oligomeric compounds comprise at least one modified sugar moiety.

30. The method of claim 29 wherein said modified sugar moiety is a 2′-O-methoxyethyl sugar moiety.

31. The method of claim 23 wherein both strands of said double-stranded oligomeric compounds comprise at least one modified nucleobase.

32. The method of claim 31 wherein said modified nucleobase is a 5-methylcytosine.

33. The method of claim 1 wherein said target is expressed in a sample capable of exhibiting said parameter wherein said sample is selected from the group consisting of: a cell culture, a cell-free extract, a tissue and an animal.

34. The method of claim 1 wherein said modulator is identified by a computer-controlled real-time polymerase chain reaction or a computer-controlled enzyme-linked immunosorbent assay.

35. The method of claim 1 wherein said parameter is the expression of at least one gene related to said biological process.

36. The method of claim 1 wherein said parameter is determined by an assay selected from the group consisting of: a caspase activity assay, a cell cycle assay, a matrix metalloproteinase activity assay and a tube formation assay.

37. The method of claim 1 wherein the value of said parameter is increased as a result of modulation of said target.

38. The method of claim 1 wherein the value of said parameter is decreased as a result of modulation of said target.

39. The method of claim 1 wherein said biological process is selected from the group consisting of apoptosis, inflammation and angiogenesis.

40. A method comprising:
(a) identifying a target;
(b) generating a plurality of virtual compounds targeted to said target;
(c) robotically synthesizing a plurality of real compounds corresponding to at least some of said virtual compounds;
(d) identifying a modulator of said target from said real compounds;
(e) contacting said modulator with said target in an assay of a biochemical or biological parameter indicative of a disease or disorder to determine one of: an effect of modulation of said target on said parameter or a lack of an effect of modulation of said target on said parameter, thereby effecting target validation.

41. The method of claim 40 wherein said plurality of real compounds corresponds to a subset of virtual compounds selected from said plurality of virtual compounds.

42. The method of claim 40 wherein said target is a gene.

43. The method of claim 42 wherein the best possible representation of the nucleotide sequence of said gene is obtained using computerized searches of available databases.

44. The method of claim 43 wherein said sequence represents a transcript isoform of said gene.

45. The method of claim 44 wherein the formation of said transcript isoform is directed by alternative splicing.

46. The method of claim 40 wherein said target is a polypeptide-encoding nucleic acid.

47. The method of claim 40 wherein said target is a non-polypeptide-encoding nucleic acid.

48. The method of claim 47 wherein said non-polypeptide-encoding nucleic acid is one of a structural RNA or an enzymatic RNA.

49. The method of claim 40 wherein said plurality of virtual compounds is targeted to functional regions of said target.

50. The method of claim 49 wherein said functional regions are selected from the group consisting of: the transcription start site, the 5′ cap, the 5′ untranslated region, the start codon, the coding region, the stop codon, the 3′ untranslated region, 5′ splice sites, 3′ splice sites, specific exons, specific introns, exon: intron junctions, intron: exon junctions, exon: exon junctions, mRNA destablization signals, mRNA destabilization signals, poly-A signals and 5′ sequences of known pre-mRNA.

51. The method of claim 41 wherein said subset is selected by evaluation of thermodynamic properties of said plurality of virtual compounds in silico.

52. The method of claim 40 wherein the accessibility of said target to said plurality of virtual compounds is evaluated in silico.

53. The method of claim 40 wherein said virtual compounds are 8 to 30 nucleobases in length targeted to a nucleic acid molecule encoding said target and specifically hybridize with said target.

54. The method of claim 53 wherein said virtual compounds are antisense compounds.

55. The method of claim 54 wherein said antisense compounds are antisense oligonucleotides.

56. The method of claim 55 wherein said antisense oligonucleotides comprise at least one modified internucleoside linkage.

57. The method of claim 56 wherein said modified internucleoside linkage is a phosphorothioate linkage.

58. The method of claim 55 wherein said antisense oligonucleotides comprise at least one modified sugar moiety.

59. The method of claim 58 wherein said modified sugar moiety is a 2′-O-methoxyethyl sugar moiety.

60. The method of claim 55 wherein said antisense oligonucleotides comprise at least one modified nucleobase.

61. The method of claim 60 wherein said modified nucleobase is a 5-methylcytosine.

62. The method of claim 53 wherein said virtual compounds are double-stranded oligomeric compounds.

63. The method of claim 62 wherein said double-stranded oligomeric compounds are double-stranded RNA oligomeric compounds.

64. The method of claim 63 wherein said double-stranded RNA oligomeric compounds are siRNAs.

65. The method of claim 62 wherein said double-stranded oligomeric compounds comprise at least one two-nucleobase overhang of deoxythymidine.

66. The method of claim 62 wherein both strands of said double-stranded oligomeric compounds comprise at least one modified internucleoside linkage.

67. The method of claim 66 wherein said modified internucleoside linkage is a phosphorothioate linkage.

68. The method of claim 62 wherein both strands of said double-stranded oligomeric compounds comprise at least one modified sugar moiety.

69. The method of claim 68 wherein said modified sugar moiety is a 2′-O-methoxyethyl sugar moiety.

70. The method of claim 62 wherein both strands of said double-stranded oligomeric compounds comprise at least one modified nucleobase.

71. The method of claim 70 wherein said modified nucleobase is a 5-methylcytosine.

72. The method of claim 40 wherein said target is expressed in a sample capable of exhibiting said parameter wherein said sample is selected from the group consisting of: a cell culture, a cell-free extract, a tissue and an animal.

73. The method of claim 40 wherein said modulator is identified by a computer-controlled real-time polymerase chain reaction or a computer-controlled enzyme-linked immunosorbent assay.

74. The method of claim 40 wherein said parameter is the expression of at least one gene related to said disease or disorder.

75. The method of claim 40 wherein said parameter is the level of a biochemical component selected from the group consisting of cholesterol, triglyceride, lipoprotein, glucose, insulin and PEPCK.

76. The method of claim 40 wherein said parameter is measured in a rodent.

77. The method of claim 76 wherein said parameter measured in a rodent is selected from the group consisting of survival rate, spleen weight, liver weight and fat pad weight.

78. The method of claim 40 wherein the valuse of said parameter is decreased as a result of modulation of said target.

79. The method of claim 40 wherein the value of said parameter is increased as a result of modulation of said target.

80. A method comprising:
(a) identifying a target;
(b) generating a plurality of virtual compounds designed to modulate said target;
(c) robotically synthesizing a plurality of real compounds corresponding to at least some of said virtual compounds;
(d) identifying at least one modulator of said target by contacting said target with said real compounds and measuring the extent of modulation of said target using an automated means;
(e) performing an automated assay of at least one biological or biochemical parameter indicative of one of: (i) a biological process, thereby effecting gene function analysis or (ii) a disease or disorder, thereby effecting target validation.

81. A method comprising:
(a) identifying a target;
(b) generating a plurality of virtual double-stranded oligomeric compounds designed to modulate said target;
(c) robotically synthesizing a plurality of real double-stranded oligomeric compounds corresponding to at least some of said virtual double-stranded oligomeric compounds;
(d) identifying at least one modulator of said target by contacting said target with said real double-stranded oligomeric compounds and measuring the extent of modulation of said target using an automated means;
(e) performing an automated assay of at least one biological or biochemical parameter indicative of one of: (i) a biological process, thereby effecting gene function analysis or (ii) a disease or disorder, thereby effecting target validation.

82. The method of claim 81 wherein said double-stranded oligomeric compounds are double-stranded RNA oligomeric compounds.

83. The method of claim 82 wherein said double-stranded RNA oligomeric compounds are siRNAs.

84. The method of claim 81 wherein said double-stranded oligomeric compounds are 15 to 30 nucleobases in length.

85. The method of claim 81 wherein said double-stranded oligomeric compounds are 20 to 25 nucleobases in length.

86. The method of claim 81 wherein said double-stranded oligomeric compounds comprise at least one two-nucleobase overhang of deoxythymidine.

87. The method of claim 81 wherein both strands of said double-stranded oligomeric compounds comprise at least one modified internucleoside linkage.

88. The method of claim 87 wherein said modified internucleoside linkage is a phosphorothioate linkage.

89. The method of claim 81 wherein both strands of said double-stranded oligomeric compounds comprise at least one modified sugar moiety.

90. The method of claim 89 wherein said modified sugar moiety is a 2′-O-methoxyethyl sugar moiety.

91. The method of claim 81 wherein both strands of said double-stranded oligomeric compounds comprise at least one modified nucleobase.

92. The method of claim 91 wherein said modified nucleobase is a 5-methylcytosine.
